(19) United States
(12) Patent Application Publication
(10) Pub. No.: US 2025/0353833 A1
(43) Pub. Date: Nov. 20, 2025

(54) DPP9 BINDING COMPOUNDS

(71) Applicant: UNIVERSITEIT ANTWERPEN, Antwerpen (BE)

(72) Inventors: Koen AUGUSTYNS, Wilrijk (BE); Siham BENRAMDANE, Wilrijk (BE); Olivier BEYENS, Wilrijk (BE); Joni DE LOOSE, Wilrijk (BE); Ingrid DE MEESTER, Wilrijk (BE); Hans DE WINTER, Wilrijk (BE); Margarida ESPADINHA, Wilrijk (BE); Nicolò FILIPPI, Wilrijk (BE); Pieter VAN DER VEKEN, Wilrijk (BE)

(21) Appl. No.: 18/871,130

(22) PCT Filed: Jun. 2, 2023
(86) PCT No.: PCT/EP2023/064881

§ 371 Date: Dec. 2, 2024

(30) Foreign Application Priority Data
Jun. 3, 2022 (EP) 22177159.5

(51) Int. Cl. C07D 403/14; A61K 31/40; A61K 31/403; A61K 31/4035; A61K 31/405; A61K 31/4245; A61K 31/427; A61K 31/4439; A61K 31/455; A61K 31/4709; C07D 207/16; C07D 207/22; C07D 209/44; C07D 209/52; C07D 401/12; C07D 403/12; C07D 409/12; C07D 409/14; C07D 413/12; C07D 417/14

(52) U.S. Cl. CPC C07D 403/14; A61K 31/40; A61K 31/403; A61K 31/4035; A61K 31/405; A61K 31/4245; A61K 31/427; A61K 31/4439; A61K 31/455; A61K 31/4709; C07D 207/16; C07D 207/22; C07D 209/44; C07D 209/52; C07D 401/12; C07D 403/12; C07D 409/12; C07D 409/14; C07D 413/12; C07D 417/14

(57) ABSTRACT

The invention relates to new compounds of the general Formula (I), which are Dipeptidyl peptidase 9 (DPP9) enzyme inhibitors. The present invention further relates to specific compounds based on Formula (I) that binds to an E3 ligase and to DPP9 and induce proteolysis of DPP9. The invention also relates to specific DPP9 binding compounds comprising a probe moiety, according to formula (I). The present invention further relates to pharmaceutical compositions and their use thereof.

### FIELD OF THE INVENTION

The invention relates to new compounds able to bind to Dipeptidyl peptidase 9 (DPP9) and, pharmaceutical compositions and their uses.

### BACKGROUND

Dipeptidyl peptidase 9 (DPP9) is a cytosolic serine protease belonging to the Dipeptidyl peptidase 4 Activity and/or Structure Homologues (DASH) family. DASH proteases are characterized by the ability to cleave peptide substrates with a proline at the penultimate position of the N-terminus.

DPP9 is ubiquitously expressed in tissues and has diverse roles in cell behavior, immune regulation, and cancer. DPP9 can interact with H-Ras, which is a key molecule of the epidermal growth factor receptor and PI3K/Akt signaling pathways; these pathways are important for cell survival, proliferation, and apoptosis. DPP9 inhibition stimulates the immune system by activating pyroptosis in multiple cell types including monocytes, and macrophages as well as in the majority of Acute Myeloid Leukemia (AML) cell lines and primary AML cells. Briefly, the inhibition of DPP9 causes the formation of multiprotein complexes called inflammasomes, which activate caspase-1 and generate pyroptosis.

DPP9 is associated with other types of cancer besides AML. For example, knockdown of DPP9 could inhibit lung cancer cell proliferation, migration, and tumorigenesis. Overexpression of DPP9 in non-small-cell-lung cancer (NSCLC) is independently associated with poor 5-year overall survival. Similarly, in colorectal cancer, greater DPP9 expression is associated with a poor prognosis. In contrast, lower DPP9 expression correlates with poor survival in patients with oral squamous cell carcinoma. These data suggest that DPP9 has different roles in various types of cancers.

To our knowledge, there are currently no known selective inhibitors of DPP9 and the existing molecules also inhibit other DASH proteases such as DPP8 and DPP4. An important hurdle in the discovery of selective inhibitors for DPP9, is the enzyme's close homology to the other family members, in particular to DPP8 due to their almost identical secondary, tertiary, and quaternary structures.

WO2014068023 discloses non-competitive allosteric peptide inhibitors that target an arm motif of Dipeptidyl peptidase 8 (DPP8) and Dipeptidyl peptidase 9 (DPP9).

WO2001096295 discloses 2-cyanopyrrolidine derivatives as DPP4 inhibitors.

It further relates to a pharmaceutical composition and a method for treating diseases associated with elevated levels of DPP4. Furthermore, the compounds do not selectively inhibit DPP9.

U.S. Pat. No. 6,617,340 describes N-(substituted glycyl)-pyrrolidines and pharmaceutical compositions containing said compounds as an active ingredient thereof, and the use of said compounds in inhibiting DPP4. Furthermore, the compounds do not selectively inhibit DPP9.

WO2005012249 and EP1664031 are disclosing adamantane derivative compounds as DPP4 inhibitors but disclosed compounds do not comprise isoindoline as the compounds disclosed herein. Furthermore, the compounds do not selectively inhibit DPP9.

US20060241146 is disclosing nitrogen-containing 5-membered ring compounds with DPP4 inhibitory action that may comprise the isoindoline group. However, disclosed compounds do not comprise adamantly-glycine moiety. Furthermore, the compounds do not selectively inhibit DPP9.

WO2099068531 discloses Adamantyl o-glucuronide derivatives as DPP4 inhibitors.

WO2005108368 discloses Adamantyl-acetamide derivatives as inhibitors of the 11-beta-hydroxysteroid dehydrogenase Type 1 enzyme.

To summarize, some inhibitors of other DASH proteases also inhibit DPP8 and DPP9 but none are DPP9 selective inhibitors. Hence, there is a need for molecules that can discriminate between DPP8 and 9 as selective DPP9 inhibitors which could be highly valuable, both as potential therapeutics, especially in cancer but also as research tools to support DPP9 research.

The invention aims to provide selective DPP9 binders with a good, selective inhibitory effect and/or as bifunctional compounds with the function to induce DPP9 intracellular proteolysis and/or carrying a marker moiety to function as DPP9 specific probes.

### SUMMARY OF THE INVENTION

The present invention relates in the first aspect to a compound or a pharmaceutically acceptable salt thereof that is eligible to bind to DPP9, according to claim 1. Said compound is a DPP9 binding compound that comprises an isoindoline-aminoacyl-adamantyl basic structure that enables the binding to DPP9. The DPP9 binding compounds can inhibit the DPP9 enzyme. In embodiments said compounds selectively inhibit DPP9 enzyme. DPP9 binding compound can also comprise an E3 ligase binding moiety wherein the said compound induces DPP9 ubiquitination and intracellular proteolysis of DPP9.

Further embodiments are described in the dependent claims.

In a second aspect, the present invention relates to a pharmaceutical composition according to claim 15.

In a third aspect, the compounds according to the invention are intended for the use in the prevention and/or treatment of a disorder, such as DPP9 enzyme-related disorders comprising cancer where the tumor cells are expressing DPP9 according to claims 15.

### DESCRIPTION OF FIGURES

The following description of the figures of specific embodiments of the invention is merely exemplary and is not intended to limit the present teachings, their application, or uses. Throughout the drawings, corresponding reference numerals indicate like or corresponding parts and features.

FIG. 1 Docked base structure (light grey) in DPP9 (dark residues and shaded backbone cartoon). The isoindoline is interacting with S1 pocket residues. Two key ligand-binding residues (GLU248 and GLU249) are shown to form an interaction with the secondary amine of the ligand.

FIG. 2 Docked UAMC-4918 (light grey) in DPP9 (dark residues and shaded backbone cartoon). The interactions are similar to those in FIG. 1.

FIG. 3 Superposition of DPP4, DPP8, and DPP9 with co-crystallized vildagliptin and manually docked analogue 5p. The residues forming the interactions between the S1 pocket and the isoindoline appear highly conserved between DPP4, DPP8, and DPP9.

FIG. 4 A) Root Mean Square Deviation (RMSD) of the isoindoline heavy atoms, aligned on the backbone atoms of the residues forming the S1 pocket. RMSD is a measure of the movement of the isoindoline relative to the S1 pocket. The 100 frame moving average is shown (full line) together with each individual frame (shaded). The dashed line marks the demarcation between the two parallel runs, and black squares mark the time points of the binding poses in B. B) Binding poses of compound 5r in DPP4, DPP8, and DPP9 are marked in A. The DPP9 binding pose serves as a reference.

FIG. 5 A) Distance between the carboxylate oxygen of E205/275/248 (DPP4/DPP8/DPP9) that is closest to the secondary amine and the nitrogen atom of the secondary amine in compound 5r B) Distance between the carboxylate oxygen of E206/276/249 (DPP4/DPP8/DPP9) that is closest to the secondary amine and the nitrogen atom of the secondary amine in compound 5r. The full lines show the 100 frames moving average, the shaded regions show every individual frame. The dashed line marks the demarcation between the two parallel runs.

FIG. 6 Isoindoline varied analogues (W-substituents)

FIG. 7 Aminoacyl-varied analogues (Y-substituents)

FIG. 8 Adamantyl-varied analogues (Z-substituents)

### DETAILED DESCRIPTION OF THE INVENTION

### Definitions

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third, and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7, etc. of said members, and up to all said members.

“Alkyl” as used herein refers to and includes, unless otherwise stated, a saturated linear (i.e., unbranched) or branched univalent hydrocarbon chain or combination thereof, having the number of carbon atoms designated (i.e., C₁-C₁₀ means one to ten carbon atoms). Particular alkyl groups are those having 1 to 20 carbon atoms (a C₁-C₂₀ alkyl”), having 1 to 10 carbon atoms (a C₁-C₁₀ alkyl), and having 6 to 10 carbon atoms (a C₆-C₁₀ alkyl), or having 1 to 4 carbon atoms (a C₁-C₄ alkyl). Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl, cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like.

“Halo” or “halogen” refers to elements of the Group 17 series having atomic numbers 9 to 85. Preferred halo groups include the radicals of fluorine, chlorine, bromine, and iodine. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halogen; thus 4-chloro-3-fluorophenyl is within the scope of dihaloaryl.

“Haloalkyl” by itself or as part of another substituent refers to an alkyl group in which one or more of the hydrogen atoms are replaced with a halogen. Thus, the term “haloalkyl” is meant to include monohaloalkyls, dihaloalkyls, trihaloalkyls, etc. up to perhaloalkyls. For example, the expression “(C1-C2) haloalkyl” includes fluoromethyl, difluoromethyl, trifluoromethyl, 1-fluoroethyl, 1,1-difluoroethyl, 1,2-difluoroethyl, 1,1,1-trifluoroethyl, perfluoroethyl, etc.

“Hydroxyalkyl” by itself or as part of another substituent refers to an alkyl group in which one or more of the hydrogen atoms are replaced with a hydroxyl substituent. Thus, the term “hydroxyalkyl” is meant to include monohydroxyalkyls, dihydroxyalkyls, trihydroxyalkyls, etc.

The term “cycloalkyl” by itself or as part of another substituent refers to a cyclic version of an “alkyl” group. A cycloalkyl group may include zero bridgehead carbon atoms or two or more bridgehead carbon atoms. Thus, a cycloalkyl may be monocyclic, bicyclic or polycyclic, depending upon the number of bridgehead and bridging carbon atoms. Examples of typical cycloalkyl groups include, but are not limited to, cyclopropyl; cyclobutyls such as cyclobutanyl and cyclobutenyl; cyclopentyls such as cyclopentanyl and cyclopentenyl; cyclohexyls such as cyclohexanyl and cyclohexenyl, adamantyl, noradamantyl, bicyclo[1.1.0]butanyl, norboranyl (bicyclo[2.2.1]heptanyl), norbornenyl (bicyclo[2.2.1]heptanyl), norbornadienyl (bicyclo[2.2.1]heptadienyl), tricyclo[2.2.1.0]heptanyl, bicyclo[3.2.1]octanyl, bicyclo[3.2.1]octanyl, bicyclo[3.2.1]octadienyl, bicyclo[2.2.2]octanyl, bicyclo[2.2.2]octenyl, bicyclo[2.2.2]octadienyl, bicyclo[5,2,0]nonanyl, bicyclo[4.3.2]undecanyl, tricyclo[5.3.1.1]dodecanyl, and the like. A cycloalkyl contains from 3 to 16 carbon atoms.

A “heterocycle” or “heterocyclic” as used herein refers to a saturated or an unsaturated non-aromatic cyclic group having a single ring or multiple condensed rings and having from 1 to 14 annular carbon atoms and from 1 to 6 annular heteroatoms, such as nitrogen, sulfur or oxygen, and the like. A heterocycle comprising more than one ring may be fused, bridged, or spiro, or any combination thereof, but excludes heteroaryl groups. The heterocyclic group may be optionally substituted independently with one or more substituents described herein. Particular heterocyclic groups are 3 to 14-membered rings having 1 to 13 annular carbon atoms and 1 to 6 annular heteroatoms independently selected from nitrogen, oxygen, and sulfur, 3 to 12-membered rings having 1 to 11 annular carbon atoms and 1 to 6 annular heteroatoms independently selected from nitrogen, oxygen and sulfur, 3 to 10-membered rings having 1 to 9 annular carbon atoms and 1 to 4 annular heteroatoms independently selected from nitrogen, oxygen and sulfur, 3 to 8-membered rings having 1 to 7 annular carbon atoms and 1 to 4 annular heteroatoms independently selected from nitrogen, oxygen and sulfur, or 3 to 6-membered rings having 1 to 5 annular carbon atoms and 1 to 4 annular heteroatoms independently selected from nitrogen, oxygen and sulfur. Particular heterocyclic groups are monocyclic 3-, 4-, 5-, 6- or 7-membered rings having from 1 to 2, 1 to 3, 1 to 4, 1 to 5, or 1 to 6 annular carbon atoms and 1 to 2, 1 to 3, or 1 to 4 annular heteroatoms independently selected from nitrogen, oxygen and sulfur. Particular heterocyclic groups are polycyclic non-aromatic rings having from 1 to 12 annular carbon atoms and 1 to 6 annular heteroatoms independently selected from nitrogen, oxygen and sulfur.

The term “aryl,” as used herein, refers to a monocyclic-ring system or a polycyclic-ring system wherein one or more of the fused rings are aromatic. Said aromatic rings may optionally comprise heteroatoms, particularly nitrogen, oxygen and/or sulphur. Representative aryl groups include, but are not limited to anthracenyl, azulenyl, fluorenyl, pyridyl, indanyl, indenyl, naphthyl, phenyl, and tetrahydronaphthyl, indole, quinolinyl, triazolyl, tetrazolyl. The aryl groups of this invention may be optionally substituted with 0, 1, 2, 3, 4 or 5 substituents.

The above-defined groups may include prefixes and/or suffixes that are commonly used in the art to create additional well-recognized substituent groups. As examples, “alkyloxy” or “alkoxy” refers to a group of the formula —OR, “alkylamine” refers to a group of the formula —NHR and “dialkylamine” refers to a group of the formula —NRR, where each R is independently an alkyl. As another example, “haloalkoxy” or “haloalkyloxy” refers to a group of the formula —OR, where R is a haloalkyl.

“Optionally substituted” unless otherwise specified means that a group may be unsubstituted or substituted by one or more (e.g., 1, 2, 3, 4, or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

As used herein, “treatment” or “treating” is an approach for obtaining beneficial or desired results including clinical results. Beneficial or desired results include, but are not limited to, one or more of the following: decreasing one or more symptoms resulting from the disease, diminishing the extent of the disease, stabilizing the disease (e.g., preventing or delaying the worsening of the disease), preventing or delaying the spread of the disease, delaying the occurrence or recurrence of the disease, delay or slowing the progression of the disease, ameliorating the disease state, providing a remission (whether partial or total) of the disease, decreasing the dose of one or more oilier medications required to treat the disease, enhancing effect of another medication, delaying the progression of the disease, increasing the quality of life, and/or prolonging survival. The methods described herein contemplate any one or more of these aspects of treatment.

As used herein the term “pharmaceutical composition” means a product comprising pharmaceutical excipients such as buffering agents, preservatives, and tonicity modifiers together with the active compound or salt thereof, the pharmaceutical composition is useful for treating or preventing a disease or disorder or to reduce the severity thereof by administering the pharmaceutical composition to a human or animal. Thus, pharmaceutical compositions are also known in the art as pharmaceutical preparations.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug Administration and/or have been approved by the administrations such as EMA and/or the United States Food and Drug Administration as being acceptable for use in humans or domestic animals.

As used herein the term “pharmaceutically acceptable salt” means an acid or base salt of the compounds of the invention that is of sufficient purity and quality for use in the formulation of a composition or medicament of the present invention and are tolerated and sufficiently non-toxic to be used in a pharmaceutical preparation. Suitable pharmaceutically acceptable salts include acid addition salts which may, for example, be formed by reacting the drug compound with a suitable pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid.

The term “pharmaceutically acceptable carrier” as used herein, refers to a carrier or a diluent that does not cause significant irritation to a subject and does not abrogate the biological activity and properties of the administered composition. Examples, without limitations, of carriers are propylene glycol, saline, emulsions and mixtures of organic solvents with water.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc; coatings include, e.g, cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for tablets include, e.g., dextrose, fructose de, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

The term “E3 ubiquitin ligase” or “ubiquitin ligase” (UL) is used herein to describe the binding site of the ubiquitin ligase moiety of a target enzyme (s) in a bifunctional compound according to the present application. E3 UL is a protein that, in combination with the E2 ubiquitin conjugating enzyme, causes ubiquitin attachment to a lysine on the target protein, which is the E3 ubiquitin ligase target-specific protein substrate for degradation by the proteasome. Therefore, E3 ubiquitin ligase alone or in combination with E2 ubiquitin ligase is involved in the transfer of ubiquitin to the target protein. In general, ubiquitin ligase is involved in polyubiquitination, such as the attachment of a second ubiquitin to a first ubiquitin, the attachment of a third ubiquitin to a second ubiquitin, and the like. Polyubiquitination labels the protein for degradation by the proteasome. However, several ubiquitination events are restricted to monoubiquitination, and only a single ubiquitin is added to the substrate molecule by ubiquitin ligase. Monoubiquitinated proteins are not targeted to the degradation by the proteasome, but instead their cell location or function can be altered by binding to other proteins with domains capable of binding ubiquitin, for example. Additionally, monoubiquitination at multiple sites of the substrate molecule by ubiquitin ligase can also lead to its degradation. Further complicating the problem is that different lysines on ubiquitin can be targeted by E3 to form chains. The most common lysine is Lys48 on the ubiquitin chain. It is the lysine used to make polyubiquitin recognized by the proteasome.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

The terms “label”, “labeling moiety”, “reporter group” and “reporter moiety” are used interchangeably wherein said terms are as used herein, refers to a chemical moiety, or protein that is directly or indirectly detectable (e.g. due to its spectral properties, conformation or activity) when attached to a target or compound and used in the present methods. As used herein, label collectively refers to a reporter group, solid support, or carrier molecule. The label can for example be directly detectable (fluorophore, radionuclides) or indirectly detectable (hapten or enzyme). The term also refers to a portion of a molecule that can effectively bind noncovalently or covalently to a molecule, biomolecule, or material of interest (e.g. biotin, chitin). The labeling moiety can be a molecule that is capable of functioning as a member of an energy transfer pair wherein the reporter molecule retains its native properties (e.g., spectral properties, conformation, and/or activity) when attached to a ligand analog. Examples of reporter molecules include but are not limited to nucleic acids, borapolyazaindacenes, coumarins, xanthenes, cyanines, and luminescent molecules, including dyes, fluorescent proteins, chromophores, and chemiluminescent compounds that are capable of producing a detectable signal upon appropriate activation. The term “dye” refers to a compound that emits light to produce an observable detectable signal. “Dye” includes phosphorescent, fluorescent, and nonfluorescent compounds that include without limitation pigments, fluorophores, chemiluminescent compounds, luminescent compounds, and chromophores. The term “chromophore” refers to a label that emits and/or reflects light in the visible spectra that can be observed without the aid of instrumentation.

The term “fluorophore” of “fluorescent moiety”, “fluorescent probe” as used herein, refers to a compound, chemical group, or composition that is inherently fluorescent. Fluorophores may contain substituents that alter the solubility, spectral properties or physical properties of the fluorophore. Numerous fluorophores are known to those skilled in the art and include, but are not limited to coumarin, cyanine, benzofuran, a quinoline, a quinazolinone, an indole, a furan, a benzazole, a borapolyazaindacene and xanthenes including fluorescein, rhodamine and rhodol as well as other fluorophores described in Richard P. Haugland, Molecular probes handbook of fluorescent probes and research chemicals (9th edition, CD-ROM, September 2002).

The term “Biotinylated moiety”, “biotinylated probe” or “biotinylated label” refers to a protein, nucleic acid, or other molecule and compound where biotin is covalently attached.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment but may. Furthermore, the particular features, structures, or characteristics may be combined in any suitable manner, as would be apparent to a person skilled in the art from this disclosure, in one or more embodiments.

Furthermore, while some embodiments described herein include some, but not other features included in other embodiments, combinations of features of different embodiments are meant to be within the scope of the invention and form different embodiments, as would be understood by those in the art. For example, in the following claims, any of the claimed embodiments can be used in any combination.

### Compounds

One aspect of the present invention is directed to a compound that is able to bind to DPP9, preferably human DPP9. In embodiments, the invention relates to the compound of Formula I or a pharmaceutically acceptable salt thereof.

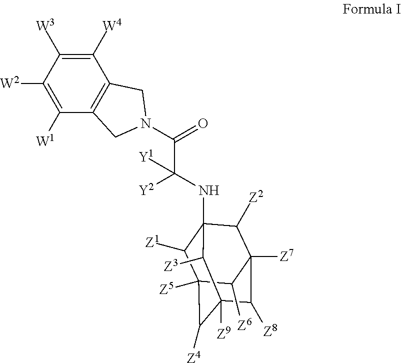

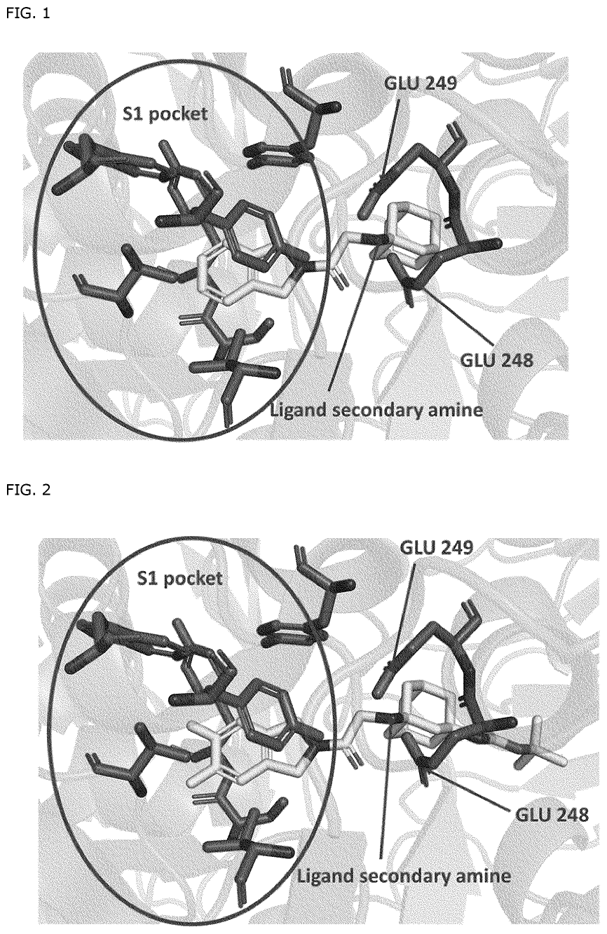

It was shown that compounds according to formula I are able to bind to DPP9. The binding is shown in FIG. 1 where the isoindoline structure of FIG. 1 is interacting with S1 pocket residues of DPP9. Two key ligand-binding residues (GLU248 and GLU249) are shown to form an interaction with the secondary amine of the ligand.

In embodiments W¹, W², W³ and W⁴ are independently selected from the group consisting of hydrogen, alkyl, alkylcarbonyl, alkylsulfonyl, cycloalkyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, halocycloalkyl, heterocycle, heterocyclealkyl, heterocycleoxyalkyl and alkoxy.

In embodiments, Y¹ and Y² are independently selected from the group consisting of hydrogen, alkyl, alkylcarbonyl, alkylsulfonyl, cycloalkyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, halocycloalkyl, heterocycle, heterocyclealkyl, heterocycleoxyalkyl, and alkoxy.

In further, more preferred embodiments Y¹ and Y² are independently selected from the group consisting of hydrogen, alkyl, halogen, and haloalkyl.

In embodiments Z¹, Z², Z³, Z⁴, Z⁵, Z⁶, Z⁷, and Z⁸ are each independently selected from the group consisting of hydrogen, halogen, dihalogen, alkyl, haloalkyl, carboxyalkyl, alkylcarboxyalkyl, alkyloxyalkyl, cycloalkyl, carboxycycloalkyl, aryl, arylalkyl, aryloxyalkyl, aryloxy, hydroxy, alkoxy, alkylamine, alkylamide, N-alkylalkylamide, cycloalkyloxy, heterocycloxy, heterocycle, heterocyclalkyl, and heterocycloxyalkyl, —NHR, —NHC(═O)R, —NHC(═O)OR, —NHC(═O)NHR, —OR, —O(C═O)NHR; —R—CN, —R-halo, —O—(C═O)-L-prolinate. The group “—O—(C═O)-L-prolinate” can be represented with the structural formula of

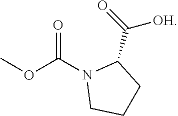

In embodiments R is independently selected from the group: hydrogen, alkyl, alkylcarbonyl, alkylsulfonyl, alkylcarboxyalkyl, cycloalkyl, (alkyl)(carboxyalkyl)sulfide, alkylamide-benzyl carbamate, cycloalkylcarbonyl, alkyl-OH, alkyl-Ar1, alkylcarboxyalkyl-Ar1, alkyl-Ar1-Ar2, Ar1, Ar1-carbonyl, Ar1-sulfonyl, alkyl-Ar1, alkoxy-Ar1, carboxyalkyl, carboxycycloalkyl, carboxyalkylbenzylcarbamate, carboxyalkyl-Ar1, hydroxycycloalkyl, cycloalkylsulfonyl, halogen, haloalkyl, halocycloalkyl, heterocycle, heterocyclecarbonyl, heterocyclesulfonyl, heterocyclealkyl, heterocycleoxyalkyl and —O-alkyl-CH2-cycloalkyl, —(CH2)p-Ar1, (CH2)p-CH—(Ar1)(Ar2), (CH2)p-heterocycle, and -(alkyl-O)n-(CH2)2-X; wherein n is independently chosen from 0 to 5. In some embodiments n is 0, in others n is 1, 2, 3, 4, or 5. In embodiments p is independently chosen from 0 to 3 where in some embodiments p can be 0, 1, 2 or 3.

In further embodiments, the compound according to formula I is coupled or linked to a second active moiety to act as a heterobifunctional molecule. In embodiments, the heterobifunctional molecule is a proteolysis-targeting chimera (PROTAC), Lysosome-targeting chimeras (LYTACs), autophagy-targeting chimeras (AUTACs) and autophagy-tethering compounds (ATTECs). In embodiments, the second active moiety is selected from a group of moieties that functions as E3 ligase ligand. In other embodiments, the second active moiety is a reporter moiety such as a fluorescent label or a biotinylated label.

In embodiments, X is a linker moiety that engages with the second active moiety and links the second active moiety to the compound disclosed herein to form heterobifunctional molecule. The linker moieties are known in the field. In embodiments, X is selected from a PEG linker, Alkyl linker, alkyne linker, or a click chemistry linker.

In some embodiments, X is selected from a group of moieties that functions as E3 ligase ligand wherein said X moiety engages with an E3 ubiquitin ligase or in complex with an E2 ubiquitin conjugating enzyme. Considering that compounds of formula I are able to bind to DPP9, the presence of the E3 ligase ligand will induce ubiquitylation of DPP9 and subsequently the proteasomal degradation of DPP9. In other words, in some embodiments where compounds comprise said X moiety, said compounds will function as proteolysis-targeting chimera (also known as PROTAC) where the compound will recruit DPP9 to the E3 ligase thus leading to the degradation of the DPP9 protein. In embodiments said E3 ligase can be any E3 such as VHL or cereblon.

In some embodiments, the E3 ligase binding compounds of the disclosed invention are used to induce DPP9 ubiquitylation and proteolysis in and treatment of a disease.

In other embodiments, X is selected from a group of reporter probes that is used as molecular labeling moieties. In some embodiments compounds of Formula I with molecular labeling moieties can be used as DPP9 protein-probes for specific labeling of DPP9 protein. Labeling probe moieties can be selected from any peptide labeling moiety known in the art. For example, said moiety can be a fluorogenic moiety or biotinylated moiety. The term “fluorescent moiety”, as used herein, refers to a compound, chemical group, or composition that is inherently fluorescent. Fluorigenic moieties include, but are not limited to coumarin, cyanine, benzofuran, a quinoline, a quinazolinone, an indole, a furan, a benzazole, a borapolyazaindacene and xanthenes including fluorescein, rhodamine and rhodol as well as other fluorophores. Biotinylated moiety refers to a part of the compounds disclosed herein where biotin is covalently attached.

In some embodiments, said protein probes can be used in biomarker research, such as for specific labeling of proteins, and can also be used for quantification, detection or kinetic studies of DPP9 protein, and the imaging of cells, and tissues.

In embodiments, each Ar¹, Ar² is independently chosen from an aromatic ring. In some embodiments said aromatic ring can be mono- or bicyclic aromatic rings, preferably monocyclic aromatic ring, more preferably 5- or 6-membered monocyclic aromatic ring. In other embodiments, Ar¹ and Ar² can optionally comprise 1 or 2 heteroatoms selected from O, N, S, adamantyl, indole or isoindole. In other embodiments each Ar¹, Ar² can optionally be substituted with 0 to 3 substituents wherein said substituents are selected from halogen, nitrile or phenyl.

In embodiments, W¹, W², W³ and W⁴ are independently selected from the group comprising hydrogen, halogen, or —O—C₁₋₆ alkyl, preferably halogen, more preferably F. In some embodiments —O—C₁₋₆alkyl preferably has 1 to 6 carbon atoms, preferably 1 to 4 carbon atoms, more preferably 1 or 3 carbon atoms. In some embodiments halogen is preferably F.

In embodiments, C₁₋₆ alkyl refers to straight or branched chain hydrocarbon groups having 1 to 6 carbon atoms, preferably 1 to 5 carbon atoms, preferably 1 to 4 carbon atoms more preferably 1 to 3 carbon atoms. Exemplary non-limiting alkyl groups include methyl, ethyl, propyl, isopropyl, n-butyl, t-butyl, isobutyl, pentyl, hexyl, and the like.

In embodiments, Z¹, Z², Z³, Z⁴, Z⁵, Z⁶, Z⁷, Z⁸ and Z⁹ are independently selected from hydrogen, halogen, —NHR, or C₁₋₆ alkyl.

In other embodiments, Z¹, Z², Z³, Z⁴, Z⁵, Z⁶, Z⁷, Z⁸ and Z⁹ are independently selected from hydrogen, —NHR, —NHC(═O)R, —NHC(═O)OR, —NHC(═O)NHR, —OR, —O(C═O)NHR or —O—(C═O)-L-prolinate.

In embodiments of the present invention, R moiety in Z¹, Z², Z³, Z⁴, Z⁵, Z⁶, Z⁷, Z⁸ and Z⁹ of Formula I can be selected from the group of hydrogen, C₁₋₆ alkyl, C₁₋₆ hydroxyalkyl, C₅₋₇ cycloalkyl, C₅₋₇ hydroxycycloalkyl or Ar¹. In other embodiments R can be selected from the group of carboxyalkyl, alkylcarboxyalkyl, (alkyl)(alkylcarboxyalkyl)sulfide, alkylamide-benzyl carbamate, carboxyalkyl-Ar¹ wherein Ar¹ is chosen from an aromatic ring as described above.

In some embodiments, W¹, W², W³ and W⁴ are independently selected from: hydrogen, halogen, haloalkyl, NH₂ or —O—C₁₋₆ alkyl; Z¹, Z², Z³, Z⁴, Z⁵, Z⁶, Z⁸ and Z⁹ are independently selected from: hydrogen, halogen, or —C₁₋₆ alkyl; and Z⁷ is selected from: —C₁₋₆ alkyl, —NHR, —NHC(═O)R, —NHC(═O)OR, —NHC(═O)NHR, —O(C═O)NHR, or —O—(C═O)-L-prolinate, wherein R is independently selected from: hydrogen, —C₁₋₆ alkyl, —CH—(Ar¹)—(Ar²), CH₃₋ₙ—(Ar¹)ₙ, or adamantyl, wherein n is 1 or 2. In some embodiments, said —C₁₋₆ alkyl is optionally substituted with hydroxy, halogen or —Ar¹, wherein each Ar¹, Ar² is independently chosen from an aromatic ring optionally comprising 1 or 2 heteroatoms selected from O, N and S, adamantyl, indole or isoindole; each Ar¹, Ar² being optionally substituted with from 0 to 3 substituents selected from halogen, nitrile or phenyl.

According to other embodiments, W¹, W², W³ and W⁴ of the compound of Formula I are independently selected from: hydrogen, halogen and —O—C₁₋₆alkyl; Z¹, Z², Z³, Z⁴, Z⁵, Z⁶, Z⁸ and Z⁹ are Hydrogen; and Z⁷ is —O(C═O)NHR where R is selected from the group of —CH—(Ar₁)₂, carboxyalkylbenzylcarbamate, carboxyalkyl, -(alkyl)(carboxyalkyl)sulfide, alkylamide-benzyl carbamate, carboxyalkyl-Ar¹, alkylcarboxyalkyl, alkylcarboxylalkylAr¹.

In other embodiments, W¹, W², W³ and W⁴ are independently selected from the group comprising hydrogen, halogen, and O—C₁₋₆ alkyl; Z¹, Z², Z³, Z⁴, Z⁵, Z⁶, Z⁸ and Z⁹ is selected from hydrogen or —C₁₋₆ alkyl; and Z³ is selected from —NHC(═O)OR or —NHC(═O)NHR, wherein R is -(alkyl-O)n-(CH₂)₂—X, and n is 0 to 6. In embodiments “alkyl” in -(alkyl-O)ₙ—(CH₂)₂—X can have 1 to 6 carbons, preferably 1 to 4 carbons, more preferably 1 to 3 carbons, even more preferably 2 carbons. In embodiments n can be 6, 5, 4, 3, 2, 1 or 0, preferably n is 5 to 1, more preferably 4 to 1, even more preferably 3 to 1. In specific embodiments X can be

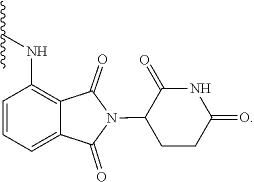 [SMILES: *Nc1cccc2c1C(=O)N(C1CCC(=O)NC1=O)C2=O]

In other embodiments X can be

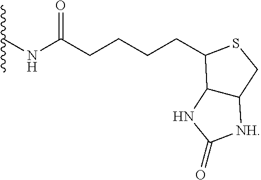 [SMILES: *NC(=O)CCCCC1SCC2NC(=O)NC21]

Non-limiting preferred compounds of some embodiments are listed in Table 3. Even more preferred compounds are

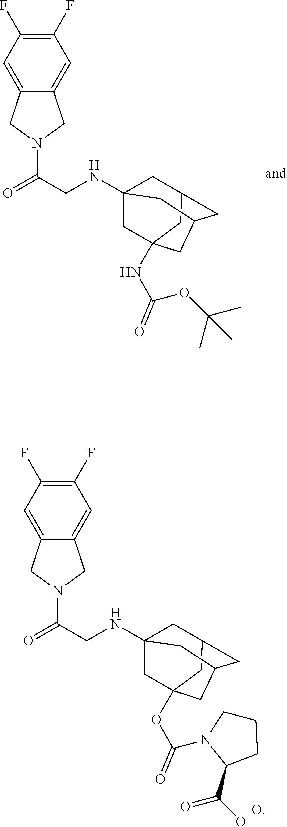

According to embodiments, the above compounds of the disclosure bind to the DPP9 enzyme. In the embodiments, said compounds of the present invention are DPP9 inhibitors, more specifically selective DPP9 inhibitors wherein said selective DPP9 inhibitors inhibit DPP9 to a greater extent than they inhibit at least DPP8 and/or DPP4. The compounds can selectively inhibit DPP9 at least 2 times, at least 5 times, at least 10 times, at least 50 times, at least 75 times, at least 100 times, at least 150 times, at least 200 times, at least 250 times, at least 300 times, at least 350 times, at least 400 times, at least 450 times, at least 500 times, at least 550 times, at least 600 times, at least 650 times, at least 700 times, at least 750 times, at least 800 times, at least 850 times, at least 900 times, at least 950 times or at least 1000 times more than they inhibit DPP8. For example, the compounds can selectively inhibit DPP9 in the range of 2 to 1000 times, 2 to 750 times, 2 to 500 times, 2 to 250 times, 2 to 200 times, 5 to 150 times, 10 to 100 times, and all the ranges and subranges therein between more efficiently than they inhibit DPP8 and/or DPP4.

Compounds described herein can also inhibit DPP9 function through the incorporation of agents that catalyze the destruction of DPP9. For example, according to some embodiments, the compounds of Formula I can be incorporated into proteolysis targeting chimeras (PROTACs). A PROTAC is a bifunctional molecule, with one portion capable of engaging an E3 ubiquitin ligase, and the other portion having the ability to bind to a target protein meant for degradation by the cellular protein quality control machinery. Recruitment of the target protein to the specific E3 ligase results in its tagging for destruction (i.e., ubiquitination) and subsequent degradation by the proteasome. Any E3 ligase can be used to induce the ubiquitination. The portion of the PROTAC that engages the E3 ligase is connected to the portion of the PROTAC that engages the target protein via a linker which consists of a variable chain of atoms.

In embodiments, the compounds of Formula I is a selective binder of DPP9 enzyme. In some embodiments, compounds of Formula I can also possess a E3 ligase ligand/binding moiety wherein said E3 ligase ligand is linked to DPP9 binding part via an (alkyl-O)n chain. Therefore resulting in the recruitment of DPP9 to the E3 ligase, thus leading to the degradation of the DPP9 protein.

According to some embodiments R1 and/or R2 moieties of compound of invention engages with an E3 ubiquitin ligase (e.g., cereblon or VHL) or in complex with an E2 ubiquitin conjugating enzyme, while another moiety of said compound binds to the DPP9 enzyme, preferably simultaneously. Consequently, ubiquitination of DPP9 and therefore the proteasomal degradation of DPP9 is induced. According to embodiments, the E3 ligase can be selected from any of the E3 ligases and any of the known and/or commercially available E3 ligands can be linked to the DPP9 binding compound disclosed herein.

According to some embodiments, compound of Formula I comprises a molecular labeling moiety wherein said compound binds to DPP9 and can be used as DPP9 protein probe for selective labeling of DPP9 protein. The labeling moiety of the said compound is preferably chosen from a fluorescent probe or a biotinylated probe.

In another embodiment, said compounds functioning as DPP9 enzyme probes can be used in biomarker research, such as for specific labeling, detection, or kinetic studies of DPP9 protein, and the imaging of cells, and tissues. In other embodiments, said DPP9 enzyme probes can be used in diagnostics and treatment of a disease such as for quantification, detection of DPP9 enzyme, and the imaging of cells, tissues, and living bodies.

A compound as detailed herein may in one embodiment be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated “substantially pure” intends a composition that contains no more than 35% impurity, wherein the impurity denotes a compound other than the compound comprising the majority of the composition or a salt thereof. In some embodiments, a composition of a substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1%, or 0.5% impurity.

The compounds depicted herein may be present as salts even if salts are not depicted and it is understood that the present disclosure embraces all salts and solvates of the compounds depicted here, as well as the non-solvate form of the compound, as is well understood by the skilled artisan. In some embodiments, the salts of the compounds provided herein are pharmaceutically acceptable salts.

Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein. The present disclosure also includes any or all the stereochemical forms, including any enantiomeric or diastereomeric forms of the compounds described. The structure or name is intended to embrace all possible stereoisomers of a compound depicted. All forms of the compounds are also embraced by the invention, such as crystalline or non-crystalline forms of the compounds. Compositions comprising a compound of the invention are also intended, such as a composition of substantially pure compound, including a specific stereochemical form thereof, or a composition comprising mixtures of compounds of the invention in any ratio, including two or more stereochemical forms, such as in a racemic or non-racemic mixture.

Preferably, the compounds detailed herein are orally bioavailable. However, the compounds may also be formulated for parenteral (e.g., intravenous) administration. One or several compounds described herein can be used in the preparation of a medicament by combining the compound or compounds as an active ingredient with a pharmacologically acceptable carrier, which are known in the art. Depending on the therapeutic form of the medication, the earner may be in various forms. In one embodiment, the manufacture of a medicament is for use in any of the methods disclosed herein, e.g., for the treatment of cancer.

### Pharmaceutical Compositions and Formulations

Another aspect of the present disclosure is directed to a pharmaceutical composition comprising a therapeutically effective amount of a compound of Formula I or a pharmaceutically acceptable salt thereof in combination with an optional pharmaceutically suitable carrier, diluent, excipient, or adjuvant. The above compounds of the disclosure can be used in a method of inhibiting the DPP9 enzyme, comprising administering to a mammal, a therapeutically effective amount of a compound of Formula I.

A compound according to the present invention may in one embodiment be in a purified form. In an embodiment, the composition comprises a compound as detailed herein or a salt thereof. In some embodiments, the composition comprises a compound as detailed herein or a salt thereof in substantially pure form.

In an embodiment, the compounds herein are synthetic compounds prepared for administration to an individual. In another embodiment, compositions are provided containing a compound in substantially pure form. In another embodiment, the present invention embraces pharmaceutical compositions comprising a compound detailed herein and a pharmaceutically acceptable carrier. In another embodiment, methods of administering a compound are provided. The purified forms, pharmaceutical compositions, and methods of administering the compounds are suitable for any compound or form thereof detailed herein.

A compound detailed herein, or salt thereof may be formulated for any available delivery route, including an oral, mucosal (e.g., nasal, sublingual, vaginal, buccal, or rectal), parenteral (e.g., intramuscular, subcutaneous, or intravenous), topical or transdermal delivery form. Pharmaceutical compositions may take a form suitable for oral, buccal, parenteral, nasal, topical, or rectal administration or a form suitable for administration by inhalation. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein, or a salt thereof can be used in the preparation of a composition, such as a pharmaceutical formulation, by combining the compound or compounds, or a salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulators, sweeteners, dyes, adjusters, and salts for the adjustment of osmotic pressure, buffers, coating agents, or antioxidants. Formulations comprising the compound may also contain other substances which have valuable therapeutic properties. Pharmaceutical formulations may be prepared by known pharmaceutical methods.

Compositions, as described herein, may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shells are, for instance, plant oils, wax, fats, semisolid and liquid poly-oils, and so on. In addition, pharmaceutical formulations may contain preservatives, solubilizers, stabilizers, re-wetting agents, emulators, sweeteners, dyes, adjusters, and salts for the adjustment of osmotic pressure, buffers, coating agents or antioxidants.

In some embodiments, the composition is for use as a human or veterinary medicament. In some embodiments, the composition is for use in a method described herein. In some embodiments, the composition is for use in the treatment of a disease or disorder described herein.

By means of non-limiting examples, such a composition may be in a form suitable for parenteral administration (such as by intravenous, intramuscular, or subcutaneous injection or intravenous infusion), for topical administration (including ocular), for administration by inhalation, a skin patch, by an implant, by a suppository, etc. Such suitable administration forms—which may be solid, semi-solid or, liquid, depending on the manner of administration—as well as methods and carriers, diluents and excipients for use in the preparation thereof, will be clear to the skilled person.

Some preferred, but non-limiting examples of preparations include tablets, pills, powders, lozenges, sachets, cachets, elixirs, suspensions, emulsions, solutions, syrups, aerosols, ointments, creams, lotions, soft and hard gelatin capsules, suppositories, eye drops, sterile injectable solutions and sterile packaged powders (which are usually reconstituted prior to use) for administration as a bolus and/or for continuous administration, which may be formulated with carriers, excipients, and diluents that are suitable per se for such compositions, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils, and mineral oils or suitable mixtures thereof. The compositions can optionally contain other pharmaceutically active substances (which may or may not lead to a synergistic effect with the compounds of the invention) and other substances that are commonly used in pharmaceutical compositions, such as lubricating agents, wetting agents, emulsifying and suspending agents, dispersing agents, desintegrants, bulking agents, fillers, preserving agents, sweetening agents, flavoring agents, flow regulators, release agents, etc. The compositions may also be formulated so as to provide a rapid, sustained or delayed release of the active compound(s) contained therein, for example using liposomes or hydrophilic polymeric matrices based on natural gels or synthetic polymers. In order to enhance the solubility and/or the stability of the compounds of a pharmaceutical composition according to the invention, it can be advantageous to employ α-, β- or γ-cyclodextrins or their derivatives.

In addition, co-solvents such as alcohols may improve the solubility and/or the stability of the compounds. In the preparation of aqueous compositions, the addition of salts of the compounds of the invention can be more suitable due to their increased water solubility.

The preparations may be prepared in a manner known per se, which usually involves mixing at least one compound according to the invention with the one or more pharmaceutically acceptable carriers, and, if desired, in combination with other pharmaceutically active compounds, when necessary, under aseptic conditions.

For an oral administration form, the compositions of the present invention can be mixed with suitable additives, such as excipients, stabilizers, or inert diluents, and brought by means of the customary methods into the suitable administration forms, such as tablets, coated tablets, hard capsules, aqueous, alcoholic, or oily solutions. Examples of suitable inert carriers are Arabic gum, magnesia, magnesium carbonate, potassium phosphate, lactose, glucose, or starch, in particular, corn starch. In this case, the preparation can be carried out both as dry and as moist granules. Suitable oily excipients or solvents are vegetal or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate and lactose and/or other excipients, binders, extenders, disintegrants, diluents and lubricants known in the art. When administered by nasal aerosol or inhalation, these compositions may be prepared according to techniques well-known in the art of pharmaceutical composition and may be prepared as solutions in saline, employing benzyl alcohol or other suitable preservatives, absorption promoters to enhance the bioavailability, fluorocarbons, and/or other solubilizing or dispersing agents known in the art. Suitable pharmaceutical compositions for administration in the form of aerosols or sprays are, for example, solutions, suspensions or emulsions of the compounds of the invention or their physiologically tolerable salts in a pharmaceutically acceptable solvent, such as ethanol or water, or a mixture of such solvents. If required, the composition can also additionally contain other pharmaceutical auxiliaries such as surfactants, emulsifiers and stabilizers as well as a propellant. For subcutaneous administration, the compound according to the invention, if desired with the substances customary therefore such as solubilizers, emulsifiers or further auxiliaries are brought into solution, suspension, or emulsion. The compounds of the invention can also be lyophilized and the lyophilizates obtained used, for example, for the production of injection or infusion preparations. Suitable solvents are, for example, water, physiological saline solution or alcohols, e.g. ethanol, propanol, glycerol, in addition also sugar solutions such as glucose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these compositions may be prepared by mixing the compounds according to the invention with a suitable non-irritating excipient, such as cocoa butter, synthetic glyceride esters, or polyethylene glycols, which are solid at ordinary temperatures, but liquefy and/or dissolve in the rectal cavity to release the drug. In a preferred embodiment, the compounds of the present invention are useful in human or veterinary medicine, in particular for use as FAP (fibroblast activation protein) inhibitors.

In an embodiment, the pharmaceutical composition may comprise a chelator selected from the group of: EUpypa, EDTA (ethylenediamine tetraacetate), EDTMP (diethylenetriaminepenta (methylenephosphonic acid)), DTPA (diethylenetriaminepentaacetate) and its derivatives, DOTA (Dodeca-1,4,7,10-tetraamine-tetraacetate), DOTAGA (2-(I, 4,7, 10-tetraazacyclododecane-4, 7,10) pentanedioic acid) and other DOTA derivatives, TRITA (trideca-1, 4,7,10-tetraamine-tetraacetate), TETA (tetradeca-I, 4,8, II-tetraamine-tetraacetate) and its derivatives, NOTA (Nona-1, 4,7-triamine-triacetate) and its derivatives such as NOTAGA (I, 4,7-triazacyclononane, I-glutaric acid, 4,7-acetate), TRAP (triazacyclononane phosphinic acid), NOPO (I, 4,7-triazacyclononane-1,4-bis [methylene (hydroxymethyl) phosphinic acid]-7-[methylene (2-carboxyethyl) phosphinic acid]), PEPA (pentadeca-1, 4,7,10,13-pentaamine pentaacetate), HEHA (hexadeca-1, 4,7,10,13,16-hexaamine-hexaacetate) and its derivatives, HBED (hydroxybenzyl-ethylene-di amine) and its derivatives, DEDPA and its derivatives, such as H2DEDPA (I, 2-[[6-(carboxylate) pyridin-2-yl]methylamine]ethane), DFO (deferoxamine) and its derivatives, trishydroxypyridinone (THP) and its derivatives such as YM103, TEAP (tetraazycyclodecanephosphinic acid) and its derivatives, AAZTA (6-amino-6-methylperhydro-1,4-diazepine-tetraacetate) and derivatives such as DATA ((6-Pentanoic acid)-6-(amino) methyl-1,4-diazepine triacetate); SarAr (IN-(4-aminobenzyl)-3,6,10,13,16,19-hexaazabicyclo [6.6.6]-eicosane-1,8-diamine) and salts thereof, (Nhh SAR (1,8-diamino-B, 6,10,13,16,19-hexaazabicyclo [6.6.6]icosane) and salts and derivatives thereof, aminothiols and their derivatives.

### Methods of Treatment and Use of Compounds or Compositions

Also disclosed herein are methods of treatment and/or diagnostic methods. More particularly, disclosed herein are compounds or pharmaceutical compositions as described herein for use as a human or veterinary. Even more particularly, the above compounds of the disclosure can be used in a method of treating or prophylactically treating disorders in a mammal by inhibiting the DPP9 enzyme, comprising administering to a mammal, a therapeutically effective amount of a compound of Formula I.

In an embodiment, a method for the diagnosis or treatment of a disorder/disease in an individual is disclosed herein, wherein said disease is a DPP9 enzyme-related disorder.

In embodiments, the above compounds, or a pharmaceutical composition thereof of the disclosure can be used in the diagnosis or treatment of a disorder by inhibiting the DPP9 enzyme for the induction of pyroptosis.

The term “DPP9 enzyme-related disorder” as used herein, means any disease or other deleterious condition in which DPP9 is known to play a role. The term “DPP9 enzyme-related disorder” also means those diseases or conditions that are alleviated by treatment with a DPP9 inhibitor. A non-limiting list of examples of DPP9 enzyme-related disorders can include proliferative diseases selected from the group of basal cell carcinoma, biliary tract cancer, bladder cancer, bone cancer, brain cancer, breast cancer, cervical cancer, choriocarcinoma, CNS cancer, colon and rectum cancer, connective tissue cancer, cancer of the digestive system, endometrial cancer, esophageal cancer, eye cancer, cancer of the head and neck, gastric cancer, intra-epithelial neoplasm, kidney cancer, larynx cancer, leukemia, acute myeloid leukemia, acute lymphoid leukemia, chronic myeloid leukemia, chronic lymphoid leukemia, liver cancer, small cell lung cancer, non-small cell lung cancer, lymphoma, Hodgkin's lymphoma, Non-Hodgkin's lymphoma, melanoma, myeloma, myeloproliferative disease, neuroblastoma, oral cavity cancer, ovarian cancer, pancreatic cancer, prostate cancer, retinoblastoma, rhabdomyosarcoma, rectal cancer, renal cancer, cancer of the respiratory system, sarcoma, skin cancer, stomach cancer, testicular cancer, thyroid cancer, uterine cancer, cancer of the urinary system, and Ewing sarcoma.

In some embodiments, the cancer is selected from the group consisting of carcinoma, sarcoma, leukemia, lymphoma, or myeloma.

In some embodiments, cancer selected from the group consisting of lung cancer, lymphomas, breast cancer, colorectal cancer, thyroid cancer, uterine cancer, pancreatic cancer, prostate cancer, skin cancer, kidney cancer, liver cancer, brain cancer, human hepatocellular carcinoma (HCC), non-small-cell lung cancer, ovarian cancer, oral squamous cell carcinoma, and Ewing sarcoma.

DPP9 overexpression is associated with poor prognosis in human hepatocellular carcinoma (HCC), in non-small-cell lung cancer, in colorectal cancer, breast cancer, ovarian cancer, and can result in tubulointerstitial fibrosis. It is also a survival factor in Ewing sarcoma. In contrast, DPP9 downregulation is associated with poor prognosis in oral squamous cell carcinoma. DPP9 inhibition also may provide interesting therapeutic prospects in reducing atherosclerosis and/or in the prevention of plaque rupture. Therefore, the term “DPP9 enzyme-related disorder” also comprises all the indications mentioned above including tubulointerstitial fibrosis, squamous cell carcinoma, atherosclerosis and/or plaque rupture.

Another aspect of the present invention relates to any one of the foregoing methods, wherein the myeloproliferative disease is acute myeloid leukemia.

Another aspect of the present invention relates to any one of the foregoing methods, wherein the myeloproliferative disease is a chronic myeloproliferative disease.

The invention also provides methods for the prevention and/or treatment of a DPP9 enzyme-related disorder; said method comprises administering to a subject in need thereof a compound according to this invention, or a composition comprising said compound. In some embodiments, said compound is administered orally or parenterally. In some embodiments, said compound is administered topically. In some embodiments, said compound is administered in a solid dosage form. In some embodiments, the solid dosage form is a tablet, capsule, or pill. In some embodiments, the solid dosage form is a tablet. In some embodiments, said compound is administered in an amount sufficient to stimulate the DPP9 inhibition without dose-limiting toxicity. In some embodiments, said compound is administered in an amount sufficient to stimulate the pyroptosis without dose-limiting toxicity.

In an embodiment, a compound or salt thereof described herein or a composition described herein may be used in a method of treating a disease or disorder characterized by proliferation. In some embodiments, a compound or salt thereof described herein or a composition described herein may be used in a method of treating cancer, such as leukemia, lung cancer, breast cancer, colorectal cancer, ovarian cancer, prostate cancer, pancreatic cancer, kidney cancer, melanoma, fibrosarcoma, bone and connective tissue sarcomas, renal cell carcinoma, giant cell carcinoma, squamous cell carcinoma, and adenocarcinoma; preferably leukemia, more preferably acute myeloid leukemia (AML).

Compounds and compositions detailed herein, such as a pharmaceutical composition containing a compound of any formula provided herein or a salt thereof and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays. Provided herein is a method of treating a disease or disorder in an individual in need thereof comprising administering a compound describes herein or any embodiment, or aspect thereof, or a pharmaceutically acceptable salt thereof. In some embodiments, the compound, pharmaceutically acceptable salt thereof, or composition is administered to the individual according to dosage and/or method of administration described herein.

In an embodiment, the administration of the compound, salt, or composition reduces tumor growth, tumor proliferation, or tumorigenicity in the individual. In some embodiments, the compound, salt, or composition may be used in a method of reducing tumor growth, tumor proliferation, or tumorigenicity in an individual in need thereof. In some embodiments, tumor growth is slowed or stopped. In some embodiments, tumor growth is reduced at least about 10%, 20%, 30%, 40%, 50%, 60%, 70%, 80%, 90% or more. In some embodiments, the tumor is reduced in size. In some embodiments, tumor metastasis is prevented or slowed.

In some embodiments, provided herein is a method of inhibiting DPP9 and/or causing DPP9 proteolysis (degradation). The compounds or salts thereof described herein, and compositions described herein are believed to be effective for inhibition or degradation. In some embodiments, the method of inhibiting DPP9 comprises inhibiting DPP9 in a cell by administering or delivering to the cell a compound described herein, or a pharmaceutically acceptable salt thereof, or a pharmaceutical composition described herein. In some embodiments, the cell is a DPP9 expressing ceil. In some embodiments, the method of inhibiting DPP9 comprises inhibiting DPP9 in a tumor or plasma by administering or delivering to the tumor or plasma a compound described herein, or a pharmaceutically acceptable salt thereof, or a pharmaceutical composition described herein. In some embodiments, the inhibition of DPP9 comprises inhibiting an endopeptidase and/or exopeptidase activity of DPP9. In some embodiments, DPP9 is inhibited by at least about 10%, 20%, 30%, 40%, 50%, 60%, 70%, 80%, 90%, 95%, 98% or more. Inhibition of DPP9 can be determined by methods known in the art.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

The compounds of the current invention, once administered to the patient, can localize to specific organs or cells allowing visualizing the extent of a disease-process in the body, based on the cellular function and physiology, rather than relying on physical changes in the tissue anatomy.

In some embodiments, the individual is an animal, preferably a mammal. In some embodiments, the individual is a primate, bovine, ovine, porcine, equine, canine, feline, or rodent. In some embodiments, the individual is a human. In some embodiments, the individual has any of tire diseases or disorders disclosed herein. In some embodiments, the individual is a risk of developing any of the diseases or disorders disclosed herein. In some embodiments, the individual is human. In some embodiments, the human is at least about or is about any of 21, 25, 30, 35, 40, 45, 50, 55, 60, 65, 70, 75, 80, or 85 years old. In some embodiments, the human is a child. In some embodiments, the human is less than about or about an 01, 18, 15, 12, 10, 8, 6, 5, 4, 3, 2, or 1 year.

The dose of a compound administered to an individual (such as a human) may vary with the particular compound or salt thereof, the method of administration, and the particular disease, such as type and stage of cancer, being treated. In some embodiments, the amount of the compound or salt thereof is a therapeutically effective amount.

The pharmaceutical preparations of the invention are preferably in a unit dosage form, and may be suitably packaged, for example in a box, blister, vial, bottle, sachet, ampoule, or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the invention, e.g. about 10, 25, 50, 100, 200, 300 or 400 mg per unit dosage.

The compounds can be administered by a variety of routes including the oral, rectal, ocular, transdermal, subcutaneous, intravenous, intramuscular, or intranasal routes, depending mainly on the specific preparation used and the condition to be treated or prevented, and with oral and intravenous administration usually being preferred. The at least one compound of the invention will generally be administered in an “effective amount”, by which is meant any amount of a compound of the Formula I, upon suitable administration, is sufficient to achieve the desired therapeutic or prophylactic effect in the individual to which it is administered. Usually, depending on the condition to be prevented or treated and the route of administration, such an effective amount will usually be between 0.01 to 1000 mg per kilogram body weight day of the patient per day, more often between 0.1 and 500 mg, such as between 1 and 250 mg, for example about 5, 10, 20, 50, 100, 150, 200 or 250 mg, per kilogram bodyweight day of the patient per day, which may be administered as a single daily dose, divided over one or more daily doses, or essentially continuously, e.g. using a drip infusion. The amount(s) to be administered, the route of administration, and the further treatment regimen may be determined by the treating clinician, depending on factors such as the age, gender and general condition of the patient and the nature and severity of the disease/symptoms to be treated.

The effective amount of the compound may in one embodiment be a dose of between about 0.01 and about 100 mg/kg. Effective amounts or doses of the compounds of the invention may be ascertained by routine methods, such as modeling, dose-escalation, or clinical trials, considering routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

A compound or composition of the invention may be administered to an individual in accordance with an effective dosing regimen for the desired period or duration, such as at least about one month, at least about 2 months, at least about 3 months, at least about 6 months, or at least about 12 months or longer, which in some embodiments may be for the duration of the individual's life. In one embodiment, the compound is administered on a daily or intermittent schedule. The compound can be administered to an individual continuously (for example, at least once daily) over a period of time. The dosing frequency can also be less than once daily, e.g., about once-weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14-day time period, such as about 2 months, about 4 months, about 6 months or more). Any of the dosing frequencies can employ any of the compounds described herein together with any of the dosages described herein.

In an embodiment, said pharmaceutical composition can be administered separately at different times during the course of therapy or concurrently in divided or single combination forms.

### Articles of Manufacture and Kits

The present disclosure further provides articles of manufacture comprising a compound described herein or a salt thereof, a composition described herein, or one or more-unit dosages described herein in suitable packaging. In certain embodiments, the article of manufacture is for use in any of the methods described herein. Suitable packaging is known in the art and includes, for example, vials, vessels, ampules, bottles, jars, flexible packaging, and the like. An article of manufacture may further be sterilized and/or sealed.

The present disclosure further provides kits for carrying out the methods of the invention, which comprises one or more compounds described herein or a composition comprising a compound described herein. The kits may employ any of the compounds disclosed herein. In one embodiment, the kit employs a compound described herein or a salt thereof. The kits may be used for any one or more of the uses described herein, and, accordingly, may contain instructions for the treatment any disease or described herein, for example for the treatment of cancer. Kits generally comprise suitable packaging. The kits may comprise one or more containers comprising any compound described herein. Each component (if there is more than one component) can be packaged in separate containers or some components can be combined in one container where cross-reactivity and shelf life permit. The kits may be in unit dosage forms, bulk packages (e.g., multi-dose packages) or sub-unit doses. For example, kits may be provided that contain sufficient dosages of a compound as disclosed herein and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

### Synthesis

The compounds of this invention may be prepared by a variety of procedures and synthetic routes. Representative procedures and synthetic routes are shown in, but are not limited to, Schemes 1-23.

In an embodiment, the synthesis of the some of the compounds disclosed here is pursued first by the synthesis of the building blocks, P1 and P2 illustrated in structure I, and followed by assembly of said synthesized building blocks, (Schemes 1-4).

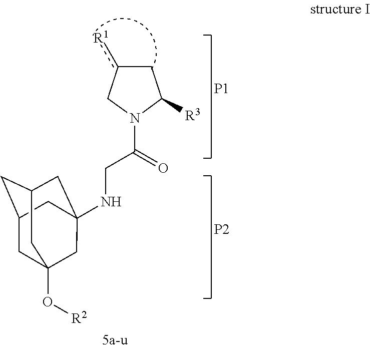

In another embodiment, the synthesis of the compounds disclose here is pursued by intermediate synthesis of varied analogues of isoindoline, aminoacyl and adamantyl moieties according to Formula I and followed by coupling of the moieties (Schemes 5-23).

The compounds and processes of the present invention will be better understood in connection with the following synthetic schemes and experiments that illustrate a means by which the compounds of the invention may be prepared.

The synthesis routes that are disclosed herein are exemplary and it will be clear to a skilled person that other routes can be explored to obtain the compounds if needed.

### EXAMPLES

The present invention will now be further exemplified with reference to the following examples. The present invention is in no way limited to the given examples or to the embodiments presented in the figures.

### Example 1: Synthesis of the Compounds Listed in Table 1 and 2

This example reports on the first set of structural modifications that were applied to engineer out DPP4 potency and to further increase the DPP9 over DPP8 selectivity of the structure I. These modification types mainly relate, respectively, to an exploration of the P1-submoiety, supplemented with a limited number of minor modifications in the P2 part of the molecule depicted in Structure I.

The P1-subset consists of the parent molecule's 2-cyanopyrrolidine moiety, supplemented with substituted 2-cyanopyrrolidine derivatives for which we earlier published a small binding preference for DPP9 over DPP8. Lastly, we selected isoindoline. Isoindoline-bearing compounds such as 1 (1G244) have been reported to deliver small molecule inhibitors that are ‘DPP8/9’ selective.

Vildagliptin's N-(3-hydroxyadamantyl)glycine) moiety was kept as a common substructure in all these P1-varied compounds. This allows straightforward comparison of the selected P1 units, both relative to each other and relative to reference vildagliptin. For the P2-varied subset, a limited number of lipophilic O-alkyl and benzyl substituents were introduced on the 3-OH group adamantyl group in the form of ethers. This was based on the in silico superposition of the X-ray structures of DPP4, DPP8 and DPP9, suggesting a larger S2 pocket size for DPP8 and DPP9.

Building blocks of the compounds were synthesized according to Scheme 1-3 and assembled according to scheme 4 as explained below.

The synthesis of the target products involved the initial preparation of several 2-cyanopyrrolidine-derived P1 fragments and a number of adamantyl building blocks. (Schemes 1-3).

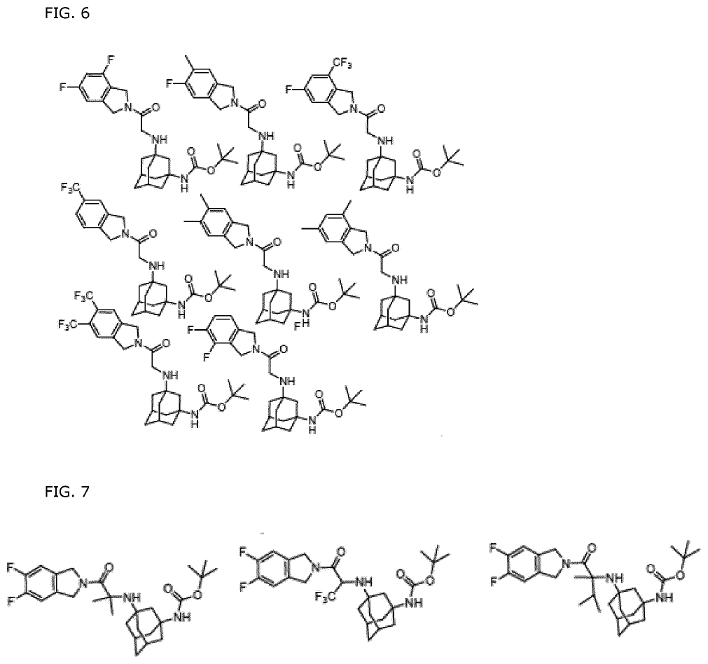

Synthesis of the P1 building blocks: The preparation of the P1 fragments is described in FIG. 6 and relies on reported synthetic strategies. In all cases, the corresponding commercially available N-Boc-protected L-proline analogs were used as the starting material. The latter was activated as N-hydroxysuccinimide esters and amidated using a methanolic ammonia solution. Dehydration with trifluoroacetic anhydride transformed the amides into the corresponding carbonitriles. Acidolytic cleavage using p-toluenesulfonic acid yielded the desired cyanopyrrolidine P1 fragments (compounds 15a-d).

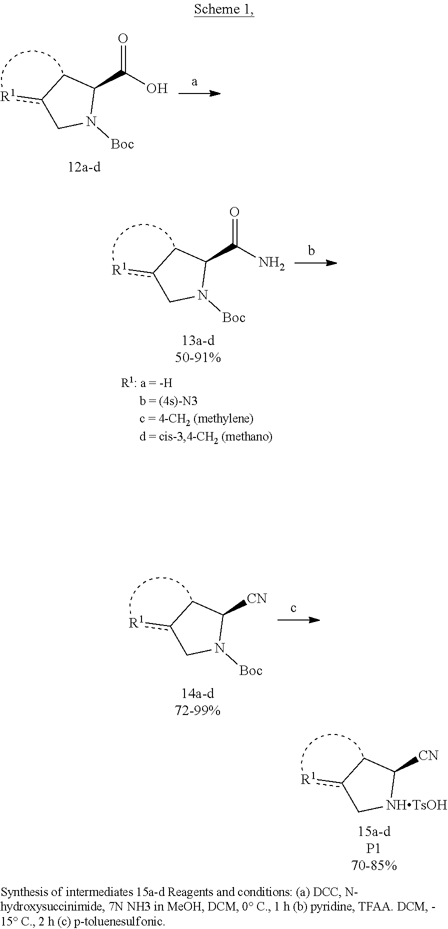

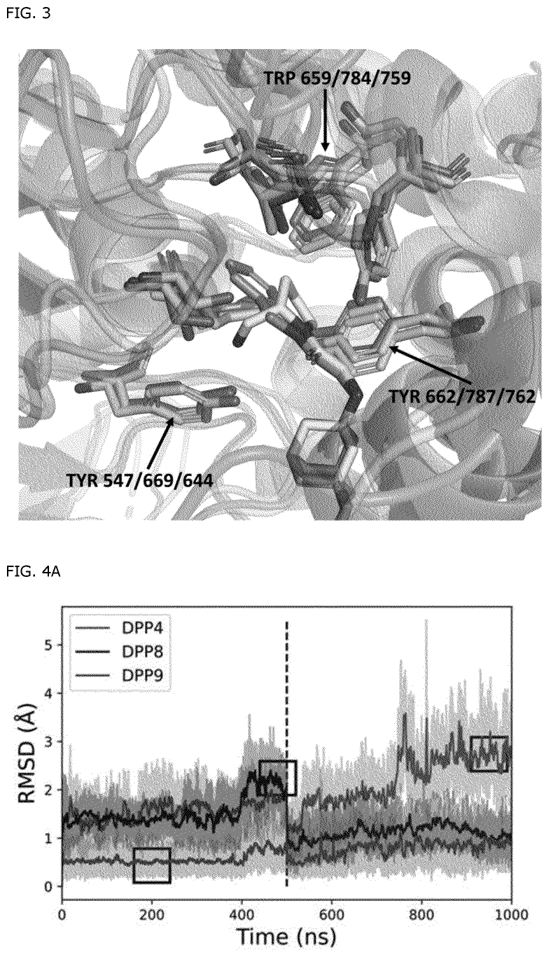

Synthesis of the adamantyl building blocks: Scheme 2 and 3 summarize the synthesis of the required adamantyl building blocks of the P2 residues. More specifically, Scheme 2 covers the synthesis of O-benzylated adamantyl fragments. It starts with phthalimide protection of commercially available 3-aminoadamantan-1-ol with phthalic anhydride, followed by Williamson ether synthesis with sodium hydride and either benzyl bromide or 2,4-difluorobenzyl bromide. Thereafter, the phthalimide group was removed in a two-step procedure consisting of sodium borohydride reduction and acidolysis, rendering the desired building blocks 19a-b. Because the Williamson-type alkylation in FIG. 3 was found to be only applicable with acceptable yields for the preparation of benzyl ethers, a different synthetic route described by Masada et al., was followed to synthesize the O-alkylated adamantyl building blocks. First, the amine of the commercially available 3-aminoadamantan-1-ol was N-Boc-protected and the hydroxy group subsequently mesylated with mesyl chloride. The mesylated product (21) was then mixed with 10 equivalents of the alcohol corresponding to the desired O-substituent and triethylamine. This yielded the N-Boc-protected aminoadamantyl ether. Acidolysis of the Boc-group with trifluoroacetic acid leads to trifluoroacetate salts of the desired 3-O-substituted adamantan-1-amine P2 subunit 23a-c.

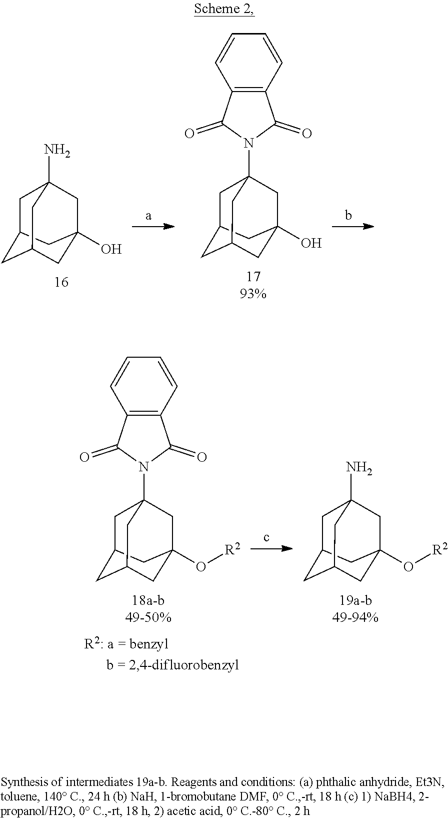

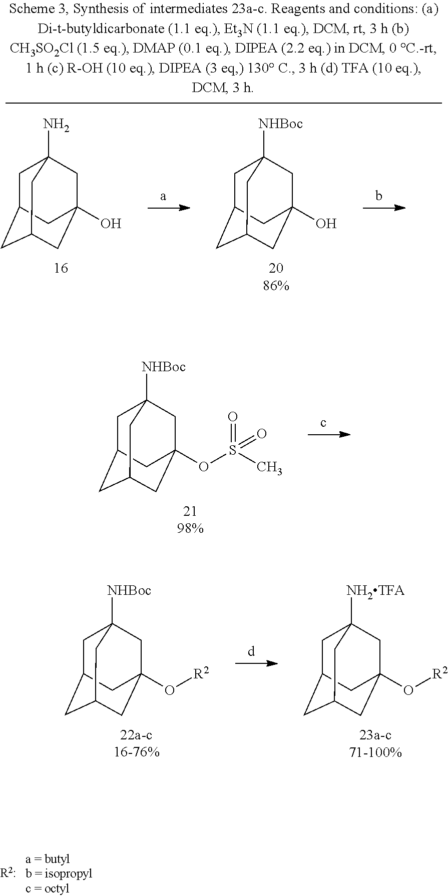

Assembly of the building blocks: After obtaining all required fragments, inhibitors were assembled following the general 2-step strategy displayed in Scheme 4. The 2-cyanopyrrolidines synthesized in FIG. 6, or commercially available isoindoline were first bromoacetylated with bromoacetyl bromide. Amination of this intermediate with either 3-aminoadamantan-1-ol or amino-adamantane ethers from Scheme 2 and 3, allowed to obtain the target compounds 5a-u.

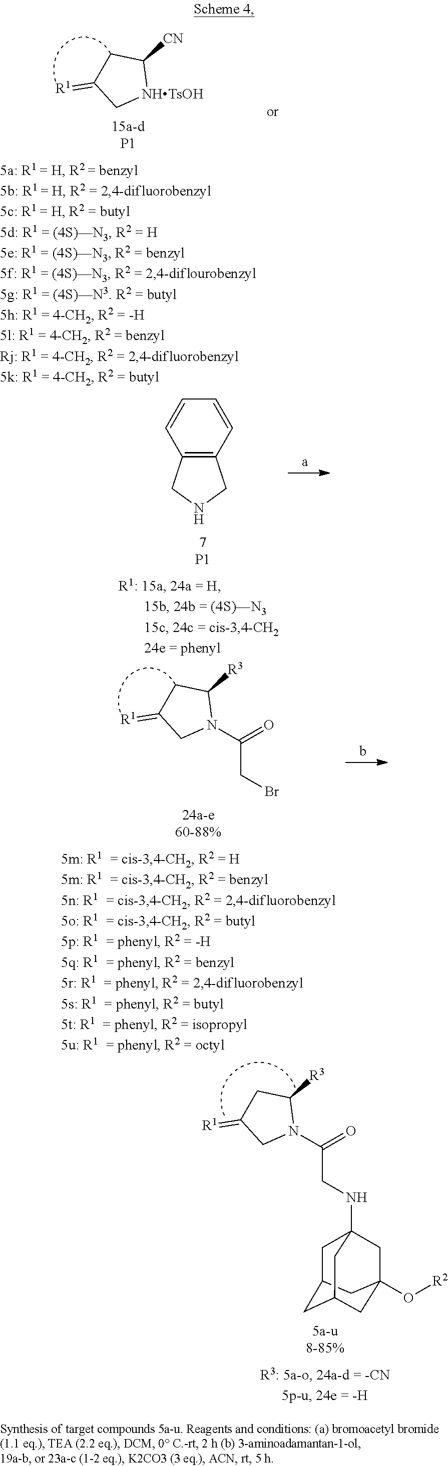

### Materials and Methods

Reagents were obtained from commercial sources and were used without further purification. Characterization of all compounds was done with 1H and 13C NMR and mass spectrometry. 1H and 13C NMR spectra were recorded on a 400 MHz Bruker Avance III Nanobay spectrometer with Ultrashield working at 400 and 100 MHz, respectively, and analyzed by use of MestReNova analytical chemistry software. Chemical shifts are in ppm, and coupling constants are in hertz (Hz). The UPLC (ultra performance liquid chromatography), used to quantify the purity of the products was an ACQUITY UPLC H-Class system with a TUV detector Waters coupled to an MS detector Waters QDa. An Acquity UPLC BEH C18 1.7 μm (2.1 mm×50 mm) column was used and as eluent a mixture of 0.1% FA in H2O, 0.1% FA in MeCN, H2O, and ACN. The wavelengths for UV detection were 254 and 214 nm. Key target compounds for the activity were analyzed by high resolution mass spectrometry: 10 μL of each sample (concentration=10-5 M) was injected using the CapLC system (Waters, Manchester, UK) and electrosprayed using a standard electrospray source. Samples were injected with an interval of 5 min. Positive ion mode accurate mass spectra were acquired using a Q-TOF II instrument (Waters, Manchester, UK). The MS was calibrated prior to use with a 0.2% H3PO4 solution. The spectra were lock mass corrected using the known mass of the nearest H3PO4 cluster. When necessary, flash column chromatography was performed on a Biotage ISOLERA One flash system equipped with an internal variable dual-wavelength diode array detector (200-400 nm). For normal phase purifications, Biotage Sf8r cartridges (5-100 g, flow rate of 10-100 mL/min) were used, and reverse phase purifications were done making use of Buchi C18 cartridges (4-30 g, flow rate of 10-50 mL/min). Dry sample loading was done by self-packing sample cartridges using Celite 545. Gradients used varied for each purification. Preparative HPLC purifications were carried out using a Waters HPLC system equipped with a UV and MS detector and using an XBridge Prep C18 5 μm OBD column (19×100 mm).

The following sections comprise the synthetic procedures and analytical data for all compounds reported in this manuscript. Every reaction was performed under N2 atmosphere if not stated otherwise. Several synthetic procedures that were used in the preparation of intermediates and final products are summarized here as “General Procedures”. Target compounds were obtained with a purity >95% and as amorphous solids unless stated otherwise.

The pyrrolidine P1 intermediates 13a-d 14a-d, and 15a-d from Scheme 1 have been prepared according to known procedures and are described in general procedures A-C. General procedures D-G describe the preparation of the O-substituted adamantyl P2 fragment from Schemes 2-3. General procedures H-I contain the protocols for the two-step assembly of the final compounds from Scheme 4.

General procedure A. To a solution of the Boc-protected proline analog (12a-d) in anhydrous DCM (0.2 M) was added DCC (1.1 eq.) followed by N-Hydroxysuccinimide (1.1 eq.). The reaction mixture was stirred for 30 min. and then a 7 N NH3 solution in MeOH (2 eq.) was added at 0° C. and the reaction was stirred for 1 h. After reaction completion, the volatiles was evaporated under reduced pressure. The residue was redissolved in cold EtoAC and filtered through a pad of celite. The filtrate was washed with a saturated sodium bicarbonate solution and the organic fraction was dried over sodium sulfate and concentrated under reduced pressure.

General procedure B. The prolinamide (13a-d) is suspended in anhydrous DCM (0.25 M) and anhydrous pyridine (10 eq.) is added. The reaction is cooled down to −15° C. and a solution of trifluoroacetic anhydride (1.3 eq.) in DCM (1 M) is added dropwise over 30 min. The reaction is stirred for 2 h, followed by a wash with 1 M solution of aqueous hydrochloric acid (3×10 mL). The organic layer was washed successively with brine (5 mL) and a saturated aqueous solution of sodium bicarbonate (5 mL). After drying over sodium sulfate, the organic layer was evaporated under reduced pressure, yielding the crude product.

General procedure C. The cyanopyrrolidne (14a-d) is dissolved in ACN and pTsOH·H2O (1.4 eq.) is added at 0° C. After 30 min. the reaction is allowed to reach rt and left stirring for 24 h. The reaction mixture was evaporated to dryness and washed with ethyl acetate to yield an off-white solid.

General procedure D. 2-(-3-hydroxyadamantan-1-yl)isoindoline-1,3-dione (17b) was dissolved in DMF (0.25 M) and cooled down to 0° C. Sodium hydride (1.1 eq.) was added to the solution. After 30 min, the desired bromide (3 eq.) was added dropwise and the reaction was left stirring overnight. The reaction mixture was quenched with MeOH, diluted with EtOAc, and washed with water. The organic phase was dried over sodium sulfate and evaporated under reduced pressure. Diethyl ether was added, the mixture was filtered and the filtrate dried. The crude was purified with flash chromatography on SiO2 with EtOAc in heptane (0-40%).

General procedure E. Compounds 19a-b were dispensed in isopropanol:water mixture (0.85:0.15) (0.2 M). sodium borohydride (5 eq.) was added to this solution. The reaction was left stirring for 24 h, cooled to 0° C. and then glacial acetic acid (0.2 M) was added carefully to pH=4 with AcOH. When the foaming subsided, the flask was stoppered and heated to 80° C. for 2 h. The crude reaction mixture was then carefully quenched with a 1 M NaOH solution and extracted to EtOAc.

General procedure F. 3-((tert-butoxycarbonyl)amino)adamantan-1-yl methanesulfonate (21) is mixed with the desired alcohol and DIPEA in a pressure tube. The mixture was heated to reflux for 2 hours. After that, the volatiles was evaporated and the product was purified with flash chromatography on SiO2 to obtain the desired boc protected adamantyl ether (22a-c).

General procedure G. The boc-protected 3-O-substituted adamantan-1-amines (21a-c) were dissolved in DCM (0.5 M) and trifluoroacetic acid (10 eq.) was added. After 2 h, the volatiles was dried and TFA was co-evaporated with toluene to yield the desired 3-O-substituted adamantan-1-amines (23a-c).

General procedure H. The cyanopyrrolidine salt (15a-d) or commercial isoindoline is suspended in anhydrous DCM (0.25 M) and cooled down to 0° C. Dry triethylamine (1.2 eq.) is added followed by bromoacetic bromide (1.2 eq.). The reaction was stirred for 3 h, then the reaction mixture was diluted with DCM and washed with a saturated sodium bicarbonate solution (3×10 mL). The organic phase was dried over sodium sulfate, concentrated under reduced pressure, and purified with flash chromatography on SiO2 with EtOAc in heptane (0-30%).

General procedure I. In a round bottom flask, 3-O-substituted adamantan-1-amine (19a-b, 23a-c) or 3-aminoadamantan-1-ol (16) was dispensed in ACN (0.25 M). Potassium carbonate (3 eq.) was added to the flask and the reaction mass was stirred. 24a-d (0.5-1 eq.) was added in 4 portions with 60 min intervals. The reaction mass was filtered and the filtrate was dried under a vacuum. Further purification is described individually.

tert-butyl (S)-2-carbamoylpyrrolidine-1-carboxylate (13a). General procedure A with (tert-butoxycarbonyl)-L-proline (2.6 g, 12.1 mmol) to yield tert-butyl (S)-2-carbamoylpyrrolidine-1-carboxylate (1.61 g, 7.51 mmol, 62% yield) as a white solid. MS (ESI) m\z 115.1 [M-Boc+H]⁺, 215.2 [M+H]⁺, 237.26 [M+Na]⁺. Characterization consistent with previously reported data.

tert-butyl (2S,4S)-4-azido-2-carbamoylpyrrolidine-1-carboxylate (13b). General procedure A with (2S,4S)-4-azido-1-(tert-butoxycarbonyl)pyrrolidine-2-carboxylic acid (0.397 g, 1.410 mmol) to yield tert-butyl (2S,4S)-4-azido-2-carbamoylpyrrolidine-1-carboxylate (0.302 g, 1.183 mmol, 84% yield) as a white solid. MS (ESI) m/z=155.1 [M-Boc+H]⁺, 256.2 [M+H]⁺, 278 [M+Na]⁺. Characterization consistent with previously reported data.

tert-butyl (S)-2-carbamoyl-4-methylenepyrrolidine-1-carboxylate (13C). General procedure A with (S)-1-(tert-butoxycarbonyl)-4-methylenepyrrolidine-2-carboxylic acid (2.00 g, 8.80 mmol) to yield tert-butyl (S)-2-carbamoyl-4-methylenepyrrolidine-1-carboxylate (1.821 g, 8.05 mmol, 91% yield) as a white solid. MS (ESI) m/z=127.2 [M-Boc+H]⁺, 249.2 [M+Na]⁺.

tert-butyl (1R,2S,5S)-2-carbamoyl-3-azabicyclo[3.1.0]hexane-3-carboxylate (13d). General procedure A with (1R,2S,5S)-3-(tert-butoxycarbonyl)-3-azabicyclo[3.1.0]hexane-2-carboxylic acid (1.30 g, 5.72 mmol). Further purification was performed by flash chromatography on SiO2 with MeOH in DCM (2-10%) to obtain tert-butyl (1R,2S,5S)-2-carbamoyl-3-azabicyclo[3.1.0]hexane-3-carboxylate (642 mg, 2.84 mmol, 50% yield) as a white solid. MS (ESI) m/z=249.27 [M+Na]⁺.

tert-butyl (S)-2-cyanopyrrolidine-1-carboxylate (14a). General procedure B with tert-butyl (S)-2-carbamoylpyrrolidine-1-carboxylate (13a) (1.1 g, 5.13 mmol) to yield tert-butyl (S)-2-cyanopyrrolidine-1-carboxylate (0.98 g, 5.00 mmol, 97%) as a yellow solid. MS (ESI) m/z=197.3 [M+H]⁺, 217.3 [M+Na]⁺. Characterization consistent with previously reported data.[27a, 27b]

tert-butyl (2S,4S)-4-azido-2-cyanopyrrolidine-1-carboxylate (14b). General procedure B with tert-butyl (2S,4S)-4-azido-2-carbamoylpyrrolidine-1-carboxylate (13b) (0.302 g, 1.183 mmol) to yield tert-butyl (2S,4S)-4-azido-2-cyanopyrrolidine-1-carboxylate (0.279 g, 1.176 mmol, 99% yield) as an off-white solid. MS (ESI) m\z 238.2 [M+H]⁺.

tert-butyl (S)-2-cyano-4-methylenepyrrolidine-1-carboxylate (14c). General procedure B with tert-butyl (S)-2-carbamoyl-4-methylenepyrrolidine-1-carboxylate (13c) (1.64 g, 7.25 mmol) to yield tert-butyl (S)-2-cyano-4-methylenepyrrolidine-1-carboxylate (1.42 g, 6.82 mmol, 94% yield) as a white solid. MS (ESI) m/z=127.2 [M-Boc+H]⁺, 249.2 [M+Na]⁺.

tert-butyl (1R,2S,5S)-2-cyano-3-azabicyclo[3.1.0]hexane-3-carboxylate (14d). General procedure B with tert-butyl (1R,2S,5S)-2-carbamoyl-3-azabicyclo[3.1.0]hexane-3-carboxylate (13d) (530 mg, 2,342 mmol) to yield tert-butyl (1R,2S,5S)-2-cyano-3-azabicyclo[3.1.0]hexane-3-carboxylate (486 mg, 2.334 mmol, 100% yield) as an off-white solid. MS (ESI) m/z=209.2 [M+H]⁺, 231.1 [M+Na]⁺.

(S)-pyrrolidine-2-carbonitrile 4-methylbenzenesulfonate (15a). General procedure C with (2S,4S)-tert-butyl 2-cyano-4-fluoropyrrolidine-1-carboxylate (14a) (0.40 g, 1.867 mmol) to yield (S)-pyrrolidine-2-carbonitrile 4-methylbenzenesulfonate (1.09 g, 4.06 mmol, 81% yield) as a white solid. 1H NMR (400 MHz, DMSO-d6) δ 9.80 (s, 2H), 7.52-7.47 (dd, J=8.5, 0.5 Hz, 2H), 7.13 (dd, J=8.5, 0.5 Hz, 2H), 4.73 (t, J=7.5 Hz, 1H), 3.37-3.19 (m, 2H), 2.39-2.32 (m, 1H), 2.29 (s, 3H), 2.19-2.05 (m, 1H), 2.06-1.87 (m, 2H). 13C NMR (101 MHz, DMSO-d6) δ 145.35, 137.91, 128.19, 125.52, 116.44, 46.28, 45.54, 29.55, 23.09, 20.85. Characterization consistent with previously reported data.[27b]

(2S,4S)-4-azido-2-cyanopyrrolidin-1-ium 4-methylbenzenesulfonate (15b). General procedure C with tert-butyl (2S,4S)-4-azido-2-cyanopyrrolidine-1-carboxylate (14b) (279 mg, 1.176 mmol) to yield (2S,4S)-4-azido-2-cyanopyrrolidin-1-ium 4-methylbenzenesulfonate (0.224 g, 0.724 mmol, 62% yield) as off-white solid. MS (ESI) m\z=138.2 [M-TsOH]⁺.

(S)-2-cyano-4-methylenepyrrolidin-1-ium 4-methylbenzenesulfonate (15c). General procedure C with tert-butyl (S)-2-cyano-4-methylenepyrrolidine-1-carboxylate (14c) (1.40 g, 6.72 mmol) to yield (S)-2-cyano-4-methylenepyrrolidin-1-ium 4-methylbenzenesulfonate (1.52 g, 5.43 mmol, 81% yield) as an off-white solid. MS (ESI) m\z 109.2 [M-TsOH+H]⁺.

(1R,2S,5S)-3-azabicyclo[3.1.0]hexane-2-carbonitrile 4-methylbenzenesulfonate (15d). General procedure C with tert-butyl (1R,2S,5S)-2-cyano-3-azabicyclo[3.1.0]hexane-3-carboxylate (14d) (450 mg, 2.163 mmol) to yield the tert-butyl (1R,2S,5S)-3-azabicyclo[3.1.0]hexane-2-carbonitrile 4-methylbenzenesulfonate (400 mg, 1.427 mmol, 66.v0% yield) as a white solid. MS (ESI) m\z 109.2 [M-TsOH+H]⁺.

2-(3-hydroxyadamantan-1-yl)isoindoline-1,3-dione (17). In a round bottom flask equipped with a Dean-Stark apparatus, a mixture of 3-aminoadamantan-1-ol (2.00 g, 11.96 mmol), isobenzofuran-1,3-dione (1.77 g, 11.96 mmol), and triethylamine (1.99 mL, 14.35 mmol) in toluene (20 mL) was heated at 125° C. for 18 h. The volatiles were dried to yield 2-(-3-hydroxyadamantan-1-yl)isoindoline-1,3-dione (3.30 g, 93% yield) as a white solid. MS (ESI) m/z=280.0 [M−H2O+H]⁺.

2-(-3-(benzyloxy)adamantan-1-yl)isoindoline-1,3-dione (18a). General procedure D with 2-(3-hydroxyadamantan-1-yl)isoindoline-1,3-dione (17) (3.00 g, 10.09 mmol) and (bromomethyl)benzene (3.62 mL, 30.3 mmol) to yield 2-(-3-(benzyloxy)adamantan-1-yl)isoindoline-1,3-dione (1.94 g, 5.01 mmol, 50% yield). MS (ESI) 280.1 [M-phenol]⁺.

2-(-3-((2,4-difluorobenzyl)oxy)adamantan-1-yl)isoindoline-1,3-dione (18b). General procedure D with with 2-(-3-hydroxyadamantan-1-yl)isoindoline-1,3-dione (17) (1.5 g, 5.04 mmol) and 2,4-Difluorobenzyl bromide (2.59 mL, 20.18 mmol) to yield 2-(-3-((2,4-difluorobenzyl)oxy)adamantan-1-yl)isoindoline-1,3-dione (1.05 g, 2.489 mmol, 49% yield). MS (ESI) m/z=280.1 [M-difluorobenzophenol]⁺, 441.1 [M+H2O]+

3-(benzyloxy)adamantan-1-amine (19a). General procedure E with 2-(-3-(benzyloxy)adamantan-1-yl)isoindoline-1,3-dione (18a) (1.9 g, 4.90 mmol). The reaction crude was dried under vacuum. The crude was redisolved in EtOAc and extracted with 1M HCl solution. After the impurities were extracted to the organic phase, the pH of the aqeuous phase was raised to 12 with a NaOH solution and the product was extracted with EtOAc to yield 3-(benzyloxy)adamantan-1-amine (620 mg, 2.020 mmol, 49% yield) as a yellow oil. MS (ESI) m/z=258.1=[M+H]⁺.

3-((2,4-difluorobenzyl)oxy)adamantan-1-amine (19b). General procedure E 2-(3-((2,4-difluorobenzyl)oxy)adamantan-1-yl)isoindoline-1,3-dione (18b) (0.89 g, 2.10 mmol). The organic layer was dried under vacuum and purified by flash chromatography on SiO2 with EtOAc (+1% triethylamine) in heptane (20-70%) to give 3-((2,4-difluorobenzyl)oxy)adamantan-1-amine (580 mg, 1.98 mmol, 94% yield) as a yellow oil. MS (ESI) m/z=294.2 [M+H]⁺.

tert-butyl (3-hydroxyadamantan-1-yl)carbamate (20). To a solution of 3-aminoadamantan-1-ol (1 g, 5.98 mmol) in DCM (19.93 mL), Triethylamine (0.829 mL, 5.98 mmol) was added followed by the addition of Di-t-butyldicarbonate (1.305 g, 5.98 mmol) portionwise. The reaction mixture was stirred for 3 h, then the volatiles were evaporated under reduced pressure. The residue was redissolved in EtOAc (150 mL) and washed with a 1 M solution of HCl (2×50 mL). The organic phase was dried over sodium sulfate and further concentrated under reduced pressure. The resulting crude was washed with ether yielding the title compound tert-butyl (3-hydroxyadamantan-1-yl)carbamate (1.376 g, 5.15 mmol, 86% yield) as a white powder. MS (ESI) m/z=150.2 [M−Boc−H2O]⁺, 194.2 [M-tert-BuOH+H]⁺. Characterization consistent with previously reported data.[29]

3-((tert-butoxycarbonyl)amino)adamantan-1-yl methanesulfonate (21). tert-butyl (-3-hydroxyadamantan-1-yl)carbamate (20) (3.0 g, 11.22 mmol) was dissolved in anhydrous DCM (45 mL). DIPEA 4.30 mL, 24.68 mmol) and 4-Dimethylaminopyridine (0.137 g, 1.122 mmol) were added to the solution at 0° C.

Methanesulfonylchloride (2.171 mL, 28.1 mmol) was added dropwise to the solution and the reaction was stirred for 2 h at rt. The volatiles were evaporated, the crude was dissolved in ethyl acetate (150 mL) and washed with HCl 1M solution and brine. After drying over sodium sulfate, the volatiles were evaporated, yielding 3-((tert-butoxycarbonyl)amino)adamantan-1-yl methanesulfonate (4.19 g, 11.05 mmol, 98% yield) as an orange oil. It was used in the next step without further purification. MS (ESI) m/z=150.2 [M−Boc−H2O]⁺, 194.2 [M-BuOH-methanosulfonic acid+H2O+H]⁺ 368.3 [M+Na]⁺. Characterization consistent with previously reported data.[29]

tert-butyl (3-butoxyadamantan-1-yl)carbamate (22a). General procedure F with butan-1-ol (2.5 mL, 30.7 mmol) to yield tert-butyl (3-butoxyadamantan-1-yl)carbamate (294 mg, 0.909 mmol, 63% yield). MS (ESI) m/z=150.1 [M-BuOH-Boc+H]⁺, 194.1 [M-n-BuOH-tert-BuOH+H2O+H]⁺, 235.2 [M+Na]⁺.

tert-butyl (3-isopropoxyadamantan-1-yl)carbamate (22b). General procedure F with propan-2-ol (40.5 mmol, 2.43 g) to obtain tert-butyl (3-isopropoxyadamantan-1-yl)carbamate (195 mg, 0.630 mmol, 16% yield) as a colourless oil. MS (ESI) m/z=194.1 [M-tert-BuOH-propanol+H2O+H]⁺.

tert-butyl 3-(octyloxy)adamantan-1-yl)carbamate (22c). General procedure F with octan-1-ol (985 μl, 6.28 mmol). The product was purified with flash chromatography on SiO2 (2% MeOH in DCM) to obtain the desired tert-butyl 3-(octyloxy)adamantan-1-yl)carbamate (182 mg, 0.479 mmol, 76% yield) as a colorless oil. MS (ESI) m/z=150.1 [M−BuOH−Boc+H]⁺, 194.1 [M-tert-BuOH-octanol+H2O+H]⁺.

3-butoxyadamantan-1-amine (23a). General procedure G with tert-butyl (3-butoxyadamantan-1-yl)carbamate (22a) (290 mg, 0.807 mmol) to yield 3-butoxyadamantan-1-aminium 2,2,2-trifluoroacetate (260 mg, 0.771 mmol, 96% yield). TFA salt. MS (ESI) m/z=223.3 [M+H]⁺.

3-isopropoxyadamantan-1-amine (23b). General procedure G with tert-butyl (3-isopropoxyadamantan-1-yl)carbamate (22b) (185 mg, 0.598 mmol) to yield 3-isopropoxyadamantan-1-amine 2,2,2-trifluoroacetate (138 mg, 0.427 mmol, 71% yield). MS (ESI) m/z=210.1 [M+H]⁺.

3-(octyloxy)adamantan-1-amine (23c). General procedure G with tert-butyl (3-(octyloxy)adamantan-1-yl)carbamate (22c) (180 mg, 0.474 mmol) to yield 3-(octyloxy)adamantan-1-amine 2,2,2-trifluoroacetate (239 mg, 0.474 mmol, 100% yield). MS (ESI) m/z=380.4 [M+H]⁺.

(S)-1-(2-bromoacetyl)pyrrolidine-2-carbonitrile (24a). General procedure H with (S)-pyrrolidine-2-carbonitrile 4-methylbenzenesulfonate (15a) (542 mg, 2.020 mmol) and to yield (S)-1-(2-bromoacetyl)pyrrolidine-2-carbonitrile (320 mg, 1.474 mmol, 73% yield) as an orange oil. MS (ESI) m/z=218.1, 219.1 [M+H]⁺. Characterization consistent with previously reported data.[30]

(2S,4S)-4-azido-1-(2-bromoacetyl)pyrrolidine-2-carbonitrile (24b). General procedure H with (2S,4S)-4-azidopyrrolidine-2-carbonitrile 4-methylbenzenesulfonate (15b) (2.6 g, 8.40 mmol) to yield (2S,4S)-4-azido-1-(2-bromoacetyl)pyrrolidine-2-carbonitrile (1.91 g, 7.40 mmol, 88% yield) as a light brown solid. 1H NMR (400 MHz, DMSO-d6) δ 4.88 (dd, J=9.1, 1.5 Hz, 1H), 4.69 (ddd, J=4.6, 3.6, 2.0 Hz, 1H), 4.28-4.13 (m, 2H), 3.76-3.65 (m, 2H), 2.46 (ddd, J=14.0, 9.2, 4.7 Hz, 1H), 2.29 (dq, J=14.3, 1.4 Hz, 1H). 13C NMR (101 MHz, DMSO-d6) δ 165.24, 118.27, 60.18, 51.88, 45.12, 34.25, 28.69. MS (ESI) m/z=259.0, 260 [M+H]⁺.

(S)-1-(2-bromoacetyl)-4-methylenepyrrolidine-2-carbonitrile (24c). General procedure H with 4-methylenepyrrolidine-2-carbonitrile 4-methylbenzenesulfonate (15c) (1.34 g, 4.99 mmol). The product was purified by flash chromatography with EtOAc in heptane (20-60%) to yield S-1-(2-bromoacetyl)-4-methylenepyrrolidine-2-carbonitrile (904 mg, 3.95 mmol, 79% yield). MS (ESI) m/z=230.1, 231.1 [M+H]⁺.

(1R,2S,5S)-3-(2-bromoacetyl)-3-azabicyclo[3.1.0]hexane-2-carbonitrile (24d). General procedure H (1R,2S,5S)-3-azabicyclo[3.1.0]hexane-2-carbonitrile 4-methylbenzenesulfonate (15d) (400 mg, 1.427 mmol). Crude was purified with flash chromatography on SiO2 with EtoAc in heptane (10-60%) to yield (1R,2S,5S)-3-(2-bromoacetyl)-3-azabicyclo[3.1.0]hexane-2-carbonitrile (230 mg, 1.00 mmol, 70% yield). MS (ESI) m/z=229.0, 233.0 [M+H]⁺, 246.0, 248.0 [M+Na]⁺.

(S)-1-((-3-(benzyloxy)adamantan-1-yl)glycyl)pyrrolidine-2-carbonitrile (5a). General procedure I with 3-(benzyloxy)adamantan-1-amine (19a) (185 mg, 0.719 mmol) and (S)-1-(2-bromoacetyl)pyrrolidine-2-carbonitrile (100 mg, 0.461 mmol). After filtration, the mixture was dried and and purified with flash chromatography on Si—C18 with 10-100% ACN in water. The desired fractions were collected and dried. The product was dissolved in DCM and washed with a 1M HCl solution. The organic phase was dried with sodium sulphate and dried under reduced pressure to yield (S)-1-((3-(benzyloxy)adamantan-1-yl)glycyl)pyrrolidine-2-carbonitrile hydrochloride (41 mg, 0.095 mmol, 20.7% yield). HCl salt. 1H NMR (400 MHz, DMSO-d6) δ 8.97 (bs, 2H), 7.40-7.13 (m, 5H), 4.85 (dd, J=7.4, 4.1 Hz, 1H), 4.47 (s, 2H), 4.01 (q, J=16.4 Hz, 2H), 3.81-3.68 (m, 1H), 3.59-3.49 (m, 1H), 2.36-2.25 (m, 2H), 2.24-2.15 (m, 2H), 2.11-1.99 (m, 2H), 1.97 (s, 2H), 1.88-1.75 (m, 6H), 1.76-1.63 (m, 2H), 1.59-1.43 (m, 2H). 13C NMR (101 MHz, CDCl3) δ 164.81, 139.07, 128.33, 127.55, 127.36, 118.41, 73.66, 63.03, 60.15, 47.16, 46.20, 42.44, 41.13, 40.00, 37.72, 37.48, 34.44, 30.05, 29.71, 25.12. MS (ESI) m/z=394.4 [M+H]⁺. HRMS: calc. 394.2489, found 394.2496 [M+H]⁺.

(S)-1-((-3-((2,4-difluorobenzyl)oxy)adamantan-1-yl)glycyl)pyrrolidine-2-carbonitrile (5b). General procedure I with 3-((2,4-difluorobenzyl)oxy)adamantan-1-amine (19b) (71 mg, 0.245 mmol) and (S)-1-(2-bromoacetyl)pyrrolidine-2-carbonitrile (24a) (29.6 mg, 0.123 mmol). After filtration, product was dissolved in EtOAc and washed with water. Diethyl ether was added to the organic phase and the organic phase was extracted with 1M HCl solution. The acidic aqeuous layer was back-extracted with DCM and dried with sodium sulphate and then under reduced pressure. The product was precipitated with diethyl ether to yield er to (S)-1-((3-((2,4-difluorobenzyl)oxy)adamantan-1-yl)glycyl)pyrrolidine-2-carbonitrile hydrochloride (12 mg, 0,026 mmol, 21% yield) as a white powder. HCl salt. 1H NMR (400 MHz, DMSO-d6) (1 to 12 cis trans amide rotamers) δ 9.26-9.01 (m, 2H), 7.47 (td, J=8.6, 6.7 Hz, 1H), 7.22 (ddd, J=10.5, 9.4, 2.6 Hz, 1H), 7.12-7.03 (m, 1H), 5.39 (d, J=7.6 Hz, 1/13H), 4.85 (dd, J=7.5, 3.9 Hz, 12/13H), 4.48 (s, 2H), 4.12-3.88 (m, 2H), 3.73 (tt, J=7.7, 3.9 Hz, 1H), 3.61-3.50 (m, 1H), 2.34-2.30 (m, 2H), 2.28-2.12 (m, 2H), 2.12-2.00 (m, 2H), 1.99 (s, 2H), 1.86 (s, 4H), 1.81 (d, J=12.1 Hz, 2H), 1.67 (d, J=10.5 Hz, 2H), 1.50 (q, J=13.0 Hz, 2H). 13C NMR (101 MHz, DMSO-d6) (main rotamer) δ 165.29, 162.62 (dd, J=157.2, 12.3 Hz), 160.17 (dd, J=159.4, 12.3 Hz), 131.91 (dd, J=9.7, 6.1 Hz), 123.49-122.98 (m), 119.39, 111.75 (dd, J=20.9, 3.7 Hz), 104.14 (t, J=25.7 Hz), 74.13, 58.99, 56.30 (d, J=2.9 Hz), 46.79, 46.03, 41.81, 41.20, 36.83, 36.80, 34.45, 30.07, 29.96, 25.06. MS (ESI) m/z=430.4 [M+H]⁺ 452.4 [M+Na]⁺. HRMS: calc. 430.2301, found: 430.2316 [M+H]⁺.

(2S)-1-((-3-butoxyadamantan-1-yl)glycyl)pyrrolidine-2-carbonitrile (5c). General procedure I with 3-butoxyadamantan-1-amine (23a) (100 mg, 0.448 mmol) and (S)-1-(2-bromoacetyl)pyrrolidine-2-carbonitrile (24a) (97 mg, 0.448 mmol). Purified with flash chromatography on Si—C18 MeOH in water (10-100%), followed by preparative HPLC to yield (S)-1-((3-butoxyadamantan-1-yl)glycyl)pyrrolidine-2-carbonitrile (13 mg, 0.036 mmol, 8% yield). 1H NMR (400 MHz, MeOD) δ (5 to 1 mixture of trans to cis amide rotamers) 5.06 (dd, J=7.1, 2.7 Hz, 1/6H), 4.80 (dd, J=6.3, 4.7 Hz, 5/6H), 3.87-3.75 (m, 2H), 3.75-3.67 (m, 5/6H), 3.67-3.62 (m, 1/6H), 3.53 (dt, J=9.7, 7.6 Hz, 1H), 3.46 (t, J=6.4 Hz, 2H), 2.40-2.31 (m, 2H), 2.31-2.24 (m, 2H), 2.23-2.17 (m, 2H), 1.85-1.69 (m, 10H), 1.61 (d, J=3.1 Hz, 2H), 1.55-1.43 (m, 2H), 1.43-1.32 (m, 2H), 0.92 (t, J=7.3 Hz, 3H). 13C NMR (101 MHz, MeOD) (main rotamer) δ 168.43, 119.40, 74.33, 61.11, 58.04, 47.89, 46.79, 44.86, 42.79, 41.13, 39.75, 35.83, 33.69, 31.71, 31.03, 30.69, 26.02, 20.39, 14.27. MS (ESI) m/z=360.3 [M+H]⁺. HRMS: calc 360.2646, found 360.2632 [M+H]⁺.

(2S,4S)-4-azido-1-((-3-hydroxyadamantan-1-yl)glycyl)pyrrolidine-2-carbonitrile (5d). General procedure I with 3-aminoadamantan-1-ol (130 mg, 0.775 mmol) and (2S,4S)-4-azido-1-(2-bromoacetyl)pyrrolidine-2-carbonitrile (24b) (100 mg, 0.387 mmol) The product was purified by flash chromatography on SiO2 with 0-20% MeOH in EtOAc to yield (2S,4S)-4-azido-1-((3-hydroxyadamantan-1-yl)glycyl)pyrrolidine-2-carbonitrile (113 mg, 0.328 mmol, 85% yield) as a white solid. 1H NMR (400 MHz, CDCl3) (3 to 1 mixture of trans to cis amide rotamers) δ 5.13 (t, J=5.0 Hz, 1/4H), 4.89 (t, J=5.4 Hz, 3/4H), 4.49 (d, J=4.0 Hz, 3/4H), 4.42 (d, J=4.0 Hz, 1/4H), 3.87-3.57 (m, 10/4H H), 3.54-3.35 (m, 6/4H), 2.52 (t, J=4.0 Hz, 3/4H), 2.48-2.42 (m, 5/4), 2.46 (t, J=4.2 Hz, 5/4H), 2.42-2.18 (m, 3H) 1.79-1.38 (m, 12H). 13C NMR (101 MHz, DMSO-d6) (main rotamer) 6 170.83, 118.73, 67.73, 60.15, 52.98, 50.70, 49.84, 44.67, 44.40, 43.19, 40.80, 35.05, 34.19, 30.19. MS (ESI) m/z=345.2 [M+H]⁺ 367.2 [M+Na]⁺ 389.3 [M+FA-H]−. HRMS: calc. 345.2033, found 345.2038 [M+H]⁺.

(2S,4S)-4-azido-1-((3-(benzyloxy)adamantan-1-yl)glycyl)pyrrolidine-2-carbonitrile (5e). General procedure I with 3-(benzyloxy)adamantan-1-amine (19a) (115 mg, 0.450 mmol) and (2S,4S)-4-azido-1-(2-bromoacetyl)pyrrolidine-2-carbonitrile (24b) (116 mg, 0.450 mmol). The reaction crude was purified on Si—C18 with MeOH (+1% FA) in water (10-100%) to yield (2S,4S)-4-azido-1-((3-(benzyloxy)adamantan-1-yl)glycyl)pyrrolidine-2-carbonitrile, formate salt (100 mg, 0.208 mmol, 46% yield) as a white powder. 1H NMR (400 MHz, CDCl3) (4 to 1 mixture of trans to cis amide rotamers) δ 8.26 (s, 1H), 7.36-7.29 (m, 4H), 7.29-7.23 (m, 1H), 6.23 (bs, 2H), 5.11 (d, J=8.4 Hz, 1/5H), 4.82 (dd, J=8.8, 2.4 Hz, 4/5H), 4.50 (s, 2H), 4.49-4.40 (m, 4/5H), 4.38-4.34 (m, 1/5H), 4.02 (d, J=15.2 Hz, 1/5H), 3.89-3.71 (m, 9/5H), 3.71-3.53 (m, 2H), 2.63-2.51 (m, 1/5H), 2.49-2.30 (m, 9/5H), 1.99-1.71 (m, 10H), 1.65-1.51 (m, 2H). 13C NMR (101 MHz, CDCl3) (main rotamer) δ 185.77, 184.88, 157.40, 146.54, 145.72, 145.56, 135.60, 91.93, 81.16, 78.15, 75.20, 69.37, 63.35, 62.34, 60.05, 58.44, 57.68, 57.40, 53.00, 52.63, 48.35. MS (ESI) m/z=435.3 [M+H]⁺, 457.3 [M+Na]⁺. HRMS: calc. 435.2503, found 435.2498 [M+H]⁺.

(2S,4S)-4-azido-1-((3-((2,4-difluorobenzyl)oxy)adamantan-1-yl)glycyl)pyrrolidine-2-carbonitrile (5f). General procedure I with 3-((2,4-difluorobenzyl)oxy)adamantan-1-amine (19b) (100 mg, 0.341 mmol) and (2S,4S)-4-azido-1-(2-bromoacetyl)pyrrolidine-2-carbonitrile (24b) (88 mg, 0.341 mmol). The product was precipitated from EtOAc to yield (2S,4S)-4-azido-1-((3-((2,4-difluorobenzyl)oxy)adamantan-1-yl)glycyl)pyrrolidine-2-carbonitrile (74 mg, 0.157 mmol, 46% yield) as a white powder. 1H NMR (400 MHz, CDCl3) (3 to 1 mixture of trans to cis amide rotamers) 7.45-7.32 (m, 1H), 6.84 (tdd, J=8.3, 2.5, 1.0 Hz, 1H), 6.76 (ddd, J=10.2, 8.9, 2.5 Hz, 1H), 5.13 (dd, J=6.3, 3.7 Hz, 1/4H), 4.85 (dd, J=6.9, 4.0 Hz, 3/4H), 4.50 (s, 2H), 4.49-4.42 (m, 1/4H), 4.40-4.36 (m, 3/4H), 3.81-3.55 (m, 5/2H), 3.55-3.33 (m, 3/2H), 2.80 (s, 1H), 2.54-2.47 (m, 1/2H), 2.48-2.39 (m, 3/2H), 2.36-2.29 (m, 2H), 1.85-1.71 (m, 6H), 1.71-1.57 (m, 4H), 1.55 (s, 2H). 13C NMR (101 MHz, CDCl3) (main rotamer) δ 170.08, 162.71 (dd, J=187.8, 12.0 Hz), 160.25 (dd, J=189.1, 12.0 Hz), 130.89 (dd, J=9.5, 6.1 Hz), 122.66 (d, J=14.7 Hz), 117.33, 111.20 (dd, J=21.0, 3.6 Hz), 103.65 (t, J=25.4 Hz), 74.53, 60.05, 56.16 (d, J=3.6 Hz), 54.22, 51.23, 45.99, 45.08, 43.55, 41.30, 41.11, 40.54, 35.40, 34.78, 30.52. MS (ESI) m/z=471.3 [M+H]⁺, 493.3 [M+Na]⁺. HRMS: calc. 471.2315, found 471.2304 [M+H]⁺.

(2S,4S)-4-azido-1-((3-butoxyadamantan-1-yl)glycyl)pyrrolidine-2-carbonitrile (5g). General procedure I with 3-butoxyadamantan-1-amine (23a) (87 mg, 0.387 mmol) and (2S,4S)-4-azido-1-(2-bromoacetyl)pyrrolidine-2-carbonitrile (24b) (100 mg, 0.387 mmol). Reaction was purified with preparative HPLC to yield (2S,4S)-4-azido-1-((3-butoxyadamantan-1-yl)glycyl)pyrrolidine-2-carbonitrile (17 mg, 0,042 mmol, 11% yield). MS (ESI) m/z=401.3 [M+H]⁺, 423.3 [M+Na]⁺. HRMS: calc. 401.2659, found 401.2656 [M+H]⁺.

(2S)-1-((3-hydroxyadamantan-1-yl)glycyl)-4-methylenepyrrolidine-2-carbonitrile (5 h). General procedure I with 3-aminoadamantan-1-ol (75 mg, 0.448 mmol) and (S)-1-(2-bromoacetyl)-4-methylenepyrrolidine-2-carbonitrile (103 mg, 0.448 mmol). The product was precipitated from EtOAc to yield (S)-1-((3-hydroxyadamantan-1-yl)glycyl)-4-methylenepyrrolidine-2-carbonitrile (24c) (63 mg, 0.200 mmol, 45% yield) as a white powder. 1H NMR (400 MHz, MeOD) (4 to 1 mixture of trans to cis amide rotamers) δ 5.28-5.17 (m, 11/5H), 4.99 (dd, J=9.1, 2.2 Hz, 4/5H), 4.31-4.16 (m, 9/5H), 4.03 (dd, J=15.8, 1.9 Hz, 1/5H), 3.58 (q, J=16.4 Hz, 2/5H), 3.45 (s, 8/5H), 3.17-3.09 (m, 1/5H), 3.08-2.97 (m, 4/5H), 2.94-2.78 (m, 1H), 2.29-2.19 (m, 2H), 1.76-1.50 (m, 12H). 13C NMR (101 MHz, MeOD) (main rotamer) δ 171.99, 142.88, 119.34, 110.63, 69.92, 54.75, 50.26, 50.01, 47.55, 44.94, 41.58, 41.51, 37.01, 36.14, 32.07. MS (ESI) m/z=316.2 [M+H]⁺, 338.2 [M+Na]⁺. HRMS: calc. 316.2020, found 316.2018 [M+H]⁺.

(S)-1-((3-(benzyloxy)adamantan-1-yl)glycyl)-4-methylenepyrrolidine-2-carbonitrile (5i). General procedure I with 3-(benzyloxy)adamantan-1-amine (19a) (112 mg, 0.437 mmol) and (S)-1-(2-bromoacetyl)-4-methylenepyrrolidine-2-carbonitrile (24c) (50 mg, 0.218 mmol). After filtration the solution was dried, redisolved in EtOAc and washed with water, then purified with flash chromatography on SiO2 with MeOH in EtOAc (0-15%) to yield (S)-1-((3-(benzyloxy)adamantan-1-yl)glycyl)-4-methylenepyrrolidine-2-carbonitrile (59 mg, 0.145 mmol, 66.7%). 1H NMR (400 MHz, CDCl3 (mixture of 4 to 1 trans to cis amide rotamers) δ 7.38-7.07 (m, 5H), 5.16 (dt, J=16.0, 2.1 Hz, 2H), 5.01 (d, J=7.3 Hz, 1/5H), 4.88 (dd, J=8.5, 2.8 Hz, 4/5H), 4.43 (s, 2H), 4.28-3.94 (m, 2H), 3.50 (d, J=8.1 Hz, 2/5H), 3.36 (s, 8/5H), 3.04-2.94 (m, 2/5H), 2.93-2.68 (m, 8/5H), 2.33-2.19 (m, 2H), 2.06 (bs, 1H), 1.85-1.63 (m, 9H), 1.61-1.45 (m, 7H). 13C NMR (101 MHz, CDCl3) (main rotamer) δ 170.49, 140.28, 139.68, 128.32, 127.52, 127.22, 111.10, 74.19, 62.81, 53.50, 49.28, 46.36, 46.16, 43.41, 41.60, 41.43, 40.74, 35.99, 35.48, 30.52. MS (ESI) m/z=406.3 [M+H]⁺ 428.3 [M+Na]⁺. HRMS: calc. 406.2489, found 406.2501 [M+H]⁺.

(2S)-1-((3-((2,4-difluorobenzyl)oxy)adamantan-1-yl)glycyl)-4-methylenepyrrolidine-2-carbonitrile (5j). General procedure I with 3-((2,4-difluorobenzyl)oxy)adamantan-1-amine (19b) (150 mg, 0,511 mmol) and (S)-1-(2-bromoacetyl)-4-methylenepyrrolidine-2-carbonitrile (24c) (117 mg, 0.511 mmol). After filtration crude was redisolved in EtOAc and washed with water, then purified with flash chromatography on SiO2 with EtOAc in heptane (80-100%) to yield (S)-1-((3-((2,4-difluorobenzyl)oxy)adamantan-1-yl)glycyl)-4-methylenepyrrolidine-2-carbonitrile (34 mg, 0.077 mmol, 15% yield). 1H NMR (400 MHz, MeOD) (4 to 1 mixture of trans to cis amide rotamers) δ 7.51-7.38 (m, 1H), 6.98-6.85 (m, 2H), 5.27-5.20 (m, 11/5H), 4.99 (dd, J=9.0, 2.3 Hz, 4/5H), 4.54 (s, 2H), 4.30-4.16 (m, 9/5H), 4.06-3.92 (m, 1/5H), 3.67-3.50 (m, 2/5H), 3.47 (s, 8/5H), 3.17-3.07 (m, 1/5H), 3.07-2.94 (m, 4/5H), 2.91 (d, J=15.5 Hz, 1/5H), 2.85 (d, J=15.7 Hz, 4/5H), 2.36-2.22 (m, 2H), 1.89-1.71 (m, 6H), 1.71-1.53 (m, 6H).) 13C NMR (101 MHz, MeOD) (main rotamer) δ 172.04, 164.24 (dd, J=174.2, 12.1 Hz), 161.78 (dd, J=175.7, 12.2 Hz), 142.86, 132.66 (dd, J=9.6, 5.9 Hz), 124.09 (dd, J=14.9, 3.9 Hz), 119.47, 112.11 (dd, J=21.2, 3.8 Hz), 110.80, 104.38 (t, J=25.8 Hz), 75.93, 57.15 (d, J=3.7 Hz), 54.93, 50.36, 47.64, 46.71, 43.40, 41.82, 41.80, 41,58, 37.05, 36.40, 32.00. MS (ESI) m/z=442.3 [M+H]⁺, 883.5 [2M+H]+

(S)-1-((-3-butoxyadamantan-1-yl)glycyl)-4-methylenepyrrolidine-2-carbonitrile (5k). General procedure I with 3-butoxyadamantan-1-amine 2,2,2-trifluoroacetate (23a) (147 mg, 0.437 mmol) and (S)-1-(2-bromoacetyl)-4-methylenepyrrolidine-2-carbonitrile (24c) (50 mg, 0.218 mmol) After filtration, crude was dissolved in EtOAc and washed with water, then dried over sodium sulphate and dried under reduced pressure, followed by purification with flash chromatography on Si—C18 with ACN in water (30-70%) to yield (S)-1-((3-butoxyadamantan-1-yl)glycyl)-4-methylenepyrrolidine-2-carbonitrile (34 mg, 0.092 mmol, 42%). 1H NMR (400 MHz, CDCl3) (4 to 1 mixture of trans and cis amide rotamers) δ 5.29-5.17 (m, 2H), 5.14-5.08 (m, 1/5H), 4.95 (dd, J=8.5, 2.8 Hz, 4/5H), 4.32-4.08 (m, 2H), 3.58 (s, 2/5H), 3.43 (d, J=2.6 Hz, 8/5H), 3.38 (t, J=6.7 Hz, 2H), 3.11-3.01 (m, 1/5H), 2.99-2.77 (m, 9/5H) 2.47-2.36 (m, 2/5H), 2.32-2.26 (m, 8/5H), 1.74-1.61 (m, 6H), 1.61-1.53 (m, 4H), 1.54-1.43 (m, 4H), 1.34 (h, J=7.3 Hz, 2H), 0.90 (t, J=7.3 Hz, 3H). 13C NMR (101 MHz, CDCl3) (main rotamer) δ 170.33, 140.24, 117.86, 111.14, 73.14, 59.95, 53.60, 49.29, 46.17, 46.13, 43.35, 41.52, 41.38, 40.60, 36.01, 35.49, 32.74, 30.44, 19.42, 13.97. MS(ESI) m/z=372 [M+H]⁺. HRMS: calc. 372.2646, found 372,2654 [M+H]⁺.

(1R,2S,5S)-3-((3-hydroxyadamantan-1-yl)glycyl)-3-azabicyclo[3.1.0]hexane-2-carbonitrile (51). General procedure I with 3-aminoadamantan-1-ol and (1R,2S,5S)-3-(2-bromoacetyl)-3-azabicyclo[3.1.0]hexane-2-carbonitrile (24d) (100 mg, 0.437 mmol). Purified with flash chromatography on Si—C18 with 20% ACN in water to yield (1R,2S,5S)-3-((3-hydroxyadamantan-1-yl)glycyl)-3-azabicyclo[3.1.0]hexane-2-carbonitrile (90 mg, 0.285 mmol, 65%. 1H NMR (400 MHz, CDCl3) δ (10 to 1 mixture of trans to cis amide rotamers) 4.98-4.89 (m, 1/9H), 4.67 (d, J=5.2 Hz, 8/9H), 3.78-3.51 (m, 2H), 3.46 (q, J=10.1 Hz, 2/9H), 3.34 (s, 16/9H), 2.46-2.16 (m, 2H), 2.02 (p, J=6.2 Hz, 1H), 1.89 (p, J=6.8 Hz, 1H), 1.77 (bs, 2H), 1.69-1.58 (m, 4H), 1.56 (s, 2H), 1.54-1.44 (m, 6H), 1.08 (q, J=7.5 Hz, 1H), 0.68 (q, J=4.9 Hz, 1H). 13C NMR (101 MHz, CDCl3) δ (main rotamer) 171.85, 116.97, 69.57, 53.39, 50.25, 49.88, 48.78, 44.38, 43.40, 41.32, 41.17, 35.11, 30.67, 19.13, 18.60, 11.23. MS (ESI) m/z=316.2 [M+H]⁺. HRMS: calc. 316.2020, found 316.2027 [M+H]⁺.

(1R,2S,5S)-3-((-3-(benzyloxy)adamantan-1-yl)glycyl)-3-azabicyclo[3.1.0]hexane-2-carbonitrile (5m). General procedure I with 3-(benzyloxy)adamantan-1-amine (19a) (90 mg, 0.350 mmol) and (1R,2S,5S)-3-(2-bromoacetyl)-3-azabicyclo[3.1.0]hexane-2-carbonitrile (24d) (40 mg, 0.175 mmol). After filtration, the crude was redissolved in EtOAc and washed with water. The organic phase was dried with sodium sulphate, dried with reduced pressure then purified by flash chromatography on SiO2 with MeOH in DCM (2-7%) to yield (1R,2S,5S)-3-((3-(benzyloxy)adamantan-1-yl)glycyl)-3-azabicyclo[3.1.0]hexane-2-carbonitrile (59 mg, 0.145 mmol, 83% yield). 1H NMR (400 MHz, CDCl3) δ (1 to 9 cis to trans rotamers) 7.36-7.31 (m, 4H), 7.28-7.23 (m, 1H), 4.98-4.89 (m, 1/10H) 4.69 (d, J=5.4 Hz, 9/10H), 4.52 (s, 2H), 3.80-3.55 (m, 2H), 3.51 (s, 4/10H), 3.39 (s, 16/10H), 2.41-2.29 (m, 2H), 2.05 (hept, J=4.6 Hz, 1H), 1.92 (hept, J=4.1 Hz, 1H), 1.90-1.77 (m, 4H), 1.73 (s, 2H), 1.65-1.55 (m, 6H), 1.11 (q, J=6.0 Hz, 1H), 0.71 (q, J=6.0 Hz, 1H). 13C NMR (101 MHz, CDCl3) (main rotamer) δ 171.80, 139.71, 128.33, 127.52, 127.23, 116.95, 74.21, 62.80, 53.27, 50.24, 48.77, 46.44, 43.40, 41.72, 41.56, 40.76, 35.51, 30.53, 19.14, 18.60, 11.23. MS (ESI) m/z=406.3 [M+H]⁺ 428.3 [M+Na]⁺. HRMS: calc. 406.2489, found 406.2501 [M+H]⁺.

(1R,2S,5S)-3-((3-((2,4-difluorobenzyl)oxy)adamantan-1-yl)glycyl)-3-azabicyclo[3.1.0]hexane-2-carbonitrile (5n). General procedure I with 3-((2,4-difluorobenzyl)oxy)adamantan-1-amine (19b) (100 mg, 0.341 mmol) and with (1R,2S,5S)-3-(2-bromoacetyl)-3-azabicyclo[3.1.0]hexane-2-carbonitrile (24d) (50 mg, 0.218 mmol). The crude was purification with flash chromatography on SiO2 with MeOH in EtOAc (0-15%) to yield (1R,2S,5S)-3-((3-((2,4-difluorobenzyl)oxy)adamantan-1-yl)glycyl)-3-azabicyclo[3.1.0]hexane-2-carbonitrile (80 mg, 0.181 mmol, 83 yield). 1H NMR (400 MHz, CDCl3) (9 to 1 mixture of trans to cis rotamers) δ 7.45-7.35 (m, 1H), 6.84 (tdd, J=8.4, 2.6, 1.0 Hz, 1H), 6.76 (ddd, J=10.1, 9.0, 2.5 Hz, 1H), 4.96-4.91 (m, 1/10H), 4.68 (d, J=5.2 Hz, 9/10H), 4.50 (s, 2H), 3.75-3.58 (m, 2H), 3.48 (s, 4/10H), 3.38 (s, 16/10H), 2.38-2.28 (m, 2H), 2.07-1.98 (m, 1H), 1.94-1.85 (m, 1H), 1.84-1.66 (m, 6H), 1.66-1.51 (m, 6H), 1.09 (q, J=7.1 Hz, 1H), 0.69 (q, J=5.8 Hz, 1H). 13C NMR (101 MHz, CDCl3) (main rotamer) δ 171.33, 162.67 (dd, J=187.8, 11.3 Hz), 160.19 (dd, J=188.4, 11.3 Hz), 130.73 (dd, J=187.2, 11.3 Hz), 122.57 (dd, J=11.5, 3.2 Hz), 116.92, 111.08 (dd, J=24.2, 3.7 Hz), 103.53 (t, J=25.9 Hz), 74.49, 56.01 (d, J=3.8 Hz), 53.77, 48.81, 45.98, 43.13, 41.28, 41.16, 40.50, 35.37, 30.45, 19.12, 18.58, 11.18. MS (ESI) m/z=442.3 [M+H]⁺ 464.3 [M+Na]⁺. HRMS: calc. 442.2301, found 442.2297 [M+H]⁺.

(1R,2S,5S)-3-((-3-butoxyadamantan-1-yl)glycyl)-3-azabicyclo[3.1.0]hexane-2-carbonitrile (5o). General procedure I with 3-butoxyadamantan-1-amine 2,2,2-trifluoroacetate (23a) (250 mg, 0.741 mmol) and (1R,2S,5S)-3-(2-bromoacetyl)-3-azabicyclo[3.1.0]hexane-2-carbonitrile (24d) (43 mg, 0.188 mmol). After filtration product was dried then redisolved in EtOAc and washed with water, dried over sodium sulphate the dried under reduced pressure. The crude was purified with flash chromatography on SiC-18 with ACN in water (+1% FA) (10-100%), followed with HCl salt formation to yield (1R,2S,5S)-3-((3-butoxyadamantan-1-yl)glycyl)-3-azabicyclo[3.1.0]hexane-2-carbonitrile hydrochloride (21 mg, 0.051 mmol, 27%) as a white solid. HCl salt. 1H NMR (400 MHz, DMSO-d6) δ 9.24-8.85 (m, 2H), 4.90 (d, J=5.1 Hz, 1H), 4.06-3.83 (m, 1H), 3.80-3.74 (m, 2H), 3.34 (t, J=6.4 Hz, 2H), 2.34-2.26 (m, 2H), 2.17-2.06 (m, 1H), 1.95 (d, J=3.9 Hz, 1H), 1.85 (s, 2H), 1.82-1.74 (m, 4H), 1.68 (d, J=10.9 Hz, 2H), 1.56 (d, J=10.9 Hz, 2H), 1.54-1.43 (m, 2H), 1.47-1.33 (m, 2H), 1.29 (dt, J=13.9, 7.0 Hz, 2H), 1.02 (td, J=8.0, 5.3 Hz, 1H), 0.87 (t, J=7.3 Hz, 3H), 0.44 (q, J=4.3 Hz, 1H). 13C NMR (101 MHz, DMSO-d6) δ 166.50, 118.20, 72.80, 59.70, 58.93, 50.46, 48.81, 41.93, 41.10, 39.86, 36.98, 36.89, 34.54, 32.55, 29.87, 19.37, 19.23, 18.42, 14.26, 10.65. MS (ESI) m/z=371.2 [M+H]⁺.

2-((-3-hydroxyadamantan-1-yl)amino)-1-(isoindolin-2-yl)ethan-1-one (5p). General method H with 3-aminoadamantan-1-ol (100 mg, 0.598 mmol) and 2-bromo-1-(isoindolin-2-yl)ethan-1-one (24e) (120 mg, 0.498 mmol). The crude was purified with flash chromatography on Si—C18 with ACN (+1% FA) in water (10-100%) to yield 2-((3-hydroxyadamantan-1-yl)amino)-1-(isoindolin-2-yl)ethan-1-one formate (23 mg, 0.064 mmol, 13% yield) a white powder. Formic acid salt. 1H NMR (400 MHz, CDCl3) δ 8.36 (s, 1H), 7.37-7.29 (m, 4H), 4.86 (s, 2H), 4.85 (s, 2H), 4.44 (bs, 2H), 3.77 (s, 2H), 2.44-2.28 (m, 2H), 1.82 (s, 2H), 1.79-1.72 (m, 4H), 1.72-1.65 (m, 4H), 1.61-1.48 (m, 2H). 13C NMR (101 MHz, CDCl3) δ 167.60, 167.00, 135.64, 135.43, 128.07, 127.79, 123.04, 122.76, 69.23, 55.99, 52.57, 51.59, 48.15, 43.92, 41.35, 39.52, 34.71, 30.42. MS (ESI) m/z=327.3 [M+H]⁺. HRMS: calc. 327,2067, found 327.2068 [M+H]⁺.

2-((3-(benzyloxy)adamantan-1-yl)amino)-1-(isoindolin-2-yl)ethan-1-one (5q). General procedure I with 3-(benzyloxy)adamantan-1-amine (19b) (129 mg, 0.500 mmol) and 2-bromo-1-(isoindolin-2-yl)ethan-1-one (24e) (100 mg, 0.416 mmol). Product was precipitated from methanol to yield 2-((3-(benzyloxy)adamantan-1-yl)amino)-1-(isoindolin-2-yl)ethan-1-one (40 mg, 0.096 mmol, 23% yield) as a white solid. 1H NMR (400 MHz, CDCl3) δ 7.37-7.21 (m, 9H), 4.82 (s, 2H), 4.78 (s, 2H), 4.51 (s, 2H), 3.50 (s, 2H), 2.34-2.29 (m, 2H), 1.97 (bs, 1H), 1.85-1.74 (m, 6H), 1.69-1.58 (m, 4H), 1.58-1.51 (m, 2H). 13C NMR (101 MHz, CDCl3) δ 170.32, 139.76, 136.21, 135.95, 128.31, 127.90, 127.59, 127.51, 127.19, 123.09, 122.68, 74.27, 62.79, 53.29, 52.32, 51.58, 46.49, 43.21, 41.72, 40.86, 35.60, 30.58. MS (ESI) m/z=417.4 [M+H]⁺, 833 [2M+H]⁺. HRMS: calc. 417.2537, found 417.2533 [M+H]⁺.

2-((-3-((2,4-difluorobenzyl)oxy)adamantan-1-yl)amino)-1-(isoindolin-2-yl)ethan-1-one (5r). General procedure I with 3-((2,4-difluorobenzyl)oxy)adamantan-1-amine (19b) (136 mg, 0.467 mmol) and 2-bromo-1-(isoindolin-2-yl)ethan-1-one (24e) (86 mg, 0.359 mmol). Product purified with flash chromatography on Si—C18 with MeOH (+1% FA) in water (15-100%) to yield 2-((3-((2,4-difluorobenzyl)oxy)adamantan-1-yl)amino)-1-(isoindolin-2-yl)ethan-1-one formate (25 mg, 0,054 mmol, 15% yield) as a white solid. 1H NMR (400 MHz, CDCl3) δ 7.45-7.34 (m, 1H), 7.39-7.26 (m, 4H), 6.86 (td, J=8.0, 1.4 Hz, 1H), 6.77 (ddd, J=10.1, 8.9, 2.5 Hz, 1H), 4.89 (s, 2H), 4.85 (s, 2H), 4.53 (s, 2H), 3.90 (s, 2H), 3.48 (bs, 2H), 2.45 (s, 2H), 2.04 (s, 2H), 2.03-1.92 (m, 4H), 1.92-1.78 (m, 4H), 1.68-1.57 (m, 2H). 13C NMR (101 MHz, CDCl3) δ 165.39, 162.97 (dd, J=193.0, 11.8 Hz), 160.22 (dd, J=193.6, 11.8 Hz), 135.22, 134.95, 130.83 (dd, J=9.7, 6.0 Hz), 128.28, 128.01, 123.05, 122.84, 122.13 (dd, J=14.3, 3.4 Hz), 111.18 (dd, J=20.9, 3.7 Hz), 103.64 (t, J=25.1 Hz), 74.00, 58.41, 56.39 (d, J=3.6 Hz), 52.85, 51.73, 43.72, 40.99, 39.96, 38.94, 34.65, 30.12. MS (ESI) m/z=453.3 [M+H]⁺. HRMS: calc. 453.2348, found 453.2354 [M+H]⁺.

2-((-3-butoxyadamantan-1-yl)amino)-1-(isoindolin-2-yl)ethan-1-one (5s). General procedure I with 3-butoxyadamantan-1-amine (23a) (93 mg, 0.416 mmol) and 2-bromo-1-(isoindolin-2-yl)ethan-1-one (24e) (100 mg, 0,416 mmol). Product purified by flash chromatography on Si—C18 with ACN (+1/a FA) in water (5-20%) to yield 2-((3-butoxyadamantan-1-yl)amino)-1-(isoindolin-2-yl)ethan-1-one formate (21 mg, 0.055 mmol, 13% yield) as a white solid. 1H NMR (400 MHz, CDCl3) δ 7.36-7.25 (m, 4H), 6.11 (bs, 2H), 4.92 (s, 2H), 4.82 (s, 2H), 3.94 (s, 2H), 3.40 (t, J=6.6 Hz, 2H), 2.45-2.35 (m, 2H), 2.09-1.92 (m, 6H), 1.80-1.67 (m, 4H), 1.65-1.53 (m, 2H), 1.54-1.42 (m, 2H), 1.34 (h, J=7.3 Hz, 2H), 0.90 (t, J=7.3 Hz, 3H). 13C NMR (101 MHz, CDCl3) δ 164.73, 135.11, 134.98, 128.19, 127.95, 122.97, 122.85, 72.69, 60.24, 59.32, 52.85, 51.84, 43.30, 41.04, 39.98, 38.55, 34.59, 32.54, 30.02, 19.36, 13.95. MS (ESI) 383.3 [M+H]⁺. HRMS: calc. 383.2693, found 383.2688 [M+H]⁺.

1-(isoindolin-2-yl)-2-((-3-isopropoxyadamantan-1-yl)amino)ethan-1-one (5t). General method H with -3-isopropoxyadamantan-1-amine 2,2,2-trifluoroacetate (23b) (118 mg, 0.365 mmol) and 2-bromo-1-(isoindolin-2-yl)ethan-1-one (24e) (88 mg, 0.365 mmol). Product was purified with flash chromatography on Si—C18 with MeOH (+1% FA) in water (10-100%) to yield 1-(isoindolin-2-yl)-2-((3-isopropoxyadamantan-1-yl)amino)ethan-1-one formate (45 mg, 0.109 mmol, 30% yield). Formic acid salt. 1H NMR (400 MHz, Acetone-d6) δ 8.24 (s, 1H), 7.34 (ddt, J=15.6, 5.7, 3.6 Hz, 4H), 5.11 (bs, 2H), 4.95 (s, 2H), 4.74 (s, 2H), 3.97 (h, J=6.1 Hz, 1H), 3.87 (s, 2H), 2.28 (s, 2H), 1.87 (s, 2H), 1.84-1.76 (m, 4H), 1.76-1.64 (m, 4H), 1.56 (d, J=3.0 Hz, 2H), 1.04 (d, J=6.1 Hz, 6H). 13C NMR (101 MHz, Acetone-d6) δ 168.36, 136.76, 136.19, 127.55, 127.45, 122.95, 122.75, 73.16, 61.48, 54.83, 51.96, 51.05, 45.85, 41.89, 41.27, 39.97, 35.10, 30.44, 24.86. MS (ESI) m/z=369.1 [M+H]⁺.

1-(isoindolin-2-yl)-2-((-3-(octyloxy)adamantan-1-yl)amino)ethan-1-one (5u). General procedure F with 3-(octyloxy)adamantan-1-amine 2,2,2-trifluoroacetate (24c) (260 mg, 0,463 mmol) and 2-bromo-1-(isoindolin-2-yl)ethan-1-one (24e) 2-bromo-1-(isoindolin-2-yl)ethan-1-one (111 mg, 0,463 mmol). The compound was purified by flash chromatography on SiO2 with MeOH in DCM (1-15%) to yield 1-(isoindolin-2-yl)-2-((3-(octyloxy)adamantan-1-yl)amino)ethan-1-one (64 mg, 0.146 mmol, 32% yield). 1H NMR (400 MHz, CDCl3) δ 7.27-7.12 (m, 4H), 4.12 (bs, 1H), 4.76 (s, 2H), 4.74 (s, 1H), 3.52 (s, 2H), 3.32 (t, J=6.8 Hz, 2H), 2.27-2.16 (m, 2H), 1.70-1.50 (m, 10H), 1.50-1.36 (m, 4H), 1.28-1.11 (m, 10H), 0.80 (t, J=6.7 Hz, 3H). 13C NMR (101 MHz, CDCl3) δ 169.33, 136.00, 135.75, 127.96, 127.66, 123.07, 122.71, 73.11, 60.34, 54.14, 52.41, 51.57, 45.71, 42.40, 41.07, 40.57, 35.43, 31.86, 30.66, 30.39, 29.50, 29.32, 26.26, 22.69, 14.14. MS (ESI) m/z=439.0 [M+H]⁺, 461.0 [M+Na]⁺. HRMS: calc. 439.3319, found 439.3298 [M+H]⁺.

### Biochemical Evaluation:

The compounds were biochemically evaluated against a panel consisting of all enzymatically active DASH enzymes (DPP4, DPP8, DPP9, DPP2, FAP) and PREP.

Potencies of the 2-cyanopyrrolidine-based compounds (5a-o) are shown in Table 1. The unsubstituted cyanopyrrolidine subset 5a-c comprises the closest analogues of vildagliptin that were synthesized. Compared to parent compound vildagliptin/4, the affinities of 5a-c are typically slightly increased across the full evaluation panel. Because of its generality, this trend can tentatively be attributed to the increased lipophilicity of the ether derivatives, compared to vildagliptin. This also indicates that the benzyl or butyl groups in these molecules are tolerated, but do not provide additional specific affinity-conferring interactions with the enzymes. Next to selectivity issues with respect to DPP4, compounds 5a-b also inhibit PREP, and compound 5a is a micromolar inhibitor of FAP as well.

TABLE 1
Enzyme inhibitory potencies for cyanopyrrolidine target compounds 5a-o.

| | | | IC₅₀^{[a]} (μM) | | | SI^{[b]} | | IC₅₀^{[a]} (μM) | | |
|---|---|---|---|---|---|---|---|---|---|---|
| Cmpd. | P1 | R² | DPP4 | DPP8 | DPP9 | DPP9/8 | DPP9/4 | DPP2 | FAP | PREP |
| 4 | 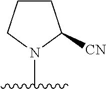 [SMILES: *N1CCC[C@H]1C#N] | —H | 0.010 ± 0.001 | 2.2 ± 0.4 | 0.23 ± 0.08 | 9.6 | 0.04 | >5 | >10 | >10 |
| 5a | 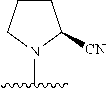 [SMILES: *N1CCC[C@H]1C#N] | 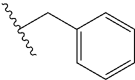 [SMILES: *Cc1ccccc1] | 0.0048 ± 0.0010 | 1.32 ± 0.2 | 0.13 ± 0.02 | 9.9 | 36 | >5 | 4 ± 4 | 4.3 ± 0.3 |
| 5b | 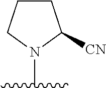 [SMILES: *N1CCC[C@H]1C#N] | 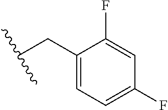 [SMILES: *Cc1ccc(F)cc1F] | 0.005 ± 0.001 | 0.54 ± 0.1 | 0.052 ± 0.008 | 10 | 0.1 | >5 | >10 | 2.2 ± 0.2 |
| 5c | 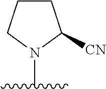 [SMILES: *N1CCC[C@H]1C#N] | 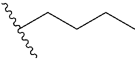 [SMILES: *CCCC] | 0.012 ± 0.002 | 1.25 ± 0.1 | 0.15 ± 0.01 | 8.5 | 0.07 | >5 | >10 | >10 |
| 5d | 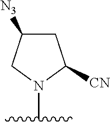 [SMILES: *N1C[C@@H](N=[N+]=[N-])C[C@H]1C#N] | —H | 0.032 ± 0.003 | 0.15 ± 0.02 | 0.016 ± 0.001 | 9.4 | 2 | >5 | >10 | >10 |
| 5e | 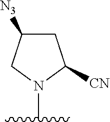 [SMILES: *N1C[C@@H](N=[N+]=[N-])C[C@H]1C#N] | 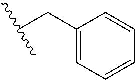 [SMILES: *Cc1ccccc1] | 0.017 ± 0.003 | 0.060 ± 0.004 | 0.010 ± 0.001 | 6.0 | 1.7 | >5 | >10 | >10 |
| 5f | 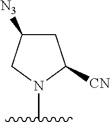 [SMILES: *N1C[C@@H](N=[N+]=[N-])C[C@H]1C#N] | 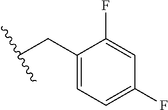 [SMILES: *Cc1ccc(F)cc1F] | 0.024 ± 0.003 | 0.041 ± 0.007 | 0.006 ± 0.001 | 6.8 | 3.3 | >5 | >10 | >10 |
| 5g | 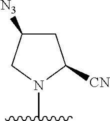 [SMILES: *N1C[C@@H](N=[N+]=[N-])C[C@H]1C#N] | 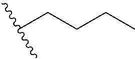 [SMILES: *CCCC] | 0,13 ± 0:05 | 0.27 ± 0.06 | 0.035 ± 0.002 | 7.8 | 3.8 | >5 | >10 | >10 |
| 5h | 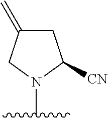 [SMILES: *N1CC(=C)C[C@H]1C#N] | —H | 0.23 ± 0.07 | 1.0 ± 0.2 | 0.26 ± 0.03 | 3.9 | 0.9 | >5 | >10 | >10 |
| 5i | 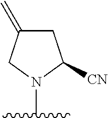 [SMILES: *N1CC(=C)C[C@H]1C#N] | 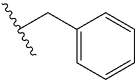 [SMILES: *Cc1ccccc1] | 0.28 ± 0.01 | 0.34 ± 0.006 | 0.12 ± 0.03 | 2.9 | 2.3 | 1.27 ± 0.2 | >10 | 8.92 ± 1 |
| 5j | 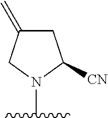 [SMILES: *N1CC(=C)C[C@H]1C#N] | 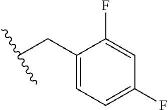 [SMILES: *Cc1ccc(F)cc1F] | 0.41 ± 0.1 | 0.37 ± 0.07 | 0.13 ± 0.02 | 2.8 | 3.1 | 1.10 ± 0.08 | >10 | >10 |
| 5k | 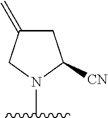 [SMILES: *N1CC(=C)C[C@H]1C#N] | 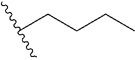 [SMILES: *CCCC] | 0.32 ± 0.01 | 0.53 ± 0.01 | 0.21 ± 0.05 | 2.5 | 1.5 | 1.2 ± 0.4 | >10 | 2.2 ± 0.2 |
| 51 | 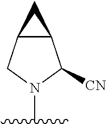 [SMILES: *N1C[C@H]2C[C@H]2[C@H]1C#N] | —H | 0.180 ± 0.005 | 21.4 ± 3.7 | 1.22 ± 0.03 | 17.5 | 0.15 | >5 | >10 | >10 |
| 5m | 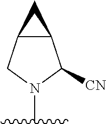 [SMILES: *N1C[C@H]2C[C@H]2[C@H]1C#N] | 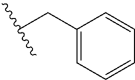 [SMILES: *Cc1ccccc1] | 0.10 ± 0.04 | 5.9 ± 0.4 | 0.67 ± 0.2 | 8.7 | 0.15 | >5 | >10 | >10 |
| 5n | 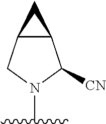 [SMILES: *N1C[C@H]2C[C@H]2[C@H]1C#N] | 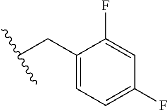 [SMILES: *Cc1ccc(F)cc1F] | 0.20 ± 0.01 | 2 ± 2 | 0.62 ± 0.01 | 3.4 | 0.3 | >5 | >10 | >10 |
| 5o | 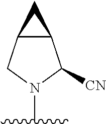 [SMILES: *N1C[C@H]2C[C@H]2[C@H]1C#N] | 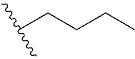 [SMILES: *CCCC] | 0.15 ± 0.01 | >10 | 0.99 ± 0.4 | >10 | 0.15 | >5 | >10 | >10 |

^{[a]}IC₅₀s are presented as the mean ± standard deviation (n = 3).
^{[b]}SI: selectivity index, calculated by dividing DPP8 IC₅₀ over the DPP9 IC₅₀ (DPP9/8).

In the subsets of the pyrrolidine-substituted derivatives 5d-o, DPP4-potency is mostly decreased compared to vildagliptin. Generally in the higher nanomolar range, it is, however, still significant. With the notable exception of the 4-azido-substituted congeners (5d-g, vide infra), potencies for DPP8 increase modestly for the substituted derivatives, while potencies for DPP9 stay roughly comparable to vildagliptin and its ether derivatives. All these findings lead to the general conclusion that introducing the selected pyrrolidine substituents diverts selectivity away from DPP4, in favor of DPP8 and 9. This is also reflected by, e.g., the DPP9/4 selectivity indices for 5d-o that are >1). Nonetheless, the DPP9/8 selectivity indices of these molecules are also mostly lower than for 4 and 5a-c.

The azido-cyanopyrrolidines 5d-g take a special position in this subseries. These compounds have notable nanomolar potencies for DPP9, with the difluorobenzylated compound 5f being the most potent DPP9 inhibitor in this paper. However, they are highly potent inhibitors for DPP4 and DPP8 as well, which makes them unfit for future optimization. The azido-cyanopyrrolidine subset did not inhibit DPP2, FAP, and PREP at the tested concentrations.

The 4-methylenecyanopyrrolidines 5 h-k exhibit the least selectivity within the 5d-o subset. In addition to DPP4 affinity, these molecules are all low micromolar inhibitors of DPP2, and the R2 substituted ethers (5i-k) inhibit PREP as well. The DPP9 over DPP8 selectivity was further attenuated compared to the azido-cyanopyrrolidines, as shown by comparing compound 5f to 5j. Continuing in Table 1, the L-cis-3,4-methanocyanopyrrolidines 5l and 5o have an unprecedented DPP9/8 selectivity index of >10. Nevertheless, all L-cis-3,4-methanocyanopyrrolidines (51-0) exhibit higher inhibitory potencies for DPP4 than for DPP8 or DPP9. Furthermore, the potencies for both DPP9 and DPP8 are the lowest among the cyanopyrrolidine-based compounds.

Table 2 summarizes data for the isoindoline-derived compounds (5p-u). None of these molecules inhibited DPP4, DPP2, FAP, or PREP at the tested concentrations. This implies that the nanomolar DPP4 inhibitor vildagliptin (4) was effectively transformed into a selective DPP8/9 inhibitor, meeting the first goal of the study. The effect of replacing a 2-cyanopyrrolidine moiety with isoindoline, can be illustrated by comparing affinity data for vildagliptin/4 and the directly analogous 5p. A DPP4 potency decrease of at least 100-fold, is observable between the two compounds. Less fortunately, a somewhat lower affinity for DPP8 and DPP9, is also present. A likely explanation for the latter might be the lack of a warhead on the molecules. We have shown earlier for the DPP8/9 inhibitor allo-Ile-isoindoline, that a carbonitrile warhead at the 2-position of isoindoline (similar to 2-cyanopyrrolidine) unexpectedly leads to a decrease in overall DPP affinity.[25] The latter indicates that carbonitrile warheads are not functional in this series and are most likely not capable of interacting with DPP8 or DPP9's catalytic serine residue. Nevertheless, exploring other warheads could still be a viable option here. Earlier, we also demonstrated that a diarylphosphonate warhead at isoindoline's 2-position, is functional and can lead to selective, irreversible inhibitors.

Also in this series, the most lipophilic compound (difluorobenzylated derivative 5r), again shows the highest DPP8/9 potency, comparable to vildagliptin's. However, the DPP9/8 selectivity index (4.1) of this molecule, is not the best in the series and also lower than the parent compounds. On the other hand, the corresponding alkylated compounds 5s-u, in our opinion show the best balance between DPP9 potency and selectivity, especially the isoindoline-derived butyl ether 5s. Extending the butyl chain of 5s to an octyl ether (5u) did not significantly affect the DPP8 potency, nor the DPP9 potency. Compound 5s or 5u could therefore be used as leads to further improve the DPP9 selectivity and to increase the DPP9 affinity.

TABLE 2
Enzyme inhibitory potencies for isoindoline-containing target compounds 5p-u.

| | | | IC₅₀^{[a]} (μM) | | | SI^{[b]} | | IC₅₀^{[a]} (μM) | | |
|---|---|---|---|---|---|---|---|---|---|---|
| Cmpd | P1 | R² | DPP4 | DPP8 | DPP9 | DPP9/8 | DPP9/4 | DPP2 | FAP | PREP |
| 5p | 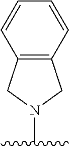 [SMILES: *N1Cc2ccccc2C1] | —H | >10 | 17.7 ± 0.8 | 4.1 ± 0.7 | 4.3 | >2.4 | >5 | >10 | >10 |
| 5q | 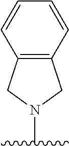 [SMILES: *N1Cc2ccccc2C1] | 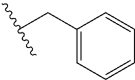 [SMILES: *Cc1ccccc1] | >10 | 6.9 ± 2 | 1.2 ± 0.2 | 5.8 | >8.5 | >5 | >10 | >10 |
| 5r | 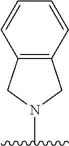 [SMILES: *N1Cc2ccccc2C1] | 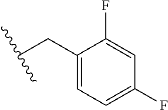 [SMILES: *Cc1ccc(F)cc1F] | >10 | 1.54 ± 0.09 | 0.38 ± 0.07 | 4.1 | >26.4 | >5 | >10 | >10 |
| 5s | 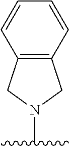 [SMILES: *N1Cc2ccccc2C1] | 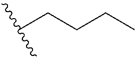 [SMILES: *CCCC] | >10 | 3.1 ± 0.1 | 0.49 ± 0.04 | 6.4 | >20.4 | >5 | >10 | >10 |
| 5t | 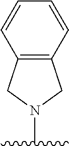 [SMILES: *N1Cc2ccccc2C1] | 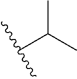 [SMILES: *C(C)C] | >10 | 4 ± 1 | 0.61 ± 0.07 | 6.5 | >16.4 | >5 | >10 | >10 |
| 5u | 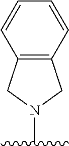 [SMILES: *N1Cc2ccccc2C1] | 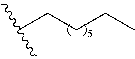 | >10 | 3.3 ± 0.5 | 0.5 ± 0.2 | 6.8 | >21 | >5 | >10 | >10 |

^{[a]}IC₅₀s are presented as the mean ± standard deviation (n = 3).
^{[b]}SI: selectivity index, calculated by dividing DPP8 IC₅₀ over the DPP9 IC₅₀ (DPP9/8).

### Example 2: Synthesis of Isoindoline Comprising Exemplary Compounds Listed in Table 3 and Table 4

General procedures A¹-Z¹ and general procedures 1-5 contain the experimental procedures for the intermediates depicted in scheme 5 to 23.

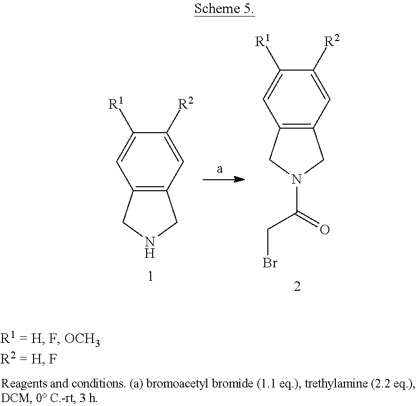

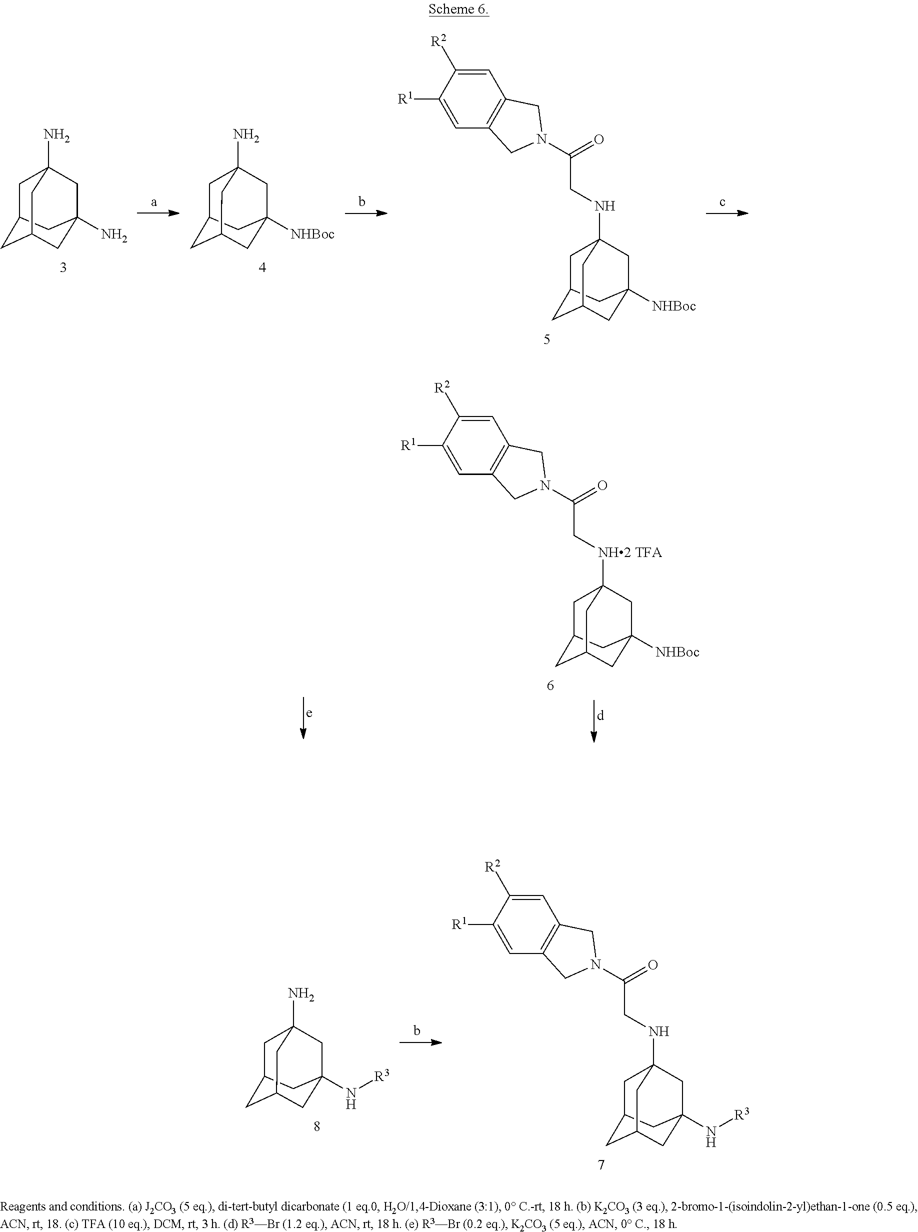

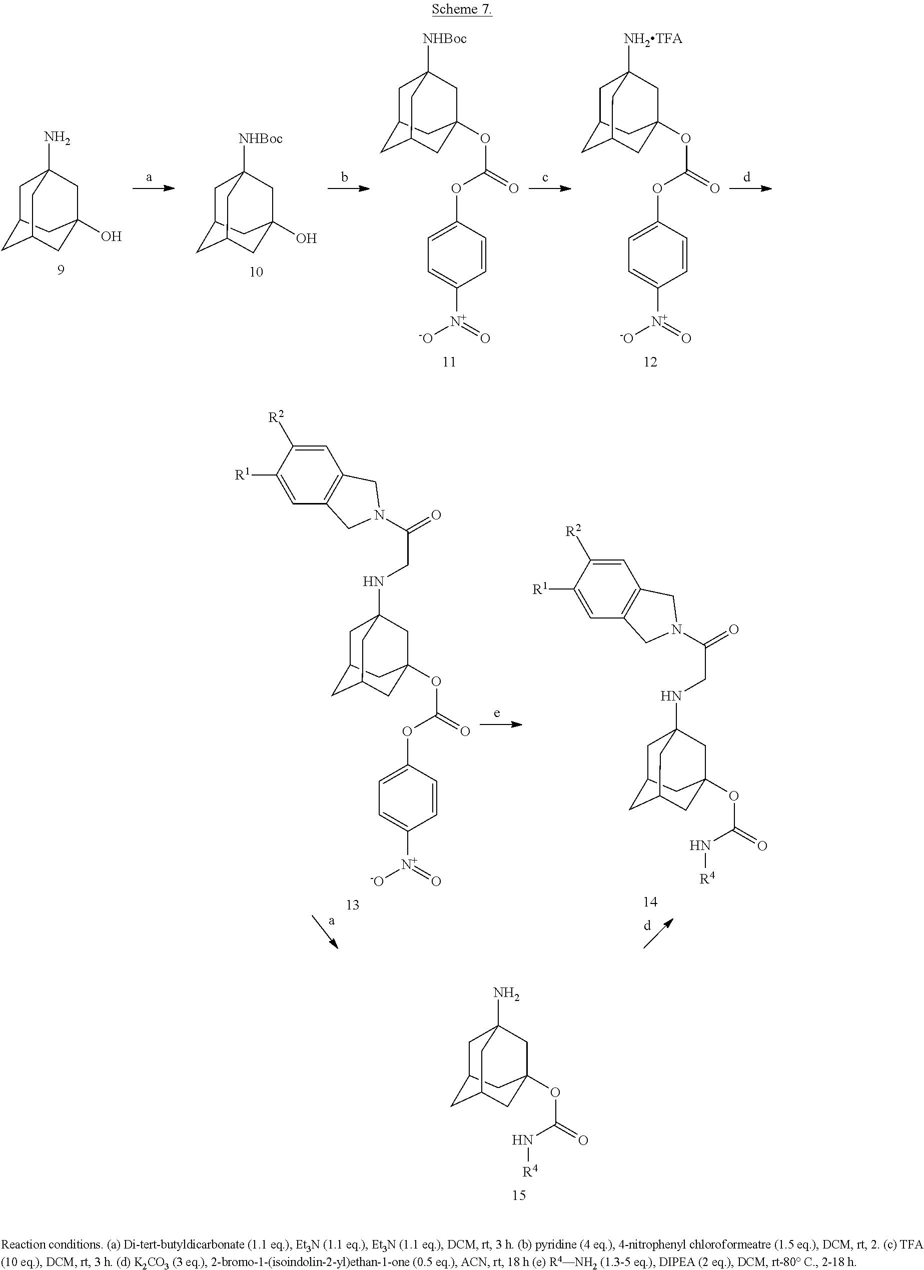

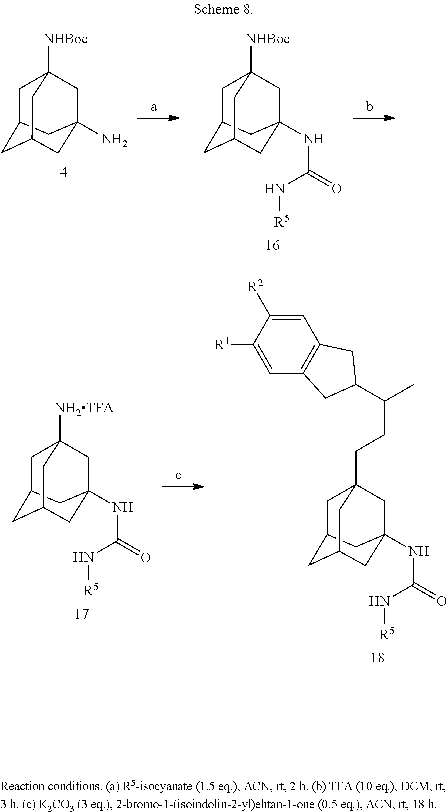

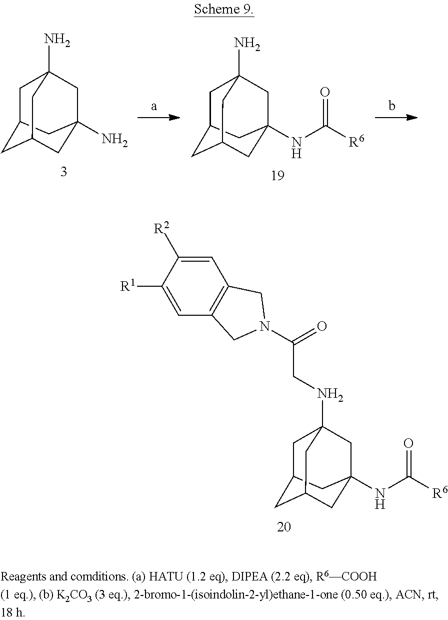

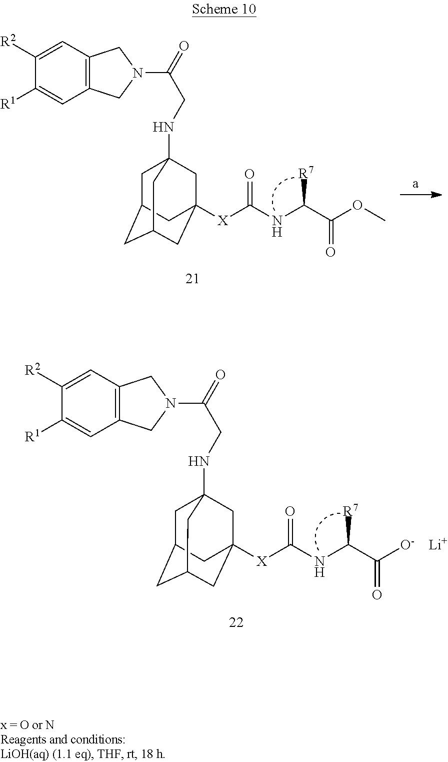

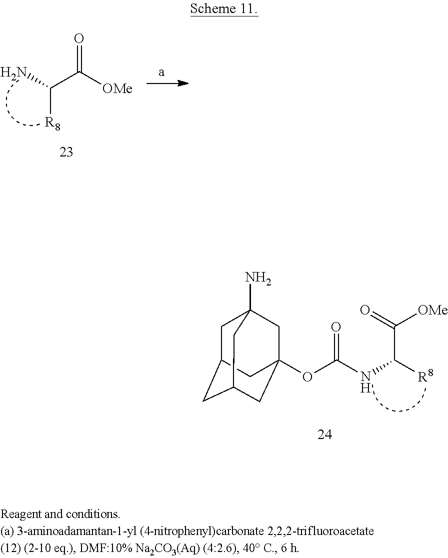

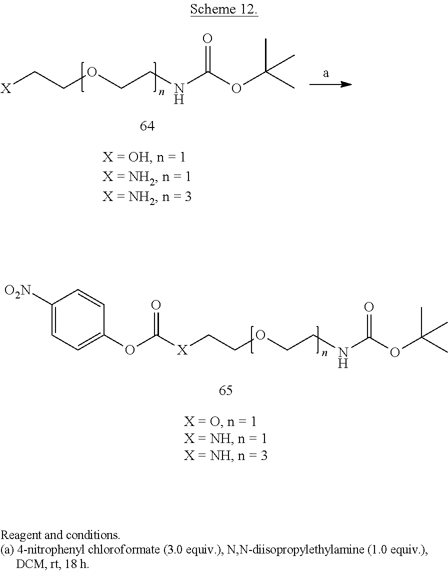

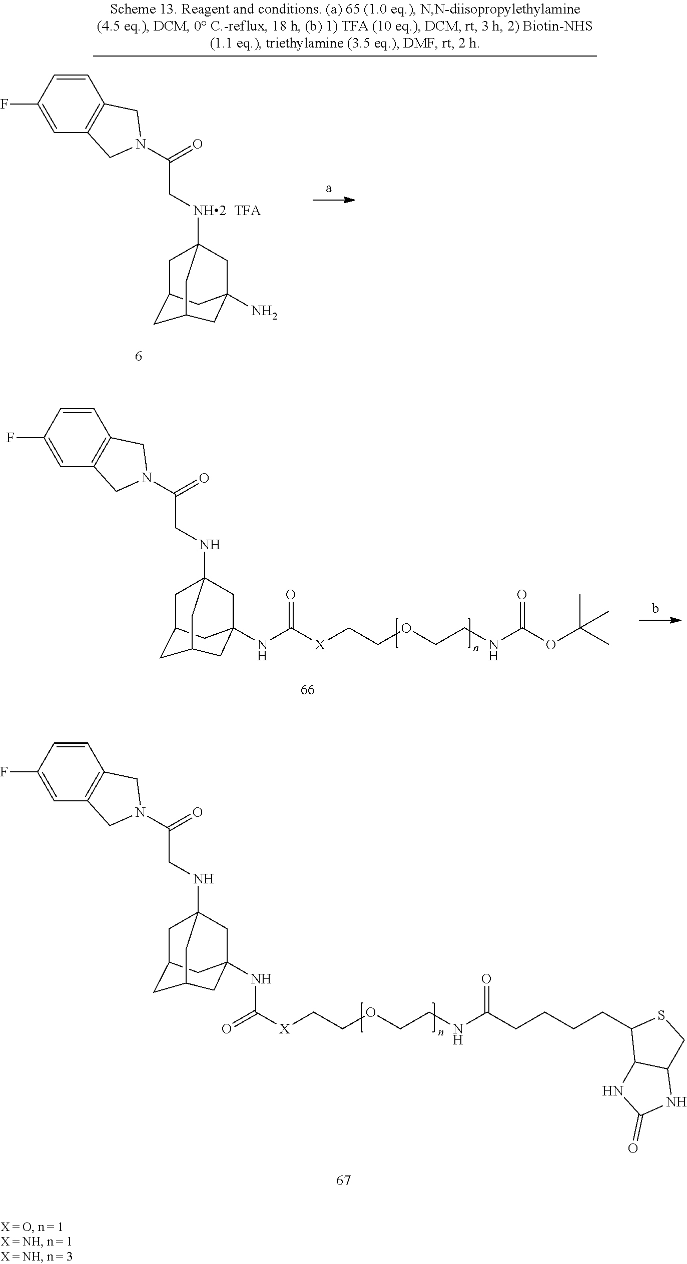

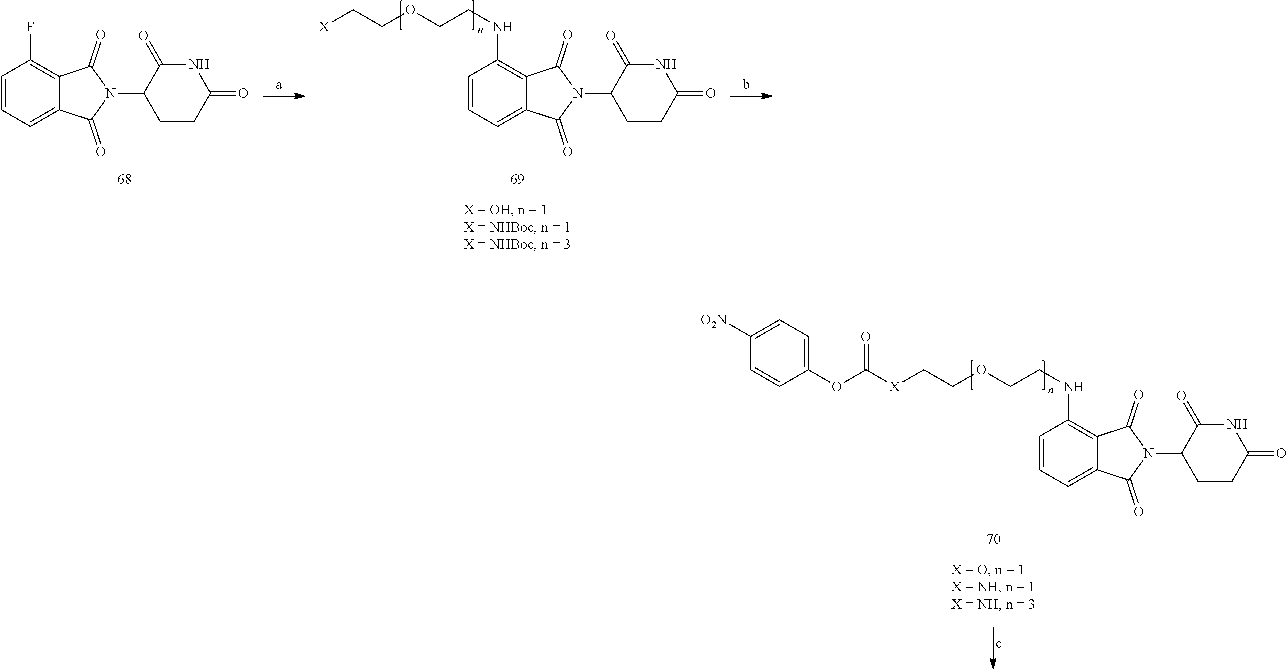

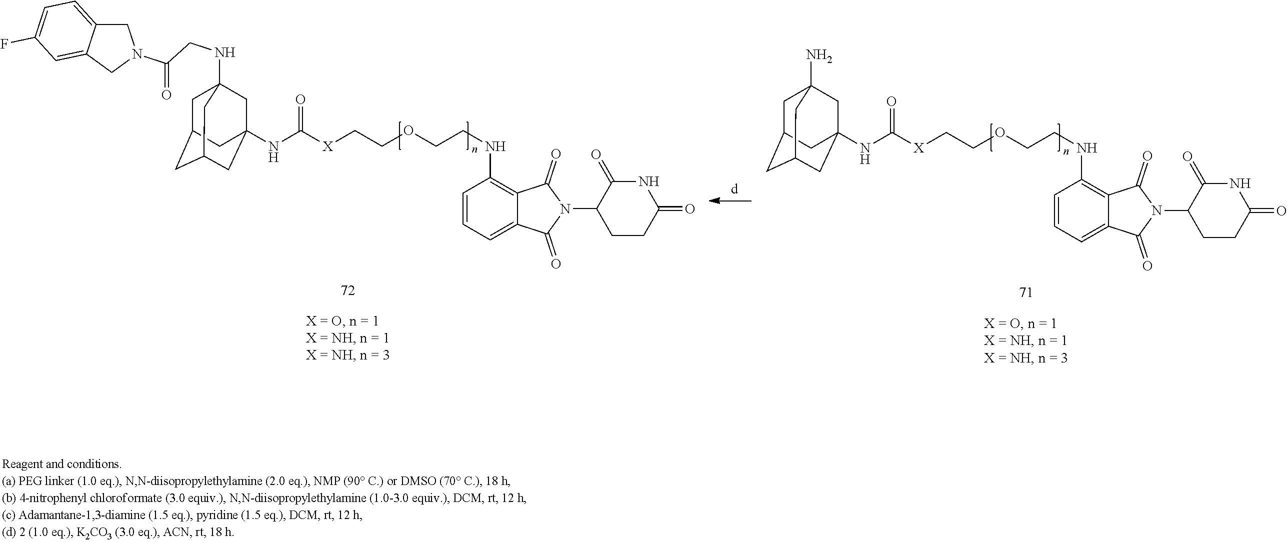

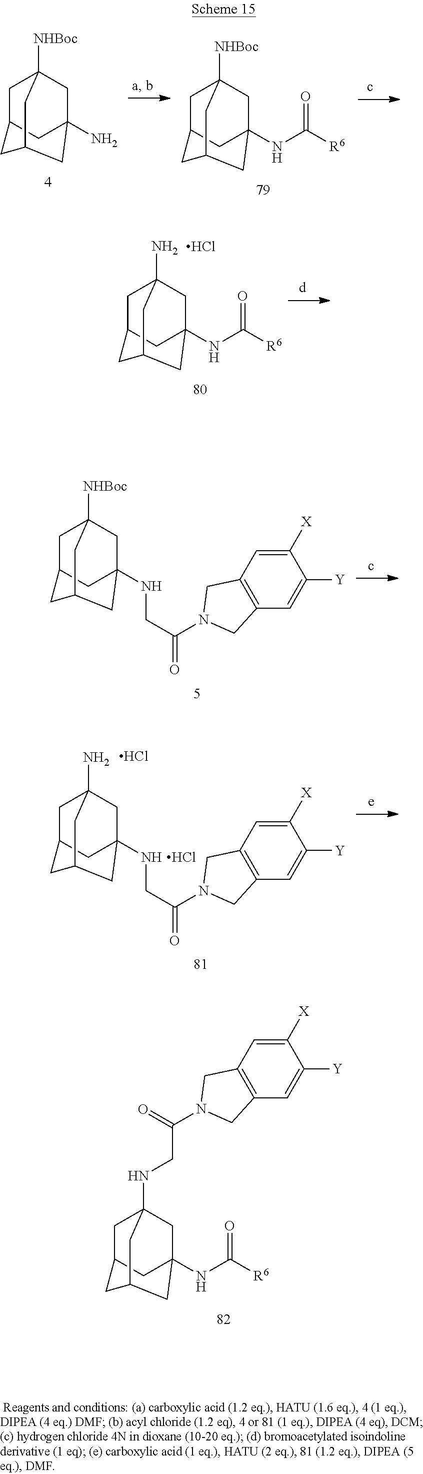

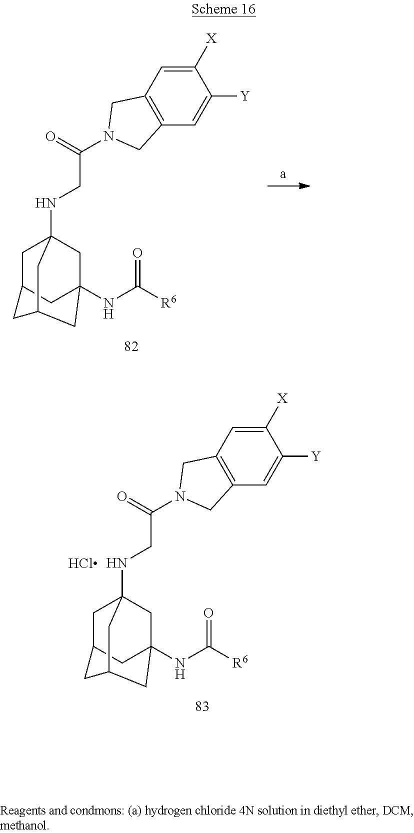

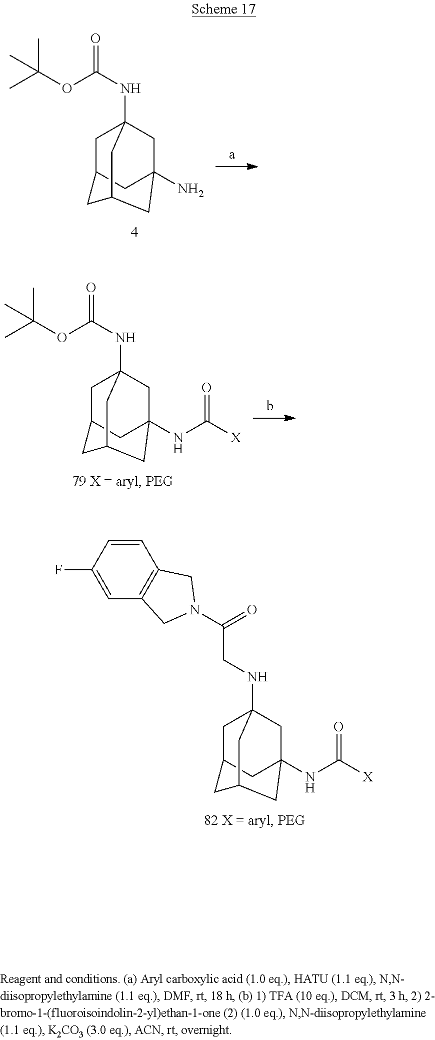

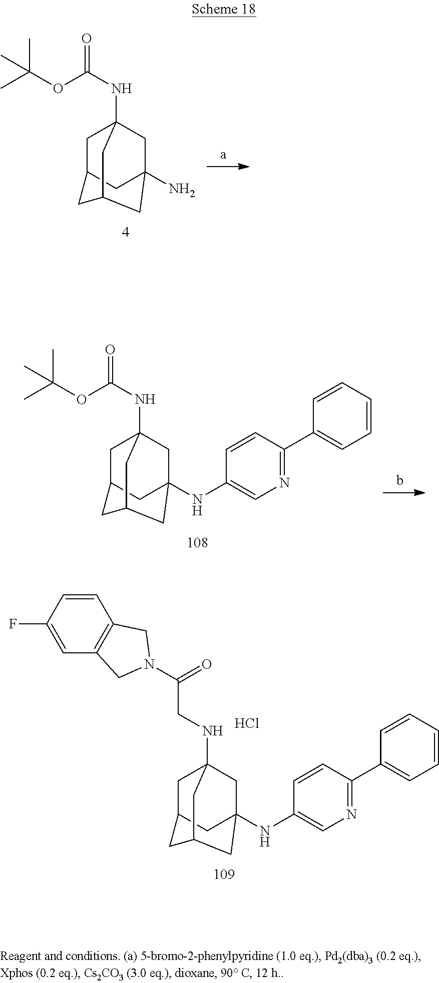 [SMILES: CC(C)(C)OC(=O)NC12CC3CC(CC(N)(C3)C1)C2 ; CC(C)(C)OC(=O)NC12CC3CC(C1)CC(Nc1ccc(-c4ccccc4)nc1)(C3)C2 ; O=C(CNC12CC3CC(C1)CC(Nc1ccc(-c4ccccc4)nc1)(C3)C2)N1Cc2ccc(F)cc2C1 ; Cl]

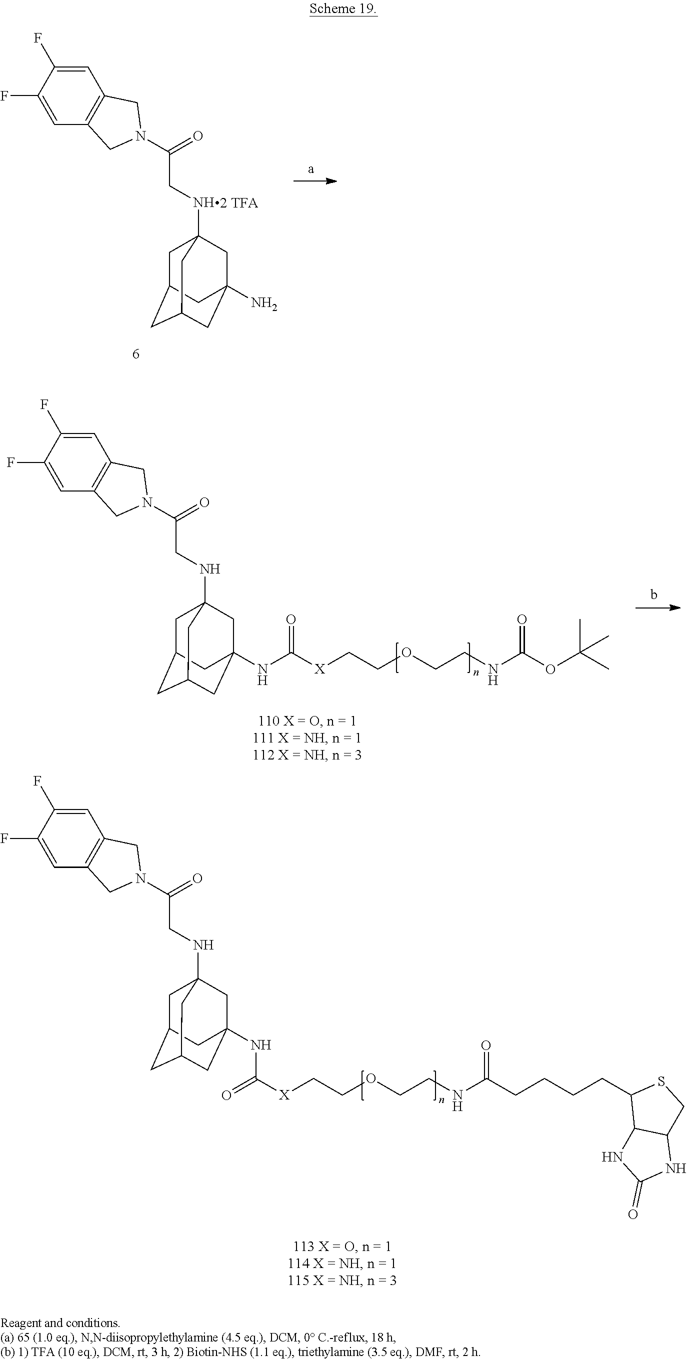

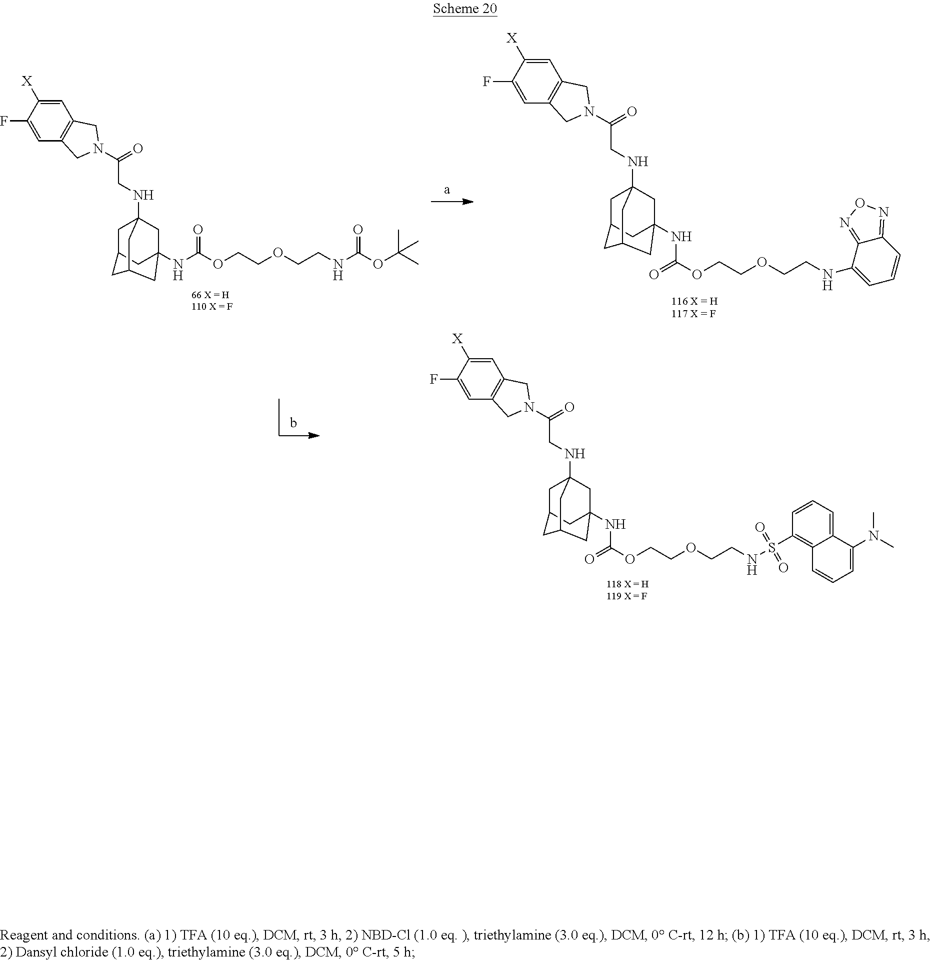

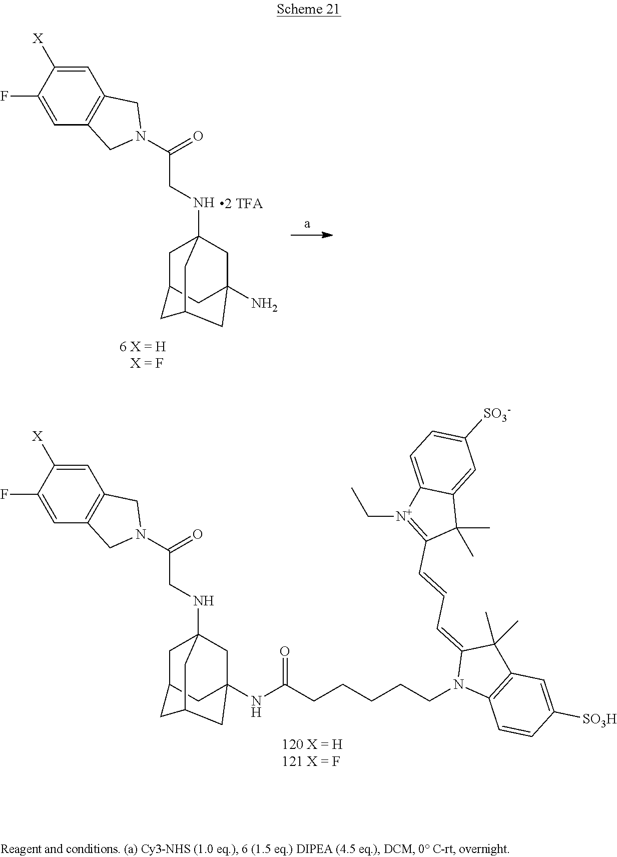

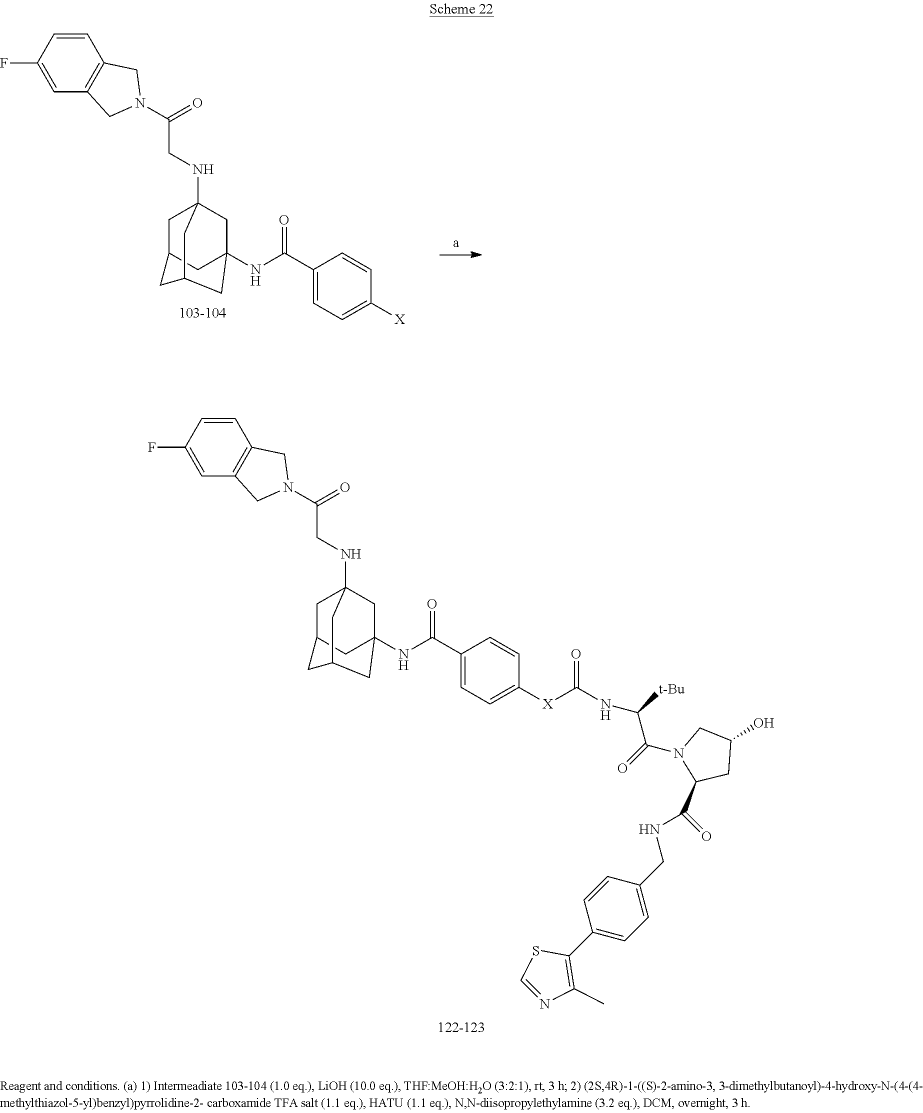

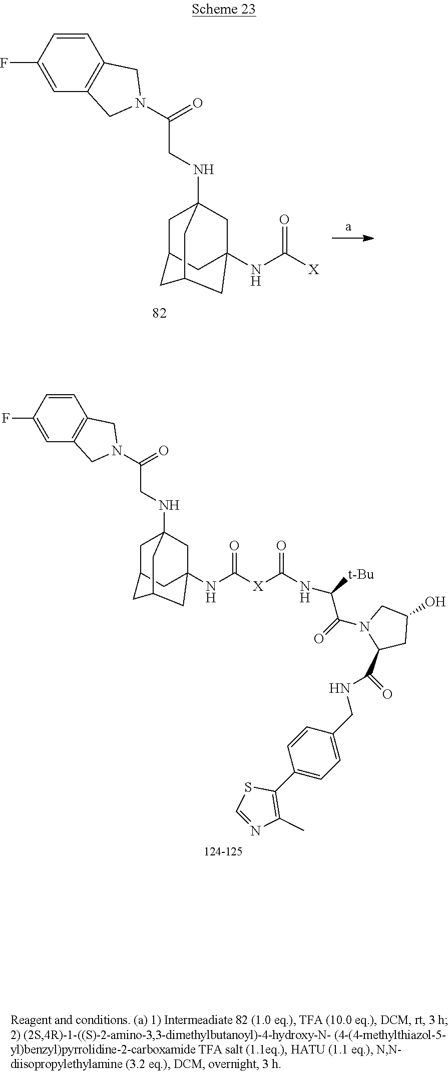

General procedure A¹. Commercial isoindolines were suspended in anhydrous DCM (0.25 M) and cooled down to 0° C. Dry triethylamine (1.2 eq.) was added, followed by dropwise addition of bromoacetic bromide (1.2 eq.). The reaction mixture was stirred for 3 h, then it was diluted with DCM and washed with a saturated NaHCO₃ solution (3×10 mL). The organic phase was dried over sodium sulfate, concentrated under reduced pressure, and purified with flash chromatography on SiO2 with EtOAc in heptane (0-30%) to yield 2-bromo-1-(isoindolin-2-yl)ethan-1-one derivatives (2).

General procedure B¹. To a stirring suspension of starting material A (individually specified) in ACN (0.05 M), potassium carbonate (3 eq.) was added. After 15 min. of vigorous stirring, the reaction was cooled down to 0° C. The 2-bromo-1-(isoindolin-2-yl)ethan-1-one derivative (2) (0.5 eq.) was dissolved in 4 mL ACN and was slowly added over 1-10 h to the stirring suspension. The reaction was allowed to reach rt and was left stirring overnight. The obtained crude was filtered and the solids were washed with acetonitrile. The filtrate was concentrated via rotary evaporator. Further purification is described individually.

General procedure C¹. The boc-protected intermediates were dissolved in DCM (0.5 M) and trifluoroacetic acid (10 eq.) was added. After 2 h, the volatiles were dried and TFA was co-evaporated with toluene to yield the desired products.

General procedure D¹. To a stirring solution of 2-((3-aminoadamantan-1-yl)amino)-1-(isoindolin-2-yl)ethan-1-one bis(2,2,2-trifluoroacetate) in ACN (0.1 M), potassium carbonate (3 eq.) was added. After 15 min. of stirring, the desired alkyl bromide (1.2 eq.) was diluted with 5 mL ACN and was slowly added over 30 min. The reaction was left stirring overnight. The mixture was filtered and washed with ACN, then dried, and purified with flash chromatography on SiO2 with MeOH in EtOAC (0-15%).

General procedure E¹. To a stirring solution of 3-((2-(isoindolin-2-yl)-2-oxoethyl)amino)adamantan-1-yl (4-nitrophenyl) carbonate in DCM (0.1 M), N-ethyl-N-isopropylpropan-2-amine (2 eq.) and the desired R4-amine (1.3-3 eq.) were added and the reaction was stirred between rt−80° C. and for 2-18 h. The reaction solution was diluted with DCM (10 mL) and washed with HCl (3×5 mL). The organic layer was dried over anhydrous Na2SO4, filtered, and concentrated under via rotary evaporator. Purification of the products was done by flash chromatography and is individually specified.

General procedure F¹. R3 substituted adamantane-1,3-diamine intermediates (8). Adamantane-1,3-diamine or adamantane-1,3-diamine bis(2,2,2-trifluoroacetate) was suspended in ACN (0.05 M) and potassium carbonate (5 eq.) was added. The reaction mixture was stirred vigorously for 15 min, then cooled down to 0° C. The desired R3-bromide was diluted with 1 mL ACN and added dropwise over 30 min. The reaction was stirred overnight, filtered, and dried under reduced pressure. The crude was purified by chromatography on SiO2 with mixture A (2% NH3, 20% MeOH, 78% DCM) in DCM (0-100%).

General procedure G¹. R4 substituted 3-aminoadamantan-1-yl carbamate intermediates (15). To a stirring suspension of 3-aminoadamantan-1-yl (4-nitrophenyl) carbonate 2,2,2-trifluoroacetate in DMF (0.1 M) was added potassium carbonate (5 eq.) and R4-amine (2 eq.). After 2 h, the reaction mixture was diluted with 10 mL DCM and washed with water. The organic layer was dried with Na2SO4, filtered, and concentrated under via rotary evaporation. The crude was purified by flash chromatography on SiO2 with MeOH in DCM (0-15%) to yield the desired intermediates.

General procedure H¹. R5 substituted tert-butyl (3-(3-ureido)adamantan-1-yl)carbamate intermediates (16). tert-butyl (3-aminoadamantan-1-yl)carbamate (4) is dissolved in ACN (0.1 M) and cooled down to 0° C. R5-isocyanate (1.5 eq.) was diluted with 2 mL ACN and added dropwise to the solution. The reaction was stirred for 2 h at 0° C. and then was filtrated and washed with ACN to yield the desired urea product (16).

General procedure I¹. R6 substituted N-(3-aminoadamantan-1-yl)amide intermediates (19). R6-carboxylicacid and O-(7-Azabenzotriazol-1-yl)-N,N,N′,N′-tetramethyluroniumPF6 (1.2 eq.) were dissolved in DMF, followed by the addition of DIPEA (1.2 eq.). After 30 min of stirring, the reaction was cooled down to 0° C. and adamantane-1,3-diamine (1 eq.) and DIPEA (1 eq.) were added. The reaction was stirred at rt overnight. The crude was diluted with 10 mL DCM and washed with a saturated NaHCO₃ solution. The organic layer was dried over sodium sulfate and DCM was evaporated under reduced pressure. The crude was purified with flash chromatography on SiC18 with MeOH in water (35-100%).

General procedure J¹. Aminoacyl methylester derivatives (21) were dissolved in THF and 1.1 eq of a 6 M aqueous solution of lithium hydroxide was added. The mixture was stirred until complete conversion of the starting material. The samples were concentrated in vacuo and lyophilized after the addition of water to yield the lithium salts (22).

General procedure K¹. A solution of Aminoacyl derivative (2-10 eq.) (23) in DMF (1 mL) and 2.60 mL of a 10% aqueous Na2CO3 was stirred at 40° C. A solution of compound (12) (1 eq.) in DMF (3.00 mL) was slowly added dropwise over the span of 6 hours. The solution was left under magnetic stirring at 40° C. until completion. The volatiles were removed via rotary evaporator and the crude product was purified via normal-phase flash chromatography in a SiO2 column eluting with a gradient of the mixture DCM:MeOH:NH3 (8:2:0.4) in DCM to give the desired intermediates (24).

General procedure L¹. To a stirring solution of 64 (1.0 eq.) in DCM (0.10 mmol/mL) was added N,N-diisopropylethylamine (1.0 eq.) at room temperature, followed by the addition of 4-nitrophenyl chloroformate (3.0 eq.) portionwise. The reaction was stirred for 18 h at the same temperature, and then the solvent was removed in vacuo. The crude solid was purified by silica gel flash chromatography (elution with AcOEt/heptane) to yield the desired intermediate 65.

General procedure M¹. To a stirring solution of 2-((3-aminoadamantan-1-yl)amino)-1-(5-fluoroisoindolin-2-yl)ethan-1-one bis(2,2,2-trifluoroacetate (6) (1.5 eq.) in DCM (0.23 mmol/mL) was added N,N-diisopropylethylamine (4.5 eq.) at 0° C. After stirring for 10 min, a solution of intermediate 65 (1.0 eq.) in DCM (0.30 mmol/mL) was added dropwise to the previous solution. The reaction was warmed up to room temperature and stirred for 18 h at reflux. After this time, the volatiles were removed in vacuo and the crude solid was purified by silica gel flash chromatography (elution with MeOH in DCM) to yield the desired intermediate 66.

General procedure N¹. The boc-protected intermediate 66 (1.0 eq.) was dissolved in DCM (0.5 M) and trifluoroacetic acid (10 eq.) was slowly added at room temperature. After 3 h, the volatiles were removed in vacuo and TFA was co-evaporated with toluene to yield the desired free amine as TFA salt. The previous crude was dissolved in DMF (0.07 mmol/mL) and triethylamine (3.5 eq.) was added. After stirring for 10 min, Biotin-NHS (1.1 eq.) was added portionwise at room temperature and the reaction was stirred for 2 h. After this time, the volatiles were removed in vacuo and the crude solid was purified by silica gel flash chromatography (elution with a mixture of DCM:MeOH:NH3 (8:2:0.2) in DCM) to yield the desired biotinylated derivative 67.

General procedure O¹. To a solution of 2-(2,6-dioxo-piperidin-3-yl)-4-fluoroisoindoline-1,3-dione (68) (1.0 eq) and PEG linker (1.1 eq) in NMP or DMSO (0.20 mmol/mL), was added N,N-diisopropylethylamine (2.0 eq). The reaction mixture was heated to 90° C. or 70° C. during 18 h. After this time, the mixture was diluted with EtOAc (60 mL), washed with a mildly basic aqueous Na₂CO₃ solution (20 mL), brine (20 mL), dried over Na₂SO₄ and concentrated in vacuo. The crude residue was purified by silica gel flash chromatography (elution with MeOH in DCM) to yield the desired derivative 69.

General procedure P¹. The N-Boc derivatives 69 (1.0 eq.) were first deprotected using trifluoroacetic acid (10 eq.) in DCM (0.5 M) at room temperature (see general procedure C). To a stirring solution of Pomalidomide-PEG (1.0 eq.) in DCM (0.10 mmol/mL) was added N,N-diisopropylethylamine (1.0-3.0 eq.) at room temperature, followed by the addition of 4-nitrophenyl chloroformate (3.0 eq.) portionwise. The reaction was stirred for 12 h at the same temperature, and then the solvent was removed in vacuo. The crude solid was purified by silica gel flash chromatography (elution with AcOEt/heptane) to yield the desired intermediate 70.

General procedure Q¹. To a stirring solution of adamantane-1,3-diamine (3) (1.5 eq.) in DCM (0.23 mmol/mL) was added pyridine (1.5 eq.) at 0° C. A solution of intermediate 70 (1.0 eq.) in DCM (0.30 mmol/mL) was added dropwise to the previous solution. The reaction was warmed up to room temperature and stirred for 12 h. After this time, the volatiles were removed in vacuo and the crude solid was purified by silica gel flash chromatography (elution with a mixture of DCM:MeOH:NH3 (8:2:0.2) in DCM) to yield the desired intermediate 71.

General procedure R¹. To a stirring solution of intermediate 71 (2.0 eq.) in ACN (0.03 mmol/mL) was added K₂CO₃ (3.0 eq.). A solution of 2-bromo-1-(5-fluoroisoindolin-2-yl)ethan-1-one (2) (1.0 eq.) in ACN (0.03 mmol/mL) was added dropwise to the previous mixture and the reaction was stirred for 18 h at room temperature. After this time, the volatiles were removed in vacuo and the crude solid was purified by by silica gel flash chromatography (elution with MeOH in DCM) followed by preparative TLC (elution with MeOH in DCM) to yield the desired compound 72.

General procedure S¹. A mixture of a specified carboxylic acid (1.2 eq.) and O-(7-azabenzotriazol-1-yl)-N,N,N′,N′-tetramethyluronium hexafluorophosphate (1.6 eq.) in dry DMF (reaction molarity 0.1 M) was stirred under argon atmosphere for 5 minutes at rt. A solution of tert-butyl (3-aminoadamantan-1-yl)carbamate (4) (1 eq.) and N,N-Diisopropylethylamine (4 eq.) in 20% of the total dry DMF used for the reaction was then added and the mixture was stirred at rt for 1.5 h. The reaction mixture was then poured in 5 times as much ultrapure water, triturated and left at 4° C. for 18 h. The suspension was then centrifuged and the supernatant discarded. The pellet was redispersed in the same amount of ultrapure water as the previous step, triturated, centrifuged and the supernatant was discarded. The pellet was then dried in vacuo and either used as-is in the following reaction or purified via normal phase column chromatography using a gradient of EtOAc in heptane.

General procedure T¹. The Boc-protected intermediate was dissolved in hydrogen chloride 4N solution in dioxane (10-20 eq.). If needed, as little methanol as necessary to solubilize the starting material was added and the mixture was stirred at room temperature until complete deprotection, between 1 h and 18 h. The volatiles were then removed via rotary evaporator and the compound was used as-is for the following reaction unless otherwise specified (quantitative yield).

General procedure U¹. A mixture of a specified carboxylic acid (1 eq.) and O-(7-azabenzotriazol-1-yl)-N,N,N′,N′-tetramethyluronium hexafluorophosphate (2 eq.) in dry DMF (reaction molarity 0.1 M) was stirred under argon atmosphere for 5 minutes at rt. A solution of 2-(((1s,3r,5R,7S)-3-aminoadamantan-1-yl)amino)-1-(5-fluoroisoindolin-2-yl)ethan-1-one dihydrochloride (1.2 eq) (81) (1 eq.) and N,N-Diisopropylethylamine (5 eq.) in 20% of the total dry DMF used for the reaction was then quickly added and the mixture was stirred at rt for 1.5 h. The reaction mixture was then poured in 5 times as much ultrapure water, triturated and left at 4° C. for 18 h. The suspension was then centrifuged and the supernatant discarded. The pellet was redispersed in the same amount of ultrapure water as the previous step, triturated, centrifuged and the supernatant was discarded. The pellet was then dried in vacuo and purified via normal phase column chromatography using a gradient of methanol in DCM.

General procedure V¹. 5 mg of the 3-N-amide derivative 82 were dissolved in 0.5 mL of dry DCM. If necessary, dry methanol was added one drop at a time followed by vortexing until complete dissolution of the starting material. The solution was then dropped into 1 mL of hydrogen chloride 4N solution in diethyl ether. The formed suspension was left to crystallize at 4° C. for 24 h and then decanted. The precipitate was washed once with 0.5 mL of diethyl ether and the residual solvent was removed in vacuo to yield the corresponding hydrochloride salt in quantitative yield.

General procedure W¹. In a rb flask, under argon atmosphere, a solution of the specified acyl chloride (1.2 eq.) in dry DCM (reaction molarity 0.1 M) was slowly dropped into a stirring solution of tert-butyl (3-aminoadamantan-1-yl)carbamate (1 eq.) and N,N-Diisopropylethylamine (4 eq.) in 80% of the total dry DCM used as solvent. The mixture was then stirred for 18 h at room temperature. The crude mixture was diluted with EtOAc and washed three times with a 1 M aqueous solution of HCl, once with brine, and twice with saturated sodium bicarbonate solution, once with brine. The organic phase was dried over sodium sulfate, evaporated to remove the solvent and purified via flash column chromatography over silica with a gradient of EtOAc in heptanes

General procedure X¹. Aryl carboxylic acid or PEG carboxylic acid (1.0 eq.) was dissolved in DMF and O-(7-Azabenzotriazol-1-yl)-N,N,N′,N′-tetramethyluronium PF6 (1.1 eq.) and N,N-diisopropylethylamine (3.0 eq.) were added. After 30 min of stirring, the reaction was cooled down to 0° C. and tert-butyl-3-aminoadamantan-1-yl)carbamate (1.1 eq.) was added. The reaction was stirred at room temperature overnight and, after that time, diluted with EtOAc and washed with a saturated NaHCO₃ solution. The organic layer was dried over Na₂SO₄, filtered and concentrated under reduced pressure. The crude was purified by silica gel flash chromatography (elution with MeOH in DCM) to yield the desired intermediate 79 as presented in scheme 15.

General procedure Y¹. Intermediate 79 (in scheme 17) was dissolved in DCM (0.5 M) and TFA (10.0 eq.) was added dropwise. The reaction was stirred at room temperature for 2 h. After this time, the volatiles were evaporated under reduced pressure to afford the free amine. The previous crude was dissolved in ACN (0.05 M) and N,N-diisopropylethylamine (1.1 eq.) was added and the mixture was stirred for 10 minutes. Potassium carbonate (3 eq.) was added and the reaction was cooled down to 0° C. The 2-bromo-1-(5-fluoroisoindolin-2-yl)ethan-1-one (2) (1.0 eq.) was dissolved in ACN (0.05M) and slowly added over to the stirring suspension. The reaction was warmed up to room temperature and and stirred overnight. After this time, the reaction mixture was concentrated under reduced pressure and the crude solid was purified by silica gel flash chromatography (elution with MeOH in DCM) to yield the desired compound 82 as presented in scheme 17. For the preparation of hydrochloride salts: Specific representatives of generic compound 82 were transformed into the corresponding hydrochloride salts 83. (Scheme 16) To this end, the specific representative of compound 82 was dissolved in DCM and MeOH was added in case the compound was not completely dissolved. A solution of HCl in diethyl ether was added dropwise to the previous solution and, after precipitation, the solvent was removed under reduced pressure to afford the desired compound as a hydrochloride salt.

General procedure Z¹. In a dry round-bottomed flask, compound 4 as presented in scheme 18 (1.5 eq.), 5-bromo-2-phenylpyridine (0.250 g, 1.068 mmol, 1.0 eq.), Pd₂(dba)₃ (0.2 eq.), Xphos (0.2 eq.), and Cs₂CO₃ (3.0 eq.) were added to dioxane (0.05 mmol/mL). The reaction mixture was degassed and stirred at 90° C. for 12 h. The reaction was cooled down, partitioned between EtOAc and H2O, and the organic layer was concentrated, and purified by silica gel flash chromatography (elution with MeOH in DCM) to yield tert-butyl (-3-((6-phenylpyridin-3-yl)amino)adamantan-1-yl)carbamate 108 as presented in scheme 18 (0.150 g, 0.358 mmol, 34% yield).

General procedure 1. To a stirring solution of 2-((3-aminoadamantan-1-yl)amino)-1-(5,6-difluoroisoindolin-2-yl)ethan-1-one bis(2,2,2-trifluoroacetate (6) (1.5 eq.) in DCM (0.23 mmol/mL) was added N,N-diisopropylethylamine (4.5 eq.) at 0° C. After stirring for 10 min, a solution of intermediate 65 (1.0 eq.) in DCM (0.30 mmol/mL) as presented in scheme 19 was added dropwise to the previous solution. The reaction was warmed up to room temperature and stirred for 18 h at reflux. After this time, the volatiles were removed in vacuo and the crude solid was purified by silica gel flash chromatography (elution with MeOH in DCM) to yield the desired intermediates 110-112 as presented in scheme 19.

General procedure 2. The Bsoc-protected intermediate 110-112 (1.0 eq.) as presented in scheme 19. were dissolved in DCM (0.5 M) and trifluoroacetic acid (10 eq.) was slowly added at room temperature. After 3 h, the volatiles were removed in vacuo and TFA was co-evaporated with toluene to yield the desired free amine as TFA salt. The previous crude was dissolved in DMF (0.07 mmol/mL) and triethylamine (3.5 eq.) was added. After stirring for 10 min, Biotin-NHS (1.1 eq.) was added portionwise at room temperature and the reaction was stirred for 2 h. After this time, the volatiles were removed in vacuo and the crude solid was purified by silica gel flash chromatography (elution with a mixture of DCM:MeOH:NH3 (8:2:0.2) in DCM) to yield the desired biotinylated derivative 113-115 as presented in Scheme 19.

General procedure 3. To a stirring solution of 2-(2-((tert-butoxycarbonyl)amino)ethoxy)ethyl-3-((2-(5-fluoroisoindolin-2-yl)-2oxoethyl)amino)adamantan-1-ylcarbamate (66) or 2-(2-((tert-butoxycarbonyl)amino)ethoxy)ethyl-3-((2-(5,6-difluoroisoindolin-2-yl)-2oxoethyl)amino)adamantan-1-ylcarbamate (110) (1.0 eq.) in DCM (0.5 M), trifluoroacetic acid (10 eq.) was slowly added at room temperature. After 3 h, the volatiles were removed in vacuo and TFA was co-evaporated with toluene to yield the desired free amine as TFA salt. The previous crude was dissolved in DCM (0.05 mmol/mL) and triethylamine (3.0 eq.) and slowly added to NBD-Cl or dansyl chloride (1.0 eq) in DCM (0.05 mmol/mL) at 0° C. After stirring for 5-12 h at room temperature, the reaction mixture was diluted with DCM, washed with H2O, dried over Na₂SO₄ and concentrated in vacuo. The crude residue was purified by silica gel flash chromatography (elution with MeOH in DCM) to yield the desired compounds 116-119 as presented in Scheme 20.

General procedure 4. To a stirring solution of Cy3-NHS (1.0 eq.) in DCM (0.018 mmol/mL) at 0° C. was added a mixture of 2-((3-aminoadamantan-1-yl)amino)-1-(5-fluoroisoindolin-2-yl)ethan-1-one bis(2,2,2-trifluoroacetate (6) or 2-((3-aminoadamantan-1-yl)amino)-1-(5,6-difluoroisoindolin-2-yl)ethan-1-one bis(2,2,2-trifluoroacetate (6) and DIPEA (4.5 eq.) in DCM (0.026 mmol/mL). The reaction mixture was warmed up to room temperature and stirred overnight. After this time, the volatiles were removed in vacuo and the crude solid was purified by silica gel flash chromatography (elution with a mixture of DCM and MeOH) to yield the desired Cy3 derivative 120-121 as presented in scheme 21.

General procedure 5. Compound 103-104 (1.0 eq.) was dissolved in a mixture of THF:MeOH:H₂O (3:2:1, 0.5 M) and LiOH (10.0 eq.) was added. The reaction mixture was stirred at room temperature for 3 h. After this time, the reaction mixture was corrected to pH 7 with HCl 1M and dried under reduced pressure. The obtained crude was used in the next step without further purification. The crude was dissolved in DMF and O-(7-Azabenzotriazol-1-yl)-N,N,N′,N′-tetramethyluronium PF6 (1.1 eq.) and N,N-diisopropylethylamine (3.2 eq.) were added. After 30 min of stirring, the reaction was cooled down to 0° C. and (2S,4R)-1-((S)-2-amino-3,3-dimethylbutanoyl)-4-hydroxy-N-(4-(4-methylthiazol-5-yl)benzyl)pyrrolidine-2-carboxamide TFA salt (1.1 eq.) was added. The reaction was stirred at room temperature overnight and, after that time, diluted with EtOAc and washed with a saturated NaHCO₃ solution. The organic layer was dried over Na₂SO₄, filtered and concentrated under reduced pressure. The crude was purified by silica gel flash chromatography (elution with MeOH in DCM) to yield the desired compound 122-123 as presented in scheme 22.

General procedure 6. Intermediate 82 (1.0 eq.) was dissolved in a DCM (0.5 M) TFA (10.0 eq.) was added. The reaction mixture was stirred at room temperature for 2 h. After this time, the reaction mixture was dried under reduced pressure. The obtained crude was used in the next step without further purification. The crude was dissolved in DMF and O-(7-Azabenzotriazol-1-yl)-N,N,N′,N′-tetramethyluroniumPF6 (1.1 eq.) and N,N-diisopropylethylamine (3.2 eq.) were added. After 30 min of stirring, the reaction was cooled down to 0° C. and (2S,4R)-1-((S)-2-amino-3,3-dimethylbutanoyl)-4-hydroxy-N-(4-(4-methylthiazol-5-yl)benzyl)pyrrolidine-2-carboxamide TFA salt (1.1 eq.) was added. The reaction was stirred at room temperature overnight and, after that time, diluted with EtOAc and washed with a saturated NaHCO₃ solution. The organic layer was dried over Na₂SO₄, filtered and concentrated under reduced pressure. The crude was purified by silica gel flash chromatography (elution with MeOH in DCM) to yield the desired compound 124-125 as presented in scheme 23.

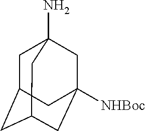 [SMILES: CC(C)(C)OC(=O)NC12CC3CC(CC(N)(C3)C1)C2]

tert-butyl (3-aminoadamantan-1-yl)carbamate (4). To a mixture of adamantane-1,3-diamine (5 g, 30.1 mmol) and potassium carbonate (5 eq.) in dioxane (200 mL) and water (200 mL) at 0° C., was added Di-t-butyldicarbonate (1 eq.) in 1,4-dioxane (10 mL) portionwise over 10 min. The reaction mixture was warmed to rt, stirred for 18 h, diluted with DCM and the layers were separated. The organic layer was dried over anhydrous Na₂SO₄, filtered, concentrated and purified by flash chromatography on SiO2 with EtOAc in heptane (80-100%) to yield tert-butyl (3-aminoadamantan-1-yl)carbamate (4.92 g, 18.47 mmol, 61% yield) as a white solid.^{[1]}

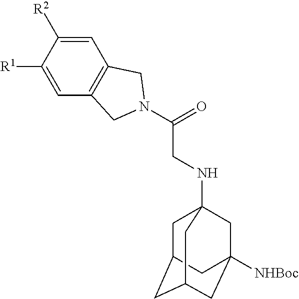

tert-butyl(3-((2-(isoindolin-2-yl)-2-oxoethyl)amino)adamantan-1-yl)carbamate derivative (5). General procedure B with tert-butyl (3-aminoadamantan-1-yl)carbamate (4.30 g, 11.37 mmol). NH Purified by flash chromatography on SiO2 with MeOH in EtOAC (0-15%) to yield the desired product (39-52% yield).

2-((3-aminoadamantan-1-yl)amino)-1-(isoindolin-2-yl)ethan-1-one bis(2,2,2-trifluoroacetate derivative (6). General procedure C with tert-butyl (3-((2-(isoindolin-2-yl)-2-oxoethyl)amino)adamantan-1-yl)carbamate derivative (5) (quantitative yield).

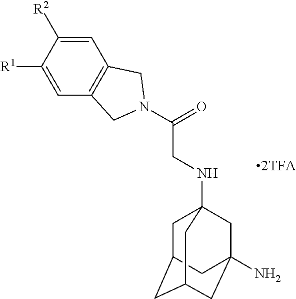

tert-butyl (3-hydroxyadamantan-1-yl)carbamate (10). To a solution of 3-aminoadamantan-1-ol (1 g, 5.98 mmol) in DCM (19.9 mL), triethylamine (0.829 mL, 5.98 mmol) was added followed by

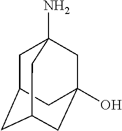 [SMILES: NC12CC3CC(C1)CC(O)(C3)C2]

the addition of di-t-butyldicarbonate (1.305 g, 5.98 mmol) portionwise. The reaction mixture was stirred for 3 h, then the volatiles were evaporated under reduced pressure. The residue was redissolved in EtOAc (150 mL) and washed with a 1 M solution of HCl (2×50 mL). The organic phase was dried over Na₂SO₄ and further concentrated under reduced pressure. The resulting crude was washed with ether yielding the title compound tert-butyl (3-hydroxyadamantan-1-yl)carbamate (1.376 g, 5.15 mmol, 86% yield) as a white solid. MS (ESI) m/z 150.2 [M−Boc−H₂O]⁺, 194.2 [M-tert-BuOH+H]⁺.^{[2]}

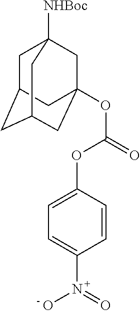

tert-butyl (3-(((4-nitrophenoxy)carbonyl)oxy)adamantan-1-yl)carbamate (11). To a solution of tert-butyl (3-hydroxyadamantan-1-yl)carbamate (500 mg, 1.870 mmol) in DCM (0.1 M), pyridine (4 eq.) and 4-nitrophenylchloroformate (1.5 eq.) were added and the reaction mixture was stirred at rt for 2 h. The reaction mixture was washed with NaHCO₃ (sat. solution). The aqueous phase was back-extracted with DCM (2×40 mL). The organic phases were combined and dried over Na₂SO₄, filtered and concentrated. The crude was purified by flash chromatography on SiO₂ with EtOAc in heptane (0-20%) to afford tert-butyl (3-(((4-nitrophenoxy)carbonyl)oxy)adamantan-1-yl)carbamate (610 mg, 1.410 mmol, 75% yield). ¹H NMR (400 MHz, CDCl₃) δ 8.23-8.17 (m, 2H), 7.33-7.26 (m, 2H), 4.42 (s, 1H), 2.36 (s, 2H), 2.29 (q, J=3.1 Hz, 2H), 2.08 (d, J=3.0 Hz, 4H), 1.92 (d, J=12.0 Hz, 2H), 1.75 (d, J=12.0 Hz, 2H), 1.54 (d, J=2.8 Hz, 2H), 1.36 (s, 9H). ¹³C NMR (101 MHz, CDCl₃) δ 155.59, 149.80, 125.21, 121.92, 84.23, 53.01, 40.49, 39.66, 34.74, 30.61, 28.45.

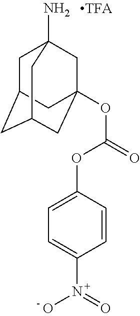

3-aminoadamantan-1-yl (4-nitrophenyl) carbonate 2,2,2-trifluoroacetate (12). General procedure C from tert-butyl (3-(((4-nitrophenoxy)carbonyl)oxy)adamantan-1-yl)carbamate (0.55 g, 0.636 mmol) to yield 3-aminoadamantan-1-yl (4-nitrophenyl) carbonate 2,2,2-trifluoroacetate (0.284 g, 0.663 mmol, 100% yield) as a white solid. MS (ESI) m/z 333.3 [M+H]⁺

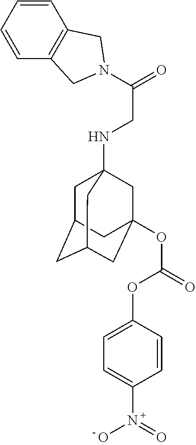

3-((2-(isoindolin-2-yl)-2-oxoethyl)amino)adamantan-1-yl (4-nitrophenyl) carbonate (13). General procedure B with 3-aminoadamantan-1-yl (4-nitrophenyl) carbonate 2,2,2-trifluoroacetate (2.3 g, 4.64 mmol). Purified with flash chromatography on SiO₂ with MeOH in DCM (0-5%) to yield 3-((2-(isoindolin-2-yl)-2-oxoethyl)amino)adamantan-1-yl (4-nitrophenyl) carbonate (1.33 g, 1.495 mmol, 72% yield). MS (ESI) m/z 492.3 [M+H]⁺.

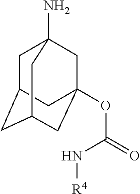

R4 substituted 3-aminoadamantan-1-yl carbamate intermediates (15). General procedure G (65-90% yield).

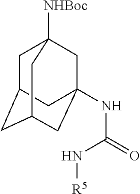

R⁵ substituted tert-butyl (3-(3-ureido)adamantan-1-yl)carbamate intermediates (16). General procedure H (54-74% yield).

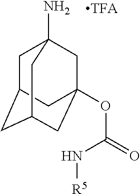

R⁵ substituted 1-(3-aminoadamantan-1-yl)-3-urea 2,2,2-trifluoroacetate derivatives (17). General procedure C with R⁵ substituted tert-butyl (3-(3-ureido)adamantan-1-yl)carbamate intermediates (16) (quantitative yield).

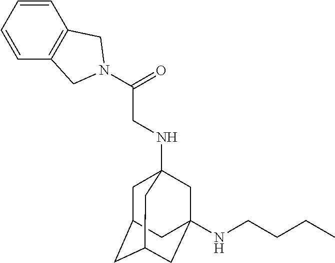 [SMILES: CCCCNC12CC3CC(C1)CC(NCC(=O)N1Cc4ccccc4C1)(C3)C2]

2-((3-(butylamino)adamantan-1-yl)amino)-1-(isoindolin-2-yl)ethan-1-one 2,2,2-trifluoroacetate (25). General procedure B with N1-butyladamantane-1,3-diamine 2,2,2-trifluoroacetate (70 mg, 0.155 mmol). After flash chromatography, the product was dissolved in DCM (0.5 M) and 5 eq. of trifluoroacetic acid were added. The reaction was stirred for 30 minutes, then coevaporated with toluene to yield 2-((3-(butylamino)adamantan-1-yl)amino)-1-(isoindolin-2-yl)ethan-1-one 2,2,2-trifluoroacetate (38 mg, 0.062 mmol, 80% yield) as a white solid. ¹H NMR (400 MHz, DMSO) δ 7.40-7.27 (m, 1H), 4.84 (s, 1H), 4.66 (s, 1H), 3.45 (s, 1H), 2.88-2.76 (m, 1H), 2.23 (s, 1H), 1.83-1.60 (m, 3H), 1.63-1.42 (m, 1H), 1.34 (q, J=7.4 Hz, 1H), 0.89 (t, J=7.3 Hz, 1H). ¹³C NMR (101 MHz, DMSO) δ 170.28, 158.50 (q, J=31.2 Hz), 137.20, 136.39, 127.87, 123.52, 123.23, 117.67 (q, J=299.7 Hz), 57.37, 52.39, 52.25, 51.29, 43.39, 43.07, 40.81, 37.92, 34.95, 29.47, 29.23, 19.96, 14.04. MS (ESI) m/z 382.3 [M+H]⁺, 404.4 [M+Na]⁺.

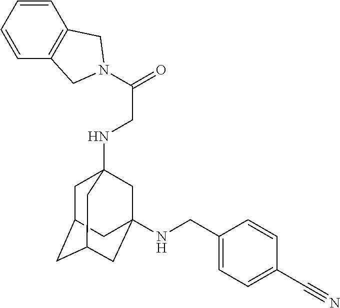 [SMILES: N#Cc1ccc(CNC23CC4CC(CC(NCC(=O)N5Cc6ccccc6C5)(C4)C2)C3)cc1]

4-(((-3-((2-(isoindolin-2-yl)-2-oxoethyl)amino)adamantan-1-yl)amino)methyl)benzonitrile (26). General procedure D with 2-((3-aminoadamantan-1-yl)amino)-1-(isoindolin-2-yl)ethan-1-one bis(2,2,2-trifluoroacetate) (150 mg, 0.271 mmol) and alpha-Bromo-p-tolunitrile (64 mg, 0.325 mmol) to yield 4-(((3-((2-(isoindolin-2-yl)-2-oxoethyl)amino)adamantan-1-yl)amino)methyl)benzonitrile (71 mg, 0.161 mmol, 60% yield) as a white powder. ¹H NMR (400 MHz, CDCl₃) δ 7.59 (d, J=8.0 Hz, 2H), 7.47 (d, J=8.1 Hz, 2H), 7.37-7.24 (m, 4H), 4.82 (s, 2H), 4.79 (s, 2H), 3.83 (s, 2H), 3.50 (s, 2H), 2.31-2.23 (m, 2H), 1.79 (s, 2H), 1.72-1.50 (m, 12H). ¹³C NMR (101 MHz, CDCl₃) δ 170.29, 147.41, 136.12, 135.85, 132.13, 127.92, 128.71, 127.60, 123.07, 122.64, 119.06, 110.41, 52.95, 52.32, 52.27, 51.54, 47.74, 44.98, 43.06, 42.06, 41.67, 35.71, 30.02. MS (ESI) m/z 491.4 [M+H]⁺, 463.4 [M+Na]⁺. HRMS: calc. 441.2649, found 441.2630 [M+H]⁺.

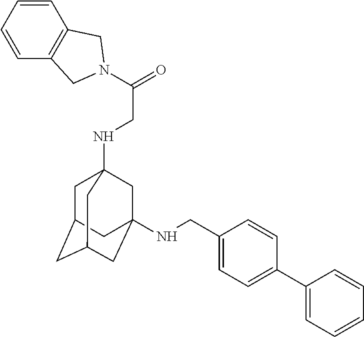 [SMILES: O=C(CNC12CC3CC(C1)CC(NCc1ccc(-c4ccccc4)cc1)(C3)C2)N1Cc2ccccc2C1]

2-((3-(([1,1′-biphenyl]-4-ylmethyl)amino)adamantan-1-yl)amino)-1-(isoindolin-2-yl)ethan-1-one (27). General procedure N_{H}D with 2-((3-aminoadamantan-1-yl)amino)-1-(isoindolin-2-yl)ethan-1-one bis(2,2,2-trifluoroacetate) (150 mg, NH 0.271 mmol) and 4-Bromomethylbiphenyl (80 mg, 0.325 mmol) to yield 2-((3-(([1,1′-biphenyl]-4-ylmethyl)amino)adamantan-1-yl)amino)-1-(isoindolin-2-yl)ethan-1-one (65 mg, 0.132 mmol, 49% yield) as a white powder. ¹H NMR (400 MHz, CDCl₃) δ 7.52 (dd, J=10.2, 8.2 Hz, 6H), 7.44-7.30 (m, 3H), 7.32-7.21 (m, 4H), 4.80 (s, 2H), 4.77 (s, 2H), 3.83 (s, 2H), 3.50 (s, 2H), 2.25 (s, 2H), 1.78 (s, 6H), 1.64 (d, J=3.8 Hz, 4H), 1.57 (s, 2H). ¹³C NMR (101 MHz, CDCl₃) δ 170.01, 140.64, 140.37, 136.08, 135.88, 129.52, 128.74, 127.90, 127.61, 127.29, 127.21, 126.98, 123.06, 122.71, 55.08 (d, J=4.4 Hz), 52.54, 52.36, 51.57, 45.92, 44.77, 42.89, 41.43, 40.69, 35.42, 29.90. MS (ESI) m/z 492.4 [M+H]⁺.

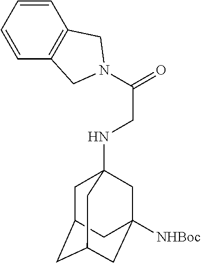 [SMILES: CC(C)(C)OC(=O)NC12CC3CC(CC(NCC(=O)N4Cc5ccccc5C4)(C3)C1)C2]

tert-butyl(3-((2-(isoindolin-2-yl)-2oxoethyl)amino)adamantan-1-yl)carbamate (28). General procedure B with tert-butyl (3-aminoadamantan-1-yl)carbamate (4.30 g, 11.37 mmol). The crude was purified by flash chromatography on SiO₂ with MeOH in EtOAc (0-15%) to yield tert-butyl (3-((2-(isoindolin-2-yl)-2-oxoethyl)amino)adamantan-1-yl)carbamate (1.935 g, 4.55 mmol, 52% yield) as white solid. ¹H NMR (400 MHz, MeOD) δ 7.36 (m, 4H), 4.93 (s, 2H), 4.82 (s, 2H), 3.92 (s, 2H), 2.35-2.23 (m, 2H), 2.14 (s, 2H), 2.02 (d, J=12.1 Hz, 2H), 1.98-1.74 (m, 6H), 1.75-1.63 (m, 2H), 1.44 (s, 9H). ¹³C NMR (101 MHz, MeOD) δ 166.31, 155.17, 135.30, 135.86, 127.60, 127.50, 122.59, 122.47, 78.33, 56.09, 52.02, 51.29, 51.19, 42.54, 41.05, 39.87, 38.13, 34.48, 29.64, 27.42. MS (ESI) m/z 426.4 [M+H]⁺. HRMS: calc. 426.2751, found 426.2736 [M+H]⁺.

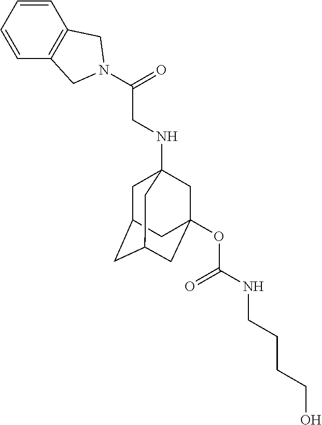 [SMILES: O=C(NCCCCO)OC12CC3CC(CC(NCC(=O)N4Cc5ccccc5C4)(C3)C1)C2]

3-((2-(isoindolin-2-yl)-2-oxoethyl)amino)adamantan-1-yl(4-hydroxybutyl)carbamate (29). General procedure E with 3-((2-(isoindolin-2-yl)-2-oxoethyl)amino)adamantan-1-yl (4-nitrophenyl) carbonate (100 mg, 0.151 mmol) and 4-aminobutan-1-ol (27 mg, 0.301 mmol). The reaction crude was purified by flash chromatography on Si—C18 with ACN in water (20-100%) to yield 3-((2-(isoindolin-2-yl)-2-oxoethyl)amino)adamantan-1-yl (4-hydroxybutyl)carbamate (48 mg, 0.109 mmol, 72% yield) as a white powder after lyophylisation. ¹H NMR (400 MHz, MeOD) δ 7.39-7.29 (m, 4H), 4.89 (s, 2H), 4.79 (s, 2H), 3.58-3.55 (m, 4H), 3.06 (t, J=5.8 Hz, 2H), 2.31 (s, 2H), 2.17-2.02 (m, 6H), 1.77-1.65 (m, 4H), 1.63-1.60 (m, 2H), 1.58-1.49 (m, 4H). ¹³C NMR (101 MHz, MeOD) δ 170.25, 136.09, 135.53, 78.86, 61.18, 53.62, 51.90, 51.17, 48.23, 48.02, 47.80, 47.59, 47.38, 47.17, 47.17, 46.96, 46.95, 45.41, 41.87, 40.46, 40.27, 39.72, 34.83, 30.75, 29.43, 25.97. MS (ESI) m/z 442.4 [M+H]⁺, 464.4 [M+Na]⁺. HRMS: calc. 442.2700, found 442.2617 [M+H]⁺.

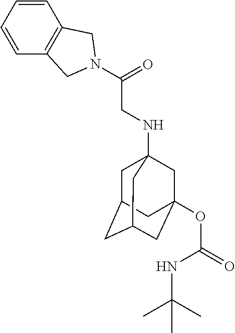 [SMILES: CC(C)(C)NC(=O)OC12CC3CC(CC(NCC(=O)N4Cc5ccccc5C4)(C3)C1)C2]

3-((2-(isoindolin-2-yl)-2-oxoethyl)amino)adamantan-1-yl butylcarbamate (30). General procedure B with 3-((2-(isoindolin-2-yl)-2-oxoethyl)amino)adamantan-1-yl (4-nitrophenyl) carbonate (128 mg, 0.260 mmol). The crude was redissolved in EtOAc and washed with water, dried under reduced pressure then purified by flash chromatography on Si—C18 with MeOH in (10-100%) to yield 3-((2-(isoindolin-2-yl)-2-oxoethyl)amino)adamantan-1-yl tert-butylcarbamate (40.2 mg, 0.094 mmol, 36% yield) as a white powder. ¹H NMR (400 MHz, Methanol-d4) δ 7.40-7.27 (m, 4H), 4.88 (s, 2H), 4.78 (s, 2H), 3.55 (s, 2H), 2.31-2.25 (m, 2H), 2.07 (s, 6H), 1.75-1.56 (m, 6H), 1.27 (s, 9H). ¹³C NMR (101 MHz, Methanol-d4) δ 170.47, 155.07, 136.11, 135.55, 127.49, 127.37, 122.55, 122.45, 78.50, 53.46, 51.91, 51.19, 49.31, 45.53, 41.93, 40.56, 40.45, 34.92, 30.73, 27.77. MS (ESI) m/z 426.4 [M+H]⁺. HRMS: calc. 425.2751, found 426.2755 [M+H]⁺.

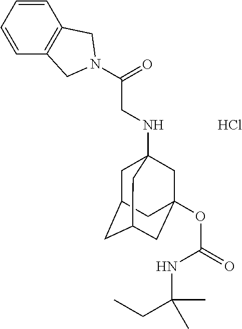 [SMILES: CCC(C)(C)NC(=O)OC12CC3CC(CC(NCC(=O)N4Cc5ccccc5C4)(C3)C1)C2 ; Cl]

3-((2-(isoindolin-2-yl)-2-oxoethyl)amino)adamantan-1-yl tert-pentylcarbamate hydrochloride (31). General method A with 3-aminoadamantan-1-yl tert-pentylcarbamate (65 mg, 0.232 mmol) The crude was purified with flash chromatography on SiO₂ with MeOH in DCM (0-15%), then 5 eq. of HCl 2M in ether was added dropwise and a white solid was precipitated to yield 3-((2-(isoindolin-2-yl)-2-oxoethyl)amino)adamantan-1-yl tert-pentylcarbamate hydrochloride (10 mg, 0.021 mmol, 14% yield) as a white powder. ¹H NMR (400 MHz, DMSO) δ 9.18 (s, 2H), 7.49-7.20 (m, 4H), 6.54 (s, 1H), 4.98 (s, 2H), 4.73 (s, 2H), 4.06-4.02 (m, 2H), 2.34 (s, 2H), 2.33-2.28 (m, 2H), 2.08-1.96 (m, 4H), 1.93-1.87 (m, 4H), 1.61-1.44 (m, 4H), 1.13 (s, 6H), 0.75 (t, J=7.5 Hz, 3H). ¹³C NMR (101 MHz, DMSO) δ 164.66, 136.75, 135.84, 128.12, 128.05, 123.62, 123.15, 77.30, 58.94, 52.49, 52.34, 51.53, 42.24, 41.06, 40.33, 36.64, 34.45, 32.45, 30.05, 26.84, 8.85. MS (ESI) m/z 440.4 [M+H]⁺, 462.4 [M+Na]⁺. HRMS: calc. 440.2908, found 440.2888 [M+H]⁺.

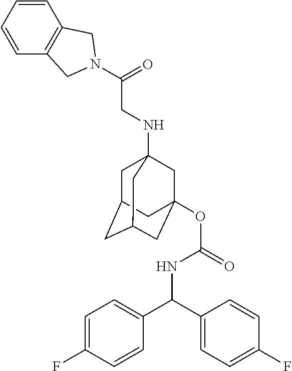 [SMILES: O=C(NC(c1ccc(F)cc1)c1ccc(F)cc1)OC12CC3CC(CC(NCC(=O)N4Cc5ccccc5C4)(C3)C1)C2]

3-((2-(isoindolin-2-yl)-2-oxoethyl)amino)adamantan-1-yl (bis(4-fluorophenyl)methyl)carbamate (32). General procedure B with 3-aminoadamantan-1-yl (bis(4-fluorophenyl)methyl)carbamate (78 mg, 0.189 mmol). The crude was purified with flash chromatography on Si—C18 with ACN in water (30-100%) to yield 3-((2-(isoindolin-2-yl)-2-oxoethyl)amino)adamantan-1-yl (bis(4-fluorophenyl)methyl)carbamate (8 mg, 0.014 mmol, 15% yield) as a white solid. ¹H NMR (400 MHz, CDCl₃) δ 7.34-7.26 (m, 4H), 7.23-7.18 (m, 4H), 7.09-6.99 (m, 4H), 5.86 (d, J=6.8 Hz, 1H), 5.19 (d, J=7.2 Hz, 1H), 4.83 (s, 2H), 4.79 (s, 2H), 3.54 (s, 2H), 2.70 (s, 1H), 2.35-2.28 (m, 2H), 2.18-2.00 (m, 6H), 1.79-1.44 (m, 6H). ¹³C NMR (101 MHz, CDCl₃) δ 169.56, 162.10 (d, J=246.1 Hz), 154.40, 137.50, 136.05, 135.82, 128.84 (d, J=8.1 Hz), 127.95, 127.66, 123.07, 122.71, 115.60 (d, J=21.3 Hz), 80.54, 57.29, 54.05, 52.39, 51.59, 45.68, 42.83, 41.10, 40.67, 35.17, 30.60. MS (ESI) m/z 572.3 [M+H]⁺. HRMS: calc. 572.2719, found 572.2294 [M+H]⁺.

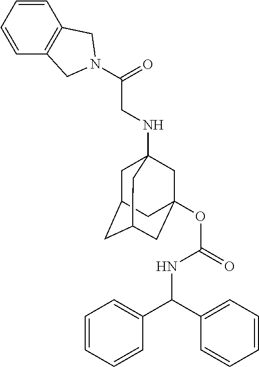 [SMILES: O=C(NC(c1ccccc1)c1ccccc1)OC12CC3CC(CC(NCC(=O)N4Cc5ccccc5C4)(C3)C1)C2]

3-((2-(isoindolin-2-yl)-2-oxoethyl)amino)adamantan-1-yl benzhydrylcarbamate (33). General procedure E with 3-((2-(isoindolin-2-yl)-2-oxoethyl)amino)adamantan-1-yl (4-nitrophenyl) carbonate (100 mg, 0.203 mmol) and alpha-Aminodiphenylmethane (112 mg, 0.610 mmol). The crude was purified by flash chromatography on Si C-18 from with ACN in water (20 to 82%) to yield 3-((2-(isoindolin-2-yl)-2-oxoethyl)amino)adamantan-1-yl benzhydrylcarbamate (38 mg, 0.071 mmol, 35% yield)) as a pale yellow oil. ¹H NMR (400 MHz, Chloroform-d) δ 7.45-7.13 (m, 14H), 5.89 (d, J=7.7 Hz, 1H), 5.29 (s, 1H), 4.81 (s, 2H), 4.76 (s, 2H), 3.47 (s, 2H), 2.27 (s, 2H), 2.05 (s, 6H), 1.76-1.42 (m, 6H). ¹³C NMR (101 MHz, Chloroform-d) δ 170.22, 154.58, 141.96, 136.17, 135.99, 128.63, 127.87, 127.58, 127.38, 127.23, 123.05, 122.69, 80.43, 58.50, 53.48, 52.31, 51.58, 46.01, 43.11, 41.51, 40.78, 35.31, 30.66. MS (ESI) m/z 536.3 [M+H]⁺, 558.4 [M+Na]⁺. HRMS: calc. 536.2908, found 536.2899 [M+H]⁺.

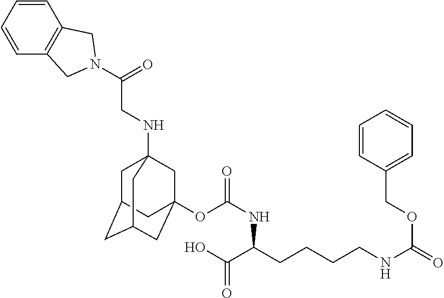 [SMILES: O=C(NCCCC[C@H](NC(=O)OC12CC3CC(CC(NCC(=O)N4Cc5ccccc5C4)(C3)C1)C2)C(=O)O)OCc1ccccc1]

N⁶-((benzyloxy)carbonyl)-N²-(((3-((2-(isoindolin-2-yl)-2-oxoethyl)amino)adamantan-1-yl)oxy)carbonyl)-L-lysine (34). General procedure E with 3-((2-(isoindolin-2-yl)-2-oxoethyl)amino)adamantan-1-yl (4-nitrophenyl) carbonate (95 mg, 0.164 mmol) and N⁶-((benzyloxy)carbonyl)-L-lysine (60 mg, 0.214 mmol). Reaction was heated up to 60° C. The crude was purified by flash chromatography on Si—C18 with MeOH in water (10-100%) to yield N⁶-((benzyloxy)carbonyl)-N²-(((3-((2-(isoindolin-2-yl)-2-oxoethyl)amino)adamantan-1-yl)oxy)carbonyl)-L-lysine (10 mg, 0.016 mmol, 10% yield) as a white solid. ¹H NMR (400 MHz, Methanol-d4) δ 7.38-7.18 (m, 9H), 5.05 (s, 2H), 4.90 (s, 2H), 4.80 (s, 2H), 4.01-3.93 (m, 1H), 3.93 (s, 2H), 3.11 (t, J=6.5 Hz, 2H), 2.37 (s, 2H), 2.31 (s, 2H), 2.17-2.03 (m, 4H), 1.93-1.70 (m, 5H), 1.65 (s, 3H), 1.51 (h, J=13.3, 6.3 Hz, 2H), 1.39 (p, J=12.0, 6.2 Hz, 2H). ¹³C NMR (101 MHz, Methanol-d4) δ 178.32, 166.16, 157.45, 155.60, 137.08, 135.83, 135.29, 128.05, 127.61, 127.51, 127.30, 122.60, 122.46, 78.19, 65.87, 57.19, 55.54, 52.03, 51.17, 43.00, 41.14, 40.32, 40.04, 37.96, 34.23, 32.35, 30.42, 29.24, 22.68. MS (ESI) m/z 633.4 [M+H]⁺. HRMS: calc. 633.3283, found 633.3263 [M+H]⁺.

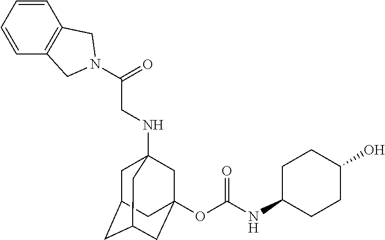 [SMILES: O=C(N[C@H]1CC[C@H](O)CC1)OC12CC3CC(CC(NCC(=O)N4Cc5ccccc5C4)(C3)C1)C2]

3-((2-(isoindolin-2-yl)-2-oxoethyl)amino)adamantan-1-yl ((1r,4R)-4-hydroxycyclohexyl)carbamate (35). General procedure E with 3-((2-(isoindolin-2-yl)-2-oxoethyl)amino)adamantan-1-yl (4-nitrophenyl) carbonate (100 mg, 0.203 mmol) and trans-4-Aminocyclohexanol (47 mg, 0.407 mmol). Reaction mixture was heated up to 60° C. The crude was purified by flash chromatography on Si—C18 with MeOH in water (10-100%) to yield 3-((2-(isoindolin-2-yl)-2-oxoethyl)amino)adamantan-1-yl ((1r,4R)-4-hydroxycyclohexyl)carbamate (49 mg, 0.105 mmol, 52% yield) as a white powder after lyophylisation. ¹H NMR (400 MHz, Methanol-d4) δ 7.40-7.14 (m, 4H), 4.89 (s, 2H), 4.79 (s, 2H), 3.55 (s, 2H), 3.54-3.40 (m, 1H), 3.33-3.21 (m, 1H), 2.30 (bs, 2H), 2.23-2.04 (m, 6H), 2.00-1.81 (m, 4H), 1.72-1.54 (m, 6H), 1.46-1.05 (m, 4H). ¹³C NMR (101 MHz, Methanol-d4) δ 170.44, 155.89, 136.10, 135.54, 127.49, 127.37, 122.54, 122.44, 78.92, 69.05, 53.51, 51.91, 51.19, 48.81, 45.50, 41.92, 40.51, 40.39, 34.87, 33.48, 30.73, 30.45. MS (ESI) m/z 468.4 [M+H]⁺, 490.4 [M+Na]⁺. HRMS: calc. 468.2857, found 468.2856 [M+H]⁺.

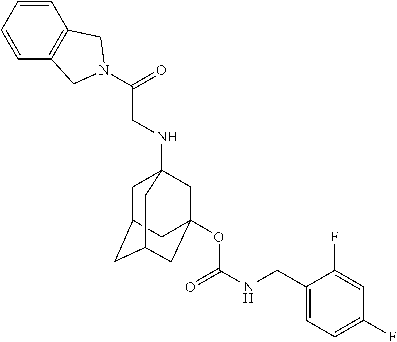 [SMILES: O=C(NCc1ccc(F)cc1F)OC12CC3CC(CC(NCC(=O)N4Cc5ccccc5C4)(C3)C1)C2]

3-((2-(isoindolin-2-yl)-2-oxoethyl)amino)adamantan-1-yl benzylcarbamate (36). General procedure E with 3-((2-(isoindolin-2-yl)-2-oxoethyl)amino)adamantan-1-yl (4-nitrophenyl) carbonate (100 mg, 0.203 mmol) and alpha-Aminotoluene (67 μl, 0.610 mmol) The reaction was heated to 65° C. The crude was purified by flash chromatography on Si—C18 with ACN in water (10-100%) to yield 3-((2-(isoindolin-2-yl)-2-oxoethyl)amino)adamantan-1-yl benzylcarbamate (28 mg, 0.060 mmol, 30% yield) as a pale yellow oil. ¹H NMR (400 MHz, Chloroform-d) δ 7.37-7.16 (m, 9H), 4.99 (t, J=6.0 Hz, 1H), 4.81 (s, 2H), 4.77 (s, 2H), 4.30 (d, J=5.9 Hz, 2H), 3.50 (s, 2H), 2.40-2.22 (m, 2H), 2.15-1.99 (m, 6H), 1.77-1.48 (m, 6H). ¹³C NMR (101 MHz, Chloroform-d) δ 170.22, 155.44, 138.79, 136.18, 135.98, 128.62, 127.88, 127.59, 127.47, 127.35, 123.06, 122.69, 80.12, 53.40, 52.31, 51.58, 46.13, 44.65, 43.12, 41.53, 40.82, 35.34, 30.68. MS (ESI) m/z 460.4 [M+H]⁺, 482.4 [M+Na]⁺. HRMS: calc. 460.2595, found 460.2615 [M+H]⁺.

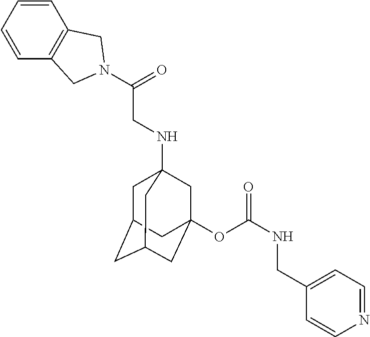 [SMILES: O=C(NCc1ccncc1)OC12CC3CC(CC(NCC(=O)N4Cc5ccccc5C4)(C3)C1)C2]

3-((2-(isoindolin-2-yl)-2-oxoethyl)amino)adamantan-1-yl (pyridin-4-ylmethyl)carbamate (37). General procedure E with 3-((2-(isoindolin-2-yl)-2-oxoethyl)amino)adamantan-1-yl (4-nitrophenyl) carbonate (100 mg, 0.203 mmol) and by 4-Pyridinemethaneamine (41 μl, 0.407 mmol). The reaction was heated to 80° C. The reaction mixture was diluted with DCM, washed with a saturated NaHCO₃ solution, dried with Na₂SO₄, then under reduced pressure. The crude was purified by flash chromatography with ACN in water (20-50%) to yield (1s,3r,5R,7S)-3-((2-(isoindolin-2-yl)-2-oxoethyl)amino)adamantan-1-yl (pyridin-4-ylmethyl)carbamate (26 mg, 0.056 mmol, 28% yield). ¹H NMR (400 MHz, CDCl₃) δ 8.54 (d, J=5.0 Hz, 2H), 7.32-7.24 (m, 4H), 7.20 (d, J=6.0 Hz, 2H), 5.33 (d, J=7.2 Hz, 1H), 4.81 (s, 2H), 4.77 (s, 2H), 4.32 (d, J=6.2 Hz, 2H), 3.49 (s, 2H), 2.30 (s, 2H), 2.14-2.01 (m, 6H), 1.72-1.48 (m, 6H). ¹³C NMR (101 MHz, Chloroform-d) δ 170.10, 155.53, 149.94, 148.12, 136.10, 135.88, 127.91, 127.61, 123.06, 122.68, 121.97, 80.54, 53.48, 52.32, 51.56, 46.17, 43.45, 43.04, 41.38, 40.73, 35.25, 30.65. MS (ESI) m/z 461.4 [M+H]⁺, 483.3 [M+Na]⁺. HRMS: calc. 461.2547, found 461.2533 [M+H]⁺.

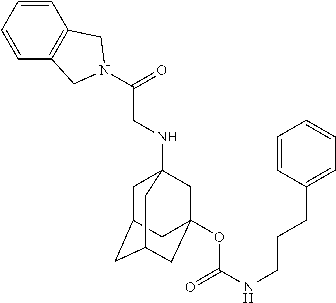 [SMILES: O=C(NCCCc1ccccc1)OC12CC3CC(CC(NCC(=O)N4Cc5ccccc5C4)(C3)C1)C2]

3-((2-(isoindolin-2-yl)-2-oxoethyl)amino)adamantan-1-yl (3-phenylpropyl)carbamate (38). General procedure E with 3-((2-(isoindolin-2-yl)-2-oxoethyl)amino)adamantan-1-yl (4-nitrophenyl) carbonate (100 mg, 0.151 mmol) and 3-Aminopropylbenzene (41 mg, 0.301 mmol). The crude was purified by flash chromatography on Si—C18 with ACN in water (20 to 100%) to yield 3-((2-(isoindolin-2-yl)-2-oxoethyl)amino)adamantan-1-yl (3-phenylpropyl)carbamate (49 mg, 0.100 mmol, 67% yield). ¹H NMR (400 MHz, Methanol-d4) δ 7.34 (q, J=2.8, 2.4 Hz, 4H), 7.25 (t, J=7.5 Hz, 2H), 7.21-7.11 (m, 3H), 4.89 (s, 2H), 4.80 (s, 2H), 3.86 (s, 2H), 3.05 (t, J=7.0 Hz, 2H), 2.61 (t, J=8.5 Hz, 2H), 2.42-2.33 (m, 2H), 2.24 (s, 2H), 2.10 (q, J=11.9 Hz, 4H), 1.84 (s, 4H), 1.82-1.68 (m, 2H), 1.70-1.60 (m, 2H). ¹³C NMR (101 MHz, Methanol-d4) δ 168.08, 157.78, 143.07, 137.25, 136.72, 129.40, 129.38, 129.01, 128.89, 126.87, 123.99, 123.84, 58.08, 79.53, 53.39, 52.55, 44.75, 42.64, 41.50, 40.97, 39.66, 35.69, 34.08, 32.81, 31.86. MS (ESI) m/z 488.4 [M+H]⁺, 510.4 [M+Na]⁺. HRMS: calc. 488.2908, found 488.2887 [M+H]⁺.

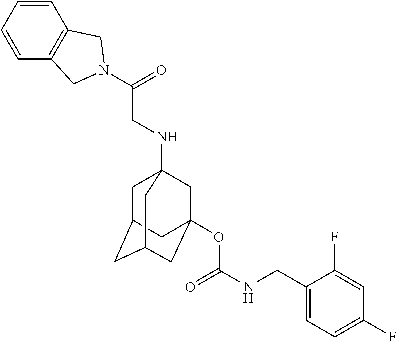 [SMILES: O=C(NCc1ccc(F)cc1F)OC12CC3CC(CC(NCC(=O)N4Cc5ccccc5C4)(C3)C1)C2]

3-((2-(isoindolin-2-yl)-2-oxoethyl)amino)adamantan-1-yl (2,4-difluorobenzyl)carbamate (39). General procedure E with 3-((2-(isoindolin-2-yl)-2-oxoethyl)amino)adamantan-1-yl (4-nitrophenyl) carbonate (100 mg, 0.151 mmol) and (2,4-difluorophenyl)methanamine (43 mg, 0.301 mmol) in ACN. The crude was purified by flash chromatography on Si C-18 from 20 to 100% ACN in water to yield 3-((2-(isoindolin-2-yl)-2-oxoethyl)amino)adamantan-1-yl (2,4-difluorobenzyl)carbamate (11 mg, 0.022 mmol, 15% yield) as a white powder after lyophylisation. ¹H NMR (400 MHz, Methanol-d4) δ 7.41-7.31 (m, 5H), 6.94 (t, J=9.3 Hz, 2H), 4.92 (s, 2H), 4.83 (s, 2H), 4.25 (s, 2H), 3.95 (s, 2H), 2.45-2.37 (m, 2H), 2.32 (s, 2H), 2.21-2.05 (m, 4H), 1.94-1.81 (m, 4H), 1.69 (q, J=13.1, 12.5 Hz, 2H). ¹³C NMR (101 MHz, Methanol-d4) δ 130.17 (dd, J=9.7, 6.0 Hz), 127.53, 127.40, 122.55, 122.44, 169.23, 162.23 (dd, J=246.3, 11.9 Hz), 160.63 (dd, J=247.9, 12.1 Hz), 156.34, 136.01, 135.46, 122.28 (d, J=3.7 Hz), 110.54 (d, J=3.7 Hz), 103.01 (t, J=25.9 Hz), 79.12, 54.52, 51.93, 51.17, 44.75, 41.70, 40.29, 39.68, 36.97 (d, J=4.4 Hz), 34.64, 30.67. MS (ESI) m/z 426.2 [M+H]⁺. HRMS: calc. 496.2406, found 496.2388 [M+H]⁺.

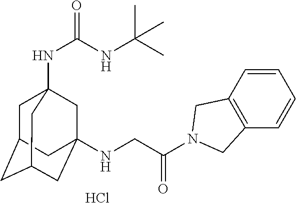 [SMILES: CC(C)(C)NC(=O)NC12CC3CC(CC(NCC(=O)N4Cc5ccccc5C4)(C3)C1)C2 ; Cl]

1-(tert-butyl)-3-(3-((2-(isoindolin-2-yl)-2-oxoethyl)amino)adamantan-1-yl)urea hydrochloride (40). General procedure B with 1-(3-aminoadamantan-1-yl)-3-(tert-butyl)urea 2,2,2-trifluoroacetate (130 mg, 0.343 mmol). The crude was redissolved in EtOAc and washed with water (10 mL). Then it was extracted with 1 M HCl solution (3×10 mL). The aqeuous phase was then extracted with DCM, dried then lyophilized to yield 1-(tert-butyl)-3-(3-((2-(isoindolin-2-yl)-2-oxoethyl)amino)adamantan-1-yl)urea hydrochloride (35 mg, 0.076 mmol, 44% yield) as a white powder. ¹H NMR (400 MHz, DMSO) δ 8.92 (s, 2H), 7.48-7.25 (m, 4H), 5.77 (s, 1H), 5.70 (s, 1H), 4.97 (s, 2H), 4.74 (s, 2H), 4.02 (t, J=6.1 Hz, 2H), 2.29-1.99 (m, 4H), 1.90 (d, J=11.0 Hz, 2H), 1.88-1.81 (m, 4H), 1.64 (d, J=9.7 Hz, 2H), 1.52 (q, J=12.5 Hz, 2H), 1.19 (s, 9H). ¹³C NMR (101 MHz, DMSO) δ 164.71, 157.07, 136.75, 135.83, 128.14, 128.06, 123.62, 123.16, 58.15, 52.49, 51.49, 51.24, 49.34, 42.52, 40.97, 40.92, 36.91, 34.87, 29.78, 29.33, 506.4. MS (ESI) m\z 425.4 [M+H]⁺, 447.4 [M+Na]⁺. HRMS: calc. 425.2911, found 425.2899 [M+H]⁺.

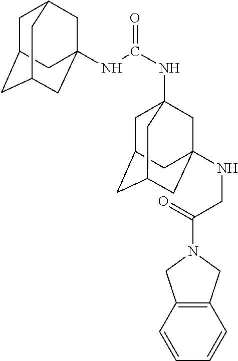 [SMILES: O=C(NC12CC3CC(CC(C3)C1)C2)NC12CC3CC(CC(NCC(=O)N4Cc5ccccc5C4)(C3)C1)C2]

1-(adamantan-1-yl)-3-(3-((2-(isoindolin-2-yl)-2-oxoethyl)amino)adamantan-1-yl)urea (41). General procedure B with 1-(adamantan-1-yl)-3-(3-aminoadamantan-1-yl)urea 2,2,2-trifluoroacetate (220 mg, 0.481 mmol). The crude was purified with flash chromatography on Si—C18 with ACN in NH water (30-100%) to yield 1-(adamantan-1-yl)-3-(3-((2-(isoindolin-2-yl)-2-oxoethyl)amino)adamantan-1-yl)urea (36 mg, 0.072 mmol, 30% yield) as a white solid. ¹H NMR (400 MHz, DMSO) δ 7.31 (s, 4H), 5.43 (s, 1H), 5.36 (s, 1H), 4.84 (s, 2H), 4.65 (s, 2H), 3.41 (s, 2H), 2.16-2.01 (m, 2H), 2.00-1.92 (m, 3H), 1.85-1.80 (m, 6H), 1.79-1.67 (m, 6H), 1.58 (d, J=3.1 Hz, 6H), 1.52-1.45 (m, 6H). ¹³C NMR (101 MHz, DMSO) δ 170.65, 156.75, 137.33, 136.45, 127.82, 123.48, 123.27, 52.23, 51.96, 51.55, 51.30, 49.73, 47.31, 43.18, 42.51, 41.76, 36.63, 35.86, 29.94, 29.40. MS (ESI) m/z 503.4 [M+H]⁺, 547.4 [M+FA−H]⁻. HRMS: calc. 503.3381, found 503.3357 [M+H]⁺.

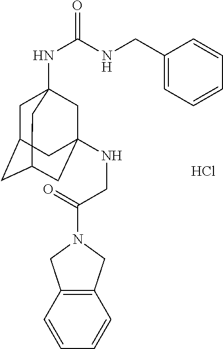 [SMILES: O=C(NCc1ccccc1)NC12CC3CC(CC(NCC(=O)N4Cc5ccccc5C4)(C3)C1)C2 ; Cl]

1-benzyl-3-(3-((2-(isoindolin-2-yl)-2-oxoethyl)amino)adamantan-1-yl)urea hydrochloride (42). General procedure B with 1-(3-aminoadamantan-1-yl)-3-benzylurea 2,2,2-trifluoroacetate (120 mg, 0.290 mmol). The crude was purified with flash chromatography on SiO₂ with MeOH in EtOAC (0-17%) and then precipitated with 2M HCl in ether solution to yield 1-benzyl-3-(3-((2-(isoindolin-2-yl)-2-oxoethyl)amino)adamantan-1-yl)urea hydrochloride (23 mg, 0.046 mmol, 24% yield) as a white powder. ¹H NMR (400 MHz, CDCl₃) δ 7.33-7.29 (m, 2H), 7.27-7.16 (m, 7H), 6.05 (s, 1H), 5.58 (s, 1H), 4.76 (s, 2H), 4.70 (s, 2H), 4.27 (d, J=5.9 Hz, 2H), 3.89 (s, 2H), 2.32 (s, 2H), 2.29-2.18 (m, 2H), 2.06-1.88 (m, 4H), 1.86-1.78 (m, 4H), 1.58 (q, J=12.2 Hz, 2H). ¹³C NMR (101 MHz, CDCl₃) δ 164.43, 157.94, 139.82, 135.04, 128.45, 128.15, 127.95, 127.16, 126.92, 122.93, 122.83, 58.41, 52.65, 51.86, 51.60, 43.74, 43.10, 40.60, 40.50, 37.92, 34.57, 29.48. MS (ESI) m/z 459.4 [M+H]⁺ 481.3 [M+Na]⁺ 503.4 [M+2Na−H]⁻. HRMS: calc. 459.2755, found 459.2739 [M+H]⁺.

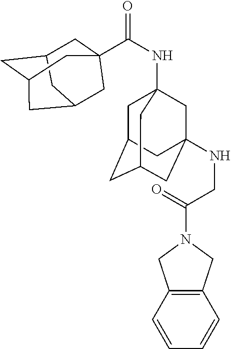 [SMILES: O=C(CNC12CC3CC(C1)CC(NC(=O)C14CC5CC(CC(C5)C1)C4)(C3)C2)N1Cc2ccccc2C1]

N-(3-((2-(isoindolin-2-yl)-2-oxoethyl)amino)adamantan-1-yl)adamantane-1-carboxamide (43). General procedure with N-(3-aminoadamantan-1-yl)adamantane-1-carboxamide (100 mg, 0.304 mmol). The crude was purified by flash chromatography on SiO₂ with MeOH in EtOAc (0-15%), then precipitated in a 9:1 diethyl ether:DCM solution to yield N-(3-((2-(isoindolin-2-yl)-2-oxoethyl)amino)adamantan-1-yl)adamantane-1-carboxamide (42 mg, 0.086 mmol, 28% yield) as a white solid. ¹H NMR (400 MHz, CDCl₃) δ 7.30-7.18 (m, 4H), 5.22 (s, 1H), 4.75 (s, 2H), 4.73 (s, 2H), 3.47 (s, 2H), 2.17 (t, J=3.2 Hz, 2H), 1.96 (p, J=3.2 Hz, 3H), 1.89 (d, J=9.7 Hz, 4H), 1.80 (d, J=11.8 Hz, 2H), 1.73 (d, J=2.9 Hz, 6H), 1.70-1.46 (m, 12H). ¹³C NMR (101 MHz, CDCl₃) δ 177.47, 169.79, 136.09, 135.93, 127.90, 127.63, 123.04, 122.72, 52.80, 52.54, 52.38, 51.62, 45.28, 42.82, 41.42, 40.92, 40.78, 39.38, 36.53, 35.51, 29.84, 28.20. MS (ESI) m/z 488.4 [M+H]⁺ 511.3 [M+Na]⁺ 976.7 [2M+H]⁺ 998.7 [2M+Na]⁺. HRMS: calc. 488.3272, found 488.3258 [M+H]⁺.

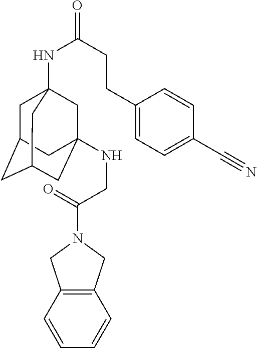 [SMILES: N#Cc1ccc(CCC(=O)NC23CC4CC(CC(NCC(=O)N5Cc6ccccc6C5)(C4)C2)C3)cc1]

3-(4-cyanophenyl)-N-(3-((2-(isoindolin-2-yl)-2-oxoethyl)amino)adamantan-1-yl)propanamide (44). General procedure B with N-(3-aminoadamantan-1-yl)-3-(4-cyanophenyl)propanamide (80 mg, 0.148 mmol). The crude was redissolved in EtOAc and washed with water (3×) followed by a saturated NaHCO₃ solution. The organic phase was dried over Na₂SO₄, then dried under reduced pressure and purified by flash chromatography on SiO₂ with MeOH in DCM (0-8%), to yield 3-(4-cyanophenyl)-N-(3-((2-(isoindolin-2-yl)-2-oxoethyl)amino)adamantan-1-yl)propanamide (7 mg, 0.015 mmol, 10% yield). ¹H NMR (400 MHz, CDCl₃) δ 7.48 (d, J=8.2 Hz, 2H), 7.28-7.15 (m, 6H), 5.23 (d, J=5.3 Hz, 1H), 4.72 (s, 4H), 3.45 (s, 2H), 2.90 (t, J=7.5 Hz, 2H), 2.72 (s, 1H), 2.33 (t, J=7.5 Hz, 2H), 2.17-2.12 (m, 2H), 1.89-1.76 (m, 6H), 1.66-1.45 (m, 6H). ¹³C NMR (101 MHz, CDCl₃) δ 170.40, 170.15, 146.86, 136.08, 135.92, 132.25, 129.31, 127.91, 127.63, 123.04, 122.71, 119.02, 110.00, 53.58, 52.33, 52.02, 51.58, 45.85, 42.99, 41.44, 40.73, 38.30, 35.38, 31.55, 29.79. MS (ESI) m/z 483.3 [M+H]⁺, 505.3 [M+Na]⁺. HRMS: calc. 519.3330, found 519.3323 [M+H]⁺.

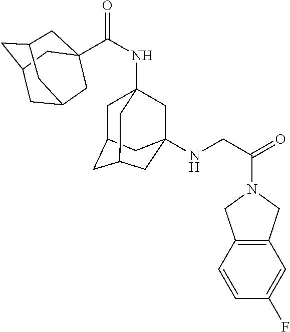 [SMILES: O=C(CNC12CC3CC(C1)CC(NC(=O)C14CC5CC(CC(C5)C1)C4)(C3)C2)N1Cc2ccc(F)cc2C1]

N-(-3-((2-(5-fluoroisoindolin-2-yl)-2-oxoethyl)amino)adamantan-1-yl)adamantane-1-carboxamide (45). General procedure B with N-(3-aminoadamantan-1-yl)adamantane-1-carboxamide (60 mg, 0.183 mmol. The crude was redissolved in EtOAc and washed with water. The organic phase was dried with Na₂SO₄, then concentrated under reduced pressure and purified by flash chromatography on SiO₂ with MeOH in ETOAc (0-10%) to yield N-(-3-((2-(5-fluoroisoindolin-2-yl)-2-oxoethyl)amino)adamantan-1-yl)adamantane-1-carboxamide (42 mg, 0.083 mmol, 68% yield). ¹H NMR (400 MHz, CDCl₃) δ 7.16 (ddd, J=12.3, 8.6, 5.0 Hz, 1H), 6.96-6.84 (m, 2H), 5.24 (s, 1H), 4.72 (d, J=3.5 Hz, 2H), 4.69 (s, 2H), 3.46 (s, 2H), 2.82 (s, 1H), 2.22-2.12 (m, 2H), 1.98-1.93 (m, 3H), 1.93-1.87 (m, 4H), 1.81-1.45 (m, 20H). ¹³C NMR (101 MHz, CDCl₃) δ 177.47, 169.89, 162.65 (dd, J=245.0, 18.7 Hz), 138.07 (dd, J=22.6, 9.0 Hz), 131.54 (dd, J=15.8, 2.6 Hz), 124.18 (dd, J=30.6, 9.0 Hz), 115.09 (t, J=23.3 Hz), 110.06 (dd, J=34.8, 23.5 Hz), 52.78, 52.41, 51.93 (dd, J=74.5, 2.6 Hz), 51.40 (d, J=72.6 Hz), 45.23 (d, J=3.3 Hz), 42.85 (d, J=5.1 Hz), 41.42, 40.90, 40.78, 39.35, 36.50, 35.50, 29.81, 28.18. MS (ESI) m/z 506.4 [M+H]⁺, 528.4 [M+Na]⁺.

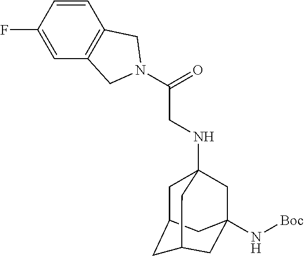 [SMILES: CC(C)(C)OC(=O)NC12CC3CC(CC(NCC(=O)N4Cc5ccc(F)cc5C4)(C3)C1)C2]

(3-((2-(5-fluoroisoindolin-2-yl)-2-oxoethyl)amino)adamantan-1-yl)carbamate (46). General procedure B with tert-butyl (3-aminoadamantan-1-yl)carbamate (0.413 g, 1.550 mmol). The crude was purified with flash chromatography on Si—C18 with MeOH in DCM (0-15%) to yield tert-butyl (3-((2-(5-fluoroisoindolin-2-yl)-2-oxoethyl)amino)adamantan-1-yl)carbamate (0.272 g, 0.613 mmol, 79% yield) as a pale yellow oil. ¹H NMR (400 MHz, CDCl₃) δ 7.25-7.19 (m, 1H), 7.05-6.94 (m, 3H), 4.83-4.73 (m, 5H), 3.50 (s, 2H), 2.23 (s, 3H), 1.93-1.79 (m, 3H), 1.72-1.51 (m, 7H), 1.42 (s, 11H).¹³C NMR (101 MHz, CDCl₃) δ 172.96, 170.16 (d, J=3.1 Hz), 162.64 (dd, J=245.3, 19.4 Hz), 154.14, 138.09 (dd, J=29.1, 8.8 Hz), 131.56 (dd, J=21.2, 2.6 Hz), 124.17 (dd, J=35.6, 8.8 Hz), 115.09 (dd, J=25.8, 22.9 Hz), 110.07 (dd, J=39.1, 23.5 Hz), 78.86, 52.15, 52.08, 51.92 (dd, J=74.5, 2.8 Hz), 51.39 (d, J=72.8 Hz), 46.01, 43.01 (d, J=5.3 Hz), 41.47, 41.05, 35.47, 29.81, 28.46. MS (ESI) m/z 444.3 [M+H]⁺. HRMS calc. 444.2657 found 444.2654 [M+H]⁺

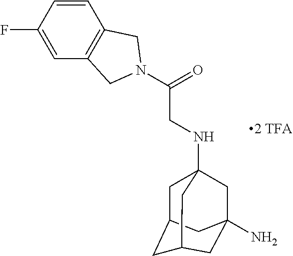 [SMILES: NC12CC3CC(C1)CC(NCC(=O)N1Cc4ccc(F)cc4C1)(C3)C2]

2-((3-aminoadamantan-1-yl)amino)-1-(5-fluoroisoindolin-2-yl)ethan-1-one bis(2,2,2-trifluoroacetate) (47). General procedure C with tert-butyl (3-((2-(5-fluoroisoindolin-2-yl)-2-oxoethyl)amino)adamantan-1-yl)carbamate (0.305 g, 0.688 mmol) to yield 2-((3-aminoadamantan-1-yl)amino)-1-(5-fluoroisoindolin-2-yl)ethan-1-one bis(2,2,2-trifluoroacetate) (0.350 g, 0.612 mmol, 89% yield). ¹H NMR (400 MHz, Methanol-d4) δ 7.38 (td, J=9.2, 4.8 Hz, 1H), 7.19-7.07 (m, 2H), 4.95 (s, 4H), 4.14 (s, 2H), 2.53-2.47 (m, 2H), 2.19 (s, 2H), 2.03 (q, J=11.5 Hz, 4H), 1.94 (d, J=2.9 Hz, 4H), 1.76 (t, J=3.5 Hz, 2H). MS (ESI) m/z 344.4 [M+H]⁺.

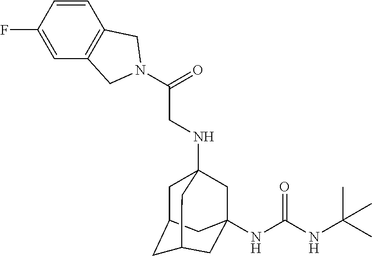 [SMILES: CC(C)(C)NC(=O)NC12CC3CC(CC(NCC(=O)N4Cc5ccc(F)cc5C4)(C3)C1)C2]

1-(tert-butyl)-3-(3-((2-(5-fluoroisoindolin-2-yl)-2-oxoethyl)amino)adamantan-1-yl)urea (48). General procedure B with 1-(3-aminoadamantan-1-yl)-3-(tert-butyl)urea 2,2,2-trifluoroacetate (100 mg, 0.211 mmol). The crude was dissolved in EtOAc and washed with water, followed by an extracted with 1 M HCl solution (3×10 mL). The aqeuous phase was backextracted with DCM and lyophilised to yield 1-(tert-butyl)-3-(3-((2-(5-fluoroisoindolin-2-yl)-2-oxoethyl)amino)adamantan-1-yl)urea (33 mg, 0.075 mmol, 71% yield) as an off-white powder. ¹H NMR (400 MHz, DMSO) δ 7.37 (td, J=9.2, 6.8 Hz, 1H), 7.26-7.19 (m, 1H), 7.19-7.08 (m, 1H), 5.49 (s, 1H), 5.43 (s, 1H), 4.82 (d, J=16.0 Hz, 2H), 4.63 (d, J=13.7 Hz, 2H), 3.43 (s, 2H), 2.14-2.06 (m, 2H), 1.84-1.71 (m, 6H), 1.62-1.39 (m, 6H), 1.18 (s, 9H). ¹³C NMR (101 MHz, DMSO) δ 170.34 (d, J=3.3 Hz), 162.31 (d, J=241.7 Hz), 157.12, 139.31 (dd, J=86.7, 9.0 Hz), 132.84 (dd, J=84.4, 2.6 Hz), 125.06 (dd, J=22.6, 9.0 Hz), 114.95 (d, J=22.7 Hz), 110.49 (dd, J=26.4, 23.5 Hz), 52.30, 51.54, 52.28-51.16 (m), 51.97-50.64 (m), 49.26, 47.12, 43.06 (d, J=4.4 Hz), 41.74, 41.47, 35.79, 29.91, 29.79. MS (ESI) m/z 443.4 [M+H]⁺. HRMS: 443.2817 found 443.2809 [M+H]⁺.

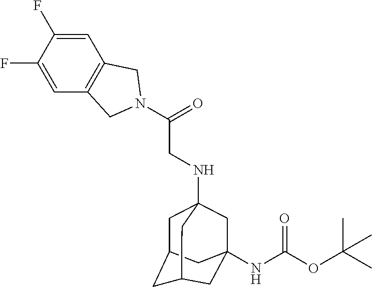 [SMILES: CC(C)(C)OC(=O)NC12CC3CC(CC(NCC(=O)N4Cc5cc(F)c(F)cc5C4)(C3)C1)C2]

Tert-butyl(3-((2-(5,6-difluoroisoindolin-2-yl)-2-oxoethyl)amino)adamantan-1-yl)carbamate (50) (26 mg, 0.056 mmol, 38% yield) was prepared according to general procedure B from tert-butyl (3-aminoadamantan-1-yl)carbamate (69.2 mg, 0.260 mmol, 1.75 eq) and 2-bromo-1-(5,6-difluoroisoindolin-2-yl)ethan-1-one (41 mg, 0.149 mmol, 1 eq). ¹H NMR (400 MHz, MeOD) δ 7.28 (m, 2H), 4.84 (s, 2H), 4.73 (s, 2H), 3.52 (s, 2H), 2.21 (m, 2H), 1.85 (m, 6H), 1.65 (m, 6H), 1.41 (s, 9H). ¹³C NMR (101 MHz, MeOD) δ 171.71, 156.55, 151.64 (ddd, J=248.0, 15.6, 5.5 Hz), 133.87 (ddd, J=45.3, 7.2, 3.4 Hz), 112.98 (dd, J=19.2, 14.1 Hz), 79.51, 53.65, 52.88, 52.57 (dd, J=78.5, 2.2 Hz), 46.35, 43.14, 41.87, 41.76, 36.46, 31.31, 28.82. MS (ESI) m/z 462.3 [M+H]⁺

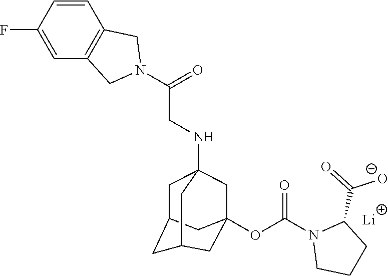 [SMILES: O=C([O-])[C@@H]1CCCN1C(=O)OC12CC3CC(CC(NCC(=O)N4Cc5ccc(F)cc5C4)(C3)C1)C2 ; [Li+]]

Lithium (((3-((2-(5-fluoroisoindolin-2-yl)-2-oxoethyl)amino)adamantan-1-yl)oxy)carbonyl)-L-prolinate (51) (17 mg, 0.035 mmol, quantitative yield) was prepared according to general procedure J from 1-(3-((2-(5-fluoroisoindolin-2-yl)-2-oxoethyl)amino)adamantan-1-yl) 2-methyl (S)-pyrrolidine-1,2-dicarboxylate (16.8 mg, 0.034 mmol). ¹H NMR (400 MHz, MeOD) δ 7.34 (dd, J=8.5, 5.0 Hz, 1H), 7.10 (d, J=8.7 Hz, 1H), 7.06 (t, J=9.1 Hz, 1H), 4.86 (d, J=14.2 Hz, 2H), 4.75 (d, J=13.5 Hz, 2H), 4.15-4.02 (m, 1H), 3.53 (s, 2H), 3.51-3.34 (m, 2H), 2.30-1.57 (m, 19H). ¹³C NMR (101 MHz, MeOD) δ 180.74, 171.87, 170.31, 156.06, 139.41 (d, J=9.0 Hz), 132.83, 125.51 (d, J=13.6 Hz), 115.84 (d, J=23.1 Hz), 110.96 (dd, J=24.4, 8.7 Hz), 81.11, 63.29, 54.85, 52.02, 47.56, 46.51, 42.07, 41.96, 41.75 (d, J=7.3 Hz), 36.34, 32.47, 32.14 (d, J=2.8 Hz), 24.77. MS (ESI) m/z 486.3 [M+H]⁺

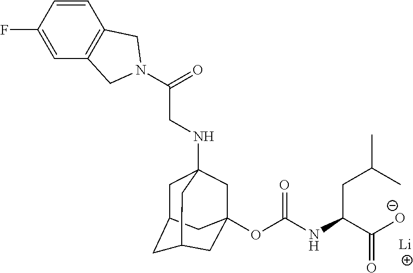 [SMILES: CC(C)C[C@H](NC(=O)OC12CC3CC(CC(NCC(=O)N4Cc5ccc(F)cc5C4)(C3)C1)C2)C(=O)[O-] ; [Li+]]

Lithium (((3-((2-(5-fluoroisoindolin-2-yl)-2-oxoethyl)amino)adamantan-1-yl)oxy)carbonyl)-D-leucinate (52) (13 mg, 0.026 mmol, quantitative yield) was prepared according to general procedure J from methyl (((3-((2-(5-fluoroisoindolin-2-yl)-2-oxoethyl)amino)adamantan-1-yl)oxy)carbonyl)-D-leucinate (13 mg, 0.025 mmol). ¹H NMR (400 MHz, MeOD) δ 7.40-7.27 (m, 1H), 7.16-6.98 (m, 2H), 4.86-4.81 (m, 2H), 4.75 (d, J=13.5 Hz, 2H), 4.02 (s, 1H), 3.52 (s, 2H), 2.28 (s, 2H), 2.18-1.96 (m, 6H), 1.83-1.40 (m, 8H), 0.93 (s, 6H). ¹³C NMR (101 MHz, MeOD) δ 180.84, 172.13, 163.99 (d, J=234.8 Hz), 157.27, 139.34 (d, J=59.1 Hz), 132.82 (d, J=52.5 Hz), 125.52, 115.92 (d, J=32.9 Hz), 110.92 (d, J=24.1 Hz), 80.76, 55.87, 54.85, 52.92 (d, J=69.3 Hz), 52.40 (d, J=67.5 Hz), 46.49, 43.48, 43.09, 41.62, 36.04, 31.90, 26.03, 23.71, 22.27. MS (ESI) m/z=502.3 [M+H]⁺.

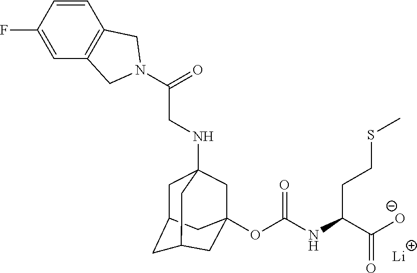 [SMILES: CSCC[C@H](NC(=O)OC12CC3CC(CC(NCC(=O)N4Cc5ccc(F)cc5C4)(C3)C1)C2)C(=O)[O-] ; [Li+]]

Lithium (((3-((2-(5-fluoroisoindolin-2-yl)-2-oxoethyl)amino)adamantan-1-yl)oxy)carbonyl)-L-methionine (15 mg, 0.029 mmol, quantitative yield) was prepared according to general procedure J from methyl (((3-((2-(5-fluoroisoindolin-2-yl)-2-oxoethyl)amino)adamantan-1-yl)oxy)carbonyl)-L-methioninate (15.6 mg, 0.029 mmol). ¹H NMR (400 MHz, MeOD) δ 7.34 (dd, J=8.5, 5.0 Hz, 1H), 7.11 (m, 1H), 7.05 (m, 1H), 4.86 (d, J=13.9 Hz, 2H), 4.75 (d, J=13.4 Hz, 2H), 4.03 (m, 1H), 3.52 (s, 2H), 2.47 (m, 2H), 2.29 (m, 2H), 2.05 (m, 10H), 1.89 (m, 1H), 1.64 (m, 6H). MS (ESI) m/z 520.3 [M+H]⁺.

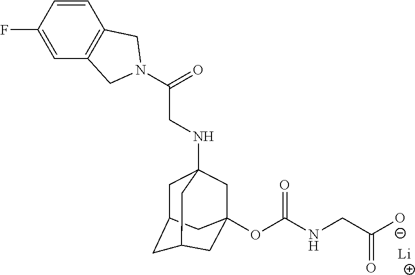

Lithium (((3-((2-(5-fluoroisoindolin-2-yl)-2-oxoethyl)amino)adamantan-1-yl)oxy)carbonyl)glycine (54) (20 mg, 0.044 mmol, quantitative yield) was prepared according to general procedure J from methyl (((3-((2-(5-fluoroisoindolin-2-yl)-2-oxoethyl)amino)adamantan-1-yl)oxy)carbonyl)glycinate (20 mg, 0.044 mmol). ¹H NMR (400 MHz, MeOD) δ 7.34 (dd, J=8.5, 5.0 Hz, 1H), 7.13-7.08 (m, 1H), 7.06 (t, J=9.1 Hz, 1H), 4.86 (d, J=14.0 Hz, 2H), 4.75 (d, J=13.5 Hz, 2H), 3.60 (s, 2H), 3.52 (d, J=7.7 Hz, 1H), 2.32-2.26 (m, 2H), 2.14-2.01 (m, 6H), 1.74-1.53 (m, 6H). ¹³C NMR (101 MHz, MeOD) δ 176.99, 171.91, 162.92, 157.55, 139.36, 133.37, 125.51, 115.84, 111.13, 80.55, 54.86, 52.90 (dd, J=71.1, 3.1 Hz), 52.38 (d, J=69.5 Hz), 46.75, 45.43, 41.91, 41.83, 36.31, 32.14. MS (ESI) m/z 446.3 [M+H]⁺, m/z=447.3 [M+2]⁺.

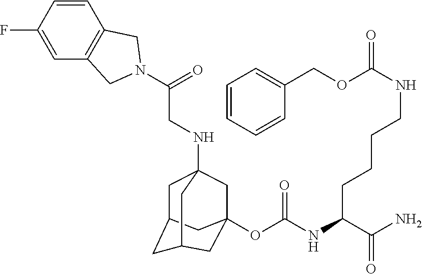 [SMILES: NC(=O)[C@H](CCCCNC(=O)OCc1ccccc1)NC(=O)OC12CC3CC(CC(NCC(=O)N4Cc5ccc(F)cc5C4)(C3)C1)C2]

benzyl (3-((2-(5-fluoroisoindolin-2-yl)-2-oxoethyl)amino)adamantan-1-yl) ((S)-6-amino-6-oxohexane-1,5-diyl)dicarbamate (55) (102 mg, 0.157 mmol, 50% yield) was prepared according to general procedure B from 3-aminoadamantan-1-yl benzyl ((S)-6-amino-6-oxohexane-1,5-diyl)dicarbamate (0.296 g, 0.626 mmol) and 2-bromo-1-(5-fluoroisoindolin-2-yl)ethan-1-one (0.081 g, 0.313 mmol). ¹H NMR (400 MHz, MeOD) δ 7.30 (m, 6H), 7.06 (dtd, J=17.5, 7.9, 2.6 Hz, 2H), 5.05 (s, 2H), 4.80 (d, J=14.4 Hz, 2H), 4.72 (d, J=12.8 Hz, 2H), 3.99 (dd, J=9.2, 4.8 Hz, 1H), 3.51 (s, 2H), 3.11 (t, J=6.7 Hz, 2H), 2.27 (m, 2H), 2.06 (s, 6H), 1.56 (m, 12H). ¹³C NMR (101 MHz, MeOD) δ 177.90, 171.59, 165.31 (d, J=8.5 Hz), 162.89 (d, J=8.3 Hz), 158.89, 157.29, 139.84 (d, J=8.9 Hz), 139.34 (d, J=8.9 Hz), 138.42, 133.01 (dd, J=51.3, 2.5 Hz), 129.45, 128.82 (d, J=20.2 Hz), 125.50 (dd, J=10.5, 9.0 Hz), 115.90 (dd, J=23.1, 10.6 Hz), 110.95 (dd, J=24.0, 13.8 Hz), 80.96, 67.29, 55.68, 55.01, 52.73 (m), 46.62, 43.25 (d, J=2.6 Hz), 41.70 (d, J=8.3 Hz), 41.42, 36.17, 33.02, 32.06, 30.46, 24.07. MS (ESI) m/z=473.4 [M+H]⁺.

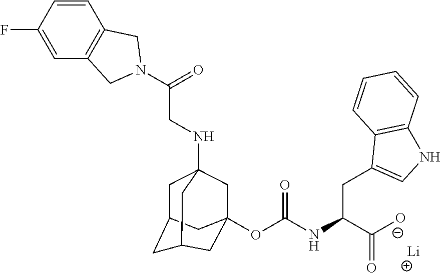 [SMILES: O=C(N[C@@H](Cc1c[nH]c2ccccc12)C(=O)[O-])OC12CC3CC(CC(NCC(=O)N4Cc5ccc(F)cc5C4)(C3)C1)C2 ; [Li+]]

lithium (((3-((2-(5-fluoroisoindolin-2-yl)-2-oxoethyl)amino)adamantan-1-yl)oxy)carbonyl)-L-tryptophanate (56) (20 mg, 0.034 mmol, quantitative yield) was prepared according to general procedure J from methyl ((((1s,3S,5R,7S)-3-((2-(5-fluoroisoindolin-2-yl)-2-oxoethyl)amino)adamantan-1-yl)oxy)carbonyl)-L-tryptophanate (20 mg, 0.034 mmol). ¹H NMR (400 MHz, MeOD) δ 7.67 (dd, J=54.3, 7.8 Hz, 1H), 7.39-7.23 (m, 2H), 7.04 (tdd, J=28.4, 13.3, 5.8 Hz, 5H), 4.85-4.69 (m, 4H), 4.32-4.23 (m, 1H), 3.50-3.33 (m, 3H), 3.13-2.72 (m, 1H), 2.24 (s, 1H), 2.09-1.82 (m, 5H), 1.70-1.11 (m, 9H). MS (ESI) m/z 574.3 [M+H]⁺.

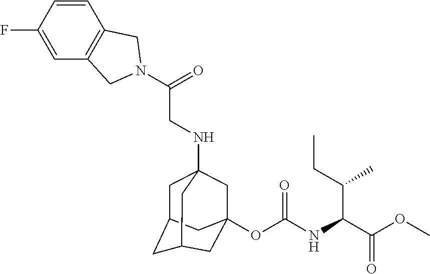 [SMILES: CC[C@H](C)[C@H](NC(=O)OC12CC3CC(CC(NCC(=O)N4Cc5ccc(F)cc5C4)(C3)C1)C2)C(=O)OC]

Methyl (((3-((2-(5-fluoroisoindolin-2-yl)-2-oxoethyl)amino)adamantan-1-yl)oxy)carbonyl)-L-isoleucinate (57) (26 mg, 0.050 mmol, 46% yield) was prepared according to general procedure B from methyl (((3-aminoadamantan-1-yl)oxy)carbonyl)-L-isoleucinate (0.075 g, 0.222 mmol) and 2-bromo-1-(5-fluoroisoindolin-2-yl)ethan-1-one (0.029 g, 0.111 mmol). ¹H NMR (400 MHz, MeOD) δ 8.36 (s, 1H), 7.36 (dt, J=9.1, 4.9 Hz, 1H), 7.18 (d, J=8.5 Hz, 1H), 7.11 (m, 2H), 4.90 (d, J=13.8 Hz, 2H), 4.80 (d, J=13.4 Hz, 2H), 4.09 (s, 2H), 4.05 (m, 1H), 3.70 (s, 3H), 2.41 (m, 4H), 2.13 (m, 4H), 1.96 (m, 4H), 1.82 (m, 1H), 1.68 (m, 2H), 1.45 (m, 1H), 1.21 (m, 1H), 0.90 (m, 6H). ¹³C NMR (101 MHz, MeOD) δ 174.15, 165.64, 164.20 (dd, J=243.7, 7.9 Hz), 157.28, 139.27 (dd, J=46.6, 9.1 Hz), 132.69 (dd, J=48.8, 2.6 Hz), 125.56 (dd, J=16.9, 8.9 Hz), 116.08 (dd, J=23.1, 9.3 Hz), 111.00 (dd, J=24.2, 20.6 Hz), 79.59, 60.18, 54.83, 59.70, 52.94 (dd, J=93.5, 2.8 Hz), 52.43 (d, J=92.2 Hz), 52.35, 43.25, 42.12, 41.09 (d, J=8.4 Hz), 38.27 (d, J=18.5 Hz), 35.26, 31.67, 26.26, 15.99, 11.68. MS (ESI) m/z 516.4 [M+H]⁺

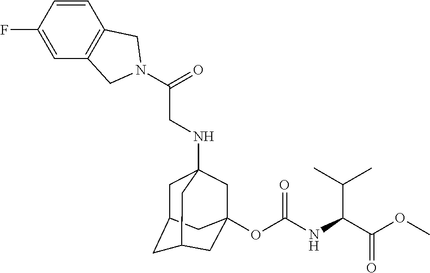 [SMILES: COC(=O)[C@@H](NC(=O)OC12CC3CC(CC(NCC(=O)N4Cc5ccc(F)cc5C4)(C3)C1)C2)C(C)C]

(((3-((2-(5-fluoroisoindolin-2-yl)-2-oxoethyl)amino)adamantan-1-yl)oxy)carbonyl)-L-valinate (58) (13 mg, 0.026 mmol, 37% yield) was prepared according to general procedure B from methyl (((3-aminoadamantan-1-yl)oxy)carbonyl)-L-valinate (0.046 g, 0.142 mmol). ¹H NMR (400 MHz, MeOD) δ 8.41 (s, 1H), 7.36 (m, 1H), 7.11 (m, 2H), 4.88 (m, 2H), 4.80 (d, J=13.4 Hz, 2H), 4.04 (s, 2H), 3.99 (d, J=6.0 Hz, 1H), 3.71 (s, 3H), 2.40 (m, 4H), 2.12 (m, 5H), 1.93 (m, 4H), 1.69 (q, J=13.2 Hz, 2H), 0.93 (m, 6H). ¹³C NMR (101 MHz, MeOD) δ 174.19, 166.01, 157.42, 139.29 (d, J=52.1 Hz), 132.71 (d, J=48.4 Hz), 125.67 (d, J=9.0 Hz), 125.47 (d, J=8.8 Hz), 116.11 (dd, J=23.5, 10.4 Hz), 111.02 (m), 79.68, 60.74, 59.82, 53.39, 52.41 (t, J=46.3 Hz), 43.45, 42.19, 41.21, 41.09, 38.44, 35.33, 31.73 (d, J=5.0 Hz), 19.49, 18.44. MS (ESI) m/z 502.4 [M+H]⁺

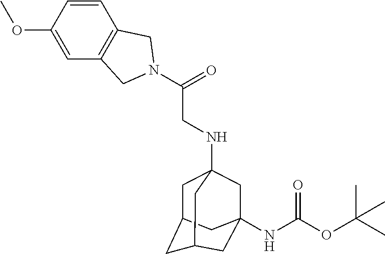 [SMILES: COc1ccc2c(c1)CN(C(=O)CNC13CC4CC(C1)CC(NC(=O)OC(C)(C)C)(C4)C3)C2]

tert-butyl (3-((2-(5-methoxyisoindolin-2-yl)-2-oxoethyl)amino)adamantan-1-yl)carbamate (59) (40 mg, 0.088 mmol, 47% yield) was prepared according to general procedure B from tert-butyl (3-aminoadamantan-1-yl)carbamate (100 mg, 0.375 mmol) and 2-bromo-1-(5-methoxyisoindolin-2-yl)ethan-1-one (50.7 mg, 0.188 mmol). ¹H NMR (400 MHz, MeOD) δ 7.21 (d, J=8.2 Hz, 1H), 6.87 (m, 2H), 4.77 (d, J=16.4 Hz, 2H), 4.68 (d, J=15.6 Hz, 2H), 3.78 (d, J=1.7 Hz, 3H), 3.52 (s, 2H), 2.21 (m, 2H), 1.85 (m, 6H), 1.65 (tt, J=15.2, 8.0 Hz, 6H), 1.41 (s, 9H). ¹³C NMR (101 MHz, MeOD) δ 170.19 (d, J=2.1 Hz), 159.91 (d, J=10.2 Hz), 137.25 (d, J=53.7 Hz), 127.58 (d, J=46.9 Hz), 123.27 (d, J=9.9 Hz), 114.00, 107.37, 54.55, 52.31, 52.06, 51.49, 51.36 (d, J=6.5 Hz), 50.69, 41.73 (d, J=4.5 Hz), 40.46, 40.37, 35.08, 29.92, 27.47. MS (ESI) m/z 456.4 [M+H]⁺.

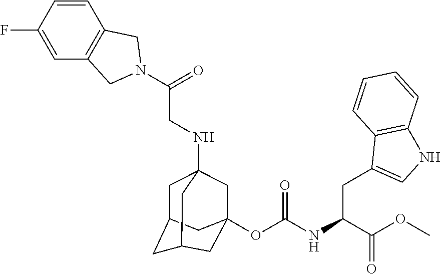 [SMILES: COC(=O)[C@H](Cc1c[nH]c2ccccc12)NC(=O)OC12CC3CC(CC(NCC(=O)N4Cc5ccc(F)cc5C4)(C3)C1)C2]

Methyl (((3-((2-(5-fluoroisoindolin-2-yl)-2-oxoethyl)amino)adamantan-1-yl)oxy)carbonyl)-L-tryptophanate (60) (124-mg, 0.211 mmol, 72% yield) was prepared according to general procedure B from methyl (((3-aminoadamantan-1-yl)oxy)carbonyl)-L-tryptophanate (0.2396 g, 0.582 mmol). ¹H NMR (400 MHz, DMSO) δ 10.87 (s, 1H), 7.47 (d, J=7.9 Hz, 1H), 7.37 (m, 1H), 7.32 (t, J=8.3 Hz, 2H), 7.18 (m, 3H), 7.06 (t, J=7.4 Hz, 1H), 6.98 (t, J=7.4 Hz, 1H), 4.80 (d, J=15.8 Hz, 2H), 4.62 (d, J=13.9 Hz, 2H), 4.17 (m, 1H), 3.58 (s, 3H), 3.38 (m, 2H), 3.05 (m, 2H), 2.17 (s, 2H), 1.87 (m, 6H), 1.47 (d, J=16.4 Hz, 6H). MS (ESI) m/z 589.4 [M+H]⁺.

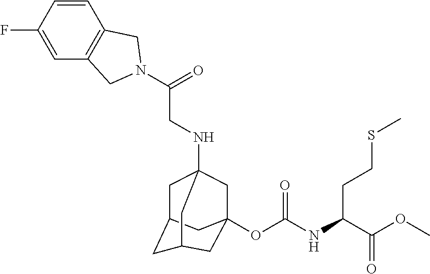 [SMILES: COC(=O)[C@H](CCSC)NC(=O)OC12CC3CC(CC(NCC(=O)N4Cc5ccc(F)cc5C4)(C3)C1)C2]

Methyl (((-3-((2-(5-fluoroisoindolin-2-yl)-2-oxoethyl)amino)adamantan-1-yl)oxy)carbonyl)-L-methioninate (61) (68 mg, 0.127 mmol, 68% yield) was prepared according to general procedure B from methyl (((3-aminoadamantan-1-yl)oxy)carbonyl)-L-methioninate (133 mg, 0.373 mmol) and 2-bromo-1-(5-fluoroisoindolin-2-yl)ethan-1-one (48 mg, 0.187 mmol). ¹H NMR (400 MHz, MeOD) δ 7.34 (m, 1H), 7.11 (m, 1H), 7.06 (m, 1H), 4.85 (d, J=14.1 Hz, 2H), 4.75 (d, J=13.3 Hz, 2H), 4.27 (m, 1H), 3.71 (s, 3H), 3.53 (s, 2H), 2.54 (m, 2H), 2.29 (m, 2H), 2.06 (m, 10H), 1.89 (m, 1H), 1.63 (m, 6H). MS (ESI) m/z 534.3 [M+H]⁺.

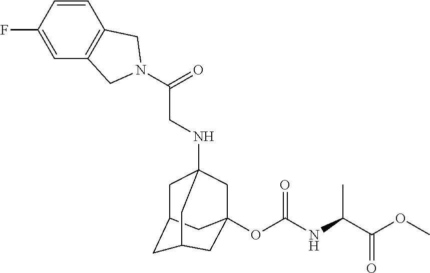 [SMILES: COC(=O)[C@H](C)NC(=O)OC12CC3CC(CC(NCC(=O)N4Cc5ccc(F)cc5C4)(C3)C1)C2]

Methyl (((3-((2-(5-fluoroisoindolin-2-yl)-2-oxoethyl)amino)adamantan-1-yl)oxy)carbonyl)-L-alaninate (62) (124 mg, 0.263 mmol, 92% yield) was prepared according to general procedure B from methyl (((3-aminoadamantan-1-yl)oxy)carbonyl)-L-alaninate (0.170 g, 0.574 mmol) and 2-bromo-1-(5-fluoroisoindolin-2-yl)ethan-1-one (0.074 g, 0.287-mmol). ¹H NMR (400 MHz, MeOD) δ 8.43 (s, 1H), 7.36 (dt, J=8.5, 5.4 Hz, 1H), 7.11 (dt, J=17.5, 8.5 Hz, 2H), 4.90 (d, J=14.0 Hz, 2H), 4.80 (d, J=13.4 Hz, 2H), 4.14 (m, 1H), 4.04 (s, 2H), 3.70 (s, 3H), 2.38 (m, 4H), 2.08 (m, 4H), 1.93 (m, 4H), 1.69 (m, 2H), 1.43 (d, J=6.9 Hz, 1H), 1.31 (d, J=7.2 Hz, 3H). ¹³C NMR (101 MHz, MeOD) δ 175.30, 168.90, 166.03, 157.03, 139.30 (dd, J=44.5, 9.0 Hz), 132.72 (dd, J=47.0, 2.6 Hz), 125.57 (dd, J=19.0, 8.9 Hz), 116.10 (dd, J=23.2, 10.0 Hz), 111.01 (dd, J=24.2, 22.4 Hz), 79.64, 59.81, 52.93 (dd, J=93.3, 2.8 Hz), 52.68, 52.42 (d, J=91.7 Hz), 51.79, 50.57, 43.46, 42.18, 41.16 (d, J=12.9 Hz), 38.42, 35.32, 31.69, 19.97, 17.52. MS (ESI) m/z 474.4 [M+H]⁺

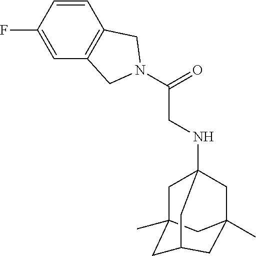 [SMILES: CC12CC3CC(C)(C1)CC(NCC(=O)N1Cc4ccc(F)cc4C1)(C3)C2]

2-((3,5-Dimethyladamantan-1-yl)amino)-1-(5-fluoroisoindolin-2-yl)ethan-1-one (63) (57 mg, 0.160 mmol, 96% yield) was prepared according to general procedure B from 2-bromo-1-(5-fluoroisoindolin-2-yl)ethan-1-one (43 mg, 0.167 mmol) and 3,5-dimethyladamantan-1-amine hydrochloride (71.9 mg, 0.333 mmol). ¹H NMR (400 MHz, MeOD) δ 7.34 (dt, J=9.0, 4.8 Hz, 1H), 7.08 (m, 2H), 4.84 (d, J=14.0 Hz, 2H), 4.75 (d, J=13.5 Hz, 2H), 3.55 (s, 2H), 2.18 (p, J=3.2 Hz, 1H), 1.58 (m, 2H), 1.35 (m, 9H), 1.17 (m, 2H), 0.88 (s, 6H). ¹³C NMR (101 MHz, MeOD) δ 170.00, 138.26 (d, J=49.5 Hz), 131.64 (d, J=50.7 Hz), 124.12 (dd, J=10.6, 8.8 Hz), 114.53 (dd, J=23.1, 10.9 Hz), 109.57 (dd, J=24.0, 14.3 Hz), 52.73, 51.49 (d, J=78.9 Hz), 50.97 (d, J=74.4 Hz), 50.38, 47.33, 42.40, 41.66, 41.64, 39.65, 31.99, 30.23, 29.28. MS (ESI) m/z 357.3 [M+H]⁺

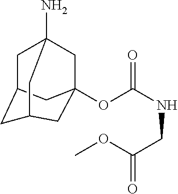

Intermediates 24: Methyl (((3-aminoadamantan-1-yl)oxy)carbonyl)glycinate (0.1483 g, 0.525 mmol, 78% yield) was prepared according to general procedure K from methyl glycinate (599 mg, 6.72 mmol, 10 eq). MS (ESI) m/z 283.1 [M+H]⁺.

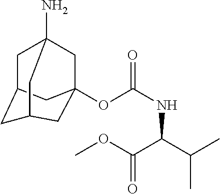 [SMILES: COC(=O)[C@@H](NC(=O)OC12CC3CC(CC(N)(C3)C1)C2)C(C)C]

Methyl (((3-aminoadamantan-1-yl)oxy)carbonyl)-L-valinate (0.053 g, 0.163 mmol, 24% yield) was prepared according to general procedure K from methyl valinate (882 mg, 6.72 mmol, 10 eq). MS (ESI) m/z 325.2 [M+H]⁺.

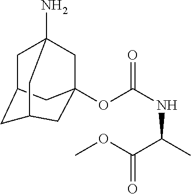 [SMILES: COC(=O)[C@H](C)NC(=O)OC12CC3CC(CC(N)(C3)C1)C2]

Methyl (((3-aminoadamantan-1-yl)oxy)carbonyl)-L-alaninate (0.173 g, 0.584 mmol, 87% yield) was prepared according to general procedure K from methyl L-alaninate (693 mg, 6.72 mmol, 10 eq). MS (ESI) m/z 297.1 [M+H]⁺.

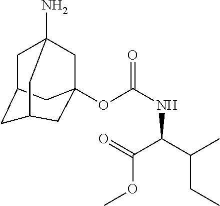 [SMILES: CCC(C)[C@H](NC(=O)OC12CC3CC(CC(N)(C3)C1)C2)C(=O)OC]

Methyl (((3-aminoadamantan-1-yl)oxy)carbonyl)-L-isoleucinate (80 mg, 0.236 mmol, 35% yield) was prepared according to general procedure K from methyl L-isoleucinate (976 mg, 6.72 mmol, 10 eq). MS (ESI) m/z 339.3 [M+H]⁺.

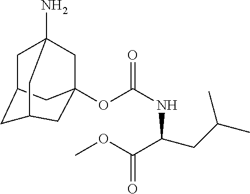 [SMILES: COC(=O)[C@H](CC(C)C)NC(=O)OC12CC3CC(CC(N)(C3)C1)C2]

Methyl (((3-aminoadamantan-1-yl)oxy)carbonyl)-L-leucinate (48 mg, 0.142 mmol, 21% yield) Was prepared according to general procedure K from methyl L-leucinate hydrochloride (1221 mg, 6.72 mmol, 10 eq). MS (ESI): m/z=339.2 [M+H]⁺.

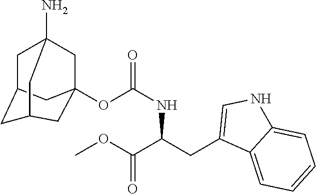 [SMILES: COC(=O)[C@H](Cc1c[nH]c2ccccc12)NC(=O)OC12CC3CC(CC(N)(C3)C1)C2]

Methyl (((3-aminoadamantan-1-yl)oxy)carbonyl)-L-tryptophanate (0.2396 g, 0.582 mmol, 87% yield) was prepared according to general procedure K from methyl tryptophanate (1027 mg, 4.70 mmol, 7 eq). MS (ESI) m/z 412.3 [M+H]⁺.

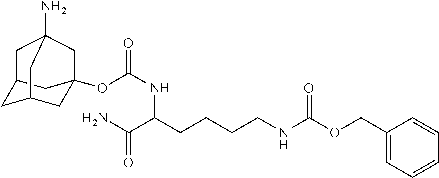 [SMILES: NC(=O)C(CCCCNC(=O)OCc1ccccc1)NC(=O)OC12CC3CC(CC(N)(C3)C1)C2]

3-Aminoadamantan-1-yl benzyl ((S)-6-amino-6-oxohexane-1,5-diyl)dicarbamate (296 mg, 0.626 mmol 93% yield) was prepared according to general H procedure K from benzyl (5,6-diamino-6-oxohexyl) carbamate (1314 mg, 4.70 mmol, 7 eq). MS (ESI) m/z 473.4 [M+H]⁺.

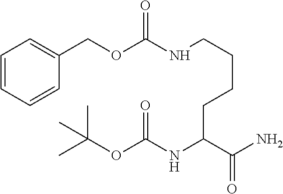 [SMILES: CC(C)(C)OC(=O)NC(CCCCNC(=O)OCc1ccccc1)C(N)=O]

Benzyl N-(5-[[(tert-butoxy)carbonyl]amino]-5-carbamoylpentyl)carbamate (2.494 g, 6.57 mmol, 75% yield). A solution of N6-((benzyloxy)carbonyl)-N2-(tert-butoxycarbonyl) lysine (2.5 g, 6.57 mmol) and DIPEA (2.98 mL, 17.09 mmol) in DMF (25 mL) under nitrogen atmosphere was cooled down to 0° C. Isobutylchloroformate (0.938 mL, 7.23 mmol) was slowly added dropwise and the mixture was stirred for 30′. An aqueous solution of ammonium chloride (3.61 mL, 7.23 mmol) was added and the mixture was stirred at RT overnight. EtOAc (100 mL) was added, and the obtained solution was washed with a saturated NaHCO₃ solution (3×50 mL) and brine (1×50 mL). The organic phase was dried over Na₂SO₄ and evaporated under reduced pressure to give a crude residue that was purified by flash column chromatography on a SiO₂ column with a gradient of methanol in DCM. MS (ESI) m/z 280.2 [M-Boc+H]⁺.

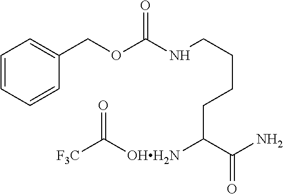

Benzyl (5,6-diamino-6-oxohexyl)carbamate 2,2,2-trifluoroacetate (1.920 g, 4.88 mmol, 99% yield) was prepared according to general procedure C from benzyl tert-butyl (6-amino-6-oxohexane-1,5-diyl)dicarbamate (1.876 g, 4.94 mmol) and precipitated from a diethyl ether-DCM mixture (4:1). MS (ESI) m/z 280.2 [M+H]⁺.

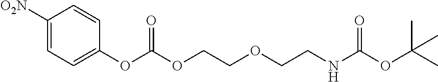 [SMILES: CC(C)(C)OC(=O)NCCOCCOC(=O)Oc1ccc([N+](=O)[O-])cc1]

Intermediates 65: tert-Butyl (2-(2-(((4-nitrophenoxy)carbonyl)oxy)ethoxy)ethyl) carbamate (0.148 g, 0.399 mmol, 55% yield) was prepared according to general procedure L from tert-butyl (2-(2-hydroxyethoxy)ethyl)carbamate (64) (0.150 g, 0.731 mmol) and purified by silica gel flash chromatography (elution with 10-50% AcOEt in heptane). LRMS m/z (ESI⁺) 271 [M+H-Boc+H]⁺.

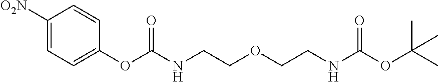 [SMILES: CC(C)(C)OC(=O)NCCOCCNC(=O)Oc1ccc([N+](=O)[O-])cc1]

4-Nitrophenyl (2-(2-((tert-butoxycarbonyl)amino)ethoxy)ethyl) carbamate (0.259 g, 0.701 mmol, 36% yield) was prepared according to general procedure L from tert-butyl (2-(2-aminoethoxy)ethyl)carbamate (64) (0.400 g, 1.958 mmol) and purified by silica gel flash chromatography (elution with 10-50% AcOEt in heptane). LRMS m/z (ESI⁺) 270 [M+H-Boc+H]⁺.

4-Nitrophenyl (2,2-dimethyl-4-oxo-3,8,11,14-tetraoxa-5-azahexadecan-16-yl)carbamate (0.044 g, 0.097 mmol, 28% yield) was prepared according to general procedure L from tert-butyl (2-(2-(2-(2-aminoethoxy)ethoxy)ethoxy)ethyl)carbamate (64) (0.100 g, 0.342 mmol) and purified by silica gel flash chromatography (elution with 40-80% AcOEt in heptane). LRMS m/z (ESI⁺) 358 [M+H-Boc+H]⁺.

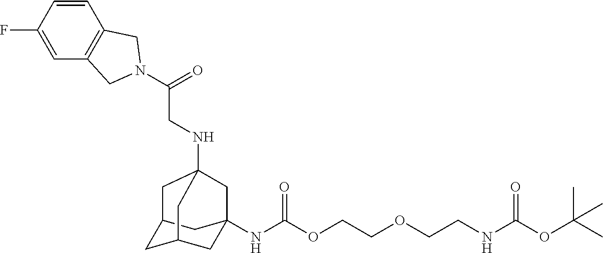 [SMILES: CC(C)(C)OC(=O)NCCOCCOC(=O)NC12CC3CC(CC(NCC(=O)N4Cc5ccc(F)cc5C4)(C3)C1)C2]

Intermediates 66: 2-(2-((tert-Butoxycarbonyl)amino)ethoxy)ethyl-3-((2-(5-fluoroisoindolin-2-yl)-2oxoethyl)amino)adamantan-1-ylcarbamate (0.012 g, 0.021 mmol, 14% yield) was prepared according to general procedure M from tert-butyl (2-(2-(((4-nitrophenoxy)carbonyl)oxy)ethoxy)ethyl) carbamate (65) (0.056 g, 0.150 mmol) and purified by silica gel flash chromatography (elution with 0-10% MeOH in DCM). LRMS m/z (ESI⁺) 575 [M+H]⁺.

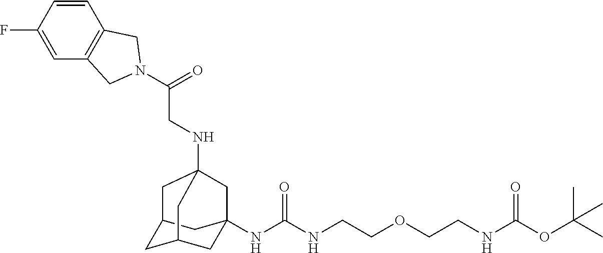 [SMILES: CC(C)(C)OC(=O)NCCOCCNC(=O)NC12CC3CC(CC(NCC(=O)N4Cc5ccc(F)cc5C4)(C3)C1)C2]

tert-Butyl (2-(2-(3-(-3-((2-(5-fluoroisoindolin-2-yl)-2-oxoethyl)amino)adamantan-1-yl)ureido)ethoxy)ethyl) carbamate (0.016 g, 0.028 mmol, 19% yield) was prepared according to general procedure M from 4-nitrophenyl (2-(2-((tert-butoxycarbonyl)amino)ethoxy)ethyl) carbamate (65) (0.056 g, 0.150 mmol) and purified by silica gel flash chromatography (elution with 0-10% MeOH in DCM). LRMS m/z (ESI⁺) 574 [M+H]⁺.

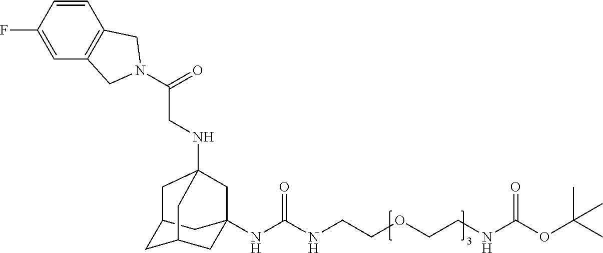

tert-Butyl (1-((3-((2-(5-fluoroisoindolin-2-yl)-2-oxoethyl) amino)adamantan-1-yl)amino)-1-oxo-5,8,11-trioxa-2-azatridecan-13-yl)carbamate (0.010 g, 0.015 mmol, 17% yield) was prepared according to general procedure M from 4-nitrophenyl (2,2-dimethyl-4-oxo-3,8,11,14-tetraoxa-5-azahexadecan-16-yl)carbamate (65) (0.040 g, 0.087 mmol) and purified by silica gel flash chromatography (elution with 0-15% MeOH in DCM). LRMS m/z (ESI⁺) 662 [M+H]⁺.

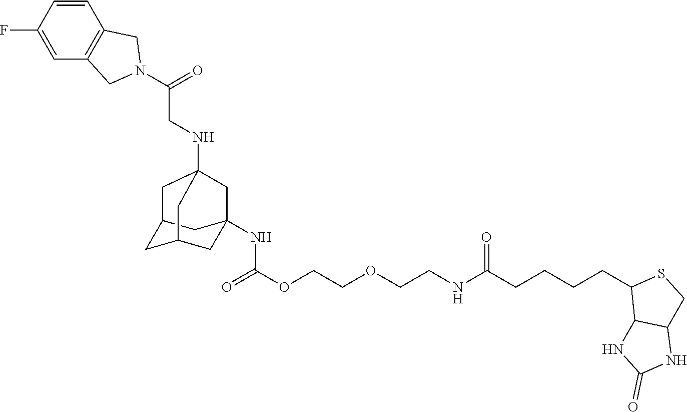 [SMILES: O=C(CCCCC1SCC2NC(=O)NC21)NCCOCCOC(=O)NC12CC3CC(CC(NCC(=O)N4Cc5ccc(F)cc5C4)(C3)C1)C2]

### Biotinylated Derivatives:

2-(2-(5-(2-Oxohexahydro-1H-thieno[3,4-d]imidazol-4-yl)pentanamido)ethoxy)ethyl (3-((2-(5-fluoroisoindolin-2-yl)-2-oxoethyl)amino)adamantan-1-yl)carbamate (73) (0.018 g, 0.025 mmol, 95% yield) was prepared according to general procedure N from 2-(2-((tert-butoxycarbonyl)amino)ethoxy)ethyl-3-((2-(5-fluoroisoindolin-2-yl)-2oxoethyl)amino)adamantan-1-ylcarbamate (66) (0.015 g, 0.026 mmol) and purified by silica gel flash chromatography (elution with a mixture of 30-80% DCM:MeOH:NH3 (8:2:0.2) in DCM). ¹H NMR (400 MHz, CDCl₃) δ_{H} 7.31-7.16 (m, 1H), 7.06-6.92 (m, 2H), 6.82 (br s, 1H), 6.78 (br s, 1H), 5.99 (br s, 1H), 5.36 (br s, 1H), 4.84-4.72 (m, 4H), 4.49 (t, J=6.3 Hz, 1H), 4.30 (t, J=6.3 Hz, 1H), 4.19-4.03 (m, 2H), 3.63 (s, 2H), 3.59-3.52 (m, 4H), 3.41 (t, J=4.9 Hz, 2H), 3.16-3.07 (m, 1H), 2.88 (dd, J=12.9, 4.8 Hz, 1H), 2.72 (d, J=12.8 Hz, 1H), 2.30-2.13 (m, 4H), 1.96-1.83 (m, 6H), 1.78-1.50 (m, 10H), 1.47-1.37 (m, 2H); ¹³C NMR (101 MHz, CDCl₃) δ_{H} 173.46, 169.48, 164.34, 162.62 (dd, J=244.4, 19.2 Hz), 154.33, 138.02 (dd, J=19.2, 9.1 Hz), 131.47 (dd, J=14.6, 2.0 Hz), 124.20 (dd, J=33.3, 8.1 Hz), 115.12 (dd, J=23.3, 23.2 Hz), 110.07 (dd, J=33.3, 23.2 Hz), 77.27, 69.77, 69.33, 62.92, 61.84, 60.26, 55.71, 52.96, 52.30, 51.95 (dd, J=79.8, 4.0 Hz), 51.42 (d, J=76.8 Hz), 45.65, 42.66 (d, J=5.1 Hz), 40.86, 40.72, 40.50, 39.09, 35.96, 35.24, 31.86, 29.78, 28.26, 28.13, 25.63, 22.67; LRMS m/z (ESI⁺) 701 [M+H]⁺; HRMS: calc. 701.3491, found 701.3516 [M+H]⁺.

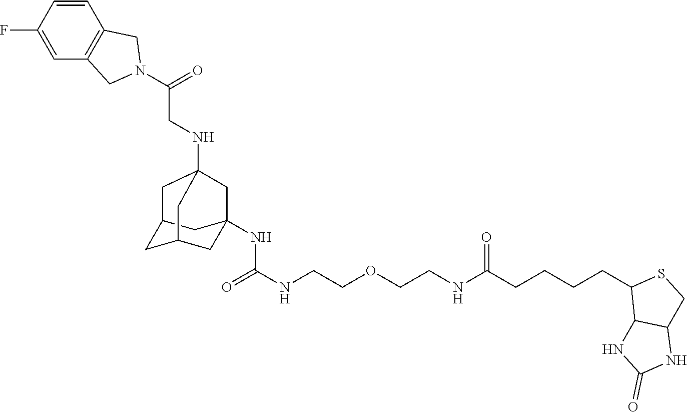 [SMILES: O=C(CCCCC1SCC2NC(=O)NC21)NCCOCCNC(=O)NC12CC3CC(CC(NCC(=O)N4Cc5ccc(F)cc5C4)(C3)C1)C2]

N-(2-(2-(3-(3-((2-(5-fluoroisoindolin-2-yl)-2-oxoethyl) amino)adamantan-1-yl)ureido)ethoxy)ethyl)-5-(2-oxohexahydro-1H-thieno[3,4-d]imidazol-4-yl) pentanamide (74) (0.025 g, 0.034 mmol, 97% yield) was prepared according to general procedure N from tert-butyl (2-(2-(3-(-3-((2-(5-fluoroisoindolin-2-yl)-2-oxoethyl)amino)adamantan-1-yl)ureido)ethoxy)ethyl) carbamate (66) (0.020 g, 0.043 mmol) and purified by silica gel flash chromatography (elution with a mixture of 30-80% DCM:MeOH:NH3 (8:2:0.2) in DCM). ¹H NMR (400 MHz, CD₃OD) δ_{H} 7.38-7.34 (m, 1H), 7.18-7.01 (m, 2H), 5.98 (br s, 1H), 4.89-4.75 (m, 4H), 4.49 (dd, J=7.9, 4.8 Hz, 1H), 4.30 (dd, J=7.9, 4.5 Hz, 1H), 3.86 (br s, 1H), 3.65 (s, 2H), 3.53-3.47 (m, 4H), 3.36 (t, J=4.0 Hz, 2H), 3.24 (t, J=4.0 Hz, 2H), 3.22-3.18 (m, 1H), 2.93 (dd, J=12.8, 5.0 Hz, 1H), 2.70 (dd, J=12.7, 1H), 2.30 (s, 2H), 2.23 (t, J=7.3 Hz, 2H), 2.14 (s, 2H), 2.08-2.01 (m, 2H), 1.87-1.52 (m, 12H), 1.44 (q, J=7.6 Hz, 2H); LRMS m/z (ESI⁺) 700 [M+H]⁺; HRMS: calc. 700.3651, found 700.3684 [M+H]⁺.

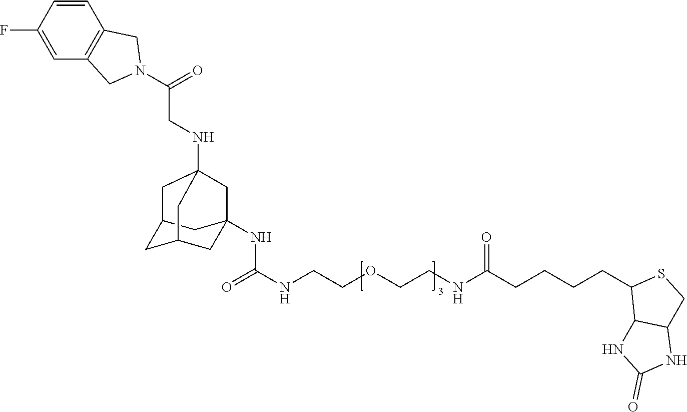

N-(1-((3-((2-(5-fluoroisoindolin-2-yl)-2-oxoethyl) amino)adamantan-1-yl)amino)-1-oxo-5,8,11-trioxa-2-azatridecan-13-yl)-5-(2-oxohexahydro-1H-thieno[3,4-d]imidazol-4-yl)pentanamide (75) (0.019 g, 0.023 mmol, 60% yield) was prepared according to general procedure N from tert-butyl (1-((3-((2-(5-fluoroisoindolin-2-yl)-2-oxoethyl) amino)adamantan-1-yl)amino)-1-oxo-5,8,11-trioxa-2-azatridecan-13-yl)carbamate (66) (0.025 g, 0.038 mmol) and purified by silica gel flash chromatography (elution with a mixture of 40-80% DCM:MeOH:NH3 (8:2:0.2) in DCM). ¹H NMR (400 MHz, CD₃OD) δ_{H} 7.54-7.46 (m, 1H), 7.31-7.14 (m, 2H), 5.01-4.83 (m, 4H), 4.63 (dd, J=7.9, 4.8 Hz, 1H), 4.44 (dd, J=7.9, 4.3 Hz, 1H), 3.90 (s, 2H), 3.69 (t, J=5.5 Hz, 2H), 3.64 (t, J=5.3 Hz, 2H), 3.51 (t, J=5.6 Hz, 2H), 3.36-3.30 (m, 1H), 3.06 (dd, J=12.8, 5.0 Hz, 1H), 2.84 (dd, J=12.7, 1H), 2.42 (s, 2H), 2.36 (t, J=7.5 Hz, 2H), 2.22 (s, 2H), 2.20-2.11 (m, 2H), 2.02-1.66 (m, 12H), 1.58 (q, J=7.6 Hz, 2H); ¹³C NMR (101 MHz, CD₃OD) δ_{H} 176.14, 169.14, 166.08, 164.19 (dd, J=241.90, 13.13 Hz), 159.98, 138.48 (dd, J=19.3, 9.0 Hz), 132.89 (dd, J=14.4, 2.3 Hz), 125.56 (dd, J=14.1, 9.1 Hz), 116.01 (dd, J=23.2, 10.1 Hz), 110.99 (dd, J=22.2, 17.2 Hz), 71.55, 71.25, 71.23, 70.56, 63.36, 61.62, 57.00, 56.16, 53.08 (d, J=52.5), 52.96, 52.28 (d, J=51.5 Hz), 45.55, 42.67 (d, J=5.1 Hz), 42.15, 41.05, 40.54, 40.40, 40.33, 36.74, 36.16, 31.19, 29.75, 29.50, 26.84; LRMS m/z (ESI⁺) 788 [M+H]⁺; HRMS: calc. 788.4175, found 788.4203 [M+H]⁺.

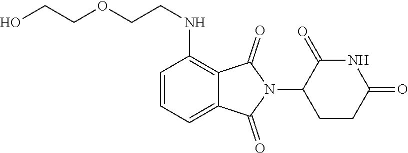 [SMILES: O=C1CCC(N2C(=O)c3cccc(NCCOCCO)c3C2=O)C(=O)N1]

Intermediates 69: 2-(2,6-Dioxopiperidin-3-yl)-4-((2-(2-hydroxyethoxy)ethyl)amino) isoindoline-1,3-dione (0.116 g, 0.322 mmol, 45% yield) was prepared according to general procedure O, using DMSO as solvent, from 2-(2,6-dioxo-piperidin-3-yl)-4-fluoroisoindoline-1,3-dione (68) (0.200 g, 0.724 mmol) and purified by silica gel flash chromatograph (elution with DCM to 5% MeOH in DCM). LRMS m/z (ESI⁺) 362 [M+H]⁺.

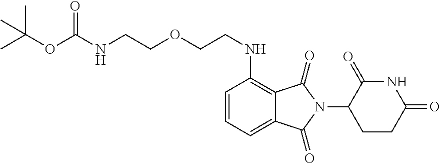 [SMILES: CC(C)(C)OC(=O)NCCOCCNc1cccc2c1C(=O)N(C1CCC(=O)NC1=O)C2=O]

tert-Butyl (2-(2-((2-(2,6-dioxopiperidin-3-yl)-1,3-dioxoisoindolin-4-yl)amino)ethoxy)ethyl)carbamate (0.312 g, 0.678 mmol, 94% yield) was prepared according to general procedure O, using NMP as solvent, from 2-(2,6-dioxo-piperidin-3-yl)-4-fluoroisoindoline-1,3-dione (68) (0.200 g, 0.724 mmol) and purified by silica gel flash chromatography (elution with DCM to 5% MeOH in DCM). LRMS m/z (ESI⁺) 483 [M+Na]⁺.

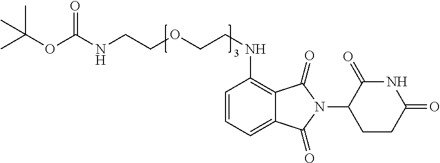

tert-Butyl (2-(2-(2-(2-((2-(2,6-dioxopiperidin-3-yl)-1,3-dioxoisoindolin-4-yl)amino)ethoxy)ethoxy)ethoxy)ethyl) carbamate (0.260 g, 0.474 mmol, 87% yield) was prepared according to general procedure O, using NMP as solvent, from 2-(2,6-dioxo-piperidin-3-yl)-4-fluoroisoindoline-1,3-dione (68) (0.150 g, 0.543 mmol) and purified by silica gel flash chromatography (elution with DCM to 5% MeOH in DCM). LRMS m/z (ESI⁺) 571 [M+Na]⁺.

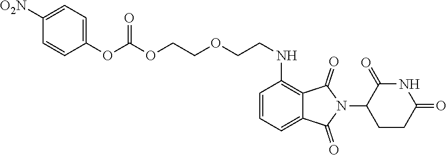 [SMILES: O=C1CCC(N2C(=O)c3cccc(NCCOCCOC(=O)Oc4ccc([N+](=O)[O-])cc4)c3C2=O)C(=O)N1]

Intermediates 70: 1,3-dioxoisoindolin-4-yl)amino)ethoxy)ethyl (4-nitrophenyl) carbonate (0.125 g, 0.237 mmol, 86% yield) was prepared according to general procedure P from 2-(2,6-dioxopiperidin-3-yl)-4-((2-(2-hydroxyethoxy)ethyl)amino) isoindoline-1,3-dione (69) (0.100 g, 0.277 mmol) and purified by silica gel flash chromatography (elution with 10-80% AcOEt in heptane). LRMS m/z (ESI⁺) 527 [M+H]⁺.

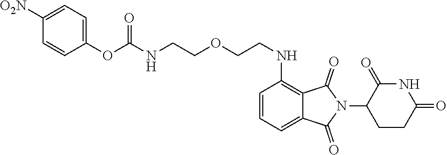 [SMILES: O=C1CCC(N2C(=O)c3cccc(NCCOCCNC(=O)Oc4ccc([N+](=O)[O-])cc4)c3C2=O)C(=O)N1]

4-Nitrophenyl (2-(2-((2-(2,6-dioxopiperidin-3-yl)-1,3-dioxoisoindolin-4-yl)amino)ethoxy)ethyl)carbamate (0.225 g, 0.429 mmol, 71% yield) was prepared according to general procedure P from tert-butyl (2-(2-((2-(2,6-dioxopiperidin-3-yl)-1,3-dioxoisoindolin-4-yl)amino)ethoxy)ethyl)carbamate (69) (0.277 g, 0.602 mmol) and purified by silica gel flash chromatography (elution with 10-80% AcOEt in heptane). LRMS m/z (ESI⁺) 526 [M+H]⁺.

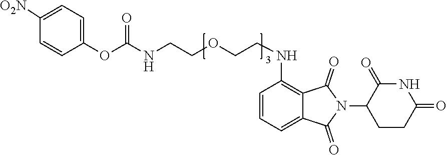

4-Nitrophenyl (2-(2-(2-(2-((2-(2,6-dioxopiperidin-3-yl)-1,3-dioxoisoindolin-4-yl)amino)ethoxy)ethoxy)ethoxy)ethyl) carbamate (0.292 g, 0.476 mmol, 93% yield) was prepared according to general procedure P from tert-butyl (2-(2-((2-(2,6-dioxopiperidin-3-yl)-1,3-dioxoisoindolin-4-yl)amino)ethoxy)ethyl)carbamate (69) (0.280 g, 0.510 mmol) and purified by silica gel flash chromatography (elution with 10-90% AcOEt in heptane). LRMS m/z (ESI⁺) 614 [M+H]⁺.

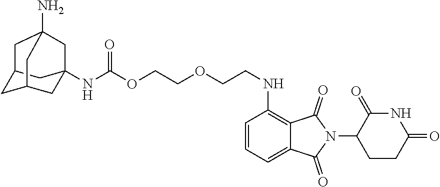 [SMILES: NC12CC3CC(C1)CC(NC(=O)OCCOCCNc1cccc4c1C(=O)N(C1CCC(=O)NC1=O)C4=O)(C3)C2]

Intermediates 71: 2-(2-((2-(2,6-Dioxopiperidin-3-yl)-1,3-dioxoisoindolin-4-yl)amino)ethoxy)ethyl(-3-aminoadamantan-1-yl)carbamate (0.029 g, 0.052 mmol, 28% yield) was prepared according to general procedure Q from 2-(2-((2-(2,6-dioxopiperidin-3-yl)-1,3-dioxoisoindolin-4-yl)amino)ethoxy)ethyl (4-nitrophenyl) carbonate (70) (0.100 g, 0.190 mmol) and purified by silica gel flash chromatography (elution with a mixture of 10-0% DCM:MeOH:NH3 (8:2:0.2) in DCM); LRMS m/z (ESI⁺) 554 [M+H]⁺.

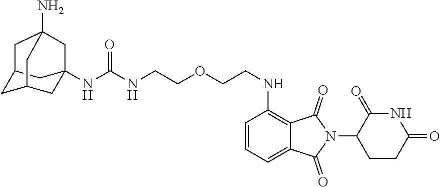 [SMILES: NC12CC3CC(C1)CC(NC(=O)NCCOCCNc1cccc4c1C(=O)N(C1CCC(=O)NC1=O)C4=O)(C3)C2]

1-(3-Aminoadamantan-1-yl)-3-(2-(2-((2-(2,6-dioxopiperidin-3-yl)-1,3-dioxoisoindolin-4-yl)amino)ethoxy)ethyl)urea (0.025 g, 0.045 mmol, 32% yield) was prepared according to general procedure Q from 4-nitrophenyl (2-(2-((2-(2,6-dioxopiperidin-3-yl)-1,3-dioxoisoindolin-4-yl)amino)ethoxy)ethyl)carbamate (70) (0.075 g, 0.143 mmol) and purified by silica gel flash chromatography (elution with a mixture of 10-70% DCM:MeOH:NH3 (8:2:0.2) in DCM); LRMS m/z (ESI⁺) 553 [M+H]⁺.

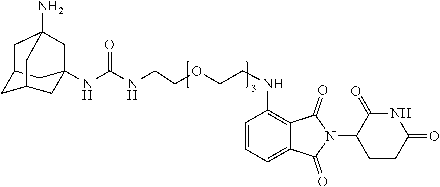

1-(3-Aminoadamantan-1-yl)-3-(2-(2-(2-(2-((2-(2,6-dioxopiperidin-3-yl)-1,3-dioxoisoindolin-4-yl)amino)ethoxy) ethoxy)ethoxy)ethyl)urea (0.080 g, 0.124 mmol, 55% yield) was prepared according to general procedure Q from 4-nitrophenyl (2-(2-(2-(2-((2-(2,6-dioxopiperidin-3-yl)-1,3-dioxoisoindolin-4-yl)amino)ethoxy)ethoxy)ethoxy)ethyl) carbamate (70) (0.140 g, 0.228 mmol) and purified by silica gel flash chromatography (elution with a mixture of 30-80% DCM:MeOH:NH3 (8:2:0.2) in DCM); LRMS m/z (ESI⁺) 641 [M+H]⁺.

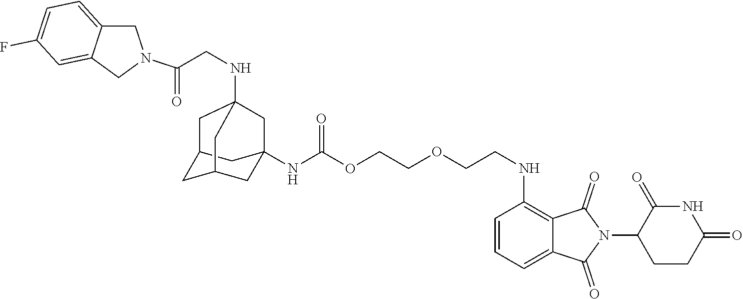 [SMILES: O=C1CCC(N2C(=O)c3cccc(NCCOCCOC(=O)NC45CC6CC(CC(NCC(=O)N7Cc8ccc(F)cc8C7)(C6)C4)C5)c3C2=O)C(=O)N1]

PROTACs 72: 2-(2-((2-(2,6-Dioxopiperidin-3-yl)-1,3-dioxoisoindolin-4-yl)amino)ethoxy)ethyl (3-((2-(5-fluoroisoindolin-2-yl)-2-oxoethyl)amino) adamantan-1-yl)carbamate (76) (0.010 g, 0.014 mmol, 51% yield) was prepared according to general procedure R from 2-bromo-1-(5-fluoroisoindolin-2-yl)ethan-1-one (2) (0.007 g, 0.027 mmol) and purified by silica gel flash chromatography (elution with 5% MeOH in DCM). ¹H NMR (400 MHz, CD₃OD) δ_{H} 7.53 (t, J=7.9 Hz, 1H), 7.37-7.28 (m, 1H), 7.14-6.96 (m, 4H), 5.05 (dd, J=12.6, 5.4 Hz, 1H), 4.84-4.71 (m, 4H), 4.17-4.07 (m, 2H), 3.68 (s, 2H), 3.74-3.60 (m, 4H), 3.48 (t, J=5.2 Hz, 2H), 2.92-2.66 (m, 3H), 2.23 (s, 2H), 2.15-2.07 (m, 1H), 2.01-1.87 (m, 4H), 1.86-1.67 (m, 6H), 1.65-1.56 (m, 2H); LRMS m/z (ESI⁺) 731 [M+H]⁺; HRMS: calc. 731.3199, found 731.3194 [M+H]⁺.

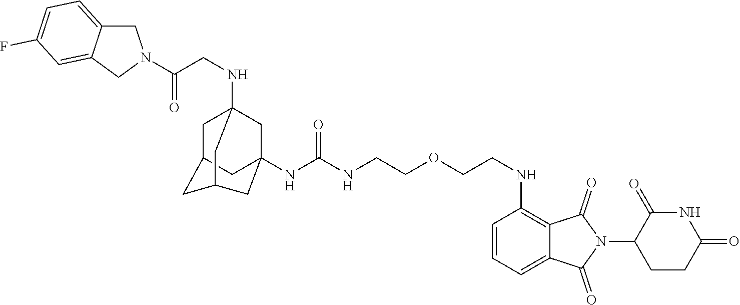 [SMILES: O=C1CCC(N2C(=O)c3cccc(NCCOCCNC(=O)NC45CC6CC(CC(NCC(=O)N7Cc8ccc(F)cc8C7)(C6)C4)C5)c3C2=O)C(=O)N1]

1-(2-(2-((2-(2,6-Dioxopiperidin-3-yl)-1,3-dioxoisoindolin-4-yl)amino)ethoxy)ethyl)-3-(3-((2-(5-fluoroisoindolin-2-yl)-2-oxoethyl)amino) adamantan-1-yl)urea (77) (0.012 g, 0.016 mmol, 71% yield) was prepared according to general procedure R fluoroisoindolin-2-yl)ethan-1-one (2) (0.006 g, 0.023 mmol) and purified by silica gel flash chromatography (elution with 5% MeOH in DCM). ¹H NMR (400 MHz, CDCl₃) δ_{H} 7.50 (t, J=7.8 Hz, 1H), 7.23-7.14 (m, 1H), 7.10 (d, J=7.1 Hz, 1H), 7.01-6.92 (m, 2H), 6.89 (d, J=8.7 Hz, 1H), 4.89 (dd, J=12.5, 5.2 Hz, 1H), 4.82-4.70 (m, 4H), 3.70 (s, 2H), 3.68-3.60 (m, 2H), 3.54 (t, J=5.4 Hz, 2H), 3.46-3.39 (m, 2H), 3.34-3.26 (m, 2H), 2.92-2.67 (m, 3H), 2.19 (s, 2H), 2.13-2.03 (m, 3H), 1.88-1.81 (m, 2H), 1.79-1.62 (m, 5H), 1.62-1.43 (m, 3H); LRMS m/z (ESI⁺) 730 [M+H]⁺; HRMS: calc. 730.3359, found 730.3361 [M+H]⁺.

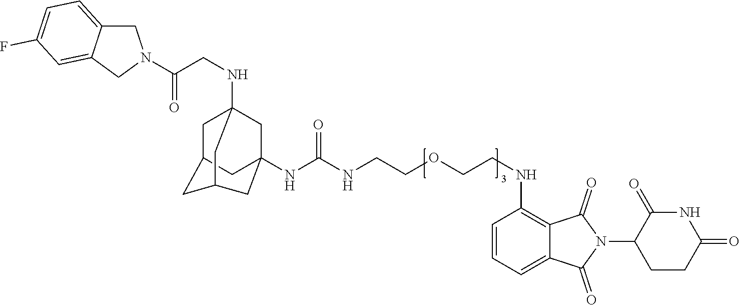

1-(2-(2-(2-(2-((2-(2,6-Dioxopiperidin-3-yl)-1,3-dioxoisoindolin-4-yl)amino)ethoxy)ethoxy) ethoxy)ethyl)-3-(3-((2-(5-fluoroisoindolin-2-yl)-2-oxoethyl)amino)adamantan-1-yl)urea (78) (0.012 g, 0.015 mmol, 47% yield) was prepared according to general procedure R from 2-bromo-1-(5-fluoroisoindolin-2-yl)ethan-1-one (2) (0.008 g, 0.031 mmol) and purified by silica gel flash chromatography (elution with 5% MeOH in DCM). ¹H NMR (400 MHz, CD₃OD) δ_{H} 7.54 (dd, J=8.6, 7.1 Hz, 1H), 7.38-7.28 (m, 1H), 7.12-7.01 (m, 4H), 5.05 (dd, J=12.4, 5.4 Hz, 1H), 4.89-4.68 (m, 4H), 3.78 (s, 2H), 3.72 (t, J=5.2 Hz, 2H), 3.68-3.61 (m, 6H), 3.60-3.56 (m, 2H), 3.53-3.44 (m, 4H), 3.22 (t, J=5.3 Hz, 2H), 2.93-2.59 (m, 3H), 2.25 (s, 2H), 2.16-2.04 (m, 3H), 2.03-1.95 (m, 2H), 1.85-1.72 (m, 6H), 1.70-1.57 (m, 2H); LRMS m/z (ESI⁺) 818 [M+H]⁺; HRMS: calc. 818.3883, found 818.3845 [M+H]⁺.

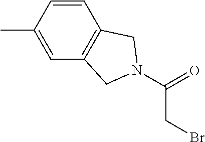

2-bromo-1-(5-methylisoindolin-2-yl)ethan-1-one (311 mg, 1.224 mmol, 65.2% yield) prepared according to general procedure A with 5-methylisoindoline (250 mg, 1.877 mmol) (ESI) m/z=254.6, 256.0 [M+H]⁺

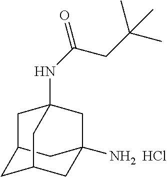 [SMILES: CC(C)(C)CC(=O)NC12CC3CC(CC(N)(C3)C1)C2 ; Cl]

hydrochloride (126 mg, 0.419 mmol, 86% yield) procedure W with 3,3-dimethylbutanoyl chloride (0.068 ml, 0.488 mmol) to produce tert-butyl ((1s,3r,5R,7S)-3-(3,3-dimethylbutanamido)adamantan-1-yl)carbamate. MS (ESI) m/z=387.4 [M+Na]⁺, followed by general procedure T. The obtained product was triturated with diethyl ether to obtain the pure compound.

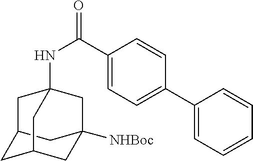 [SMILES: CC(C)(C)OC(=O)NC12CC3CC(C1)CC(NC(=O)c1ccc(-c4ccccc4)cc1)(C3)C2]

tert-butyl ((1s,3r,5R,7S)-3-([1,1′-biphenyl]-4-carboxamido)adamantan-1-yl)carbamate (225 mg, 0,504 mmol, 58,4% yield) prepared according to general procedure W with 4-Biphenylcarbonylchloride (187 mg, 0,863 mmol, 1 eq.) MS (ESI) m/z=469.3 [M+Na]⁺

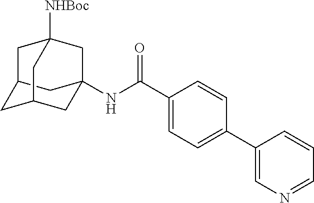 [SMILES: CC(C)(C)OC(=O)NC12CC3CC(C1)CC(NC(=O)c1ccc(-c4cccnc4)cc1)(C3)C2]

tert-butyl (3-(4-(pyridin-4-yl)benzamido)adamantan-1-yl)carbamate (126 mg, 0,282 mmol, 61,7% yield) prepared according to procedure S with 6-phenylnicotinic acid (100 mg, 0,502 mmol).). MS (ESI) m/z=448.2 [M+H]⁺

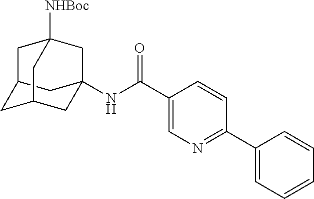 [SMILES: CC(C)(C)OC(=O)NC12CC3CC(C1)CC(NC(=O)c1ccc(-c4ccccc4)nc1)(C3)C2]

tert-butyl (3-(4-(pyridin-4-yl)benzamido)adamantan-1-yl)carbamate (103 mg, 0,230 mmol, 50,4% yield) prepared according to procedure S with 4-(pyridin-3-yl)benzoic acid (100 mg, 0,502 mmol). MS (ESI) m/z=448.3 [M+H]⁺

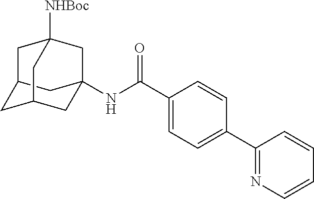 [SMILES: CC(C)(C)OC(=O)NC12CC3CC(C1)CC(NC(=O)c1ccc(-c4ccccn4)cc1)(C3)C2]

tert-butyl (3-(4-(pyridin-4-yl)benzamido)adamantan-1-yl)carbamate (123 mg, 0,275 mmol, 60,2% yield) prepared

according to procedure S with 4-(pyridin-2-yl)benzoic acid (100 mg, 0,502 mmol). MS (ESI) m/z=448.2 [M+H]⁺

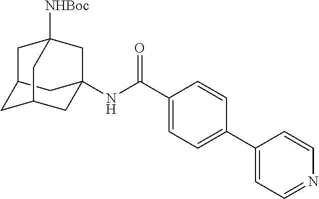 [SMILES: CC(C)(C)OC(=O)NC12CC3CC(C1)CC(NC(=O)c1ccc(-c4ccncc4)cc1)(C3)C2]

tert-butyl (3-(4-(pyridin-4-yl)benzamido)adamantan-1-yl)carbamate (100 mg, 0,223 mmol, 49,0% yield) prepared according to procedure S with 4-(pyridin-4-yl)benzoic acid (100 mg, 0,502 mmol).

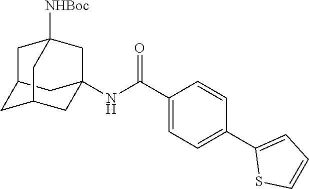 [SMILES: CC(C)(C)OC(=O)NC12CC3CC(C1)CC(NC(=O)c1ccc(-c4cccs4)cc1)(C3)C2]

tert-butyl ((1s,3r,5R,7S)-3-(4-(thiophen-2-yl)benzamido)adamantan-1-yl)carbamate (184 mg, 0,407 mmol, 91% yield) prepared according to procedure S with 4-(thiophen-2-yl)benzoic acid (100 mg, 0,490 mmol).

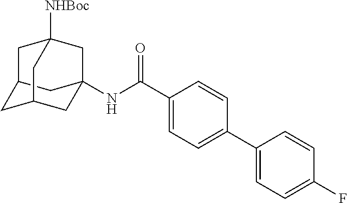 [SMILES: CC(C)(C)OC(=O)NC12CC3CC(C1)CC(NC(=O)c1ccc(-c4ccc(F)cc4)cc1)(C3)C2]

tert-butyl ((1s,3r,5R,7S)-3-(4′-fluoro-[1,1′-biphenyl]-4-carboxamido)adamantan-1-yl)carbamate (111 mg, 0,239 mmol, 63,6% yield) prepared according to procedure S with 4′-fluoro-[1,1′-biphenyl]-4-carboxylic acid (89 mg, 0,413 mmol).

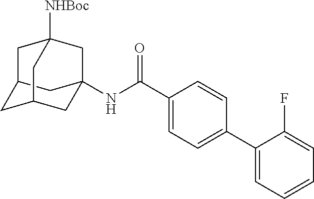 [SMILES: CC(C)(C)OC(=O)NC12CC3CC(C1)CC(NC(=O)c1ccc(-c4ccccc4F)cc1)(C3)C2]

tert-butyl ((1s,3r,5R,7S)-3-(2′-fluoro-[1,1′-biphenyl]-4-carboxamido)adamantan-1-yl)carbamate (102 mg, 0,220 mmol, 58,5% yield) prepared according to procedure S with 2′-fluoro-[1,1′-biphenyl]-4-carboxylic acid (106 mg, 0,488 mmol).

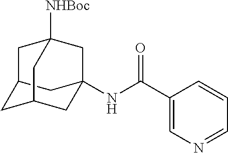 [SMILES: CC(C)(C)OC(=O)NC12CC3CC(C1)CC(NC(=O)c1cccnc1)(C3)C2]

tert-butyl ((1s,3r,5R,7S)-3-(nicotinamido)adamantan-1-yl)carbamate (70 mg, 0,188 mmol, 50,2% yield) prepared according to procedure S with nicotinic acid (50.8 mg, 0,413 mmol).

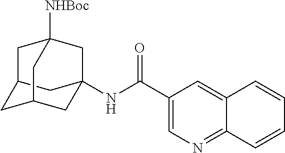 [SMILES: CC(C)(C)OC(=O)NC12CC3CC(C1)CC(NC(=O)c1cnc4ccccc4c1)(C3)C2]

tert-butyl ((1s,3r,5R,7S)-3-(quinoline-3-carboxamido)adamantan-1-yl)carbamate (116 mg, 0,275 mmol, 73,3% yield) prepared according to procedure S using quinoline-3-carboxylic acid (71.5 mg, 0,413 mmol).

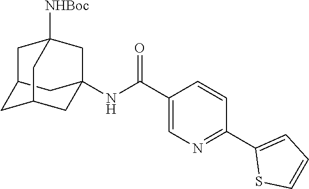 [SMILES: CC(C)(C)OC(=O)NC12CC3CC(C1)CC(NC(=O)c1ccc(-c4cccs4)nc1)(C3)C2]

tert-butyl ((1s,3r,5R,7S)-3-(6-(thiophen-2-yl)nicotinamido)adamantan-1-yl)carbamate (105 mg, 0,231 mmol, 61,7% yield) prepared according to procedure S using 6-(thiophen-2-yl)nicotinic acid (100 mg, 0,488 mmol).

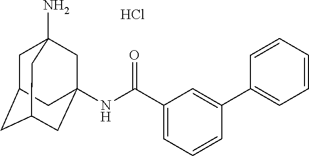 [SMILES: NC12CC3CC(C1)CC(NC(=O)c1cccc(-c4ccccc4)c1)(C3)C2 ; Cl]

N-((1s,3r,5R,7S)-3-aminoadamantan-1-yl)-[1,1′-biphenyl]-3-carboxamide hydrochloride (90 mg, 0,235 mmol, 64,4% yield) prepared from the product of procedure S with [1,1′-biphenyl]-3-carboxylic acid (97 mg, 0,488 mmol) MS (ESI) m/z=469.3 [M+Na]⁺ used as-is without column chromatography for procedure T, followed by trituration with diethyl ether, decanting, and drying in vacuo.

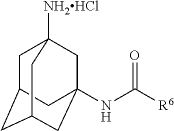

R⁶ substituted N-(3-aminoadamantan-1-yl)amide hydrochloride derivatives (80). Produced according to general procedure T with R⁶ substituted tert-butyl (3-amidoadamantan-1-yl)carbamate intermediates (79) (quantitative yield).

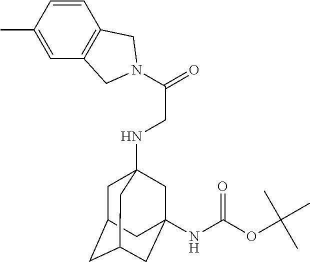 [SMILES: Cc1ccc2c(c1)CN(C(=O)CNC13CC4CC(C1)CC(NC(=O)OC(C)(C)C)(C4)C3)C2]

tert-butyl ((1s,3r,5R,7S)-3-((2-(5-methylisoindolin-2-yl)-2-oxoethyl)amino)adamantan-1-yl)carbamate (84) Procedure R with 2-bromo-1-(5-methylisoindolin-2-yl)ethan-1-one (47.7 mg, 0.188 mmol) and tert-butyl ((1s,3r,5R,7S)-3-aminoadamantan-1-yl)carbamate (100 mg, 0.375 mmol). Purification via normal-phase flash chromatography using a H 1 gradient of methanol in DCM to yield tert-butyl tert-butyl ((1s,3r,5R,7S)-3-((2-(5-methylisoindolin-2-yl)-2-oxoethyl)amino)adamantan-1-yl)carbamate (40 mg, 0.091 mmol, 48.5% yield).

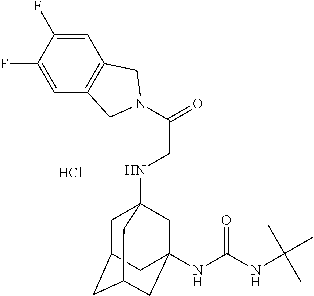 [SMILES: CC(C)(C)NC(=O)NC12CC3CC(CC(NCC(=O)N4Cc5cc(F)c(F)cc5C4)(C3)C1)C2 ; Cl]

1-(tert-butyl)-3-((1s,3r,5R,7S)-3-((2-(5,6-difluoroisoindolin-2-yl)-2-oxoethyl)amino)adamantan-1-yl)urea hydrochloride (85) Procedure R with 2-bromo-1-(5,6-difluoroisoindolin-2-yl)ethan-1-one (15 mg, 0,054 mmol) and 1-((1s,3r,5R,7S)-3-aminoadamantan-1-yl)-3-(tert-butyl)urea hydrochloride (28,7 mg, 0,095 mmol). After partial purification via column chromatography using a gradient of methanol in DCM, all the obtained compound was dissolved in dry dcm and the solution was dropped in hydrogen chloride 4 N solution in diethyl ether. The suspension was left to crystallize at 4° C. for 24 h and then decanted. The obtained precipitate was washed once with diethyl ether and the remaining solvent was removed in vacuo to yield 1-(tert-butyl)-3-((1s,3r,5R,7S)-3-((2-(5,6-difluoroisoindolin-2-yl)-2-oxoethyl)amino)adamantan-1-yl)urea hydrochloride (1.2 mg, 2,414 μmol, 4,4% yield).

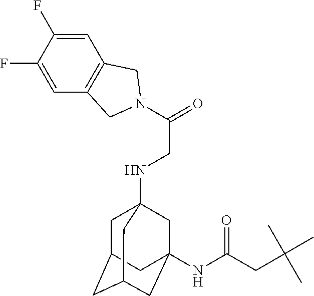 [SMILES: CC(C)(C)CC(=O)NC12CC3CC(CC(NCC(=O)N4Cc5cc(F)c(F)cc5C4)(C3)C1)C2]

N-((1s,3r,5R,7S)-3-((2-(5,6-difluoroisoindolin-2-yl)-2-oxoethyl)amino)adamantan-1-yl)-3,3-dimethylbutanamide R with 2-bromo-1-(5,6-difluoroisoindolin-2-yl)ethan-1-one (25 mg, 0.091 mmol) and N-((1s,3r,5R,7S)-3-aminoadamantan-1-yl)-3,3-dimethylbutanamide N hydrochloride (47.7 mg, 0.158 mmol). Purified via normal-phase flash chromatography using a gradient of methanol in DCM to yield N-((1s,3r,5R,7S)-3-((2-(5,6-difluoroisoindolin-2-yl)-2-oxoethyl)amino)adamantan-1-yl)-3,3-dimethylbutanamide (26 mg, 0.057 mmol, 62.5% yield). ¹H NMR (400 MHz, MeOD) δ 7.28 (m, 2H), 4.84 (s, 2H), 4.73 (s, 2H), 3.52 (s, 2H), 2.21 (m, 2H), 1.85 (m, 6H), 1.65 (m, 6H), 1.41 (s, 9H). ¹³C NMR (101 MHz, MeOD) δ 171.71, 156.55, 151.64 (ddd, J=248.0, 15.6, 5.5 Hz), 133.87 (ddd, J=45.3, 7.2, 3.4 Hz), 112.98 (dd, J=19.2, 14.1 Hz), 79.51, 53.65, 52.88, 52.57 (dd, J=78.5, 2.2 Hz), 46.35, 43.14, 41.87, 41.76, 36.46, 31.31, 28.82. MS (ESI) m/z 462.3 [M+H]⁺

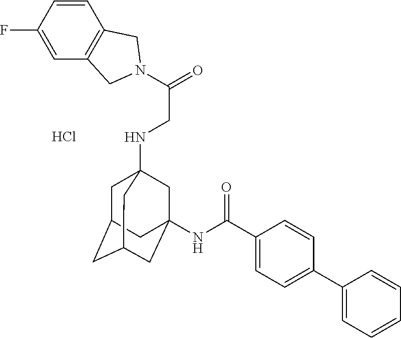 [SMILES: O=C(NC12CC3CC(CC(NCC(=O)N4Cc5ccc(F)cc5C4)(C3)C1)C2)c1ccc(-c2ccccc2)cc1 ; Cl]

N-((1s,3r,5R,7S)-3-((2-(5-fluoroisoindolin-2-yl)-2-oxoethyl)amino)adamantan-1-yl)-[1,1′-biphenyl]-4-carboxamide hydrochloride (87) Procedure R with 2-bromo-1-(5-fluoroisoindolin-2-yl)ethan-1-one (10.93 mg, 0,042 mmol) and N-((1s,3r,5R,7S)-3-aminoadamantan-1-yl)-[1,1′-biphenyl]-4-carboxamide 2,2,2-trifluoroacetate (39 mg, 0,085 mmol). Purified via normal-phase flash chromatography using a gradient of methanol in DCM to yield N-((1s,3r,5R,7S)-3-((2-(5-fluoroisoindolin-2-yl)-2-oxoethyl)amino)adamantan-1-yl)-[1,1′-biphenyl]-4-carboxamide (15 mg, 0,029 mmol, 67,6% yield).

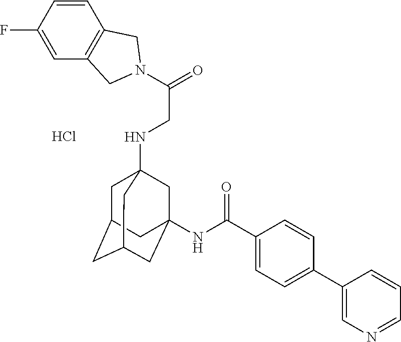 [SMILES: O=C(NC12CC3CC(CC(NCC(=O)N4Cc5ccc(F)cc5C4)(C3)C1)C2)c1ccc(-c2cccnc2)cc1 ; Cl]

N-((1s,3r,5R,7S)-3-((2-(5-fluoroisoindolin-2-yl)-2-oxoethyl)amino)adamantan-1-yl)-4-(pyridin-3-yl)benzamide hydrochloride (88) Procedure R with 2-bromo-1-(5-fluoroisoindolin-2-yl)ethan-1-one (29 mg, 0,112 mmol) and N-((1s,3r,5R,7S)-3-aminoadamantan-1-yl)-4-(pyridin-3-yl)benzamide hydrochloride (94 mg, 0,245 mmol). Purified via normal-phase flash chromatography using a gradient of methanol in DCM to yield N-((1s,3r,5R,7S)-3-((2-(5-fluoroisoindolin-2-yl)-2-oxoethyl)amino)adamantan-1-yl)-4-(pyridin-3-yl)benzamide (16 mg, 0,030 mmol, 27,1% yield). Followed by general procedure V. ¹H NMR (400 MHz, MeOD) δ 9.27-9.22 (m, 1H), 8.96 (ddd, J=8.3, 2.2, 1.4 Hz, 1H), 8.88 (d, J=5.6 Hz, 1H), 8.20 (dd, J=8.3, 5.7 Hz, 1H), 8.00 (d, J=8.6 Hz, 2H), 7.94 (d, J=8.6 Hz, 2H), 7.37 (dd, J=8.5, 4.9 Hz, 1H), 7.17-7.03 (m, 2H), 4.94 (d, J=14.7 Hz, 2H), 4.81 (d, J=13.3 Hz, 2H), 4.14 (s, 2H), 2.54 (s, 2H), 2.48-2.42 (m, 2H), 2.38-2.31 (m, 2H), 2.11-1.97 (m, 6H), 1.85-1.72 (m, 2H). ¹³C NMR (101 MHz, MeOD) δ 169.30, 165.44, 163.00 (d, J=8.3 Hz), 145.54, 141.89, 141.44, 140.88, 139.25 (dd, J=47.4, 9.0 Hz), 138.17, 137.79, 132.68 (d, J=46.9 Hz), 129.73, 128.79, 128.64, 125.59 (dd, J=13.2, 8.9 Hz), 116.10 (dd, J=23.0, 10.1 Hz), 111.02 (dd, J=24.2, 16.7 Hz), 59.64, 54.84, 53.00 (dd, J=85.6, 2.2 Hz), 52.49 (d, J=84.5 Hz), 42.18, 42.10, 40.56, 38.23, 35.53, 30.96. MS (ESI) m/z=525.3 [M+H]⁺

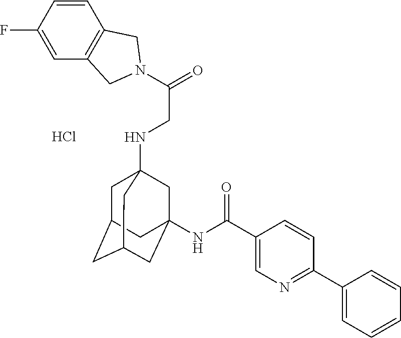 [SMILES: O=C(NC12CC3CC(CC(NCC(=O)N4Cc5ccc(F)cc5C4)(C3)C1)C2)c1ccc(-c2ccccc2)nc1 ; Cl]

N-((1s,3r,5R,7S)-3-((2-(5-fluoroisoindolin-2-yl)-2-oxoethyl)amino)adamantan-1-yl)-6-phenylnicotinamide hydrochloride (89) Procedure R using 2-bromo-1-(5-fluoroisoindolin-2-yl)ethan-1-one (34.7 mg, 0,134 mmol) and N-((1s,3r,5R,7S)-3-aminoadamantan-1-yl)-6-phenylnicotinamide dihydrochloride (113 mg, 0,269 mmol). Purified via normal-phase flash chromatography using a gradient of methanol in DCM to yield N-((1s,3r,5R,7S)-3-((2-(5-fluoroisoindolin-2-yl)-2-oxoethyl)amino)adamantan-1-yl)-6-phenylnicotinamide (62 mg, 0,118 mmol, 88% yield). MS (ESI) m/z=525.4 [M+H]⁺ Followed by general procedure V. ¹H NMR (400 MHz, MeOD) δ 9.18 (dd, J=2.1, 0.7 Hz, 1H), 8.90 (dd, J=8.5, 2.1 Hz, 1H), 8.42 (d, J=8.4 Hz, OH), 8.06-7.98 (m, 2H), 7.78-7.65 (m, 3H), 7.37 (dt, J=8.8, 4.5 Hz, 1H), 7.11 (dtd, J=17.2, 8.6, 2.1 Hz, 2H), 4.94 (d, J=14.1 Hz, 2H), 4.82 (d, J=13.4 Hz, 2H), 4.14 (s, 2H), 2.55 (s, 2H), 2.49-2.44 (m, 2H), 2.39-2.32 (m, 2H), 2.13-1.96 (m, 6H), 1.86-1.72 (m, 2H). ¹³C NMR (101 MHz, MeOD) δ 165.38, 164.11, 163.09, 156.50, 145.09, 143.80, 139.27 (dd, J=43.6, 9.1 Hz), 133.73, 133.54, 132.93, 132.68 (d, J=43.8 Hz), 130.95, 129.39, 126.26, 125.59 (dd, J=19.1, 8.8 Hz), 116.14 (dd, J=23.1, 10.8 Hz), 111.04 (t, J=23.3 Hz), 59.57, 55.34, 52.99 (dd, J=88.5, 2.6 Hz), 52.48 (d, J=87.7 Hz), 42.15, 42.11, 40.42, 38.17, 35.47, 30.96. MS (ESI) m/z=525.3 [M+H]⁺

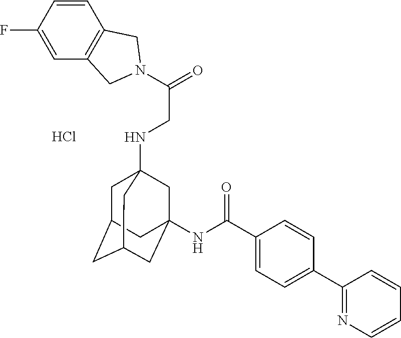 [SMILES: O=C(NC12CC3CC(CC(NCC(=O)N4Cc5ccc(F)cc5C4)(C3)C1)C2)c1ccc(-c2ccccn2)cc1 ; Cl]

N-((1s,3r,5R,7S)-3-((2-(5-fluoroisoindolin-2-yl)-2-oxoethyl)amino)adamantan-1-yl)-4-(pyridin-2-yl)benzamide hydrochloride (90) Procedure R with 2-bromo-1-(5-fluoroisoindolin-2-yl)ethan-1-one (34.5 mg, 0,134 mmol) and N-((1s,3r,5R,7S)-3-aminoadamantan-1-yl)-4-(pyridin-2-yl)benzamide bis(2,2,2-trifluoroacetate) (154 mg, 0,268 mmol). Purified via normal-phase flash chromatography using a gradient of methanol in DCM to yield N-((1s,3r,5R,7S)-3-((2-(5-fluoroisoindolin-2-yl)-2-oxoethyl)amino)adamantan-1-yl)-4-(pyridin-2-yl)benzamide (42 mg, 0,080 mmol, 59,8% yield). ¹H NMR (400 MHz, DMSO) δ 8.69 (ddd, J=4.8, 1.9, 0.9 Hz, 1H), 8.18-8.11 (m, 2H), 8.07-8.00 (m, 1H), 7.95-7.86 (m, 3H), 7.80 (s, 1H), 7.43-7.33 (m, 2H), 7.22 (td, J=9.2, 2.5 Hz, 1H), 7.14 (ddd, J=10.8, 6.6, 2.0 Hz, 1H), 4.84 (d, J=15.9 Hz, 2H), 4.64 (d, J=13.8 Hz, 2H), 3.48 (s, 2H), 2.19 (q, J=3.3 Hz, 2H), 2.04-1.96 (m, 6H), 1.67-1.48 (m, 6H). ¹³C NMR (101 MHz, DMSO) δ 166.38 (d, J=672.3 Hz), 165.70, 160.64, 155.11, 149.66, 140.66, 138.84 (dd, J=85.2, 9.1 Hz), 137.35, 136.08, 132.37 (dd, J=83.1, 2.4 Hz), 127.85, 126.06, 124.61 (dd, J=23.8, 9.1 Hz), 123.06, 120.63, 114.51 (d, J=22.8 Hz), 110.04 (dd, J=28.1, 23.6 Hz), 53.27, 51.92, 51.35 (dd, J=93.0, 2.4 Hz), 50.77 (d, J=92.6 Hz), 45.39, 42.54 (d, J=4.0 Hz), 40.94, 35.23, 29.42. MS (ESI) m/z=525.3 [M+H]⁺ Followed by procedure V. MS (ESI) m/z=525.3 [M+H]⁺

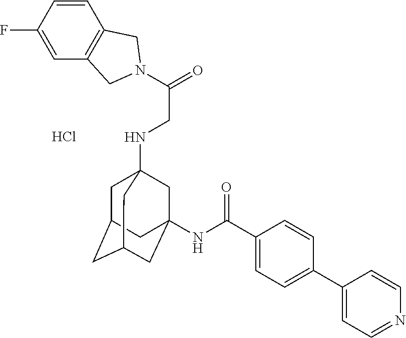 [SMILES: O=C(NC12CC3CC(CC(NCC(=O)N4Cc5ccc(F)cc5C4)(C3)C1)C2)c1ccc(-c2ccncc2)cc1 ; Cl]

N-((1s,3r,5R,7S)-3-((2-(5-fluoroisoindolin-2-yl)-2-oxoethyl)amino)adamantan-1-yl)-4-(pyridin-4-yl)benzamide hydrochloride (91) Procedure R with 2-bromo-1-(5-fluoroisoindolin-2-yl)ethan-1-one (30.7 mg, 0,119 mmol) and N-((1s,3r,5R,7S)-3-aminoadamantan-1-yl)-4-(pyridin-4-yl)benzamide dihydrochloride (90 mg, 0,214 mmol). Purified via normal-phase flash chromatography using a gradient of methanol in DCM to yield N-((1s,3r,5R,7S)-3-((2-(5-fluoroisoindolin-2-yl)-2-oxoethyl)amino)adamantan-1-yl)-4-(pyridin-4-yl)benzamide (9 mg, 0,017 mmol, 14,42% yield). ¹H NMR (400 MHz, CDCl₃) δ 8.71-8.65 (m, 1H), 7.87-7.79 (m, 1H), 7.72-7.64 (m, 1H), 7.54-7.48 (m, 1H), 7.25-7.19 (m, OH), 7.06-6.93 (m, 1H), 5.99 (s, OH), 4.79 (d, J=11.1 Hz, 2H), 3.62 (s, 1H), 2.36-2.30 (m, 1H), 2.21 (s, 1H), 2.19-2.11 (m, 1H), 2.08-2.01 (m, 1H), 1.88-1.80 (m, 1H), 1.78-1.59 (m, 2H). MS (ESI) m/z=525.4 [M+H]⁺ followed by procedure V. MS (ESI) m/z=525.3 [M+H]⁺

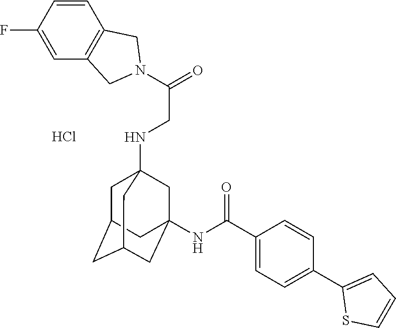 [SMILES: O=C(NC12CC3CC(CC(NCC(=O)N4Cc5ccc(F)cc5C4)(C3)C1)C2)c1ccc(-c2cccs2)cc1 ; Cl]

N-((1s,3r,5R,7S)-3-((2-(5-fluoroisoindolin-2-yl)-2-oxoethyl)amino)adamantan-1-yl)-4-(thiophen-2-yl)benzamide hydrochloride (92) Procedure R with 2-bromo-1-(5-fluoroisoindolin-2-yl)ethan-1-one (64,4 mg, 0,249 mmol) and N-((1s,3r,5R,7S)-3-aminoadamantan-1-yl)-4-(thiophen-2-yl)benzamide hydrochloride (194 mg, 0,499 mmol). Purified via normal-phase flash chromatography using a gradient of methanol in DCM to yield N-((1s,3r,5R,7S)-3-((2-(5-fluoroisoindolin-2-yl)-2-oxoethyl)amino)adamantan-1-yl)-4-(thiophen-2-yl)benzamide (83 mg, 0,157 mmol, 62,8% yield). MS (ESI) m/z 530.3 [M+H]⁺ followed by procedure V. ¹H NMR (400 MHz, MeOD) δ 7.84-7.76 (m, 1H), 7.76-7.68 (m, 1H), 7.50 (dd, J=3.7, 1.1 Hz, 1H), 7.45 (dd, J=5.1, 1.1 Hz, 1H), 7.36 (dt, J=9.1, 4.7 Hz, 1H), 7.17-7.03 (m, 2H), 4.92 (d, J=14.3 Hz, 1H), 4.80 (d, J=13.2 Hz, 1H), 4.12 (s, 1H), 2.52 (s, 1H), 2.47-2.41 (m, 1H), 2.38-2.31 (m, 1H), 2.08-1.96 (m, 3H), 1.84-1.70 (m, 1H). ¹³C NMR (101 MHz, MeOD) δ 165.38, 164.11, 163.09, 156.50, 145.09, 143.80, 139.27 (dd, J=43.6, 9.1 Hz), 133.73, 133.54, 132.93, 132.68 (d, J=43.8 Hz), 130.95, 129.39, 126.26, 125.59 (dd, J=19.1, 8.8 Hz), 116.14 (dd, J=23.1, 10.8 Hz), 111.04 (t, J=23.3 Hz), 59.57, 55.34, 52.99 (dd, J=88.5, 2.6 Hz), 52.48 (d, J=87.7 Hz), 42.15, 42.11, 40.42, 38.17, 35.47, 30.96. MS (ESI) m/z 530.3 [M+H]⁺

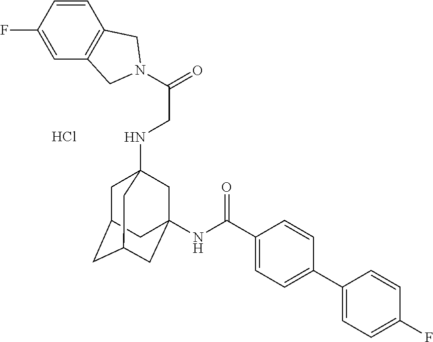 [SMILES: O=C(NC12CC3CC(CC(NCC(=O)N4Cc5ccc(F)cc5C4)(C3)C1)C2)c1ccc(-c2ccc(F)cc2)cc1 ; Cl]

4′-fluoro-N-((1s,3r,5R,7S)-3-((2-(5-fluoroisoindolin-2-yl)-2-oxoethyl)amino)adamantan-1-yl)-[1,1′-biphenyl]-4-carboxamide hydrochloride (93) Procedure R with 2-bromo-1-(5-fluoroisoindolin-2-yl)ethan-1-one (41.2 mg, 0,160 mmol) and N-((1s,3r,5R,7S)-3-aminoadamantan-1-yl)-4′-fluoro-[1,1′-biphenyl]-4-carboxamide hydrochloride (96 mg, 0,239 mmol). Purified via normal-phase flash chromatography using a gradient of methanol in DCM to yield 4′-fluoro-N-((1s,3r,5R,7S)-3-((2-(5-fluoroisoindolin-2-yl)-2-oxoethyl)amino)adamantan-1-yl)-[1,1′-biphenyl]-4-carboxamide (41.3 mg, 0,076 mmol, 47,8% yield). MS (ESI) m/z 542.3 [M+H]⁺, followed by procedure V. ¹H NMR (400 MHz, MeOD) δ 7.85 (d, J=8.2 Hz, 2H), 7.68 (dd, J=8.3, 4.4 Hz, 4H), 7.37 (dt, J=9.5, 5.1 Hz, 1H), 7.25-7.16 (m, 2H), 7.16-7.01 (m, 2H), 4.94 (s, 1H), 4.81 (d, J=13.2 Hz, 2H), 4.13 (s, 2H), 2.53 (s, 2H), 2.47-2.41 (m, 2H), 2.39-2.32 (m, 2H), 2.09-1.97 (m, 6H), 1.85-1.71 (m, 2H). ¹³C NMR (101 MHz, MeOD) δ 170.05, 165.41, 164.31 (d, J=246.1 Hz), 144.44, 139.26 (dd, J=43.7, 8.9 Hz), 137.55 (d, 3=3.3 Hz), 135.40, 132.68 (dd, J=46.6, 2.5 Hz), 130.02 (d, J=8.3 Hz), 129.04, 127.83, 125.58 (dd, J=20.2, 9.0 Hz), 116.86, 116.65, 116.13 (dd, J=23.1, 10.3 Hz), 111.03 (t, J=23.9 Hz), 59.59, 54.63, 53.53-52.37 (m), 52.45 (d, J=90.5 Hz), 42.16, 42.03, 40.67, 38.26, 35.60, 30.97. MS (ESI) m/z=542.3 [M+H]⁺

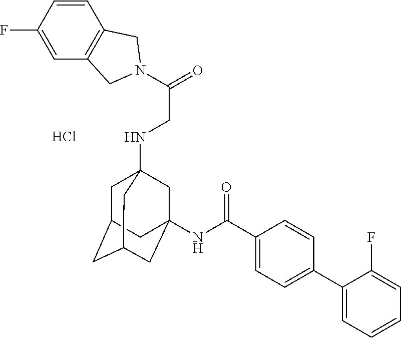 [SMILES: O=C(NC12CC3CC(CC(NCC(=O)N4Cc5ccc(F)cc5C4)(C3)C1)C2)c1ccc(-c2ccccc2F)cc1 ; Cl]

2′-fluoro-N-((1s,3r,5R,7S)-3-((2-(5-fluoroisoindolin-2-yl)-2-oxoethyl)amino)adamantan-1-yl)-[1,1′-biphenyl]-4-carboxamide hydrochloride (94) Procedure R with 2-bromo-1-(5-fluoroisoindolin-2-yl)ethan-1-one (39.9 mg, 0,155 mmol) and N-((1s,3r,5R,7S)-3-aminoadamantan-1-yl)-2′-fluoro-[1,1′-biphenyl]-4-carboxamide hydrochloride (93 mg, 0,232 mmol). Purified via normal-phase flash chromatography using a gradient of methanol in DCM to yield 2′-fluoro-N-((1s,3r,5R,7S)-3-((2-(5-fluoroisoindolin-2-yl)-2-oxoethyl)amino)adamantan-1-yl)-[1,1′-biphenyl]-4-carboxamide (27 mg, 0,050 mmol, 32,2% yield). MS (ESI) m/z=542.3 [M+H]⁺, followed by general procedure V. ¹H NMR (400 MHz, MeOD) δ 8.02 (s, 1H), 7.86 (d, J=8.3 Hz, 2H), 7.67-7.60 (m, 2H), 7.52 (td, J=7.8, 1.8 Hz, 1H), 7.44-7.33 (m, 2H), 7.28 (t, J=7.5 Hz, 1H), 7.22 (dd, J=11.1, 8.2 Hz, 1H), 7.17-7.04 (m, 2H), 4.94 (s, 1H), 4.81 (d, J=13.3 Hz, 2H), 4.13 (s, 2H), 2.53 (s, 2H), 2.47-2.41 (m, 2H), 2.40-2.32 (m, 2H), 2.09-1.97 (m, 6H), 1.85-1.71 (m, 2H). ¹³C NMR (101 MHz, MeOD) δ 170.04, 165.41, 163.04 (d, J=8.7 Hz), 161.09 (d, 3=247.0 Hz), 140.32, 139.26 (dd, J=43.3, 9.1 Hz), 135.85, 132.68 (dd, J=46.2, 2.5 Hz), 131.81 (d, J=3.2 Hz), 131.06 (d, J=8.4 Hz), 130.05 (d, J=3.2 Hz), 129.22 (d, J=13.2 Hz), 128.55, 125.87 (d, J=3.7 Hz), 125.58 (dd, J=20.3, 8.9 Hz), 117.15 (d, J=22.8 Hz), 116.13 (dd, J=23.2, 10.4 Hz), 111.03 (t, J=23.9 Hz), 59.60, 54.65, 52.96 (dd, J=91.5, 2.8 Hz), 52.45 (d, J=90.6 Hz), 42.15, 42.03, 40.66, 38.27, 35.59, 30.97. MS (ESI) m/z=542.3 [M+H]⁺

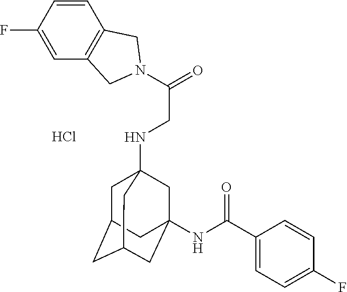 [SMILES: O=C(NC12CC3CC(CC(NCC(=O)N4Cc5ccc(F)cc5C4)(C3)C1)C2)c1ccc(F)cc1 ; Cl]

4-fluoro-N-((1s,3r,5R,7S)-3-((2-(5-fluoroisoindolin-2-yl)-2-oxoethyl)amino)adamantan-1-yl)benzamide hydrochloride (95) procedure U with 4-fluorobenzoic acid (28.0 mg, 0,200 mmol) and 2-(((1s,3r,5R,7S)-3-aminoadamantan-1-yl)amino)-1-(5-fluoroisoindolin-2-yl)ethan-1-one dihydrochloride (100 mg, 0,240 mmol). Purified via normal phase column chromatography using a gradient of methanol in DCM. Repurified using a gradient of isopropanol in DCM+1% methanol to yield 4-fluoro-N-((1s,3r,5R,7S)-3-((2-(5-fluoroisoindolin-2-yl)-2-oxoethyl)amino)adamantan-1-yl)benzamide (32 mg, 0,069 mmol, 34,3% yield) MS (ESI) m/z=466.3 [M+H]⁺ followed by general procedure V. ¹H NMR (400 MHz, MeOD) δ 7.96 (s, 1H), 7.87-7.77 (m, 2H), 7.41-7.32 (m, 1H), 7.23-7.04 (m, 4H), 4.91 (s, 2H), 4.81 (d, J=13.3 Hz, 2H), 4.11 (s, 2H), 2.49 (s, 2H), 2.46-2.40 (m, 2H), 2.36-2.29 (m, 2H), 2.07-1.94 (m, 6H), 1.83-1.70 (m, 2H). ¹³C NMR (101 MHz, MeOD) δ 169.23, 166.10 (d, J=250.0 Hz), 165.41, 163.04 (d, J=8.8 Hz), 139.27 (dd, J=43.9, 8.9 Hz), 133.06 (d, J=3.2 Hz), 132.69 (dd, J=46.7, 2.7 Hz), 130.95 (d, J=8.9 Hz), 125.58 (dd, J=20.2, 8.9 Hz), 116.25 (d, J=22.1 Hz), 116.13 (dd, J=23.1, 10.2 Hz), 111.03 (t, J=23.9 Hz), 59.56, 54.61, 52.96 (dd, J=91.7, 2.5 Hz), 52.45 (d,J=90.5 Hz), 42.11, 42.02, 40.62, 38.24, 35.57, 30.95. MS (ESI) m/z=466.3 [M+H]⁺

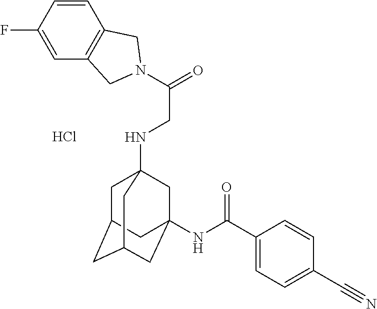 [SMILES: N#Cc1ccc(C(=O)NC23CC4CC(CC(NCC(=O)N5Cc6ccc(F)cc6C5)(C4)C2)C3)cc1 ; Cl]

4-cyano-N-((1s,3r,5R,7S)-3-((2-(5-fluoroisoindolin-2-yl)-2-oxoethyl)amino)adamantan-1-yl)benzamide hydrochloride (96) procedure U with 4-cyanobenzoic acid (30.3 mg, 0,206 mmol) and 2-(((1s,3r,5R,7S)-3-aminoadamantan-1-yl)amino)-1-(5-fluoroisoindolin-2-yl)ethan-1-one dihydrochloride (103 mg, 0,247 mmol) to yield 4-cyano-N-((1s,3r,5R,7S)-3-((2-(5-fluoroisoindolin-2-yl)-2-oxoethyl)amino)adamantan-1-yl)benzamide 4-cyano-N-((1s,3r,5R,7S)-3-((2-(5-fluoroisoindolin-2-yl)-2-oxoethyl)amino)adamantan-1-yl)benzamide (37 mg, 0,078 mmol, 38,0% yield) MS (ESI) m/z=473.3 [M+H]⁺ followed by general procedure V. ¹H NMR (400 MHz, MeOD) δ 8.20 (s, 1H), 7.90 (d, J=8.1 Hz, 2H), 7.82 (d, J=8.3 Hz, 2H), 7.41-7.32 (m, 1H), 7.18-7.04 (m, 2H), 4.96-4.92 (m, 2H), 4.81 (d, J=13.3 Hz, 2H), 4.12 (s, 2H), 2.50 (s, 2H), 2.46-2.40 (m, 2H), 2.36-2.28 (m, 2H), 2.08-1.95 (m, 6H), 1.83-1.70 (m, 2H). ¹³C NMR (101 MHz, MeOD) δ 168.56, 165.39, 163.03 (d, J=8.4 Hz), 140.91, 139.26 (dd, J=44.4, 9.0 Hz), 133.42, 132.68 (dd, J=47.0, 2.6 Hz), 129.29, 125.58 (dd, J=18.5, 8.9 Hz), 119.05, 116.12 (dd, J=23.2, 10.1 Hz), 115.88, 111.02 (dd, J=24.2, 22.0 Hz), 59.57, 54.87, 52.97 (dd, J=90.2, 2.9 Hz), 52.45 (d, J=89.2 Hz), 42.05, 40.50, 38.21, 35.51, 30.94. MS (ESI) m/z=473.4 [M+H]⁺

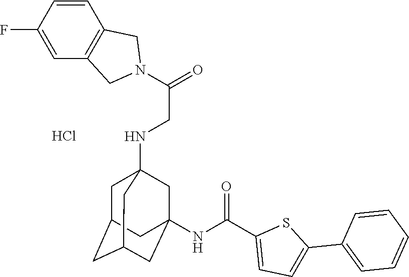 [SMILES: O=C(NC12CC3CC(CC(NCC(=O)N4Cc5ccc(F)cc5C4)(C3)C1)C2)c1ccc(-c2ccccc2)s1 ; Cl]

N-((1s,3r,5R,7S)-3-((2-(5-fluoroisoindolin-2-yl)-2-oxoethyl)amino)adamantan-1-yl)-5-phenylthiophene-2-carboxamide hydrochloride (97) procedure U with 5-phenylthiophene-2-carboxylic acid (42.1 mg, 0,206 mmol) and 2-(((1s,3r,5R,7S)-3-aminoadamantan-1-yl)amino)-1-(5-fluoroisoindolin-2-yl)ethan-1-one dihydrochloride (103 mg, 0,247 mmol) to yield N-((1s,3r,5R,7S)-3-((2-(5-fluoroisoindolin-2-yl)-2-oxoethyl)amino)adamantan-1-yl)-5-phenylthiophene-2-carboxamide (47 mg, 0,089 mmol, 43,0% yield) MS (ESI) m/z=530.3 [M+H]⁺ followed by general procedure V. ¹H NMR (400 MHz, MeOD) δ 7.73-7.63 (m, 3H), 7.46-7.31 (m, 5H), 7.18-7.04 (m, 2H), 4.93 (s, 1H), 4.81 (d, J=13.4 Hz, 2H), 4.11 (s, 2H), 2.49 (s, 2H), 2.46-2.40 (m, 2H), 2.37-2.29 (m, 2H), 2.06-1.94 (m, 6H), 1.84-1.70 (m, 2H). ¹³C NMR (101 MHz, MeOD) δ 165.39, 164.06, 150.42, 139.90, 139.09, 134.88, 132.67 (d, J=45.8 Hz), 130.77, 130.22, 129.67, 126.95, 125.58 (dd, J=21.7, 8.9 Hz), 124.77, 116.14 (dd, J=23.2, 10.5 Hz), 111.03 (t, J=24.8 Hz), 59.55, 54.80, 53.50-52.40 (m), 52.44 (d, J=91.2 Hz), 42.23, 42.01, 40.75, 38.24, 35.57, 30.97. MS (ESI) m/z=530.3 [M+H]⁺

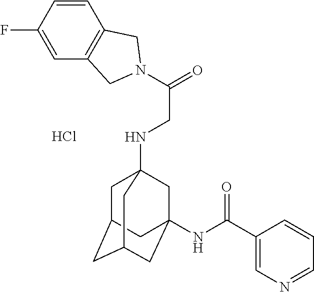 [SMILES: O=C(NC12CC3CC(CC(NCC(=O)N4Cc5ccc(F)cc5C4)(C3)C1)C2)c1cccnc1 ; Cl]

N-((1s,3r,5R,7S)-3-((2-(5-fluoroisoindolin-2-yl)-2-oxoethyl)amino)adamantan-1-yl)nicotinamide hydrochloride (98) Procedure R with 2-bromo-1-(5-fluoroisoindolin-2-yl)ethan-1-one (32.4 mg, 0,126 mmol) and N-((1s,3r,5R,7S)-3-aminoadamantan-1-yl)nicotinamide hydrochloride (58 mg, 0,188 mmol). Purified via normal-phase flash chromatography using a gradient of methanol in DCM to yield N-((1s,3r,5R,7S)-3-((2-(5-fluoroisoindolin-2-yl)-2-oxoethyl)amino)adamantan-1-yl)nicotinamide (30 mg, 0,067 mmol, 53,2% yield) MS (ESI) m/z=449.4 [M+H]⁺ followed by general procedure V. ¹H NMR (400 MHz, MeOD) δ 9.30 (s, OH), 9.04-8.96 (m, 2H), 8.20 (dd, J=8.1, 5.8 Hz, 1H), 7.43-7.35 (m, 1H), 7.19-7.05 (m, 2H), 4.99 (s, 1H), 4.83 (d, J=13.4 Hz, 2H), 4.16 (s, 2H), 2.54 (s, 2H), 2.51-2.44 (m, 2H), 2.38-2.30 (m, 2H), 2.07 (m, 6H), 1.86-1.73 (m, 2H). ¹³C NMR (101 MHz, MeOD) δ 165.40, 163.66, 163.02 (d, J=8.3 Hz), 146.11, 144.89, 142.96, 139.28 (dd, J=48.3, 8.9 Hz), 136.19, 132.70 (dd, J=50.2, 2.5 Hz), 128.39, 125.58 (dd, J=15.0, 9.0 Hz), 116.10 (dd,J=23.1, 9.5 Hz), 111.02 (dd,J=24.1, 18.6 Hz), 59.57, 55.37, 53.00 (dd, 3=84.4, 2.5 Hz), 52.49 (d, J=83.6 Hz), 42.15, 42.10, 40.32, 38.14, 35.42, 30.94. MS (ESI) m/z=449.4 [M+H]⁺

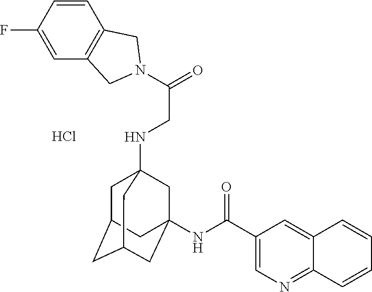 [SMILES: O=C(NC12CC3CC(CC(NCC(=O)N4Cc5ccc(F)cc5C4)(C3)C1)C2)c1cnc2ccccc2c1 ; Cl]

N-((1s,3r,5R,7S)-3-((2-(5-fluoroisoindolin-2-yl)-2-oxoethyl)amino)adamantan-1-yl)quinoline-3-carboxamide hydrochloride (99) Procedure R with 2-bromo-1-(5-fluoroisoindolin-2-yl)ethan-1-one (43.3 mg, 0,168 mmol) and N-((1s,3r,5R,7S)-3-aminoadamantan-1-yl)quinoline-3-carboxamide hydrochloride (90 mg, 0,251 mmol. Purified via normal-phase flash chromatography using a gradient of methanol in DCM to yield N-((1s,3r,5R,7S)-3-((2-(5-fluoroisoindolin-2-yl)-2-oxoethyl)amino)adamantan-1-yl)quinoline-3-carboxamide (24 mg, 0,048 mmol, 28,7% yield). MS (ESI) m/z=499.3 [M+H]⁺, followed by general procedure V. ¹H NMR (400 MHz, MeOD) δ 9.62-9.55 (m, 2H), 8.45 (d, J=8.3 Hz, 1H), 8.34-8.22 (m, 2H), 8.10-8.02 (m, 1H), 7.42-7.33 (m, 1H), 7.18-7.04 (m, 2H), 4.96 (s, 1H), 4.82 (d, J=13.4 Hz, 2H), 4.15 (s, 2H), 2.57 (s, 2H), 2.51-2.45 (m, 2H), 2.42-2.35 (m, 2H), 2.18-2.00 (m, 6H), 1.80 (q, J=13.2 Hz, 2H). ¹³C NMR (101 MHz, MeOD) δ 165.39, 164.28, 163.05 (d, J=8.9 Hz), 146.17, 146.09, 140.70, 139.27 (dd, J=43.5, 9.0 Hz), 137.39, 132.68 (d, J=43.6 Hz), 131.78, 131.52, 130.52, 129.52, 126.26-124.73 (m), 122.49, 116.14 (dd, J=23.0, 10.5 Hz), 111.54-110.29 (m), 59.60, 55.36, 52.99 (dd, J=88.2, 2.2 Hz), 52.48 (d, J=87.0 Hz), 42.24, 42.13, 40.47, 38.18, 35.48, 30.98. MS (ESI) m/z=499.3 [M+H]⁺

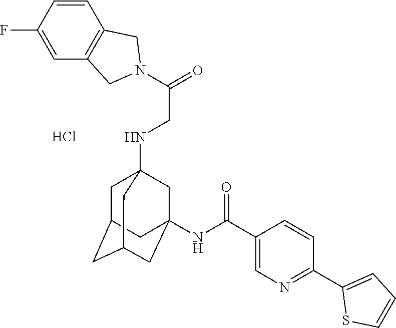 [SMILES: O=C(NC12CC3CC(CC(NCC(=O)N4Cc5ccc(F)cc5C4)(C3)C1)C2)c1ccc(-c2cccs2)nc1 ; Cl]

N-((1s,3r,5R,7S)-3-((2-(5-fluoroisoindolin-2-yl)-2-oxoethyl)amino)adamantan-1-yl)-6-(thiophen-2-yl)nicotinamide hydrochloride (100) Procedure R with 2-bromo-1-(5-fluoroisoindolin-2-yl)ethan-1-one (39.7 mg, 0,154 mmol) and N-((1s,3r,5R,7S)-3-aminoadamantan-1-yl)-6-(thiophen-2-yl)nicotinamide hydrochloride (90 mg, 0,231 mmol). Purified via normal-phase flash chromatography using a gradient of methanol in DCM to yield N-((1s,3r,5R,7S)-3-((2-(5-fluoroisoindolin-2-yl)-2-oxoethyl)amino)adamantan-1-yl)-6-(thiophen-2-yl)nicotinamide (39 mg, 0,073 mmol, 47,8% yield). ¹H NMR (400 MHz, MeOD) δ 8.82 (d, J=2.2 Hz, 1H), 8.11 (dd, J=8.4, 2.3 Hz, 1H), 7.86 (d, J=8.3 Hz, 1H), 7.77 (d, J=3.7 Hz, 1H), 7.57 (d, J=5.0 Hz, 1H), 7.33 (ddd, J=8.1, 4.9, 2.7 Hz, ¹H), 7.18-7.14 (m, 1H), 7.12-7.02 (m, 2H), 4.87 (d, J=14.1 Hz, 2H), 4.75 (d, J=13.4 Hz, 2H), 3.72 (s, 2H), 2.32 (q, J=3.2 Hz, 2H), 2.21 (d, J=21.8 Hz, 4H), 2.10-2.03 (m, 2H), 1.87-1.63 (m, 7H). ¹³C NMR (101 MHz, MeOD) δ 170.09, 167.70, 164.17 (dd, J=243.5, 8.4 Hz), 155.89, 149.50, 144.69, 139.53 (dd, J=48.3, 9.0 Hz), 137.38, 132.94 (dd, J=49.6, 2.6 Hz), 130.67, 130.14, 129.45, 127.52, 125.52 (dd, J=13.3, 8.9 Hz), 119.48, 115.97 (dd, J=23.1, 10.0 Hz), 110.98 (dd, J=24.1, 17.0 Hz), 55.19, 55.14, 52.92 (dd, J=78.5, 2.5 Hz), 52.39 (d, J=77.3 Hz), 44.96, 42.90 (d, J=2.4 Hz), 41.09, 40.93, 36.22, 31.27. MS (ESI) m/z=531.3 [M+H]⁺, followed by general procedure V. MS (ESI) m/z=531.3 [M+H]⁺

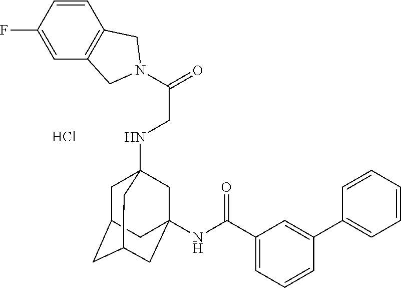 [SMILES: O=C(NC12CC3CC(CC(NCC(=O)N4Cc5ccc(F)cc5C4)(C3)C1)C2)c1cccc(-c2ccccc2)c1 ; Cl]

N-((1s,3r,5R,7S)-3-((2-(5-fluoroisoindolin-2-yl)-2-oxoethyl)amino)adamantan-1-yl)-[1,1′-biphenyl]-3-carboxamide hydrochloride (101) Procedure R with 2-bromo-1-(5-fluoroisoindolin-2-yl)ethan-1-one (40.4 mg, 0,157 mmol) and N-((1s,3r,5R,7S)-3-aminoadamantan-1-yl)-[1,1′-biphenyl]-3-carboxamide hydrochloride (90 mg, 0,235 mmol). Purified via normal-phase flash chromatography using a gradient of methanol in DCM to yield N-((1s,3r,5R,7S)-3-((2-(5-fluoroisoindolin-2-yl)-2-oxoethyl)amino)adamantan-1-yl)-[1,1′-biphenyl]-3-carboxamide (32 mg, 0,061 mmol, 39,0% yield). MS (ESI) m/z=524.3 [M+H]⁺, followed by general procedure V. ¹H NMR (400 MHz, MeOD) δ 8.02-7.97 (m, 1H), 7.78 (d, J=7.7 Hz, OH), 7.74 (d, J=8.0 Hz, 1H), 7.66 (d, J=7.5 Hz, 2H), 7.53 (t, J=7.7 Hz, 1H), 7.47 (t, J=7.6 Hz, 2H), 7.42-7.32 (m, 2H), 7.17-7.03 (m, 2H), 4.94 (s, 1H), 4.80 (d, J=13.3 Hz, 2H), 4.13 (s, 2H), 2.54 (s, 2H), 2.47-2.40 (m, 2H), 2.40-2.32 (m, 2H), 2.09-1.96 (m, 6H), 1.85-1.71 (m, 2H). ¹³C NMR (101 MHz, MeOD) δ 170.48, 165.41, 163.03 (d, J=8.4 Hz), 142.78, 141.59, 139.26 (dd, J=45.0, 9.0 Hz), 137.40, 132.68 (dd, J=47.7, 2.6 Hz), 131.01, 130.04, 130.01, 128.84, 128.09, 127.19, 126.98, 125.58 (dd,J=18.1, 8.7 Hz), 116.12 (dd,J=23.1, 9.9 Hz), 111.02 (dd, J=24.1, 21.5 Hz), 59.61, 54.68, 52.96 (dd, J=89.8, 3.0 Hz), 52.45 (d, J=88.9 Hz), 42.13, 42.04, 40.61, 38.27, 35.58, 30.98. MS (ESI) m/z=524.3 [M+H]⁺

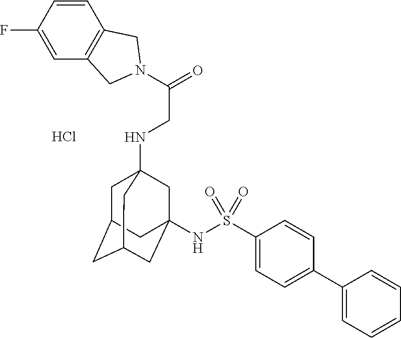 [SMILES: O=C(CNC12CC3CC(C1)CC(NS(=O)(=O)c1ccc(-c4ccccc4)cc1)(C3)C2)N1Cc2ccc(F)cc2C1 ; Cl]

N-((1s,3r,5R,7S)-3-((2-(5-fluoroisoindolin-2-yl)-2-oxoethyl)amino)adamantan-1-yl)-[1,1′-biphenyl]-4-sulfonamide hydrochloride (102) Procedure R with 2-bromo-1-(5-fluoroisoindolin-2-yl)ethan-1-one (82 mg, 0,319 mmol) and N-((1s,3r,5R,7S)-3-aminoadamantan-1-yl)-[1,1′-biphenyl]-4-sulfonamide hydrochloride (174 mg, 0,415 mmol). Purified via normal-phase flash chromatography using a gradient of methanol in DCM to yield N-((1s,3r,5R,7S)-3-((2-(5-fluoroisoindolin-2-yl)-2-oxoethyl)amino)adamantan-1-yl)-[1,1′-biphenyl]-4-sulfonamide (42 mg, 0,075 mmol, 23,49% yield). MS (ESI) m/z=560.3 [M+H]⁺, followed by general procedure V. ¹H NMR (400 MHz, MeOD) δ 8.03-7.94 (m, 2H), 7.86-7.78 (m, 2H), 7.72-7.63 (m, 2H), 7.49-7.43 (m, 2H), 7.41-7.25 (m, 2H), 7.12-7.01 (m, 2H), 4.83 (d, J=11.8 Hz, 2H), 4.76 (d, J=12.9 Hz, 2H), 3.98 (d, J=2.0 Hz, 2H), 2.33-2.27 (m, 2H), 2.13 (s, 2H), 1.96-1.76 (m, 8H), 1.61 (s, 2H). ¹³C NMR (101 MHz, MeOD) δ 165.21, 164.19 (dd, J=243.7, 10.3 Hz), 146.32, 144.32, 140.45, 139.58 138.12 (m), 132.56 (d, J=43.1 Hz), 130.17, 129.54, 128.57, 128.42, 128.19, 126.22-124.90 (m), 116.09 (dd, J=23.1, 12.3 Hz), 111.01 (dd, J=24.2, 14.2 Hz), 59.59, 56.29, 52.95 (dd, J=91.5, 2.8 Hz), 52.44 (d, J=89.9 Hz), 44.75, 42.13, 41.95, 37.78, 34.98, 30.86. MS (ESI) m/z=560.3 [M+H]⁺

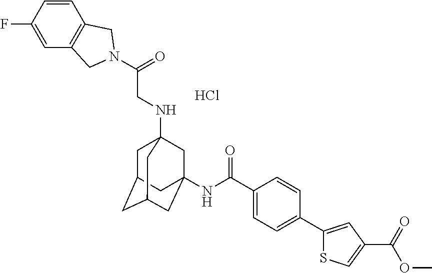 [SMILES: COC(=O)c1csc(-c2ccc(C(=O)NC34CC5CC(CC(NCC(=O)N6Cc7ccc(F)cc7C6)(C5)C3)C4)cc2)c1 ; Cl]

Methyl 5-(4-((-3-((2-(5-fluoroisoindolin-2-yl)-2-oxoethyl)amino)adamantan-1-yl)carbamoyl)phenyl)thiophene-3-carboxylate hydrochloride (103) (0.01 g, 0.017 mmol, 21% yield) was prepared according to general procedure X and Y from tert-butyl-3-aminoadamantan-1-yl)carbamate (4) (0.065 g, 0.242 mmol) and purified by silica gel flash chromatography (elution with 0-10% MeOH in DCM). ¹H NMR (400 MHz, DMSO-d6) δ 9.01 (s, 2H), 8.40 (d, J=1.3 Hz, 1H), 8.00 (s, 1H), 7.94 (d, J=1.2 Hz, 1H), 7.87 (d, J=8.4 Hz, 2H), 7.81 (d, J=8.4 Hz, 2H), 7.50-7.33 (m, 1H), 7.31-7.07 (m, 2H), 4.95 (d, J=16.7 Hz, 2H), 4.72 (d, J=13.6 Hz, 2H), 4.06 (s, 2H), 3.83 (s, 3H), 2.38 (s, 2H), 2.30 (s, 2H), 2.17 (dd, J=14.8, 9.4 Hz, 2H), 1.90 (d, J=12.1 Hz, 5H), 1.69-1.42 (m, 3H), 1.23 (s, 5H). ¹H NMR (400 MHz, DMSO-de) δ 9.01 (s, 2H), 8.40 (d, J=1.3 Hz, 1H), 8.00 (s, 1H), 7.94 (d, J=1.2 Hz, 1H), 7.87 (d, J=8.4 Hz, 2H), 7.81 (d, J=8.4 Hz, 2H), 7.50-7.33 (m, 1H), 7.29-7.23 (m, 1H), 7.21-7.14 (m, 1H), 4.95 (d, J=16.7 Hz, 2H), 4.72 (d, J=13.6 Hz, 2H), 4.06 (br s, 2H), 3.83 (s, 3H), 2.38 (s, 2H), 2.34-2.21 (m, 2H), 2.20-2.10 (m, 2H), 1.90 (br s, 5H), 1.69-1.42 (m, 2H); LRMS m/z (ESI⁺) 588 [M+H]⁺.

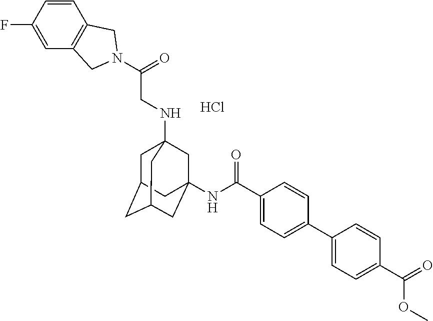 [SMILES: COC(=O)c1ccc(-c2ccc(C(=O)NC34CC5CC(CC(NCC(=O)N6Cc7ccc(F)cc7C6)(C5)C3)C4)cc2)cc1 ; Cl]

Methyl 4′-((-3-((2-(5-fluoroisoindolin-2-yl)-2-oxoethyl)amino)adamantan-1-yl)carbamoyl)-[1,1′-biphenyl]-4-carboxylate hydrochloride (104) (0.130 g, 0.223 mmol, 77% yield) was prepared according to general procedure X and Y from tert-butyl-3-aminoadamantan-1-yl)carbamate (4) (0.206 g, 0.773 mmol) and purified by silica gel flash chromatography (elution with 0-10% MeOH in DCM). ¹H NMR (400 MHz, DMSO-de) δ_{H} 9.02 (s, 2H), 8.06 (d, J=8.4 Hz, 2H), 8.03 (s, 1H), 7.94 (d, J=8.4 Hz, 2H), 7.89 (d, J=8.4 Hz, 2H), 7.83 (d, J=8.4 Hz, 2H), 7.45-7.38 (m, 1H), 7.28-7.24 (m, 1H), 7.20-7.15 (m, 1H), 4.96 (d, J=16.7 Hz, 2H), 4.72 (d, J=13.6 Hz, 2H), 4.06 (br s, 2H), 3.88 (s, 3H), 2.40 (s, 2H), 2.30 (s, 2H), 2.16 (d, J=11.9 Hz, 2H), 1.92 (br s, 5H), 1.73-1.41 (m, 2H); ¹³C NMR (101 MHz, DMSO-de) δ_{H} 166.0, 165.9, 164.3, 162.0 (dd, J=243.4, 1.0 Hz), 143.7, 141.1, 138.3 (dd, J=84.8, 9.1 Hz), 135.2, 131.6 (dd, J=86.9, 3.0 Hz), 129.9, 128.9, 128.26, 128.0, 127.2, 126.7, 126.4, 124.7 (dd, J=46.5, 8.1 Hz), 114.8 (dd, J=22.2, 6.1 Hz), 110.1 (dd, J=48.5, 23.2 Hz), 62.9, 57.7, 52.9, 52.3, 52.1, 51.5, 51.1, 50.5, 40.9, 40.5, 36.4, 34.4, 28.9; LRMS m/z (ESI⁺) 582 [M+H]⁺.

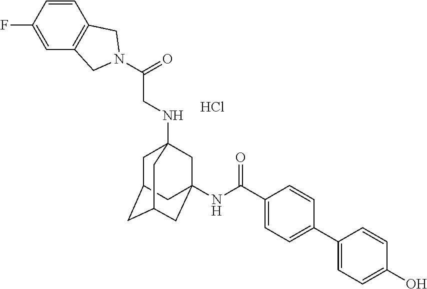 [SMILES: O=C(NC12CC3CC(CC(NCC(=O)N4Cc5ccc(F)cc5C4)(C3)C1)C2)c1ccc(-c2ccc(O)cc2)cc1 ; Cl]

N-(-3-((2-(5-fluoroisoindolin-2-yl)-2-oxoethyl)amino)adamantan-1-yl)-4′-hydroxy-[1,1′-biphenyl]-4-carboxamide hydrochloride NH HCl (105) (0.050 g, 0.093 mmol, 66% yield) was prepared according to general procedure X and Y from tert-butyl-3-aminoadamantan-1-yl)carbamate (4) (0.597 g, 2.241 mmol) and purified by silica gel flash chromatography (elution with 0-10% MeOH in DCM). ¹H NMR (400 MHz, DMSO-de) δ_{H} 9.71 (s, 1H), 8.96 (br s, 1H), 7.91 (s, 1H), 7.84 (d, J=8.4 Hz, 2H), 7.65 (d, J=8.4 Hz, 2H), 7.55 (d, J=8.6 Hz, 2H), 7.49-7.36 (m, 1H), 7.30-7.24 (m, 1H), 7.22-7.12 (m, 1H), 6.87 (d, J=8.6 Hz, 2H), 4.95 (d, J=16.7 Hz, 2H), 4.72 (d, J=13.6 Hz, 2H), 4.06 (br s, 2H), 2.39 (s, 2H), 2.30 (br s, 2H), 2.16 (d, J=11.7 Hz, 2H), 1.97-1.84 (m, 5H), 1.70-1.49 (m, 2H); ¹³C NMR (101 MHz, DMSO-de) δ_{H} 166.1, 164.3, 157.7, 143.0, 137.8 (d, J=13.1 Hz), 133.2, 131.8 (d, J=87.9 Hz), 129.8, 128.0, 127.9, 125.4, 124.7 (dd, 3=46.5, 8.1 Hz), 115.8, 114.8 (dd, J=22.2, 6.1 Hz), 110.1 (dd, J=48.5, 23.2 Hz), 57.8, 52.8, 51.9, 50.5, 40.9, 40.4, 36.5, 34.3, 30.7, 28.9; LRMS m/z (ESI⁺) 540 [M+H]⁺.

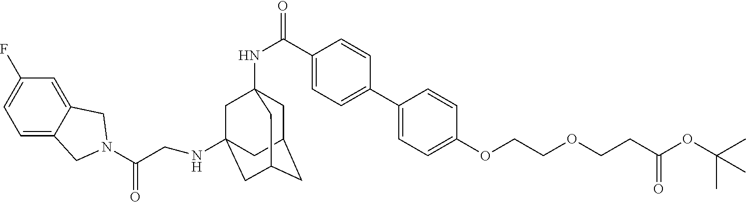 [SMILES: CC(C)(C)OC(=O)CCOCCOc1ccc(-c2ccc(C(=O)NC34CC5CC(CC(NCC(=O)N6Cc7ccc(F)cc7C6)(C5)C3)C4)cc2)cc1]

tert-Butyl 3-(2-((4′-((-3-((2-(5-fluoroisoindolin-2-yl)-2-oxoethyl)amino)adamantan-1-yl)carbamoyl)-[1,1′-biphenyl]-4-yl)oxy)ethoxy)propanoate (106) (0.020 g, 0.028 mmol, 73% yield) was prepared according to general procedure X and Y from tert-butyl-3-aminoadamantan-1-yl)carbamate (4) (0.597 g, 2.241 mmol) and purified by silica gel flash chromatography (elution with 0-10% MeOH in DCM). LRMS m/z (ESI⁺) 712 [M+H]⁺.

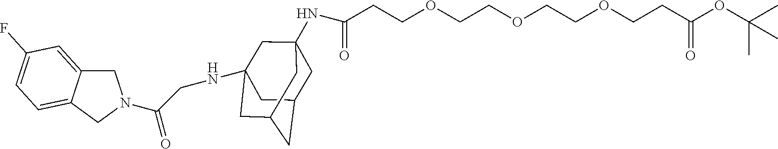 [SMILES: CC(C)(C)OC(=O)CCOCCOCCOCCC(=O)NC12CC3CC(CC(NCC(=O)N4Cc5ccc(F)cc5C4)(C3)C1)C2]

tert-Butyl 3-(2-(2-(3-((-3-((2-(5-fluoroisoindolin-2-yl)-2-oxoethyl)amino)adamantan-1-yl)amino)-3-oxopropoxy)ethoxy)ethoxy)propanoate (107) (0.100 g, 0.158 mmol, 97% yield) was prepared according to general procedure X and Y from tert-butyl-3-aminoadamantan-1-yl)carbamate (4) (0.206 g, 0.773 mmol) and purified by silica gel flash chromatography (elution with 0-10% MeOH in DCM). LRMS m/z (ESI⁺) 632 [M+H]⁺.

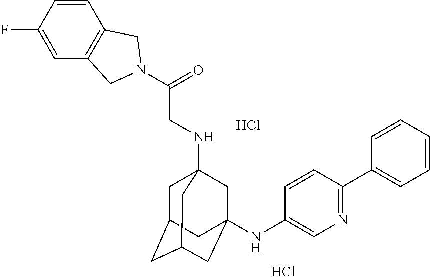 [SMILES: O=C(CNC12CC3CC(C1)CC(Nc1ccc(-c4ccccc4)nc1)(C3)C2)N1Cc2ccc(F)cc2C1 ; Cl ; Cl]

1-(5-fluoroisoindolin-2-yl)-2-((-3-((6-phenylpyridin-3-yl)amino)adamantan-1-yl)amino)ethan-1-one dihydrochloride (109) (0.060 g, 0.121 mmol, 52% yield) was prepared according to general procedure Z and Y from tert-butyl-3-aminoadamantan-1-yl)carbamate (4) (0.427 g, 1.602 mmol) and purified by silica gel flash chromatography (elution with 0-10% MeOH in DCM). ¹H NMR (400 MHz, DMSO-d6) δ_{H} 9.18 (br s, 2H), 8.36 (br s, 1H), 8.13-7.82 (m, 4H), 7.64-7.48 (m, 3H), 7.45-7.35 (m, 1H), 7.31-7.13 (m, 2H), 4.95 (d, J=16.4 Hz, 1H), 4.71 (d, J=13.6 Hz, 1H), 4.08 (br s, 2H), 2.35 (br s, 2H), 2.25 (s, 2H), 2.05-1.83 (m, 7H), 1.73-1.50 (m, 2H); ¹³C NMR (101 MHz, DMSO-d6) δ_{H} 164.2 (d, J=1.0 Hz), 162.0 (dd, J=242.4, 6.1 Hz), 138.70 (d, J=9.1 Hz), 137.9 (d, J=9.1 Hz), 132.2 (d, J=2.0 Hz), 131.4 (d, J=2.0 Hz), 130.0, 129.2, 126.7, 124.7 (dd, J=48.5, 3.1 Hz), 114.8 (dd, J=22.2, 5.0 Hz), 110.1 (dd, J=50.5, 24.2 Hz), 64.9, 57.7, 52.1, 51.5, 51.1, 50.6, 40.6, 36.0, 33.7, 28.8, 15.2; LRMS m/z (ESI⁺) 497 [M+H]⁺.

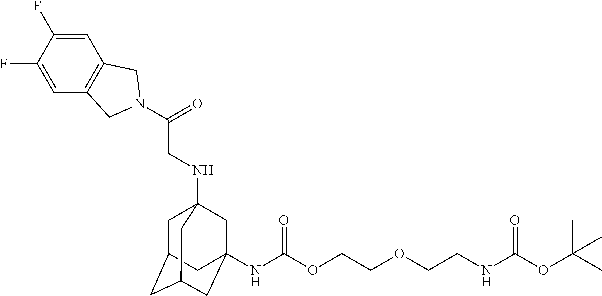 [SMILES: CC(C)(C)OC(=O)NCCOCCOC(=O)NC12CC3CC(CC(NCC(=O)N4Cc5cc(F)c(F)cc5C4)(C3)C1)C2]

2-(2-((tert-Butoxycarbonyl)amino)ethoxy)ethyl-3-((2-(5,6-difluoroisoindolin-2-yl)-2oxoethyl)amino)adamantan-1-ylcarbamate (110)-(0.035 g, 0.059 mmol, 55% yield) was prepared according to general procedure 1 from tert-butyl (2-(2-(((4-nitrophenoxy)carbonyl)oxy)ethoxy)ethyl) carbamate (65) (0.040 g, 0.108 mmol) and purified by silica gel flash chromatography (elution with 0-10% MeOH in DCM).

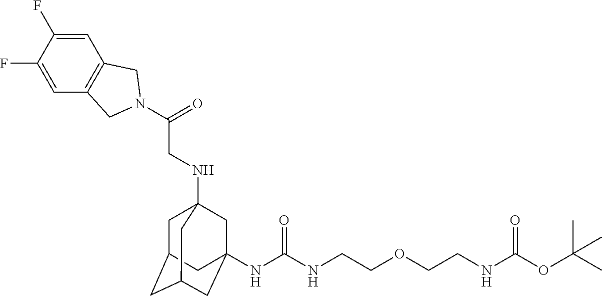 [SMILES: CC(C)(C)OC(=O)NCCOCCNC(=O)NC12CC3CC(CC(NCC(=O)N4Cc5cc(F)c(F)cc5C4)(C3)C1)C2]

tert-Butyl (2-(2-(3-(-3-((2-(5,6-difluoroisoindolin-2-yl)-2-oxoethyl)amino)adamantan-1-yl)ureido)ethoxy)ethyl) carbamate (111) (0.040 g, 0.068 mmol, 50% yield) was prepared according to general procedure 1 from 4-nitrophenyl (2-(2-((tert-butoxycarbonyl)amino)ethoxy)ethyl) carbamate (65) (0.050 g, 0.135 mmol) and purified by silica gel flash chromatography (elution with 0-10% MeOH in DCM); LRMS m/z (ESI⁺) 592 [M+H]⁺.

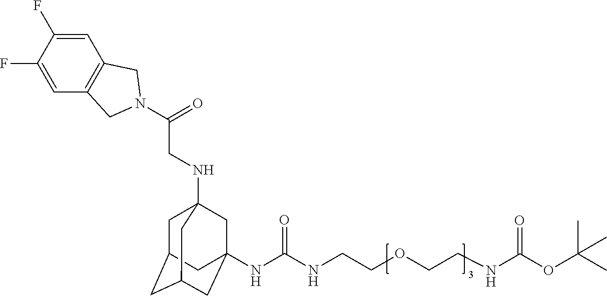

tert-Butyl (1-((3-((2-(5,6-difluoroisoindolin-2-yl)-2-oxoethyl) amino)adamantan-1-yl)amino)-1-oxo-5,8,11-trioxa-2-azatridecan-13-yl)carbamate (112) (0.025 g, 0.037 mmol, 67% yield) was prepared according to general procedure 1 from 4-nitrophenyl (2,2-dimethyl-4-oxo-3,8,11,14-tetraoxa-5-azahexadecan-16-yl)carbamate (65) (0.025 g, 0.055 mmol) and purified by silica gel flash chromatography (elution with 0-15% MeOH in DCM).

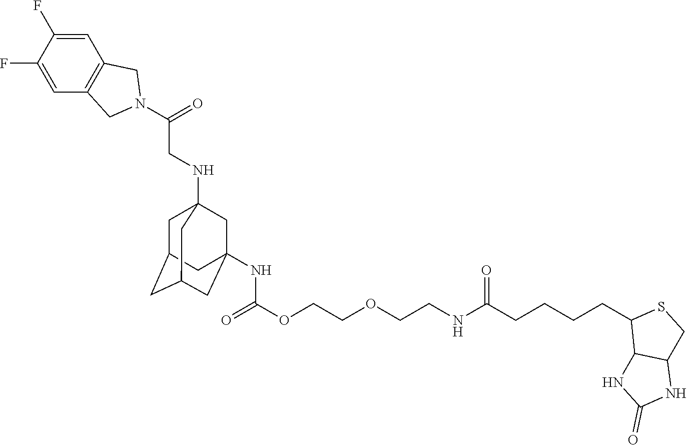 [SMILES: O=C(CCCCC1SCC2NC(=O)NC21)NCCOCCOC(=O)NC12CC3CC(CC(NCC(=O)N4Cc5cc(F)c(F)cc5C4)(C3)C1)C2]

2-(2-(5-(2-oxohexahydro-1H-thieno[3,4-d]imidazol-4-yl)pentanamido)ethoxy)ethyl (3-((2-(5,6-difluoroisoindolin-2-yl)-2-oxoethyl)amino)adamantan-1-yl)carbamate (113) (0.030 g, 0.042 mmol, 71% yield) was prepared according to general procedure 2 from 2-(2-((tert-butoxycarbonyl)amino)ethoxy)ethyl-3-((2-(5,6-difluoroisoindolin-2-yl)-2oxoethyl)amino)adamantan-1-ylcarbamate (110) (0.035 g, 0.059 mmol) and purified by silica gel flash chromatography (elution with a mixture of 30-80% DCM:MeOH:NH3 (8:2:0.2) in DCM). ¹H NMR (400 MHz, Methanol-d4) δ_{H} 7.28 (ddd, J=10.0, 7.4, 2.5 Hz, 2H), 4.85 (s, 2H), 4.74 (s, 2H), 4.50 (dd, J=7.9, 4.8 Hz, 1H), 4.31 (dd, J=7.9, 4.4 Hz, 1H), 4.09 (br s, 2H), 3.64-3.62 (m, 2H), 3.54 (t, J=5.5 Hz, 2H), 3.35 (t, J=5.5 Hz, 2H), 3.20 (ddd, J=8.8, 6.0, 4.5 Hz, 1H), 2.93 (dd, J=12.8, 4.9 Hz, 1H), 2.71 (d, J=12.7 Hz, 1H), 2.31-2.18 (m, 4H), 2.04-1.92 (m, 4H), 1.86 (dd, J=12.6, 2.9 Hz, 2H), 1.79-1.54 (m, 10H), 1.49-1.23 (m, 4H); ¹³C NMR (101 MHz, Methanol-d4) δ_{H} 176.0, 170.3, 166.0, 156.7, 151.6 (ddd, J=249.5, 16.2, 6.1 Hz), 133.7 (ddd, J=43.4, 7.1, 8.1 Hz), 113.0 (dd, J=19.2, 12.1 Hz), 70.5, 70.3, 64.3, 63.3, 61.5, 57.0, 54.8, 53.0, 52.2, 45.6, 42.9, 41.5, 41.0 (d, J=5.1 Hz), 40.3, 36.7, 36.2, 31.1, 29.7, 29.4, 26.8; LRMS m/z (ESI⁺) 719 [M+H]⁺.

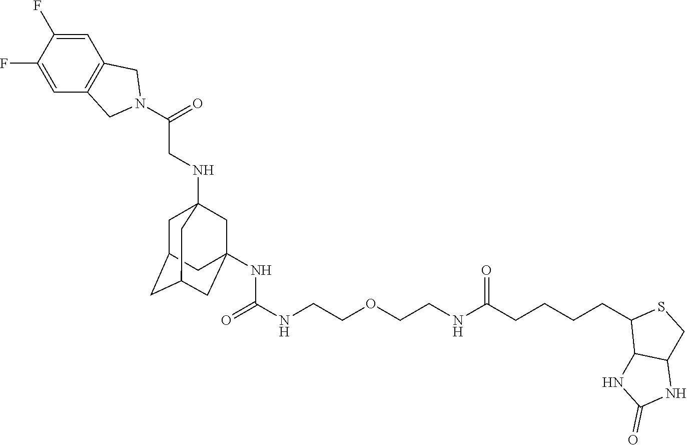 [SMILES: O=C(CCCCC1SCC2NC(=O)NC21)NCCOCCNC(=O)NC12CC3CC(CC(NCC(=O)N4Cc5cc(F)c(F)cc5C4)(C3)C1)C2]

N-(2-(2-(3-(3-((2-(5,6-difluoroisoindolin-2-yl)-2-oxoethyl) amino)adamantan-1-yl)ureido)ethoxy)ethyl)-5-(2-oxohexahydro-1H-thieno[3,4-d]imidazol-4-yl) pentanamide (114) (0.058 g, 0.081 mmol, 94% yield) was prepared according to general procedure 2 from tert-Butyl (2-(2-(3-(-3-((2-(5,6-difluoroisoindolin-2-yl)-2-oxoethyl)amino)adamantan-1-yl)ureido)ethoxy)ethyl) carbamate (111) (0.051 g, 0.086 mmol) and purified by silica gel flash chromatography (elution with a mixture of 30-80% DCM:MeOH:NH3 (8:2:0.2) in DCM). ¹H NMR (400 MHz, Methanol-d₄) δ_{H} 7.27 (dd, J=10.0, 7.4 Hz, 2H), 4.74 (s, 2H), 4.47 (dd, J=7.8, 4.8 Hz, 1H), 4.28 (dd, J=7.9, 4.4 Hz, 1H), 3.97 (s, 2H), 3.46 (dt, J=13.5, 5.4 Hz, 4H), 3.33 (t, J=5.5 Hz, 2H), 3.25-3.13 (m, 3H), 2.90 (dd, J=12.7, 4.9 Hz, 1H), 2.67 (d, J=12.6 Hz, 1H), 2.00 (br s, 2H), 2.55-2.16 (m, 4H), 2.04 (d, J=12.0 Hz, 2H) 1.89 (br s, 4), 1.83-1.48 (m, 8H), 1.46-1.35 (m, 2H); ¹³C NMR (101 MHz, Methanol-d₄) δ_{H} 176.1, 166.5, 166.0, 159.9, 151.6 (ddd,J=249.5, 16.2, 5.1 Hz), 133.5 (ddd, J=46.5, 7.1, 3.0 Hz), 113.0 (dd, J=12.2, 11.1 Hz), 79.5, 71.2, 70.4, 63.3, 61.56, 58.65, 57.0, 53.1, 52.8, 52.2, 44.1, 42.2, 41.8, 41.1, 40.5, 40.3, 38.8 (d, J=3.0 Hz), 36.7, 35.7, 31.0, 29.7, 29.5, 26.9; LRMS m/z (ESI⁺) 718 [M+H]⁺.

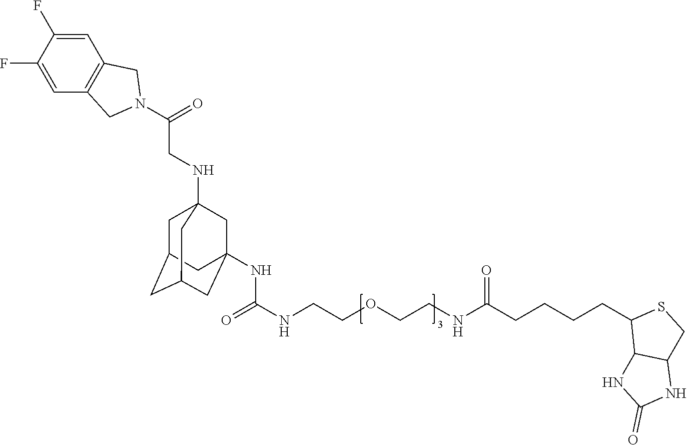

N-(1-((3-((2-(5,6-difluoroisoindolin-2-yl)-2-oxoethyl) amino)adamantan-1-yl)amino)-1-oxo-5,8,11-trioxa-2-azatridecan-13-yl)-5-(2-oxohexahydro-1H thieno[3,4-d]imidazol-4-yl)pentanamide (115) (0.015 g, 0.019 mmol, 86% yield) was prepared according to general procedure 2 from tert-butyl (1-((3-((2-(5,6-difluoroisoindolin-2-yl)-2-oxoethyl) amino)adamantan-1-yl)amino)-1-oxo-5,8,11-trioxa-2-azatridecan-13-yl)carbamate (112) (0.017 g, 0.025 mmol) and purified by silica gel flash chromatography (elution with a mixture of 40-80% DCM:MeOH:NH3 (8:2:0.2) in DCM). ¹H NMR (400 MHz, Methanol-d4) δ_{H} 7.28 (ddd, 3=10.6, 7.4, 3.8 Hz, 2H), 4.75 (s, 2H), 4.47 (dd, J=7.9, 4.8 Hz, 1H), 4.28 (dd, J=7.8, 4.4 Hz, 1H), 3.95 (s, 2H), 3.66-3.56 (m, 8H), 3.53 (t, J=5.5 Hz, 2H), 3.48 (t, J=5.4 Hz, 2H), 3.34 (t, J=5.5 Hz, 2H), 3.30-3.27 (m, 1H), 3.22 (t, J=5.4 Hz, 2H), 3.20-3.14 (m, 1H), 2.90 (dd, J=12.8, 4.9 Hz, 1H), 2.68 (d, J=12.7 Hz, 1H), 2.30 (br s, 2H), 2.23-2.14 (m, 4H), 2.05 (d, J=12.1 Hz, 2H), 1.94-1.82 (m, 4H), 1.81-1.32 (m, 11H); ¹³C NMR (101 MHz, Methanol-d4) δ_{H} 174.73, 165.34, 164.67, 158.51, 150.3 (ddd, J=249.5, 16.2, 5.1 Hz), 132.2 (ddd, J=43.4, 7.1, 4.0 Hz), 111.7 (dd, J=18.2, 16.2 Hz), 70.18, 70.13, 69.9 (d, J=3.0 Hz), 69.2, 62.0, 60.3, 57.1, 55.6, 51.7, 51.5, 50.9, 42.8, 40.9, 40.5, 39.7, 39.2, 39.0, 37.6, 35.4, 34.4, 29.7, 28.4, 28.2, 25.5; LRMS m/z (ESI⁺) 806 [M+H]⁺.

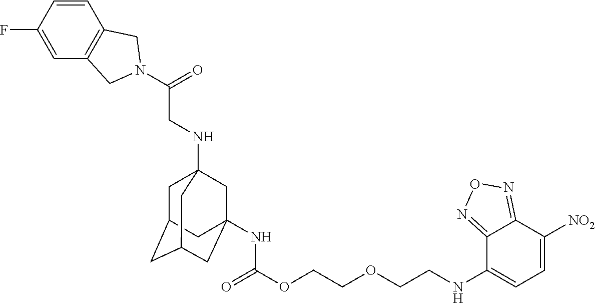 [SMILES: O=C(NC12CC3CC(CC(NCC(=O)N4Cc5ccc(F)cc5C4)(C3)C1)C2)OCCOCCNc1ccc([N+](=O)[O-])c2nonc12]

2-(2-((7-nitrobenzo[c][1,2,5]oxadiazol-4-yl)amino)ethoxy)ethyl-3-((2 (5-fluoroisoindolin-2-yl)-2-oxoethyl)amino)adamantan-1-yl)carbamate (116) (0.023 g, 0.035 mmol, 37% yield) was prepared according to general procedure 3 from 2-(2-((tert-butoxycarbonyl)amino)ethoxy)ethyl-3-((2-(5-fluoroisoindolin-2-yl)-2oxoethyl)amino)adamantan-1-ylcarbamate (66) (0.055 g, 0.095 mmol) and purified by silica gel flash chromatography (elution with a mixture of 1-10% MeOH in DCM). ¹H NMR (400 MHz, CD₃OD) δ_{H} 7.28 (ddd, 3=10.6, 7.4, 3.8 Hz, 2H), 4.79 (br s, 1H), 4.85 (d, J=15.0 Hz, 4H), 4.20 (br s, 2H), 3.76 (t, J=5.0 Hz, 2H), 3.80-3.63 (m, 4H), 3.51 (s, 2H), 2.25 (br s, 2H), 2.00 (s, 3H), 1.97-1.77 (m, 6H), 1.72-1.49 (m, 6H); LRMS m/z (ESI⁺) 638 [M+H]⁺.

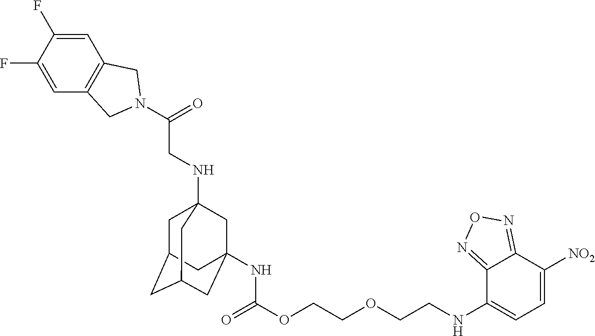 [SMILES: O=C(NC12CC3CC(CC(NCC(=O)N4Cc5cc(F)c(F)cc5C4)(C3)C1)C2)OCCOCCNc1ccc([N+](=O)[O-])c2nonc12]

2-(2-((7-nitrobenzo[c][1,2,5]oxadiazol-4-yl)amino)ethoxy)ethyl-3-((2-(5,6-difluoroisoindolin-2-yl)-2-oxoethyl)amino)adamantan-1-yl)carbamate (116) (0.030 g, 0.046 mmol, 42% yield) was prepared according to general procedure 3 from 2-(2-((tert-butoxycarbonyl)amino)ethoxy)ethyl-3-((2-(5,6-difluoroisoindolin-2-yl)-2oxoethyl)amino)adamantan-1-ylcarbamate (110) (0.109 g, 0.196 mmol) and purified by silica gel flash chromatography (elution with a mixture of 1-10% MeOH in DCM ¹H NMR (400 MHz, CD₃OD) δ_{H} 8.48 (d, J=8.6 Hz, 1H), 7.15-7.05 (m, 2H), 6.21 (d, J=8.7 Hz, 1H), 5.00 (br s, 1H), 4.77 (d, J=15.0 Hz, 4H), 4.20 (br s, 2H), 3.86 (t, J=5.0 Hz, 2H), 3.77-3.60 (m, 4H), 3.51 (s, 2H), 2.23 (br s, 2H), 2.04 (s, 3H), 1.97-1.77 (m, 6H), 1.72-1.49 (m, 6H); ¹³C NMR (101 MHz, CD₃OD)⁶c 176.1, 151.6 (ddd, J=249.5, 16.2, 5.1 Hz), 144.3, 144.1, 136.6, 132.0 (ddd, J=46.5, 7.1, 3.0 Hz), 111.9 (dd,J=12.2, 11.1 Hz), 69.7, 62.3, 52.5, 52.2, 52.0, 51.2, 42.9, 41.3, 40.8, 35.3, 29.8; LRMS m/z (ESI⁺) 656 [M+H]⁺.

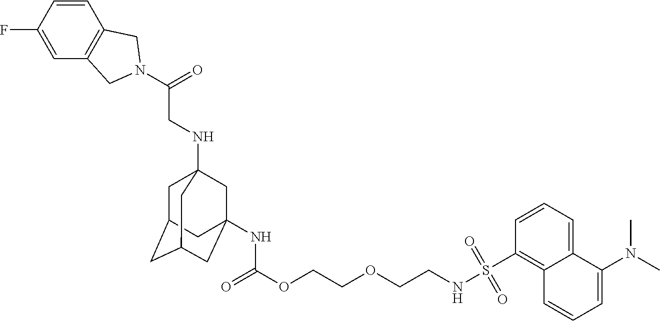 [SMILES: CN(C)c1cccc2c(S(=O)(=O)NCCOCCOC(=O)NC34CC5CC(CC(NCC(=O)N6Cc7ccc(F)cc7C6)(C5)C3)C4)cccc12]

2-(2-((5-(dimethylamino)naphthalene)-1-sulfonamido)ethoxy)ethyl-3-((2-(5-fluoroisoindolin-2-yl)-2-oxoethyl)amino)adamantan-1-yl)carbamate (118) (0.058 g, 0.098 mmol, 54% yield) was prepared according to general procedure 3 from 2-(2-((tert-butoxycarbonyl)amino)ethoxy)ethyl-3-((2-(5-fluoroisoindolin-2-yl)-2oxoethyl)amino)adamantan-1-ylcarbamate (66) (0.105 g, 0.181 mmol) and purified by silica gel flash chromatography (elution with a mixture of 1-10% MeOH in DCM). 1H NMR (400 MHz, CD₃OD) δ_{H} 8.49 (d, J=8.5 Hz, 1H), 8.29 (dd, J=8.6, 2.2 Hz, 1H), 8.19 (d, J=7.3 Hz, 1H), 7.51 (dt, J=21.5, 8.0 Hz, 2H), 7.23-6.72 (m, 4H), 5.87 (br s, 1H), 5.14 (br s, 1H), 4.68 (dd, J=18.6, 8.7 Hz, 4H), 4.00 (br s, 2H), 3.50 (s, 2H), 3.42 (br s, 2H), 3.37 (t, J=5.0 Hz, 2H), 3.05 (br s, 2H), 2.84 (s, 6H), 2.65 (br s, 2H), 2.21 (s, 2H), 2.00-1.76 (m, 5H), 1.72-1.63 (m, 1H), 1.58-1.49 (m, 1H); ¹³C NMR (101 MHz, CD₃OD)⁶c 170.0, 162.7 (dd, J=246.4, 19.2 Hz), 154.3, 152.0, 138.1 (dd, J=12.1, 9.1 Hz), 134.9, 131.6 (dd, J=5.1, 2.0 Hz), 130.5, 129.9, 129.7, 129.5, 128.4, 124.2 (dd, J=25.3, 8.1 Hz), 123.2, 119.0, 115.3, 115.0, 114.8, 110.1 (dd, J=31.3, 24.2 Hz), 77.4, 69.5 (d, J=36.4 Hz), 63.2, 52.5, 52.3, 51.8, 51.5, 51.0, 45.5, 43.0, 42.8, 41.1 (d, J=40.4 Hz), 35.4, 29.8; LRMS m/z (ESI⁺) 708 [M+H]⁺.

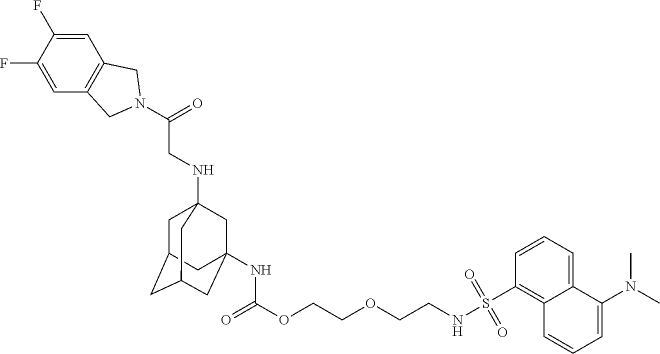 [SMILES: CN(C)c1cccc2c(S(=O)(=O)NCCOCCOC(=O)NC34CC5CC(CC(NCC(=O)N6Cc7cc(F)c(F)cc7C6)(C5)C3)C4)cccc12]

2-(2-((5-(dimethylamino)naphthalene)-1-sulfonamido)ethoxy)ethyl-3-((2-(5,6-difluoroisoindolin-2-yl)-2-oxoethyl)amino)adamantan-1-yl)carbamate (119) (0.035 g, 0.048 mmol, 71% yield) was prepared according to general procedure 3 from 2-(2-((tert-butoxycarbonyl)amino)ethoxy)ethyl-3-((2-(5,6-difluoroisoindolin-2-yl)-2oxoethyl)amino)adamantan-1-ylcarbamate (110) (0.038 g, 0.067 mmol) and purified by silica gel flash chromatography (elution with a mixture of 1-10% MeOH in DCM). ¹H NMR (400 MHz, Chloroform-d) δ_{H} 8.49 (d, J=8.5 Hz, 1H), 8.28 (d, J=8.6 Hz, 1H), 8.19 (d, J=7.3 Hz, 1H), 7.51 (dt, J=18.5, 7.9 Hz, 2H), 7.15 (d, J=7.5 Hz, 1H), 7.01 (dd, J=9.7, 7.2 Hz, 1H), 6.86 (dd, J=9.6, 7.2 Hz, 1H), 5.95 (br s, 1H), 5.17 (br s, 1H), 4.64 (d, J=16.2 Hz, 4H), 4.01 (s, 2H), 3.50 (s, 2H), 3.46-3.31 (m, 4H), 3.05 (s, 2H), 2.85 (s, 6H), 2.70 (br s, 1H), 2.27-2.14 (m, 2H), 2.01-1.87 (m, 4H), 1.81 (d, J=12.0 Hz, 2H), 1.69 (d, J=12.0 Hz, 2H), 1.63-1.49 (m, 4H); ¹³C NMR (101 MHz, Chloroform-d) δ_{H} 170.0, 154.4, 152.0, 150.4 (dd, J=247.5, 30.3 Hz), 134.91, 132.1 (ddd, J=17.2, 7.1, 3.0 Hz), 130.49, 129.92, 129.66, 129.49, 128.35, 123.22, 118.95, 115.29, 111.8 (dd, J=26.3, 19.2 Hz), 77.36, 69.80, 69.38, 63.16, 52.63, 52.56, 51.97, 51.14, 45.46, 42.9 (d, J=12.1 Hz), 41.33, 40.91, 35.43, 29.84; LRMS m/z (ESI⁺) 726 [M+H]⁺.

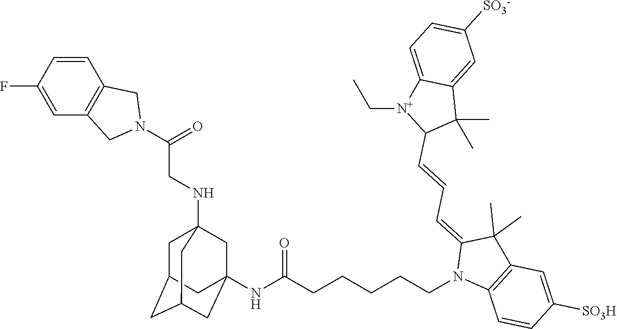

1-ethyl-2-((E)-3-((E)-1-(6-((-3-((2-(5-fluoroisoindolin-2-yl)-2-oxoethyl)amino)adamantan-1-yl)amino)-6-oxohexyl)-3,3-dimethyl-5-sulfoindolin-2-ylidene)prop-1-en-1-yl)-3,3-dimethyl-3H-indol-1-ium-5-sulfonate (120) (0.013 g, 0.014 mmol, 77% yield) was prepared according to general procedure 4 from 2-((3-aminoadamantan-1-yl)amino)-1-(5-fluoroisoindolin-2-yl)ethan-1-one bis(2,2,2-trifluoroacetate (6) (0.011 g, 0.018 mmol) and purified by silica gel flash chromatography (elution with a mixture of 1-15% MeOH in DCM). ¹H NMR (400 MHz, Methanol-d₄) δ_{H} 8.45 (t, J=13.3 Hz, 1H), 8.19 (d, J=7.3 Hz, 2H), 7.65-7.52 (m, 1H), 7.32 (dd, J=8.3, 1.6 Hz, 1H), 7.22 (d, J=8.4 Hz, 1H), 7.25 (p, J=9.1, 8.2 Hz, 2H), 6.45 (t, J=14.2 Hz, 2H), 5.51 (s, 2H), 4.70 (s, 2H), 4.87-4.79 (m, 4H), 4.70 (s, 2H), 4.65 (s, 1H), 4.26-4.10 (m, 3H), 3.65 (s, 1H), 3.32 (s, 1H), 3.30 (s, 1H), 2.5 (br s, H), 2.02 (q, J=7.8, 6.9 Hz, 2H), 1.93-1.80 (m, 4H), 1.76 (s, 12H), 1.73-1.50 (m, 8H), 1.45 (t, J=7.3 Hz, 4H); LRMS m/z (ESI⁺) 956 [M+H]⁺.

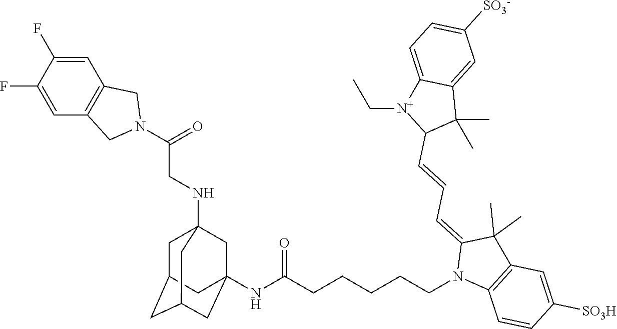

1-ethyl-2-((E)-3-((E)-1-(6-((-3-((2-(5,6-difluoroisoindolin-2-yl)-2-oxoethyl)amino)adamantan-1-yl)amino)-6-oxohexyl)-3,3-dimethyl-5-sulfoindolin-2-ylidene)prop-1-en-1-yl)-3,3-dimethyl-3H-indol-1-ium-5-sulfonate (121) (0.011 g, 0.011 mmol, 70% yield) was prepared according to general procedure 4 from 2-((3-aminoadamantan-1-yl)amino)-1-(5,6-difluoroisoindolin-2-yl)ethan-1-one bis(2,2,2-trifluoroacetate (6) (0.010 g, 0.016 mmol) and purified by silica gel flash chromatography (elution with a mixture of 1-15% MeOH in DCM). ¹H NMR (400 MHz, Methanol-d₄) δ_{H} 8.54 (t, J=13.4 Hz, 1H), 7.95-7.92 (m, 1H), 7.89 (dd, J=8.3, 1.6 Hz, 1H), 7.39 (d, J=8.4 Hz, 1H), 7.25 (p, J=9.1, 8.2 Hz, 2H), 6.50 (t, J=14.2 Hz, 2H), 5.46 (s, 2H), 4.71 (s, 2H), 4.87-4.79 (m, 4H), 4.71 (s, 2H), 4.60 (s, 1H), 4.26-4.13 (m, 3H), 3.67 (s, 1H), 3.31 (s, 1H), 3.31 (s, 1H), 2.18 (br s, H), 2.09 (q, J=7.8, 6.9 Hz, 2H), 1.93-1.80 (m, 4H), 1.76 (s, 12H), 1.74-1.53 (m, 8H), 1.40 (t, J=7.3 Hz, 4H); LRMS m/z (ESI⁺) 974 [M+H]⁺.

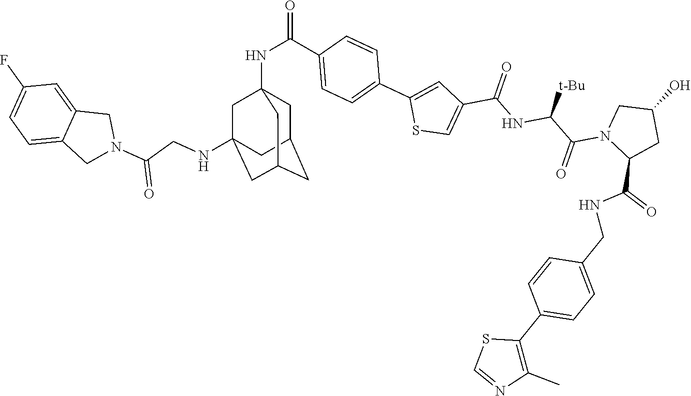 [SMILES: Cc1ncsc1-c1ccc(CNC(=O)[C@@H]2C[C@@H](O)CN2C(=O)[C@@H](NC(=O)c2csc(-c3ccc(C(=O)NC45CC6CC(CC(NCC(=O)N7Cc8ccc(F)cc8C7)(C6)C4)C5)cc3)c2)C(C)(C)C)cc1]

(2S,4R)-1-((2S)-2-(5-(4-((3-((2-(5-fluoroisoindolin-2-yl)-2-oxoethyl)amino)adamantan-1-yl)carbamoyl)phenyl)thiophene-3-carboxamido)-3,3-dimethylbutanoyl)-4-hydroxy-N-(4-(4-methylthiazol-5-yl)benzyl)pyrrolidine-2-carboxamide (122) (0.030 g, 0.030 mmol, 27% yield) was prepared according to general procedure 5 from methyl 5-(4-((-3-((2-(5-fluoroisoindolin-2-yl)-2-oxoethyl)amino)adamantan-1-yl)carbamoyl)phenyl)thiophene-3-carboxylate (103) (0.067 g, 0.114 mmol) and purified by silica gel flash chromatography (elution with a mixture of 1-15% MeOH in DCM). ¹H NMR (400 MHz, Methanol-d₄) δ_{H} 8.86 (s, 1H), 8.14 (br s, 1H), 7.89 (s, 3H), 7.83-7.76 (m, 2H), 7.74-7.66 (m, 2H), 7.47 (d, J=8.1 Hz, 2H), 7.41 (d, J=8.2 Hz, 2H), 7.33 (ddd, J=8.0, 5.1, 2.0 Hz, 1H), 7.13-7.00 (m, 2H), 4.75 (d, J=13.2 Hz, 2H), 4.65-4.57 (m, 2H), 4.56-4.50 (m, 2H), 4.33 (d, J=15.5 Hz, 1H), 3.96 (d, J=11.1 Hz, 1H), 3.88-3.80 (m, 3H), 3.33 (s, 1H), 2.46 (s, 3H), 2.37-2.26 (m, 5H), 2.26-2.17 (m, 3H), 2.14-1.99 (m, 4H), 1.94-1.81 (m, 5H), 1.74-1.62 (m, 2H), 1.10 (s, 9H); ¹³C NMR (101 MHz, Methanol-d₄) δ_{H} 174.4, 172.3, 169.5, 168.7, 164.8, 164.2 (dd,J=244.4, 11.1 Hz), 152.9, 149.0, 145.2, 140.3, 139.4 (dd, J=53.5, 9.1 Hz), 138.7, 137.6, 136.2, 133.4, 132.9 (dd, J=53.5, 2.0 Hz), 131.5, 130.6, 130.4, 130.4, 129.5, 129.3, 129.0, 126.5, 125.5 (dd,J=8.1, 6.1 Hz), 124.4, 116.0 (dd,J=23.2, 9.1 Hz), 111.0 (dd, J=24.2, 10.1 Hz), 79.5, 71.1, 61.0, 59.2, 58.3, 58.2, 56.6, 55.0, 53.4, 52.8, 52.6, 52.1, 44.2, 43.7, 42.7, 41.0, 40.1, 39.0, 37.2, 36.0, 31.2, 27.1, 18.4, 15.8. LRMS m/z (ESI⁺) 986 [M+H]⁺.

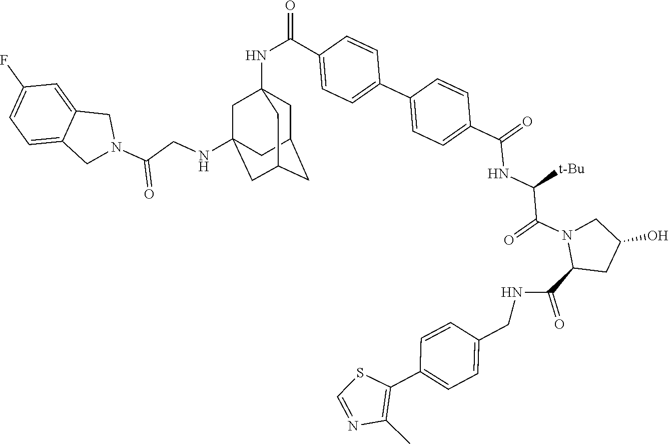 [SMILES: Cc1ncsc1-c1ccc(CNC(=O)[C@@H]2C[C@@H](O)CN2C(=O)[C@@H](NC(=O)c2ccc(-c3ccc(C(=O)NC45CC6CC(CC(NCC(=O)N7Cc8ccc(F)cc8C7)(C6)C4)C5)cc3)cc2)C(C)(C)C)cc1]

N4-(3-((2-(5-fluoroisoindolin-2-yl)-2-oxoethyl)amino)adamantan-1-yl)-N4′-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-[1,1′-biphenyl]-4,4′-dicarboxamide (123) (0.027 g, 0.028 mmol, 19% yield) was prepared according to general procedure 5 from methyl 4′-((-3-((2-(5-fluoroisoindolin-2-yl)-2-oxoethyl)amino)adamantan-1-yl)carbamoyl)-[1,1′-biphenyl]-4-carboxylate (104) (0.085 g, 0.146 mmol) and purified by silica gel flash chromatography (elution with a mixture of 1-15% MeOH in DCM).¹H NMR (400 MHz, Methanol-d₄) δ_{H} 8.72-8.70 (m, 2H), 7.83-7.76 (m, 4H), 7.74-7.63 (m, 4H), 7.45 (d, J=8.1 Hz, 2H), 7.35 (d, J=8.2 Hz, 2H), 7.32 (ddd, J=8.0, 5.1, 2.0 Hz, 1H), 4.75 (d, J=13.2 Hz, 2H), 4.65-4.57 (m, 2H), 4.56-4.50 (m, 2H), 4.35 (d, J=15.5 Hz, 1H), 4.00 (d, J=11.1 Hz, 1H), 3.80-3.72 (m, 3H), 3.35 (s, 1H), 2.46 (s, 3H), 2.37-2.30 (m, 5H), 2.26-2.15 (m, 3H), 2.14-2.00 (m, 4H), 1.94-1.82 (m, 5H), 1.74-1.62 (m, 2H), 1.10 (s, 9H); ¹³C NMR (101 MHz, Methanol-d₄)⁶c 174.4, 172.3, 171.4, 169.7, 169.4, 163.8 (dd, J=244.4, 9.1 Hz), 152.8, 149.0, 144.7, 143.9, 140.3, 139.6 (dd, J=49.5, 9.1 Hz), 136.6, 134.5, 133.4, 132.4 (dd, J=50.5, 47.5 Hz), 130.5, 130.4, 129.5, 129.2, 129.1, 129.1, 129.0, 128.2, 128.1, 125.5 (dd, J=11.1, 9.1 Hz), 115.9 (dd, J=23.2, 10.1 Hz), 111.0 (dd, J=24.2, 15.2 Hz), 71.1, 61.0, 59.42, 58.2, 55.12, 54.1, 53.3 (d, J=3.0 Hz), 52.8, 52.5 (d, J=2.0 Hz), 52.0, 45.7, 43.7, 43.1, 41.7, 41.3, 39.0, 37.3, 36.4, 31.4, 27.1, 15.8; LRMS m/z (ESI⁺) 980 [M+H]⁺.

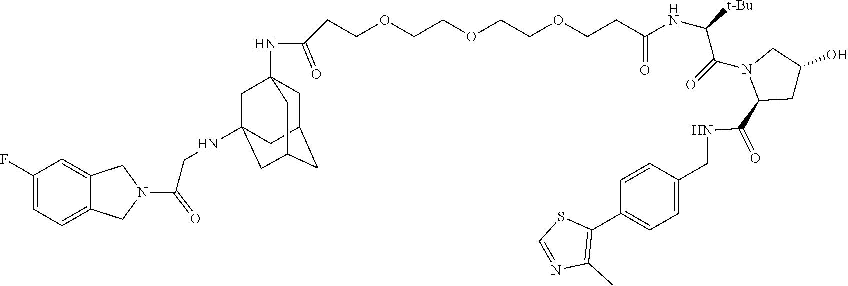 [SMILES: Cc1ncsc1-c1ccc(CNC(=O)[C@@H]2C[C@@H](O)CN2C(=O)[C@@H](NC(=O)CCOCCOCCOCCC(=O)NC23CC4CC(CC(NCC(=O)N5Cc6ccc(F)cc6C5)(C4)C2)C3)C(C)(C)C)cc1]

(2S,4R)-1-((2S)-2-(tert-butyl)-16-((3-((2-(5-fluoroisoindolin-2-yl)-2-oxoethyl)amino)adamantan-1-yl)amino)-4,16-dioxo-7,10,13-trioxa-3-azahexadecanoyl)-4-hydroxy-N-(4-(4-methylthiazol-5-yl)benzyl)pyrrolidine-2-carboxamide (124) (0.033 g, 0.033 mmol, 30% yield) was prepared according to general procedure 6 from tert-butyl 3-(2-(2-(3-((-3-((2-(5-fluoroisoindolin-2-yl)-2-oxoethyl)amino)adamantan-1-yl)amino)-3-oxopropoxy)ethoxy)ethoxy) propanoate (82) (0.070 g, 0.111 mmol) and purified by silica gel flash chromatography (elution with a mixture of 1-10% MeOH in DCM); 8.82 (s, 1H), 7.93 (s, 1H), 7.65-7.59 (m, 2H), 7.58-7.52 (m, 2H), 7.48-7.35 (m, 2H), 4.82-4.75 (m, 4H), 4.66 (s, 1H), 4.63-4.47 (m, 3H), 4.32 (d, J=15.6 Hz, 1H), 4.17 (t, J=4.7 Hz, 2H), 3.94-3.75 (m, 10H), 3.71-3.59 (m, 5H), 2.66-2.47 (m, 3H), 2.43 (s, 3H), 2.31 (br s, 2H), 2.26-2.14 (m, 4H), 2.12-2.02 (m, 3H), 1.85-1.61 (m, 6H), 1.03 (s, 9H); LRMS m/z (ESI⁺) 988 [M+H]⁺.

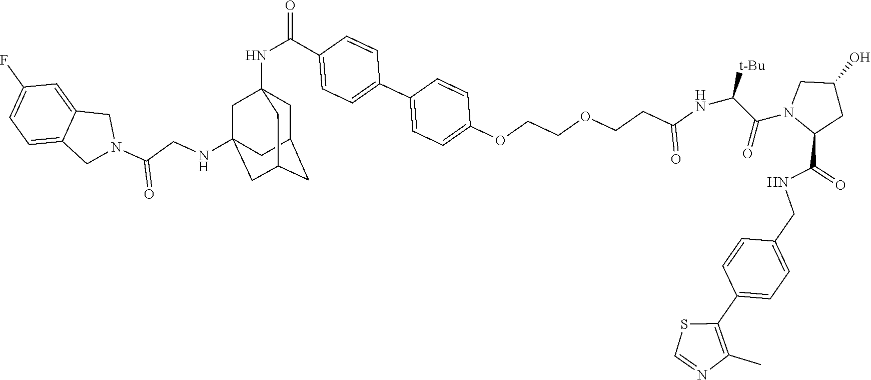 [SMILES: Cc1ncsc1-c1ccc(CNC(=O)[C@@H]2C[C@@H](O)CN2C(=O)[C@@H](NC(=O)CCOCCOc2ccc(-c3ccc(C(=O)NC45CC6CC(CC(NCC(=O)N7Cc8ccc(F)cc8C7)(C6)C4)C5)cc3)cc2)C(C)(C)C)cc1]

(2S,4R)-1-((2S)-2-(3-(2-((4′-((3-((2-(5-fluoroisoindolin-2-yl)-2-oxoethyl)amino)adamantan-1-yl)carbamoyl)-[1,1′-biphenyl]-4-yl)oxy)ethoxy)propanamido)-3,3-dimethylbutanoyl)-4-hydroxy-N-(4-(4-methylthiazol-5-yl)benzyl)pyrrolidine-2-carboxamide (125) (0.027 g, 0.025 mmol, 50% yield) was prepared according to general procedure 6 from tert-butyl 3-(2-((4′-((-3-((2-(5-fluoroisoindolin-2-yl)-2-oxoethyl)amino)adamantan-1-yl)carbamoyl)-[1,1′-biphenyl]-4-yl)oxy)ethoxy)propanoate (82) (0.036 g, 0.051 mmol) and purified by silica gel flash chromatography (elution with a mixture of 1-10% MeOH in DCM). ¹H NMR (400 MHz, Methanol-d4) δ_{H} ¹H NMR (400 MHz, Methanol-d4) δ 8.83 (s, 1H), 7.91 (s, 1H), 7.87-7.74 (m, 2H), 7.66-7.58 (m, 2H), 7.58-7.52 (m, 2H), 7.48-7.29 (m, 5H), 7.13-6.94 (m, 4H), 4.86-4.70 (m, 4H), 4.66 (s, 1H), 4.63-4.45 (m, 3H), 4.34 (d, J=15.5 Hz, 1H), 4.17 (t, J=4.7 Hz, 2H), 3.94-3.75 (m, 5H), 3.71-3.59 (m, 2H), 2.66-2.47 (m, 2H), 2.43 (s, 3H), 2.31 (br s, 2H), 2.26-2.14 (m, 4H), 2.12-2.02 (m, 3H), 1.85-1.61 (m, 6H), 1.03 (s, 9H); ¹³C NMR (101 MHz, Methanol-d4) δ_{H} 174.4, 173.7, 172.1, 170.6, 169.8, 164.1 (dd,J=247.5, 7.1 Hz), 160.4, 152.8, 149.0, 144.8, 140.2, 139.5 (dd, J=51.5, 10.1 Hz), 135.0, 133.7, 133.4, 132.9 (dd, J=48.5, 2.0 Hz), 131.5, 130.3, 129.2, 128.9, 128.9, 127.3, 125.0 (dd, J=15.2, 7.1 Hz), 116.1, 116.0-115.8 (m), 111.0 (dd, J=27.3, 15.2 Hz), 79.5, 71.1, 70.7, 68.6, 68.4, 60.8, 59.0, 58.0, 55.0, 54.7, 53.3, 52.8, 52.0, 45.4, 43.7, 41.2, 39.0, 37.4, 36.8, 36.3, 31.3, 27.0, 15.8; LRMS m/z (ESI⁺) 1068 [M+H]⁺.

### Biochemical Evaluation.

DPP4 was purified from human seminal plasma as described previously.^{[3]} Recombinant human (rh) DPP8 and rhDPP9 were expressed in Sf9 insect cells using the N-terminal BaculoDirect insect cell expression system (Invitrogen) and were purified as described by De Decker et al.[2]rhDPP2 was purchased from R&D (3438-SE). rhFAP (extracellular domain, amino acid 27-760) with a C-terminal His-tag was expressed and purified in Sf9 insect cells as described before.^{[4]} rhPREP was expressed in BL21(DE3) cells and purified as described by De Decker et al.^{[5]}

IC50 measurements. Enzyme activities were determined kinetically in 96-well half area plates (Greiner Bio-One) in a final volume of 100 μL for at least 15 min. at 37° C. by measuring the initial velocities of pNA release (405 nm) or AMC release (λₑₓ=380 nm, λₑₘ=465 nm) from the substrate using an Infinite™ M200 reader (Tecan Benelux). The Magellan software was used to process the data, which was then fitted using a non-linear fit model in GraFit 7. The chromogenic substrate Ala-Pro-paranitroanilide (pNA) (Bachem) was used for DPP4 (25 μM), DPP8 (300 μM) and DPP9 (150 μM) at pH 7.4 (0.05 M HEPES-NaOH buffer with 0.10/Tween-20, 0.1 mg/mL BSA and 150 mM NaCl) and Lys-Ala-pNA (Bachem) was used for DPP2 (1 mM) at pH 5.5 (100 mM NaAc, 10 mM EDTA, 14 μg/mL aprotinin). The fluorogenic substrate Z-Gly-Pro-7-amino-4-methylcoumarine (AMC) (Bachem) was used for FAP (50 μM) at pH 8 (0.05 M Tris-HCl buffer with 1 mg/mL BSA and 140 mM NaCl) and N-succinyl-Gly-Pro-AMC (Bachem) was used for PREP (250 μM) at pH 7.4 (0.1 M K-phosphate, 1 mM EDTA, 1 mM DTT and 1 mg/mL BSA). As a blank, the assay buffer was used instead of the enzyme. Screening measurements were carried out in duplo and IC50 values were determined in triplo with at least eight different inhibitor concentrations as described by Van Goethem et al.

None of the compounds inhibited the other DASH proteins DPP4, fibroblast activation protein (FAP), and prolyl oligopeptidase (PREP) at concentrations of 10 mM, and DPP2 at concentrations of 5 μM.

TABLE 3
isoindoline-containing examplary compounds

| | | | DPP8 | DPP9 | SI^{[b]} |
|---|---|---|---|---|---|
| | UAMC- | | IC₅₀^{[a]} ± | IC₅₀^{[a]} ± | DPP9/ |
| | 000 | STRUCTURE | SD (μM) | SD (μM) | 8 |
| 25 | 4805 | 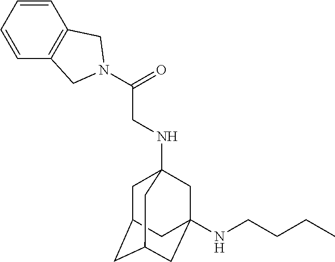 [SMILES: CCCCNC12CC3CC(C1)CC(NCC(=O)N1Cc4ccccc4C1)(C3)C2] | >10 | 0.477 ± 0.028 | >21 |
| 26 | 3916 | 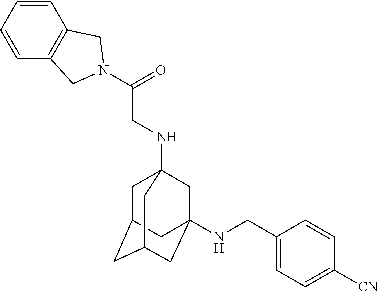 [SMILES: N#Cc1ccc(CNC23CC4CC(CC(NCC(=O)N5Cc6ccccc6C5)(C4)C2)C3)cc1] | 0.672 ± 0.412 | 0.0506 ± 0.0287 | 13.3 |

| | | | | | |
|---|---|---|---|---|---|
| 27 | 3929 | 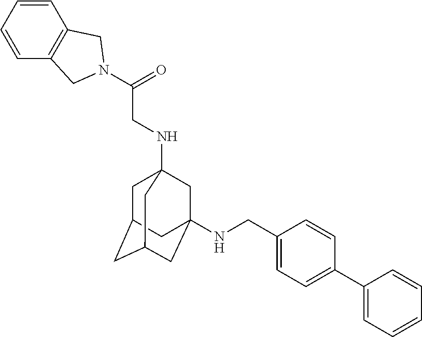 [SMILES: O=C(CNC12CC3CC(C1)CC(NCc1ccc(-c4ccccc4)cc1)(C3)C2)N1Cc2ccccc2C1] | 1.54 ± 0.101 | 0.0932 ± 0.0390 | 16.5 |

| | | | | | |
|---|---|---|---|---|---|
| 28 | 3838 | 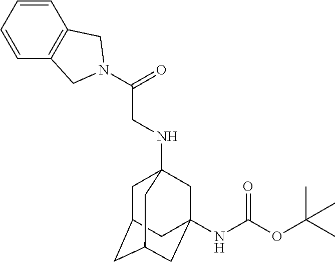 [SMILES: CC(C)(C)OC(=O)NC12CC3CC(CC(NCC(=O)N4Cc5ccccc5C4)(C3)C1)C2] | 1.14 ± 0.150 | 0.0367 ± 4.73E−03 | 31.7 |

| | | | | | |
|---|---|---|---|---|---|
| 29 | 3821 | 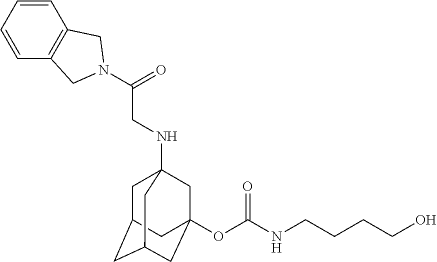 [SMILES: O=C(NCCCCO)OC12CC3CC(CC(NCC(=O)N4Cc5ccccc5C4)(C3)C1)C2] | 2.60 ± 0.577 | 0.243 ± 0.107 | 10.7 |

| | | | | | |
|---|---|---|---|---|---|
| 30 | 4068 | 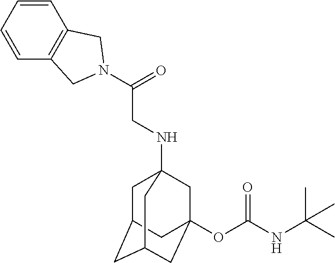 [SMILES: CC(C)(C)NC(=O)OC12CC3CC(CC(NCC(=O)N4Cc5ccccc5C4)(C3)C1)C2] | 2.59 ± 0.190 | 0.169 ± 1.98E−03 | 15.3 |

| | | | | | |
|---|---|---|---|---|---|
| 31 | 4289 | 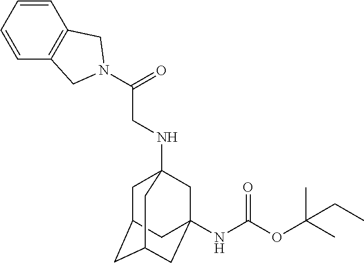 [SMILES: CCC(C)(C)OC(=O)NC12CC3CC(CC(NCC(=O)N4Cc5ccccc5C4)(C3)C1)C2] | 4.71 ± 1.59 | 0.378 ± 0.173 | 12.5 |

| | | | | | |
|---|---|---|---|---|---|
| 32 | 4291 | 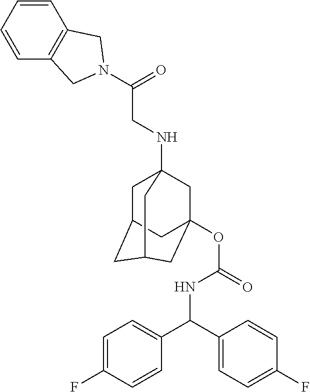 [SMILES: O=C(NC(c1ccc(F)cc1)c1ccc(F)cc1)OC12CC3CC(CC(NCC(=O)N4Cc5ccccc5C4)(C3)C1)C2] | 0.667 ± 0.0311 | 0.0477 ± 7.66E−03 | 14.0 |

| | | | | | |
|---|---|---|---|---|---|
| 33 | 3925 | 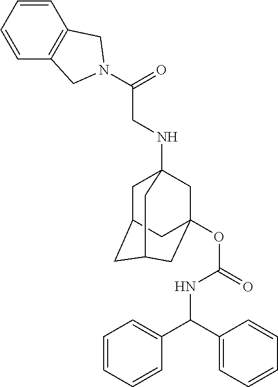 [SMILES: O=C(NC(c1ccccc1)c1ccccc1)OC12CC3CC(CC(NCC(=O)N4Cc5ccccc5C4)(C3)C1)C2] | 0.175 ± 0.0331 | 0.0143 ± 2.52E−03 | 12.2 |

| | | | | | |
|---|---|---|---|---|---|
| 34 | 4060 | 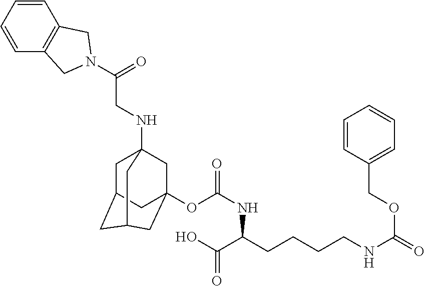 [SMILES: O=C(NCCCC[C@H](NC(=O)OC12CC3CC(CC(NCC(=O)N4Cc5ccccc5C4)(C3)C1)C2)C(=O)O)OCc1ccccc1] | 0.0394 ± 9.06E−03 | 1.74E−03 ± 3.70E−06 | 22.5 |

| | | | | | |
|---|---|---|---|---|---|
| 35 | 4059 | 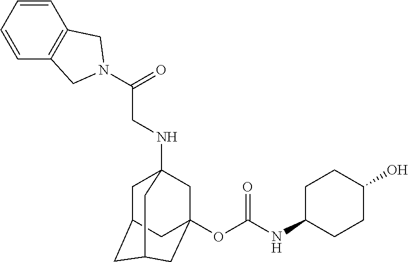 [SMILES: O=C(N[C@H]1CC[C@H](O)CC1)OC12CC3CC(CC(NCC(=O)N4Cc5ccccc5C4)(C3)C1)C2] | 0.399 ± 0.0388 | 0.0366 ± 7.31E−03 | 10.9 |

| | | | | | |
|---|---|---|---|---|---|
| 36 | 3910 | 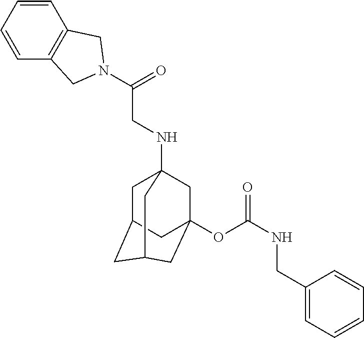 [SMILES: O=C(NCc1ccccc1)OC12CC3CC(CC(NCC(=O)N4Cc5ccccc5C4)(C3)C1)C2] | 1.15 ± 0.151 | 0.0738 + 0.0191 | 15.6 |

| | | | | | |
|---|---|---|---|---|---|
| 37 | 3914 | 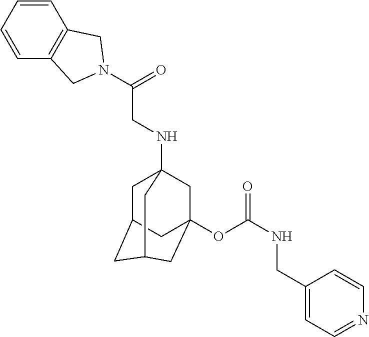 [SMILES: O=C(NCc1ccncc1)OC12CC3CC(CC(NCC(=O)N4Cc5ccccc5C4)(C3)C1)C2] | 1.11 ± 0.335 | 0.0908 ± 0.0135 | 12.2 |
| 38 | 3824 | 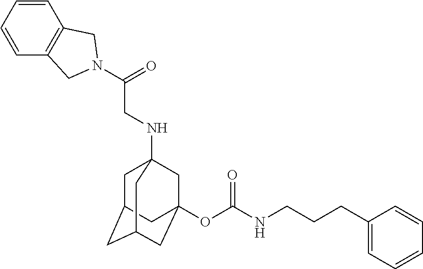 [SMILES: O=C(NCCCc1ccccc1)OC12CC3CC(CC(NCC(=O)N4Cc5ccccc5C4)(C3)C1)C2] | 1.01 ± 0.132 | 0.100 ± 3.79E−03 | 10.1 |
| 39 | 3823 | 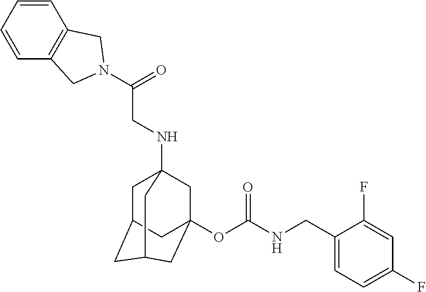 [SMILES: O=C(NCc1ccc(F)cc1F)OC12CC3CC(CC(NCC(=O)N4Cc5ccccc5C4)(C3)C1)C2] | 0.823 ± 0.185 | 0.0483 ± 6.03E−03 | 17.0 |
| 40 | 4295 | 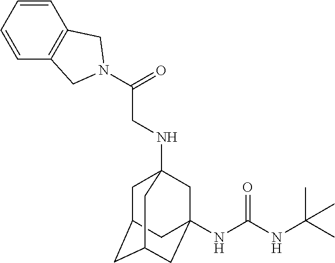 [SMILES: CC(C)(C)NC(=O)NC12CC3CC(CC(NCC(=O)N4Cc5ccccc5C4)(C3)C1)C2] | 1.05 ± 0.133 | 0.0518 ± 0.0157 | 20.3 |
| 41 | 4292 | 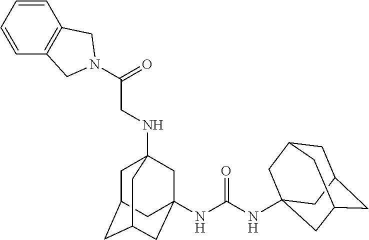 [SMILES: O=C(NC12CC3CC(CC(C3)C1)C2)NC12CC3CC(CC(NCC(=O)N4Cc5ccccc5C4)(C3)C1)C2] | 0.592 ± 0.0877 | 0.0328 ± 1.05E−03 | 18.0 |
| 42 | 488 | 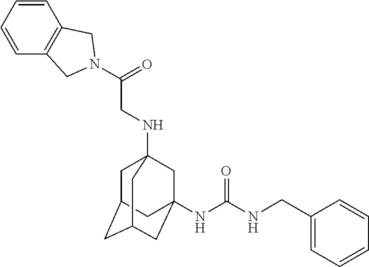 [SMILES: O=C(NCc1ccccc1)NC12CC3CC(CC(NCC(=O)N4Cc5ccccc5C4)(C3)C1)C2] | 0.505 ± 0.0257 | 0.0378 ± 0.0133 | 13.4 |
| 43 | 4151 | 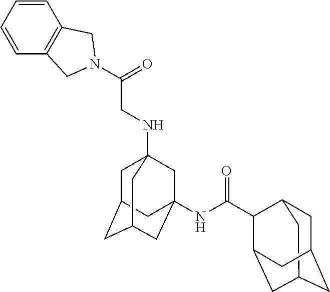 [SMILES: O=C(NC12CC3CC(CC(NCC(=O)N4Cc5ccccc5C4)(C3)C1)C2)C1C2CC3CC(C2)CC1C3] | 5.70 ± 1.10 | 0.186 ± 0.0536 | 30.6 |
| 44 | 4159 | 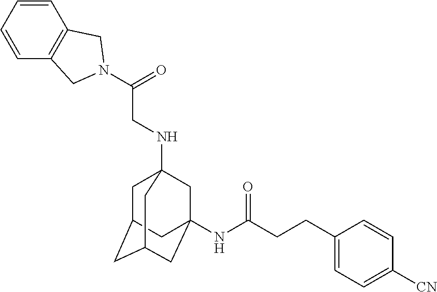 [SMILES: N#Cc1ccc(CCC(=O)NC23CC4CC(CC(NCC(=O)N5Cc6ccccc6C5)(C4)C2)C3)cc1] | 0.638 ± 0.0464 | 0.0320 ± 1.80E−03 | 19.9 |
| 45 | 4290 | 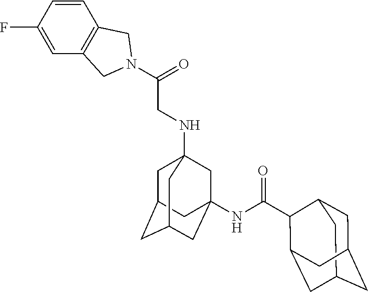 [SMILES: O=C(NC12CC3CC(CC(NCC(=O)N4Cc5ccc(F)cc5C4)(C3)C1)C2)C1C2CC3CC(C2)CC1C3] | 1.176 ± 0.138 | 0.038 ± 0.010 | 31 |
| 46 | 3955 | 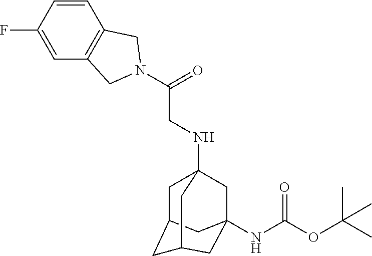 [SMILES: CC(C)(C)OC(=O)NC12CC3CC(CC(NCC(=O)N4Cc5ccc(F)cc5C4)(C3)C1)C2] | 0.824 ± 0.148 | 0.0143 ± 2.44E−03 | 57.6 |
| 47 | 3954 | 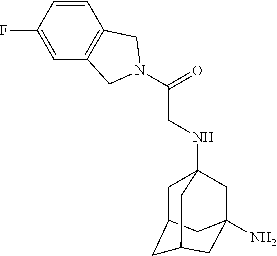 [SMILES: NC12CC3CC(C1)CC(NCC(=O)N1Cc4ccc(F)cc4C1)(C3)C2] | 2.62 ± 0.394 | 0.259 ± 0.0358 | 10.1 |
| 48 | 4804 | 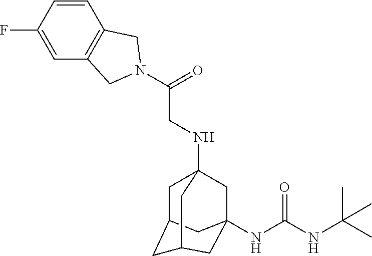 [SMILES: CC(C)(C)NC(=O)NC12CC3CC(CC(NCC(=O)N4Cc5ccc(F)cc5C4)(C3)C1)C2] | 0.369 ± 0.030 | 0.004 ± 0.001 | 85 |
| 49 | 4918 | 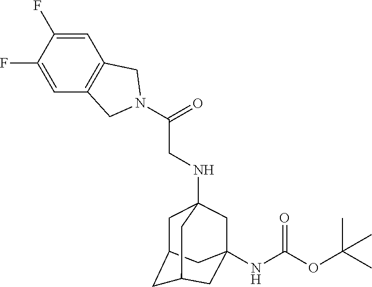 [SMILES: CC(C)(C)OC(=O)NC12CC3CC(CC(NCC(=O)N4Cc5cc(F)c(F)cc5C4)(C3)C1)C2] | 0.613 ± 0.154 | 3.40E−03 ± 591E−06 | 180.3 |
| 50 | 4913 | 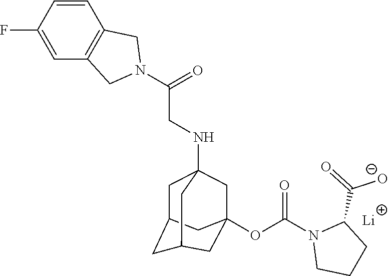 [SMILES: O=C([O-])[C@@H]1CCCN1C(=O)OC12CC3CC(CC(NCC(=O)N4Cc5ccc(F)cc5C4)(C3)C1)C2 ; [Li+]] | 0.920 ± 0.121 | 8.03E−03 ± 2.13E−03 | 114.6 |
| 51 | 4912 | 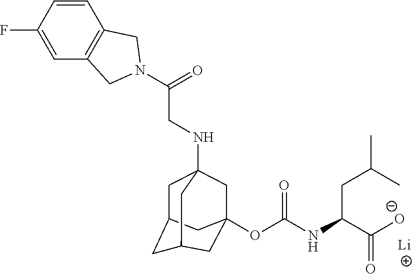 [SMILES: CC(C)C[C@H](NC(=O)OC12CC3CC(CC(NCC(=O)N4Cc5ccc(F)cc5C4)(C3)C1)C2)C(=O)[O-] ; [Li+]] | 1.08 ± 0.0470 | 0.0135 ± 2.79E−03 | 80 |
| 52 | 4911 | 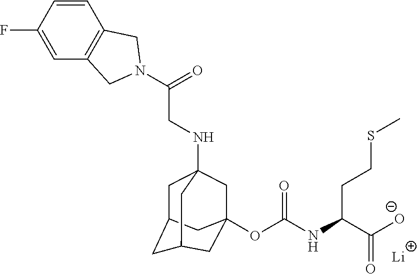 [SMILES: CSCC[C@H](NC(=O)OC12CC3CC(CC(NCC(=O)N4Cc5ccc(F)cc5C4)(C3)C1)C2)C(=O)[O-] ; [Li+]] | 0.504 ± 0.0516 | 9.13E−03 ± 2.10E−03 | 55.2 |
| 53 | 4909 | 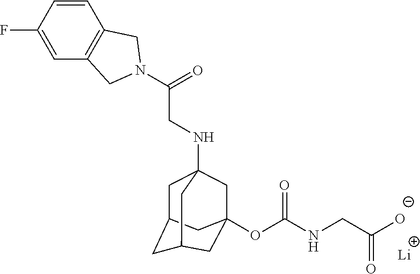 [SMILES: O=C([O-])CNC(=O)OC12CC3CC(CC(NCC(=O)N4Cc5ccc(F)cc5C4)(C3)C1)C2 ; [Li+]] | 1.74 ± 0.0591 | 0.0396 ± 4.70E−03 | 43.9 |
| 54 | 4801 | 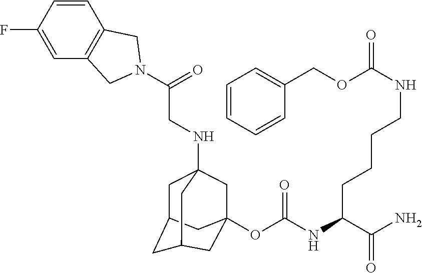 [SMILES: NC(=O)[C@H](CCCCNC(=O)OCc1ccccc1)NC(=O)OC12CC3CC(CC(NCC(=O)N4Cc5ccc(F)cc5C4)(C3)C1)C2] | 0.243 ± 0.0464 | 8.88E−03 ± 2.42E−03 | 27.4 |
| 55 | 4910 | 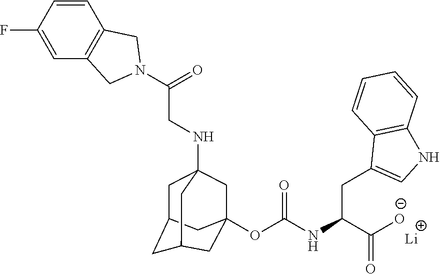 [SMILES: O=C(N[C@@H](Cc1c[nH]c2ccccc12)C(=O)[O-])OC12CC3CC(CC(NCC(=O)N4Cc5ccc(F)cc5C4)(C3)C1)C2 ; [Li+]] | 0.164 ± 4.37E−03 | 6.55E−03 ± 2.51E−03 | 25.0 |
| 56 | 4798 | 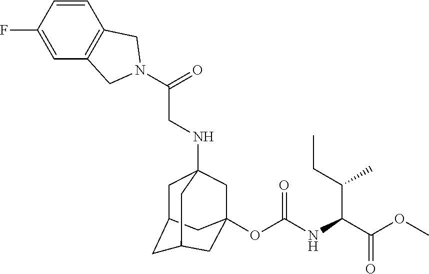 [SMILES: CC[C@H](C)[C@H](NC(=O)OC12CC3CC(CC(NCC(=O)N4Cc5ccc(F)cc5C4)(C3)C1)C2)C(=O)OC] | 2.08 ± 0.184 | 0.0946 ± 2.29E−03 | 22.0 |
| 57 | 4796 | 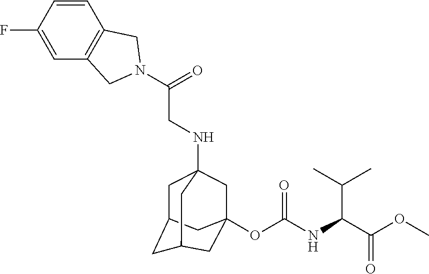 [SMILES: COC(=O)[C@@H](NC(=O)OC12CC3CC(CC(NCC(=O)N4Cc5ccc(F)cc5C4)(C3)C1)C2)C(C)C] | 2.18 ± 0.345 | 0.107 ± 1.72E−03 | 20.4 |
| 59 | 4927 | 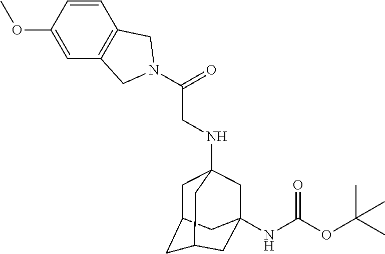 [SMILES: COc1ccc2c(c1)CN(C(=O)CNC13CC4CC(C1)CC(NC(=O)OC(C)(C)C)(C4)C3)C2] | 5.22 ± 0.321 | 0.309 ± 0.0604 | 16.9 |
| 60 | 4800 | 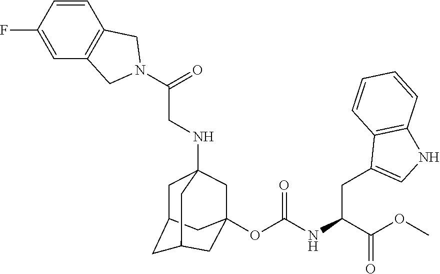 [SMILES: COC(=O)[C@H](Cc1c[nH]c2ccccc12)NC(=O)OC12CC3CC(CC(NCC(=O)N4Cc5ccc(F)cc5C4)(C3)C1)C2] | 0.689 ± 0.0323 | 0.0438 ± 4.71E−03 | 15.7 |
| 61 | 4794 | 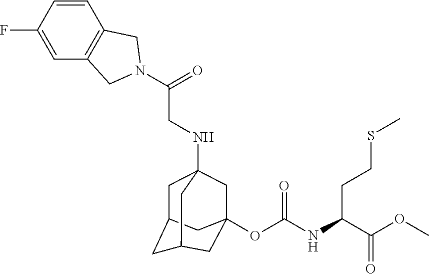 [SMILES: COC(=O)[C@H](CCSC)NC(=O)OC12CC3CC(CC(NCC(=O)N4Cc5ccc(F)cc5C4)(C3)C1)C2] | 0.705 ± 0.0760 | 0.0461 ± 6.12E−03 | 15.3 |
| 62 | 4797 | 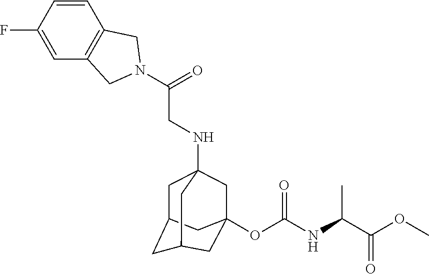 [SMILES: COC(=O)[C@H](C)NC(=O)OC12CC3CC(CC(NCC(=O)N4Cc5ccc(F)cc5C4)(C3)C1)C2] | 1.29 ± 0.186 | 0.0938 ± 0.0173 | 13.8 |
| 63 | 4803 | 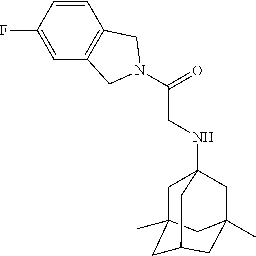 [SMILES: CC12CC3CC(C)(C1)CC(NCC(=O)N1Cc4ccc(F)cc4C1)(C3)C2] | 4.78 ± 0.670 | 0.443 ± 0.168 | 10.8 |
| 73 | 4924 | 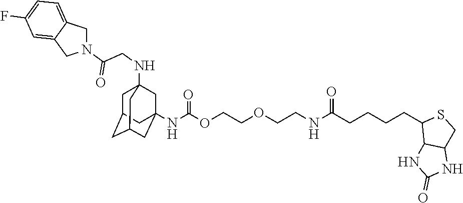 [SMILES: O=C(CCCCC1SCC2NC(=O)NC21)NCCOCCOC(=O)NC12CC3CC(CC(NCC(=O)N4Cc5ccc(F)cc5C4)(C3)C1)C2] | 0.457 ± 0.055 | 16E−03 ± 3E−03 | 29 |
| 74 | 4926 | 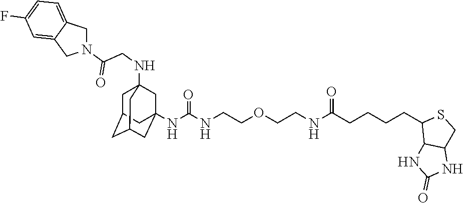 [SMILES: O=C(CCCCC1SCC2NC(=O)NC21)NCCOCCNC(=O)NC12CC3CC(CC(NCC(=O)N4Cc5ccc(F)cc5C4)(C3)C1)C2] | 0.310 ± 0.027 | 19E−03 ± 3E−03 | 16 |
| 75 | 4925 | 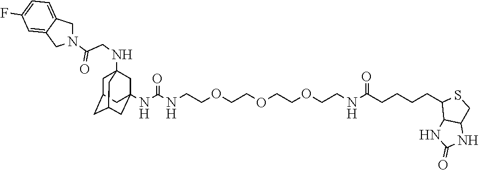 [SMILES: O=C(CCCCC1SCC2NC(=O)NC21)NCCOCCOCCOCCNC(=O)NC12CC3CC(CC(NCC(=O)N4Cc5ccc(F)cc5C4)(C3)C1)C2] | 0.495 ± 0.072 | 43E−03 ± 6E−03 | 12 |
| 76 | 4923 | 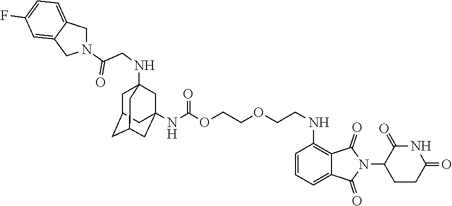 [SMILES: O=C1CCC(N2C(=O)c3cccc(NCCOCCOC(=O)NC45CC6CC(CC(NCC(=O)N7Cc8ccc(F)cc8C7)(C6)C4)C5)c3C2=O)C(=O)N1] | 8E−03 ± 1E−03 | 1.4E−03 ± 0.8E−03 | 6 |
| 77 | 4921 | 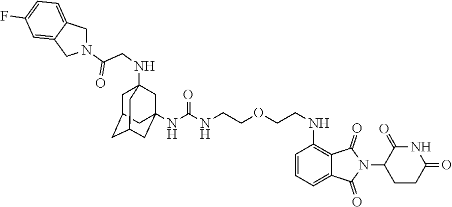 [SMILES: O=C1CCC(N2C(=O)c3cccc(NCCOCCNC(=O)NC45CC6CC(CC(NCC(=O)N7Cc8ccc(F)cc8C7)(C6)C4)C5)c3C2=O)C(=O)N1] | 21E−03 ± 4E−03 | 2.4E−03 ± 0.7E−03 | 9 |
| 78 | 4922 | 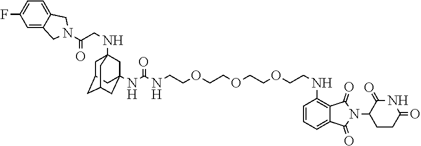 [SMILES: O=C1CCC(N2C(=O)c3cccc(NCCOCCOCCOCCNC(=O)NC45CC6CC(CC(NCC(=O)N7Cc8ccc(F)cc8C7)(C6)C4)C5)c3C2=O)C(=O)N1] | 79E−03 ± 4E−03 | 12E−03 ± 5E−03 | 7 |

^{[a]}IC₅₀s are presented as the mean ± standard deviation (n = 3).
^{[b]}SI: selectivity index, calculated by dividing DPP8 IC₅₀ over the DPP9 IC₅₀ (DPP9/8).

TABLE 4
isoindoline-containing examplary compounds

| | | | | | | SI^{[b]} |
|---|---|---|---|---|---|---|
| | | | | | | DPP |
| | UA | | | IC₅₀^{[a]} | IC₅₀^{[a]} | 9/ |
| In- | MC- | | | (μM) | (μM) | DPP |
| hibitors | 000 | nr. | Compound | DPP8 | DPP9 | 8 |
| NF- 2022- 071 UAMC 0005 033 | 50 33 | 84 | 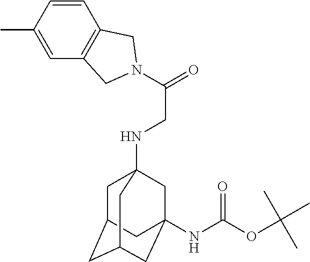 [SMILES: Cc1ccc2c(c1)CN(C(=O)CNC13CC4CC(C1)CC(NC(=O)OC(C)(C)C)(C4)C3)C2] | 4.71 ± 0.444 | 0.208 ± 0.048 | 23 |
| UAMC 0005 036 | 50 36 | 85 | 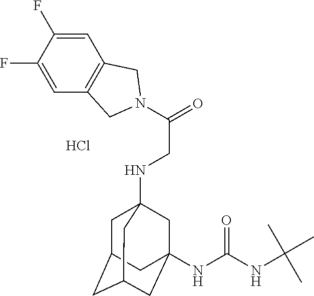 [SMILES: CC(C)(C)NC(=O)NC12CC3CC(CC(NCC(=O)N4Cc5cc(F)c(F)cc5C4)(C3)C1)C2 ; Cl] | 1.33 ± 0.097 | 0.032 ± 6E−3 | 41 |
| UAMC 0005 037 | 50 37 | 86 | 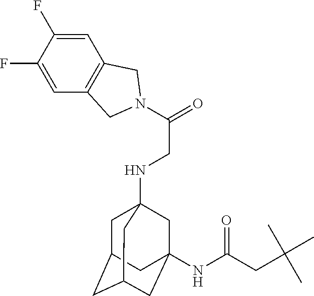 [SMILES: CC(C)(C)CC(=O)NC12CC3CC(CC(NCC(=O)N4Cc5cc(F)c(F)cc5C4)(C3)C1)C2] | 1.3 ± 0.127 | 0.058 ± 6E−3 | 22 |
| UAMC 0005 035 | 50 35 | 87 | 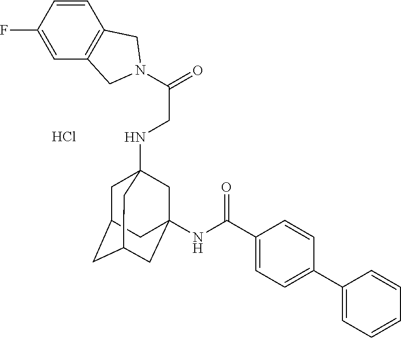 [SMILES: O=C(NC12CC3CC(CC(NCC(=O)N4Cc5ccc(F)cc5C4)(C3)C1)C2)c1ccc(-c2ccccc2)cc1 ; Cl] | 0.276* ± 0.016 | 0.0051* ± 0.0008 | 54 |
| UAMC 0005 111 | 51 11 | 88 | 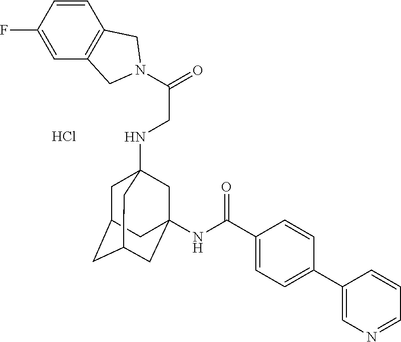 [SMILES: O=C(NC12CC3CC(CC(NCC(=O)N4Cc5ccc(F)cc5C4)(C3)C1)C2)c1ccc(-c2cccnc2)cc1 ; Cl] | 0.051 ± 0.025 | 2E−3 ± 1E−3 | 24 |
| NF- 2023- 048 UAMC- 0005 112- 003 | 51 12 | 89 | 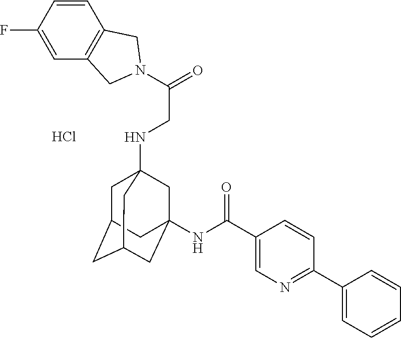 [SMILES: O=C(NC12CC3CC(CC(NCC(=O)N4Cc5ccc(F)cc5C4)(C3)C1)C2)c1ccc(-c2ccccc2)nc1 ; Cl] | 0.145* ± 0.019 | 5E−3* ± 0.3E−3 | 28 |
| NF- 2023- 069 UAMC- 0005 113 | 51 13 | 90 | 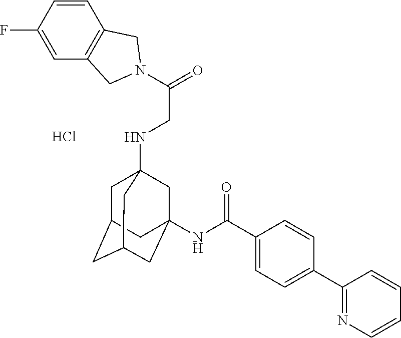 [SMILES: O=C(NC12CC3CC(CC(NCC(=O)N4Cc5ccc(F)cc5C4)(C3)C1)C2)c1ccc(-c2ccccn2)cc1 ; Cl] | 0.127 ± 0.023 | 3E−3 ± 1E−3 | 37 |
| UAMC- 0005 114 | 51 14 | 91 | 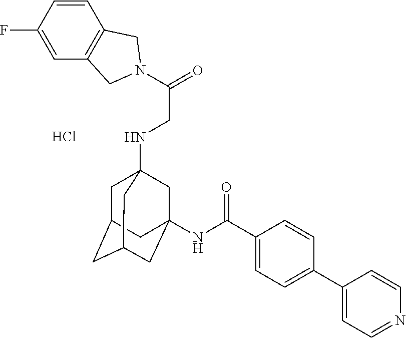 [SMILES: O=C(NC12CC3CC(CC(NCC(=O)N4Cc5ccc(F)cc5C4)(C3)C1)C2)c1ccc(-c2ccncc2)cc1 ; Cl] | 0.102 ± 0.028 | 8E−3 ± 0.6E−3 | 12 |
| NF- 2023- 049 UAMC- 0005 116 | 51 16 | 92 | 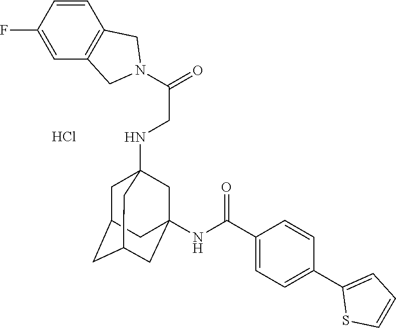 [SMILES: O=C(NC12CC3CC(CC(NCC(=O)N4Cc5ccc(F)cc5C4)(C3)C1)C2)c1ccc(-c2cccs2)cc1 ; Cl] | 0.222* ± 0.03 | 8E−3* ± 0.6E−3 | 27 |
| NF- 2023- 050 UAMC- 0005 356 | 53 56 | 93 | 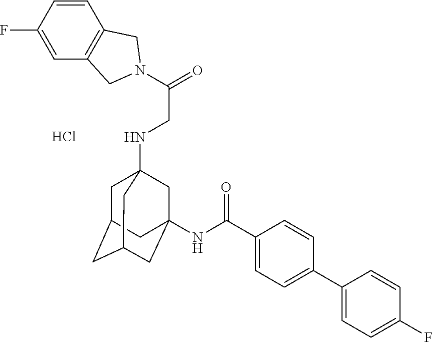 [SMILES: O=C(NC12CC3CC(CC(NCC(=O)N4Cc5ccc(F)cc5C4)(C3)C1)C2)c1ccc(-c2ccc(F)cc2)cc1 ; Cl] | 0.757* ± 0.111 | 0.013* ± 0.8E−3 | 59 |
| NF- 2023- 051 UAMC- 0005 357 | 53 57 | 94 | 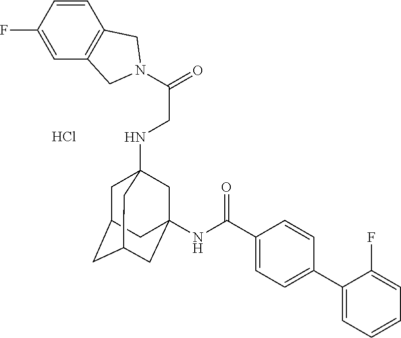 [SMILES: O=C(NC12CC3CC(CC(NCC(=O)N4Cc5ccc(F)cc5C4)(C3)C1)C2)c1ccc(-c2ccccc2F)cc1 ; Cl] | 0.403* ± 0.052 | 0.015* ± 2E−3 | 26 |
| NF- 2023- 053 UAMC- 0005 359 | 53 59 | 95 | 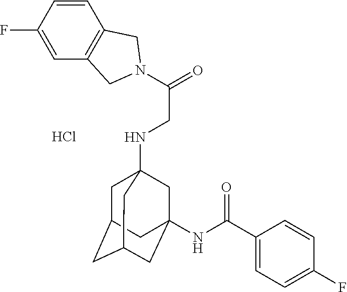 [SMILES: O=C(NC12CC3CC(CC(NCC(=O)N4Cc5ccc(F)cc5C4)(C3)C1)C2)c1ccc(F)cc1 ; Cl] | 0.295 ± 0.015 | 0.042 ± 7E−3 | 7 |
| NF- 2023- 054 UAMC- 0005 360 | 53 60 | 96 | 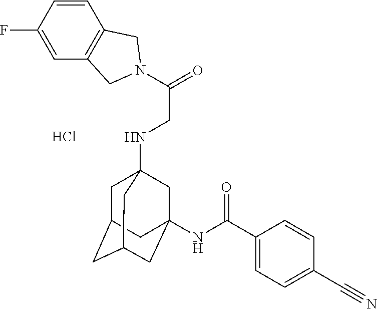 [SMILES: N#Cc1ccc(C(=O)NC23CC4CC(CC(NCC(=O)N5Cc6ccc(F)cc6C5)(C4)C2)C3)cc1 ; Cl] | 0.161* ± 0.027 | 0.014* ± 0.8E−3 | 12 |
| NF- 2023- 055 UAMC- 0005 361 | 53 61 | 97 | 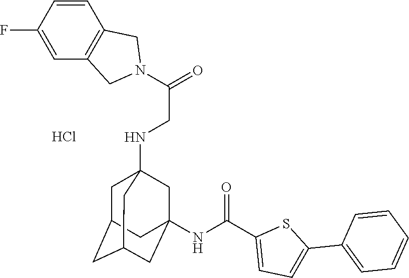 [SMILES: O=C(NC12CC3CC(CC(NCC(=O)N4Cc5ccc(F)cc5C4)(C3)C1)C2)c1ccc(-c2ccccc2)s1 ; Cl] | 0.244* ± 0.019 | 0.023* ± 2E−3 | 11 |
| NF- 2023- 057 UAMC- 0005 363 | 53 63 | 98 | 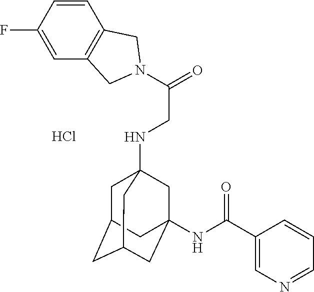 [SMILES: O=C(NC12CC3CC(CC(NCC(=O)N4Cc5ccc(F)cc5C4)(C3)C1)C2)c1cccnc1 ; Cl] | 0.270 ± 0.033 | 0.088 ± 0.023 | 3 |
| NF- 2023- 058 UAMC- 0005 364 | 53 64 | 99 | 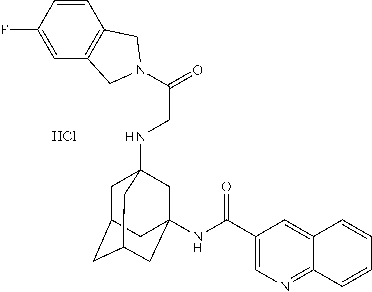 [SMILES: O=C(NC12CC3CC(CC(NCC(=O)N4Cc5ccc(F)cc5C4)(C3)C1)C2)c1cnc2ccccc2c1 ; Cl] | 0.039* ± 0.6E−3 | 3E−3* ± 0.2E−3 | 13 |
| NF- 2023- 059 UAMC- 0005 365 | 53 65 | 100 | 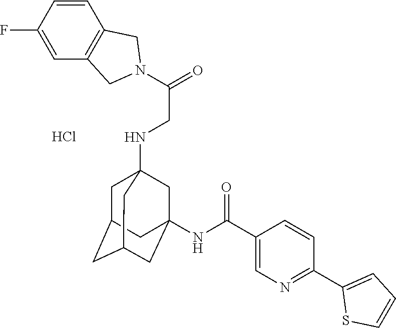 [SMILES: O=C(NC12CC3CC(CC(NCC(=O)N4Cc5ccc(F)cc5C4)(C3)C1)C2)c1ccc(-c2cccs2)nc1 ; Cl] | 0.117* ± 0.01 | 5E−3* ± 0.3E−3 | 22 |
| NF- 2023- 060 UAMC- 0005 366 | 53 66 | 101 | 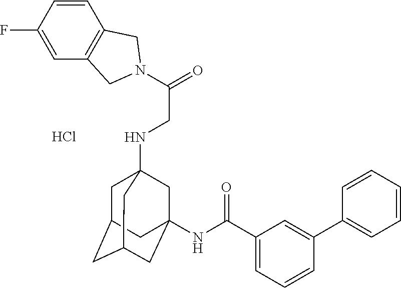 [SMILES: O=C(NC12CC3CC(CC(NCC(=O)N4Cc5ccc(F)cc5C4)(C3)C1)C2)c1cccc(-c2ccccc2)c1 ; Cl] | 0.093* ± 6E−3 | 0.022* ± 4E−3 | 4 |
| UAMC- 0005 444 | 54 44 | 102 | 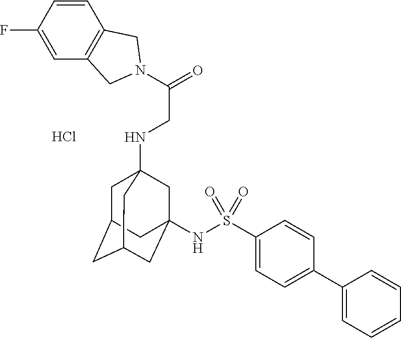 [SMILES: O=C(CNC12CC3CC(C1)CC(NS(=O)(=O)c1ccc(-c4ccccc4)cc1)(C3)C2)N1Cc2ccc(F)cc2C1 ; Cl] | 5.12* ± 0.185 | 1.25* ± 0.1 | 4 |
| MFE- 2022- 192 | 53 77 | 103 | 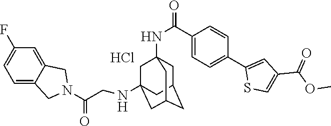 [SMILES: COC(=O)c1csc(-c2ccc(C(=O)NC34CC5CC(CC(NCC(=O)N6Cc7ccc(F)cc7C6)(C5)C3)C4)cc2)c1 ; Cl] | 0.051* ± 5E−3 | 2E−3* ± 0.5E−3 | 25 |
| MFE- 2022- 186 | 53 76 | 104 | 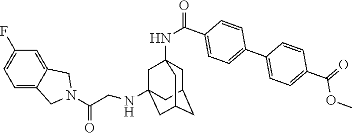 [SMILES: COC(=O)c1ccc(-c2ccc(C(=O)NC34CC5CC(CC(NCC(=O)N6Cc7ccc(F)cc7C6)(C5)C3)C4)cc2)cc1] | 0.048* ± 5E−3 | 1E−3* | 46 |
| MFE- 2022- 203 | 53 78 | 105 | 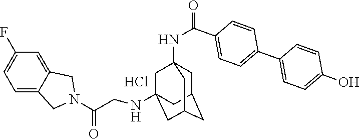 [SMILES: O=C(NC12CC3CC(CC(NCC(=O)N4Cc5ccc(F)cc5C4)(C3)C1)C2)c1ccc(-c2ccc(O)cc2)cc1 ; Cl] | 0.476* ± 0.064 | 0.018* ± 1E−3 | 27 |
| MFE- 2022- 212 | 53 79 | 109 | 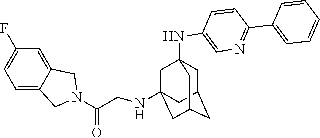 [SMILES: O=C(CNC12CC3CC(C1)CC(Nc1ccc(-c4ccccc4)nc1)(C3)C2)N1Cc2ccc(F)cc2C1] | 0.076* ± 9E−3 | 0.049* ± 6E−3 | 2 |
| MFE- 2022- 119 | 50 30 | 113 | 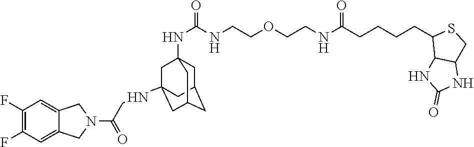 [SMILES: O=C(CCCCC1SCC2NC(=O)NC21)NCCOCCNC(=O)NC12CC3CC(CC(NCC(=O)N4Cc5cc(F)c(F)cc5C4)(C3)C1)C2] | 0.541* ± 0.035 | 0.050* ± 0.019 | 11 |
| MFE- 2022- 122 UAMC- 0005 031 | 50 31 | 114 | 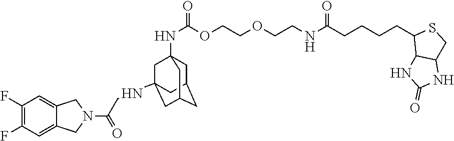 [SMILES: O=C(CCCCC1SCC2NC(=O)NC21)NCCOCCOC(=O)NC12CC3CC(CC(NCC(=O)N4Cc5cc(F)c(F)cc5C4)(C3)C1)C2] | 0.876* ± 0.052 | 0.036* ± 4E−3 | 24 |
| MFE- 2022- 124 UAMC- 0005 032 | 50 32 | 115 | 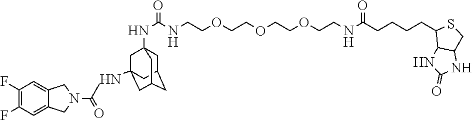 [SMILES: O=C(CCCCC1SCC2NC(=O)NC21)NCCOCCOCCOCCNC(=O)NC12CC3CC(CC(NCC(=O)N4Cc5cc(F)c(F)cc5C4)(C3)C1)C2] | 0.812* ± 0.141 | 0.113* ± 0.024 | 7 |
| MFE- 2022- 080 UAMC- 0005 016 | 50 16 | 116 | 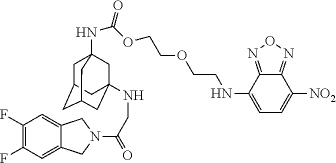 [SMILES: O=C(NC12CC3CC(CC(NCC(=O)N4Cc5cc(F)c(F)cc5C4)(C3)C1)C2)OCCOCCNc1ccc([N+](=O)[O-])c2nonc12] | 0.210* ± 0.079 | 0.015* ± 4E−3 | 14 |
| MFE- 2022- 109 UAMC- 0005 018 | 50 18 | 117 | 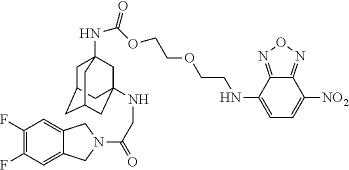 [SMILES: O=C(NC12CC3CC(CC(NCC(=O)N4Cc5cc(F)c(F)cc5C4)(C3)C1)C2)OCCOCCNc1ccc([N+](=O)[O-])c2nonc12] | 0.418* ± 0.047 | 0.036* ± 2E−3 | 12 |
| MFE- 2022- 101 UAMC- 0005 017 | 50 17 | 118 | 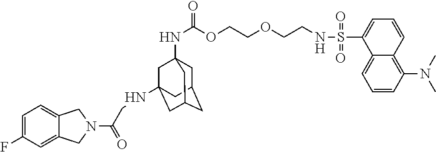 [SMILES: CN(C)c1cccc2c(S(=O)(=O)NCCOCCOC(=O)NC34CC5CC(CC(NCC(=O)N6Cc7ccc(F)cc7C6)(C5)C3)C4)cccc12] | 0.066* ± 0.014 | 3.6E−3 ± 0.3E−3 | 18 |
| MFE- 2022- 114 UAMC- 0005 019 | 50 19 | 119 | 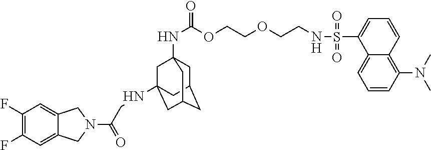 [SMILES: CN(C)c1cccc2c(S(=O)(=O)NCCOCCOC(=O)NC34CC5CC(CC(NCC(=O)N6Cc7cc(F)c(F)cc7C6)(C5)C3)C4)cccc12] | 0.122* ± 7E−3 | 7E−3 ± 2E−3 | 18 |
| MFE- 2022- 126 UAMC- 0005 156 | 51 56 | 120 | 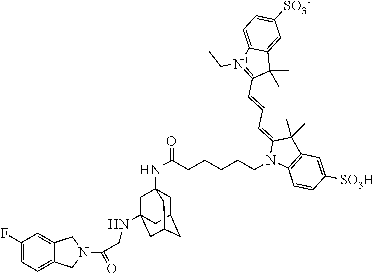 | 0.835* ± 0.046 | 0.040* ± 5E−3 | 21 |
| MFE- 2022- 136 UAMC- 0005 157 PROT ACs | 51 57 | 121 | 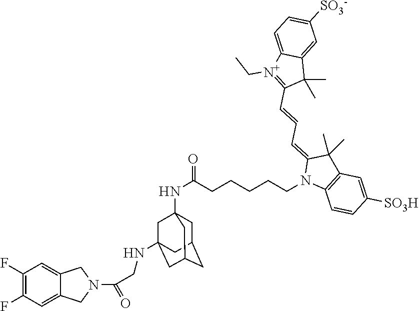 | 1.12 ± 0.059 | 0.054* ± 0.01 | 21 |
| MFE- 2022- 197 UAMC- 0005 381 | 53 81 | 122 | 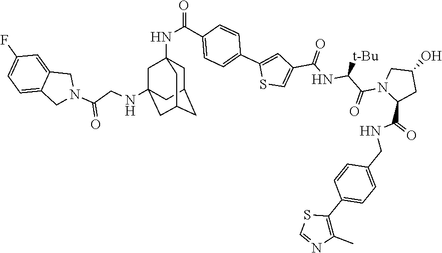 [SMILES: Cc1ncsc1-c1ccc(CNC(=O)[C@@H]2C[C@@H](O)CN2C(=O)[C@@H](NC(=O)c2csc(-c3ccc(C(=O)NC45CC6CC(CC(NCC(=O)N7Cc8ccc(F)cc8C7)(C6)C4)C5)cc3)c2)C(C)(C)C)cc1] | 0.793* ± 0.053 | 0.070* ± 0.011 | 11 |
| MFE- 2022- 189 UAMC- 0005 380 | 53 80 | 123 | 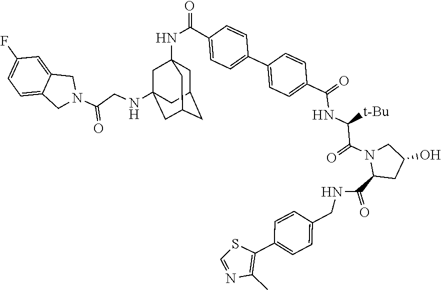 [SMILES: Cc1ncsc1-c1ccc(CNC(=O)[C@@H]2C[C@@H](O)CN2C(=O)[C@@H](NC(=O)c2ccc(-c3ccc(C(=O)NC45CC6CC(CC(NCC(=O)N7Cc8ccc(F)cc8C7)(C6)C4)C5)cc3)cc2)C(C)(C)C)cc1] | 0.443* ± 8E−3 | 0.173* ± 0.022 | 3 |
| MFE- 2022- 147 UAMC- 0005 215 | 52 15 | 124 | 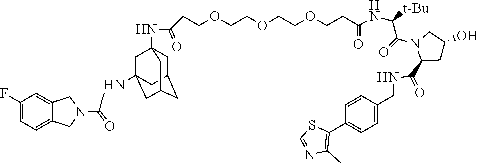 [SMILES: Cc1ncsc1-c1ccc(CNC(=O)[C@@H]2C[C@@H](O)CN2C(=O)[C@@H](NC(=O)CCOCCOCCOCCC(=O)NC23CC4CC(CC(NCC(=O)N5Cc6ccc(F)cc6C5)(C4)C2)C3)C(C)(C)C)cc1] | 0.094 ± 0.025 | 0.020 ± 9E−3 | 5 |
| MFE- 2022- 217 UAMC- 0005 382 | 53 82 | 125 | 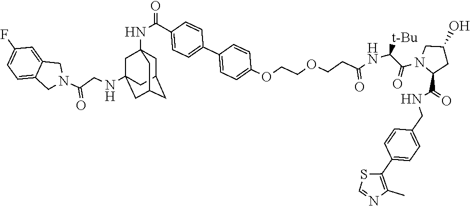 [SMILES: Cc1ncsc1-c1ccc(CNC(=O)[C@@H]2C[C@@H](O)CN2C(=O)[C@@H](NC(=O)CCOCCOc2ccc(-c3ccc(C(=O)NC45CC6CC(CC(NCC(=O)N7Cc8ccc(F)cc8C7)(C6)C4)C5)cc3)cc2)C(C)(C)C)cc1] | 0.127* ± 0.016 | 0.015* ± 1E−3 | 9 |

*Measured with an adapted method because of poor solubility in HEPES buffer pH 7.4: compound was dissolved (at a concentration of 100 mM) in DMSO, then further diluted in DMSO guided by solubility, then diluted in buffer with 10% DMSO, and further dilution series were made in buffer with 1% DMSO. The corresponding DMSO in buffer dilutions were used to determine the 100% enzymatic activity.
**Measured as follows: compound was dissolved (at a concentration of 100 mM) in DMSO, than diluted 10 times in water, and further dilution series were made in water with 1% DMSO. The corresponding DMSO in water dilutions were used to determine the 100% enzymatic activity.

### Example 3: Other Exemplary Isoindoline Varied Analogues (W-Substituents), Glycine Varied Analogues (Y-Substituents) and Adamantyl-Varied Analogues (Z-Substituents) According to Formula I

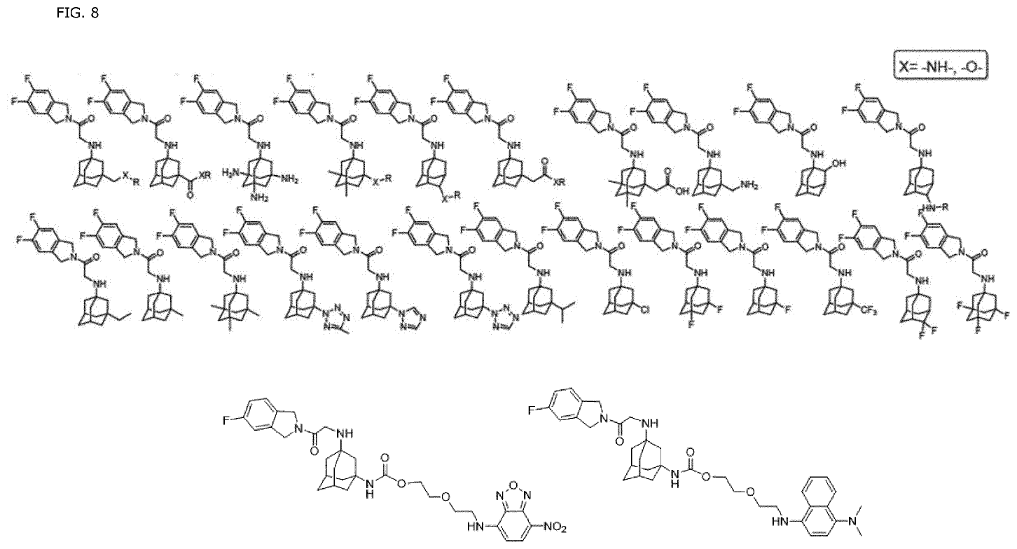

General experimental procedures described herein in example 1 and example 2 are used to synthesized additional compounds of Formula I. The compounds obtained with DPP9 inhibitory effect are shown in FIGS. 6, 7 and 8.

### Example 4: Molecular Dynamics and Molecular Modelling

To rationalize the DPP9/4 selectivity index (SI) of the isoindoline-containing compounds, a computational study was launched. A published co-crystal structure of DPP4 and vildagliptin (PDB-code 6B1E) was relied on, along with the co-crystal structures of DPP9 with 1/1G244 (PDB-code 6EOR) and of DPP8 with a peptide ligand (PDB-code 6EOP, methods provided as Supporting Information). In the latter two structures (DPP8 and DPP9), the co-crystallized ligands were removed, followed by manual docking of vildagliptin's direct isoindoline analogue 5p, to obtain a binding pose similar to the parent compound in DPP4. FIG. 3 shows the superposition of the three enzyme structures with either co-crystallized vildagliptin (DPP4) or the manually docked 5p (DPP8 and 9). As described earlier by Huber et al., the structure and dimensions of the catalytic sites of the three enzymes are almost identical. In addition, their respective S1-pocket residues are highly conserved. Not surprisingly, both vildagliptin and 5p can therefore effectively be docked/accommodated in all three enzyme models. This finding thus fails to explain the experimental DPP8/9-selectivity of 5p and other isoindoline-containing compounds.

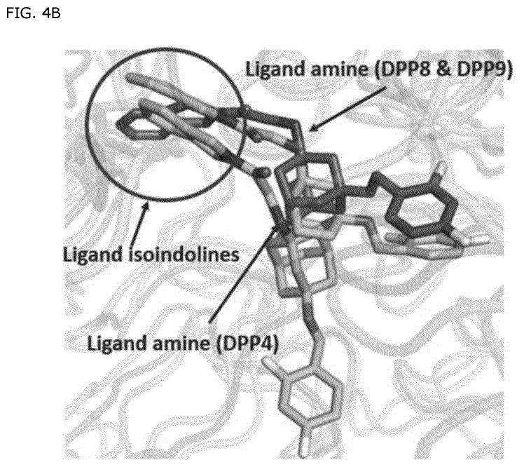

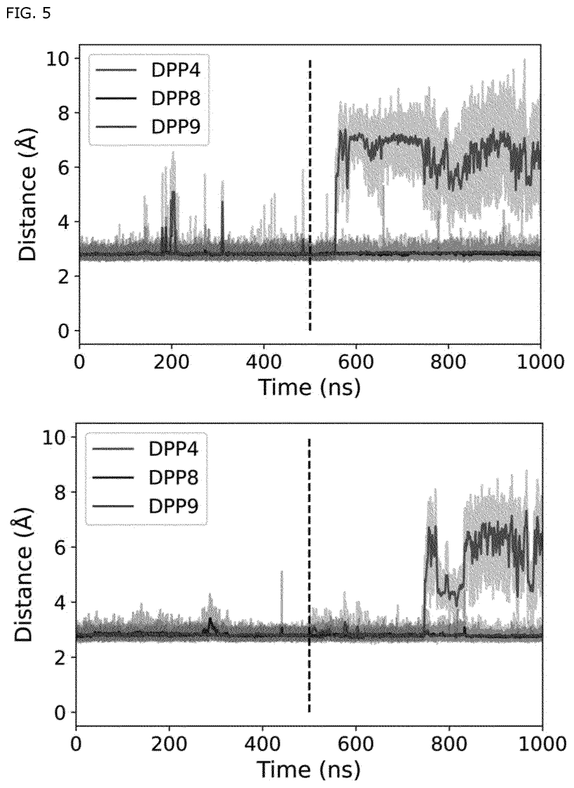

Intrigued by the lack of hypothesis when using static crystal structures, we decided to launch a Molecular Dynamics (MD) study of the monomers of DPP4, DPP8 and DPP9 in complex with 5r, the most potent DPP9 inhibitor in the series. For each of the three proteins, the protein-ligand complex was simulated for 1 μs, divided over two MD simulations of 500 ns. FIG. 4A and FIG. 4B illustrate that during the second part of the simulation of the DPP4 protein-ligand complex, the isoindoline of target compound 5r tilts and translates out of the S1 pocket, leading to a ligand conformation that strongly differs from the binding poses of co-crystallized vildagliptin. The observed movement of 5r's isoindoline ring in DPP4 did not stem from contraction or dynamic behaviour of the S1 pocket (discussed further in the Supporting Information file). Noteworthy, ligand translation in DPP4 also affects the position of the secondary, protonated amine of 5r. Moreover, the latter's electrostatic stabilisation with glutamate residues E205 and E206 is also abolished during the process. This is a potentially relevant observation because this interaction is generally considered essential for ligand recognition in DPPs. More specifically, E205 will subsequently engage in hydrogen bonding with H126 and S209 (also discussed in more detail in the Supporting Information). The region of H126 (DPP4) is not conserved between DPP4 on the one hand and DPP8 and DPP9 on the other hand, possibly explaining why this state is not observed in the DPP8-5r and DPP9-5r simulations. Furthermore, the disruption of the interaction between E206 of DPP4 and the secondary amine coincides with the change in the binding pose of the ligand shown in FIG. 4B suggesting a correlation between these two events. Of note, the positioning of the isoindoline in the S1 pocket of the DPP8-5r complex also changes. However, as illustrated in FIG. 4B this positional change remains limited to a tilt without a translation of the ligand and also the electrostatic stabilization of 5r's protonated amine is maintained throughout the simulation.

Although we currently do not have a clear explanation for the higher in silico flexibility and mobility of 5r in DPP4's active site, we do hypothesize that comparable compound behavior could be involved in the lower DPP4-affinity that is typically observed experimentally for isonindoline-containing inhibitors.

The present invention is in no way limited to the embodiments described in the examples and/or shown in the figures. On the contrary, methods according to the present invention may be realized in many different ways without departing from the scope of the invention.

1. A compound able to bind to human DPP9, wherein said compound is according to Formula I 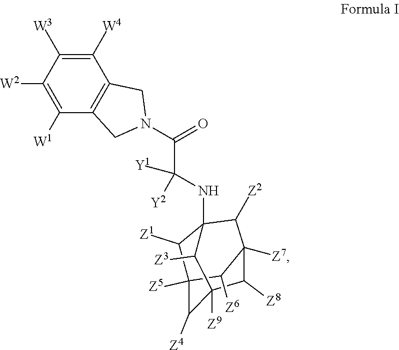
or a pharmaceutically acceptable salt thereof,
wherein
W¹, W², W³ and W⁴ are independently selected from the group consisting of hydrogen, alkyl, alkylcarbonyl, alkylsulfonyl, cycloalkyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, halocycloalkyl, heterocycle, heterocyclealkyl, heterocycleoxyalkyl and alkoxy;
Y¹, Y² are independently selected from the group consisting of hydrogen, alkyl, alkylcarbonyl, alkylsulfonyl, cycloalkyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, halocycloalkyl, heterocycle, heterocyclealkyl, heterocycleoxyalkyl, and alkoxy;
Z¹, Z², Z³, Z⁴, Z⁵, Z⁶, Z⁷, and Z⁸ are each independently selected from the group consisting of hydrogen, halogen, dihalogen, alkyl, haloalkyl, carboxyalkyl, alkylcarboxyalkyl, alkyloxyalkyl, cycloalkyl, carboxycycloalkyl, aryl, arylalkyl, aryloxyalkyl, aryloxy, hydroxy, alkoxy, alkylamine, alkylamide, N-alkylalkylamide, cycloalkyloxy, heterocycleoxy, heterocycle, heterocyclealkyl, and heterocycleoxyalkyl, —NHR, —NHC(═O)R, —NHC(═O)OR, —NHC(═O)NHR, —OR, —O(C═O)NHR; —R—CN, —R-halo, and —O—(C═O)-L-prolinate;
R is independently selected from the group consisting of hydrogen, alkyl, alkylcarbonyl, alkylsulfonyl, alkylcarboxyalkyl, cycloalkyl, (alkyl)(carboxyalkyl)sulfide, alkylamide-benzyl carbamate, cycloalkylcarbonyl, alkyl-OH, alkyl-Ar¹, alkylcarboxyalkyl-Ar¹, alkyl-Ar¹—Ar², Ar¹, Ar¹-carbonyl, Ar¹-sulfonyl, alkyl-Ar¹, alkoxy-Ar¹, carboxyalkyl, carboxycycloalkyl, carboxyalkylbenzylcarbamate, carboxyalkyl-Ar¹, hydroxycycloalkyl, cycloalkylsulfonyl, halogen, haloalkyl, halocycloalkyl, heterocycle, heterocyclecarbonyl, heterocyclesulfonyl, heterocyclealkyl, heterocycleoxyalkyl and —O-alkyl-CH₂-cycloalkyl, —(CH₂)ₚ—Ar¹, (CH₂)ₚ—CH—(Ar¹)(Ar²), (CH₂)ₚ—heterocycle, and -(alkyl-O)ₙ—(CH₂)₂—X, wherein n is independently chosen from 0 to 5, wherein p is independently chosen from 0 to 3, wherein
each Ar¹, Ar² is independently chosen from an aromatic ring optionally comprising 1 or 2 heteroatoms selected from O, N and S, adamantyl, indole or isoindole; each Ar¹, Ar² being optionally substituted with 0 to 3 substituents selected from halogen, nitrile or phenyl.

2. The compound according to claim 1 wherein, W¹, W², W³ and W⁴ are independently selected from hydrogen, halogen, or —O—C₁₋₆ alkyl.

3. The compound according to claim 1, wherein Z¹, Z², Z³, Z⁴, Z⁵, Z⁶, Z⁷, Z⁸ and Z⁹ are independently selected from hydrogen, halogen, —NHR, or C₁₋₆ alkyl.

4. The compound according to claim 1, wherein Z¹, Z², Z³, Z⁴, Z⁵, Z⁶, Z⁷, Z⁸ and Z⁹ are independently selected from hydrogen, —NHR, —NHC(═O)R, —NHC(═O)OR, —NHC(═O)NHR, —OR, —O(C═O)NHR or —O—(C═O)-L-prolinate.

5. The compound according to claim 1, wherein R is selected from hydrogen, C₁₋₆ alkyl, C₁₋₆ hydroxyalkyl, C₅₋₇ cycloalkyl, C₅0.7 hydroxycycloalkyl or Ar¹.

6. The compound according to claim 1, wherein R is selected from: carboxyalkyl, alkylcarboxyalkyl, (alkyl)(alkylcarboxyalkyl)sulfide, alkylamide-benzyl carbamate, or carboxyalkyl-Ar¹.

7. The compound according to claim 1,
wherein,
W¹, W², W³ and W⁴ are independently selected from hydrogen, halogen, or —O—C₁₋₆ alkyl;
Z¹, Z², Z³, Z⁴, Z⁵, Z⁶, Z⁸ and Z⁹ are independently selected from hydrogen, halogen, haloalkyl, NH₂ or —C₁₋₆ alkyl; and
Z⁷ is selected from —C₁₋₆ alkyl, —NHR, —NHC(═O)R, —NHC(═O)OR, —NHC(═O)NHR, —O(C═O)NHR, or —O—(C═O)-L-prolinate;
wherein
R is independently selected from hydrogen, —C₁₋₆ alkyl, —CH—(Ar¹)—(Ar²), CH₃₋ₙ— (Ar¹)ₙ, or adamantyl,
wherein
n is 1 or 2,
wherein
—C₁₋₆ alkyl is optionally substituted with hydroxy, halogen or —Ar¹,
wherein
each Ar¹, Ar² is independently chosen from an aromatic ring optionally comprising 1 or 2 heteroatoms selected from O, N and S, adamantyl, indole or isoindole; each Ar¹, Ar² being optionally substituted with from 0 to 3 substituents selected from halogen, nitrile or phenyl.

8. The compound according to claim 1,
wherein
W¹, W², W³ and W⁴ are independently selected from hydrogen, halogen and —O—C₁₋₆ alkyl;
Z¹, Z², Z³, Z⁴, Z⁵, Z⁶, Z⁸ and Z⁹ are —H; and
Z⁷ is —O(C═O)NHR,
wherein
R is —CH—(Ar¹)₂, carboxyalkylbenzylcarbamate, carboxyalkyl, (alkyl)(carboxyalkyl)sulfide, alkylamide-benzyl carbamate, carboxyalkyl-Ar¹, alkylcarboxyalkyl, alkylcarboxylalkylAr¹.

9. The compound according to claim 1,
wherein
W¹, W², W³ and W⁴ are independently selected from the group comprising hydrogen, halogen, and O—C₁₋₆ alkyl;
Z¹, Z², Z³, Z⁴, Z⁵, Z⁶, Z⁸ and Z⁹ are independently selected from hydrogen or —C₁₋₆ alkyl; and
Z⁷ is selected from —NHC(═O)OR or —NHC(═O)NHR, wherein
R is -(alkyl-O)ₙ—(CH₂)₂—X, and
n is 0 to 6.

10. The compound according to claim 1, wherein X is independently selected from:

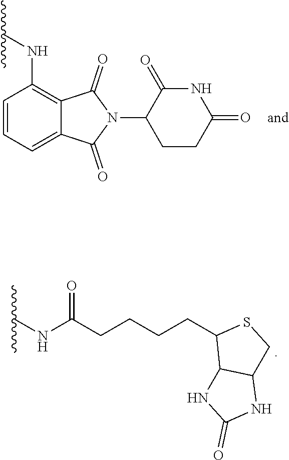 [SMILES: *Nc1cccc2c1C(=O)N(C1CCC(=O)NC1=O)C2=O ; *NC(=O)CCCCC1SCC2NC(=O)NC21]

11. The compound according to claim 1, wherein said compound is a DPP9 inhibitor.

12. The compound according to claim 1, wherein said X moiety is selected from an E3 ligase ligand moiety or from a molecular reporter moiety.

13. The compound according to claim 12, wherein X moiety of said compound engages with an E3 ubiquitin ligase.

14. The compound according to claim 12, wherein X moiety of said compound comprises a reporter moiety.

15. A pharmaceutical composition comprising a compound according to claim 1 or a pharmaceutically acceptable salt thereof and optionally at least one pharmaceutically acceptable carrier, diluent, excipient, or adjuvant.

16. (canceled)

17. A method for the prevention and/or treatment of a disorder, said method comprises administering to a subject in need thereof a compound or a pharmaceutical composition according to claim 1.

18. The method according to claim 17, wherein the disorder is a DPP9 enzyme-related disorder.
